(19) United States
(12) Patent Application Publication
(10) Pub. No.: US 2026/0356842 A1
(43) Pub. Date: Nov. 12, 2026

(54) COMPOSITIONS AND METHODS OF USING THE SAME FOR TREATMENT OF NEURODEGENERATIVE AND MITOCHONDRIAL DISEASE

(71) Applicant: MITOKININ, INC., North Chicago, IL (US)

(72) Inventors: Nicholas Thomas HERTZ, San Francisco, CA (US); Dara DITSWORTH, Pacifica, CA (US); Johan BARTHOLOMEUS, Montreal (CA); Shawn JOHNSTONE, Saint-Lazare (CA); Randall Marcelo CHIN, Union City, CA (US); Robert DEVITA, Westfield, NY (US); Philippe MCGEE, Lachine (CA); Julien DANSEREAU, Saint-Zotique (CA); Rishi RAKHIT, Berkeley, CA (US)

(21) Appl. No.: 19/734,252

(22) Filed: Jul. 8, 2026

Related U.S. Application Data
(63) Continuation of application No. 17/801,450, filed on Feb. 22, 2021, now abandoned.
(60) Provisional application No. 62/980,143, filed on Feb. 21, 2020.

(51) Int. Cl. C07D 487/04; C07D 519/00; G01N 33/50

(52) U.S. Cl. CPC C07D 487/04; C07D 519/00; G01N 33/5044; G01N 33/5079

(57) ABSTRACT

The present disclosure is directed to adenine analogs, methods of making adenine analogs, and methods of treating disorders associated with PINK1 kinase activity including, but not limited to, neurodegenerative diseases, mitochondrial diseases, fibrosis, and/or cardiomyopathy using these analogs. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 62/980,143, filed on Feb. 21, 2020, the contents of which are hereby incorporated by reference in their entirety.

### REFERENCE TO SEQUENCE LISTING

The Sequence Listing submitted Feb. 22, 2021 as a text file named “37930_0006P1_ST25.txt,” created on Feb. 22, 2021, and having a size of 20,293 bytes is hereby incorporated by reference pursuant to 37 C.F.R. § 1.52(e)(5).

### BACKGROUND

Maintenance of mitochondrial function is essential for the health and survival of numerous cell types, including cardiomyoctes, hepatocytes, renal cells and neurons. Aberrant mitochondrial quality control has been demonstrated to be an important factor in the development of neurodegenerative diseases, kidney disease, and cardiomyopathy (Schapira, A.H. Mitochondrial disease. Lancet 379, 1825-1834, (2012) and Chen, Y. and Dom, G, PINK1-Phosphorylated Mitofusin-2 Is a Parkin Receptor for Culling Damaged Mitochondria. Science 340, 471-475, (2013)). The mitochondrial kinase PTEN Induced Kinase 1 (PINK1) plays an important role in the mitochondrial quality control processes by responding to damage at the level of individual mitochondria. The PINK1 pathway has also been linked to the induction of mitochondrial biogenesis and, critically, to the reduction of mitochondrially-induced apoptosis. See e.g., Narendra, D. P. et al. PINK1 is selectively stabilized on impaired mitochondria to activate Parkin. PLoS Biol 8, e1000298 (2010), Wang, X., (2011). et al. PINK1 and Parkin target Miro for phosphorylation and degradation to arrest mitochondrial motility. Cell 147, 893-906, (2011), and Shin, J. H. et al. PARIS (ZNF746) repression of PGC-1alpha contributes to neurodegeneration in Parkinson's disease. Cell 144, 689-702, (2011).

Parkinson's Disease (PD) is one of the most common neurodegenerative disorders; however, no disease modifying therapies are currently approved to treat PD. Both environmental and genetic factors lead to progressive apoptosis of dopaminergic neurons, lowered dopamine levels, and, ultimately, PD. PINK1 kinase activity appears essential to mediate its neuroprotective activity. The regulation of mitochondrial movement, distribution, and clearance is a key part of neuronal oxidative stress response. Disruptions to these regulatory pathways have been shown to contribute to chronic neurodegenerative disease. See Schapira and Chen cited above.

Cardiomyopathy refers to a disease of cardiac muscle tissue, and it is estimated that cardiomyopathy accounts for 5-10% of the 5-6 million patients already diagnosed with heart failure in the United States. Based on etiology and pathophysiology, the World Health Organization created a classification of cardiomyopathy types which includes dilated cardiomyopathy, hypertrophic cardiomyopathy, restrictive cardiomyopathy, arrhythmogenic right ventricular cardiomyopathy, and unclassified cardiomyopathy. See e.g., Richardson P, et al. Report of the 1995 World Health Organization/International Society and Federation of Cardiology Task Force on the Definition and Classification of cardiomyopathies. Circulation 1996; 93:841. PINK kinase activity appears to mediate its cardio-protective activity. The regulation of mitochondrial movement, distribution, and clearance is a part of cardiac cell oxidative stress response. Disruptions to these regulatory pathways have been shown to contribute to cardiomyopathy. See Schapira and Chen cited above. Wang, X., (2011) et al. PINK1 and Parkin target Miro for phosphorylation and degradation to arrest mitochondrial motility Cell 147, 893-906, (2011) and Richardson P, et al. Report of the 1995 World Health Organization/International Society and Federation of Cardiology Task Force on the Definition and Classification of cardiomyopathies. Circulation 1996; 93:841. Several cases of adult-onset LS have also been reported recently. See e.g., Longo, D, et al. Harrison's Internal Medicine. 18^{th} ed. (online), Ch. 238 (2011), Koh, H. & Chung, I PINK1 as a molecular checkpoint in the maintenance of mitochondrial function and integrity, Mol Cells 34, 7-13, (2012), Martins-Branco, D. et al. Ubiquitin proteasome system in Parkinson's disease: a keeper or a witness? Exp Neurol 238, 89-99, (2012), and Geisler, S. et al. The PINK1/Parkin-mediated mitophagy is compromised by PD-associated mutations. Autophagy 6, 871-878, (2010).

In vivo imaging techniques such as MRI reveal bilateral hyperintense lesions in the basal ganglia, thalamus, substantia nigra, brainstem, cerebellar white matter and cortex, cerebral white matter, or spinal cord of LS patients. See e.g., Longo cited above and Shin, J. H. et al. PARIS (ZNF746) repression of PGC-1alpha contributes to neurodegeneration in Parkinson's disease. Cell 144, 689-702, (2011), Henchcliffe, C. & Beal, M. F. Mitochondrial biology and oxidative stress in Parkinson disease pathogenesis. Nat Clin Pract Neurol 4, 600-609 (2008), Pridgeon, J. W., Olzmann, J. A, Chin, L. S. & Li, L. PINK1 Protects against Oxidative Stress by Phosphorylating Mitochondrial Chaperone TRAP1. PLoS Biol 5, e172 (2007), and Haque, M. E. et al. Cytoplasmic Pink1 activity protects neurons from dopaminergic neurotoxin MPTP. Proc Natl Acad Sci USA 105, 1716-1721 (2008). The lesions usually correlate with gliosis, demyelination, capillary proliferation, and/or necrosis See Geisler, S. et al. The PINK1/Parkin-mediated mitophagy is compromised by PD-associated mutations. Autophagy 6, 871-878, (2010) and Gautier, C. A., Kitada, T. & Shen, J. Loss of PINK1 causes mitochondrial functional defects and increased sensitivity to oxidative stress. Proc Natl Acad Sci USA 105, 11364-11369 (2008). Behavioral symptoms of LS patients can include (with a wide variety of clinical presentation) developmental retardation, hypotonia, ataxia, spasticity, dystonia, weakness, optic atrophy, defects in eye or eyelid movement, hearing impairment, breathing abnormalities, dysartbria, swallowing difficulties, failure to thrive, and gastrointestinal problems. See e.g., Wang and Richardson cited above, and Samaranch, L. et al. PINK1-linked parkinsonism is associated with Lewy body pathology. Brain 133, 1128-1142, (2010) and Merrick, K. A. et al. Switching Cdk2 on or off with small molecules to reveal requirements in human cell proliferation. Mol Cell 42, 624-636, (2011). The cause of death in most LS cases is unclear, and the lack of a genetic model to study the disease progression and cause of death has impeded the development of adequate treatment. Prognosis for LS (and most diseases resulting from mitochondrial dysfunction) is very poor; there is no cure and treatment is often ineffective.

Parkinson's Disease (PD) is one of the most common neurodegenerative disorders; however, no disease modifying therapies are currently approved to treat PD. Both environmental and genetic factors lead to progressive apoptosis of dopaminergic neurons, lowered dopamine levels, and, ultimately, PD. PINK1 kinase activity appears to mediate its neuroprotective activity. The regulation of mitochondrial movement, distribution, and clearance is a key part of neuronal oxidative stress response. Disruptions to these regulatory pathways have been shown to contribute to chronic neurodegenerative disease. See Schapira and Chen cited above.

Despite the widespread prevalence of disorders associated with PINK1 pathway, compounds capable of selectively targeting this pathway and, thus, treating disorders associated with this pathway have remained elusive. Accordingly, there remains a need for compounds and compositions capable of modulating PINK1 kinase activity and methods of making and using same.

### SUMMARY

In accordance with the purpose(s) of the invention, as embodied and broadly described herein, the invention, in some embodiments, relates to adenine compounds useful in the treatment of disorders associated with PINK1 kinase activity such as, for example, a neurodegenerative disease, a mitochondrial disease, fibrosis, and/or cardiomyopathy.

Thus, provided herein are compounds having a structure represented by a formula:

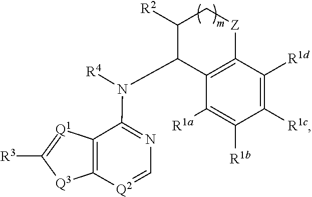

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4haloalkoxy, C1-C4 alkoxy, C1-C4 alkylamino, and (C1-C4)(C1-C4) dialkylamino; wherein R² is selected from —(CH₂)ⁿCy¹, —O(CH₂)ₙCy¹, —NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof.

Also described are compounds having a structure represented by a formula:

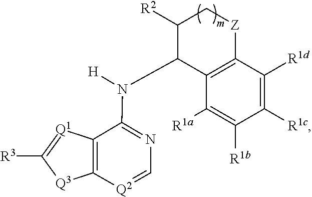

wherein m is 0 or 1; wherein each of Q¹ and Q² is independently N or CH; wherein Q³ is CH₂ or NH; wherein Z is CR^{11a}R^{11b}, R¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, —NR¹³(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R³ is a 3- to 6-membered cycloalkyl, a C1-C6 haloalkyl, C1-C6 haloalkoxy, or C1-C6 halohydroxyalkyl, or a pharmaceutically acceptable salt thereof.

Without wishing to be bound by theory, an advantage of the presently described compounds is that they possess improved potency and reduced toxicity. For example, the disclosed compounds can exhibit an EC₅₀ of less than 0.3 μM with a toxicity of less than 10%. See, e.g., Tables 2A and 2B and FIG. 1A-F, compound no. EP-0038098.

Also provided are methods for making a disclosed compound.

Also provided are pharmaceutical compositions comprising a therapeutically effective amount of a disclosed compound and a pharmaceutically acceptable carrier.

Also provided are methods of modulating PINK1 kinase activity in a subject in need thereof, the method comprising administering to the subject in need thereof an effective amount of at least one disclosed compound.

Also disclosed are methods of modulating PINK1 kinase activity in at least one cell, the method comprising contacting the cell with an effective amount of at least one disclosed compound.

Also provided are methods for treating a disorder in a subject in need thereof, the method comprising administering to the subject in need thereof an effective amount of at least one disclosed compound, wherein the disorder is a neurodegenerative disorder, a mitochondrial disorder, a fibrosis, or cardiomyopathy.

Also provided are kits comprising a disclosed compound and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several embodiments and together with the description serve to explain the principles of the invention.

FIG. 1A-F show representative data demonstrating the potency and toxicity of the compounds nos. EP-0035985, EP-0037821, EP-0038098, and EP-0038099 in the presence of 1 μM FCCP/oligomycin or with no toxin (no FO) for 6 hrs. H₂O₂ treatment was performed as a control for cell death as measured by DAPI staining,

FIG. 2A-F show representative data demonstrating the potency and toxicity of the compounds nos. EP-0035985, EP-0038461, EP-0038463, and EP-0038503 in the presence of 1 μM FCCP/oligomycin or with no toxin (no FO) for 6.5 hrs. H₂O₂ treatment was performed as a control for cell death as measured by DAPI staining.

FIG. 3A-F show representative data demonstrating the potency and toxicity of the compounds nos. EP-0035985, EP-0038504, EP-0038508, and EP-0038521 in the presence of 1 μM FCCP/oligomycin or with no toxin (no FO) for 6.5 hrs. H₂O₂ treatment was performed as a control for cell death as measured by DAPI staining.

FIG. 4A and FIG. 4B show representative data demonstrating the potency and toxicity of the compounds nos. EP-0035985, EP-0038461, EP-0038463, EP-0038503, EP-0038504, EP-0038508, and EP-0038521 in the presence of 1 μM FCCP/oligomycin or with no toxin (no FO) for 6.5 hrs. H₂O₂ treatment was performed as a control for cell death as measured by DA staining.

FIG. 5A-C show representative data demonstrating that compounds nos. EP-0038504 and EP-0038461 exhibit low mitotoxicity.

FIG. 6A-C show representative data demonstrating that compounds nos. EP-0038508 and EP-0038463 exhibit low mitotoxicity.

FIG. 7A-C show representative data demonstrating that compounds nos. EP-0038503 and EP-00338521 exhibit low mitotoxicity.

FIG. 8A-C show representative data demonstrating that compounds nos. EP-0035985 and EP-0038098 can reduce pathological α-synuclein levels in primary neurons. (NOTE: MTK458=35985; MWK898=38098).

FIG. 9A and FIG. 9B show representative in vivo data of EP-0040180 in an α-syn (PFF) model. Specifically, FIG. 9A shows that oral BID dosing of EP-0040180 at 50, 25, 12.5, and 6.25 mg/kg significantly reduces pathologic (pS129) α-syn (250-12) (FIG. 9A) and pathologic (pS129) α-syn (monomer) (FIG. 9B) from the striatum induced by PFF challenge.

FIG. 10A-C show representative data illustrating that increasing doses of EP-0040503 reduces pS129 α-syn (FIG. 10A), pS129 α-syn (250-12) (FIG. 10B), and pS129 α-syn (monomer) (FIG. 10C) in primary neuron culture.

FIG. 11A-C show representative data illustrating that increasing doses of EP-0040850 reduces pS129 α-syn (FIG. 11A), pS129 α-syn (250-12) (FIG. 11B), and pS129 α-syn (monomer) (FIG. 11C) in primary neuron culture.

FIG. 12 shows representative data illustrating that treatment with toxins cause an increase in cleaved caspase-3 levels while treatment with EP-0040850 does not.

FIG. 13A-C show representative data illustrating that increasing doses of EP-0040857 reduces pS129 α-syn (FIG. 13A), pS129 α-syn (250-12) (FIG. 13B), and pS129 α-syn (monomer) (FIG. 13C) in primary neuron culture.

FIG. 14A-C show representative data illustrating that increasing doses of EP-0040270 reduces pS129 α-syn (FIG. 14A), pS129 α-syn (250-12) (FIG. 14B), and pS129 α-syn (monomer) (FIG. 14C) in primary neuron culture.

FIG. 15 shows representative data illustrating that treatment with toxins and high dose of EP-0040270 increase cleaved caspase-3 levels.

FIG. 16A-C show representative data illustrating that increasing doses of EP-0040587 reduces pS129 α-syn (FIG. 16A), pS129 α-syn (250-12) (FIG. 16B), and pS129 α-syn (monomer) (FIG. 16C) in primary neuron culture.

FIG. 17A-C show representative data illustrating that increasing doses of EP-0040180 reduces pS129 α-syn (FIG. 17A), pS129 α-syn (250-12) (FIG. 17B), and pS129 α-syn (monomer) (FIG. 17C) in primary neuron culture.

FIG. 18 shows representative data illustrating that treatment with toxins and EP-0040180 do not significantly alter the levels of cleaved caspase-3.

FIG. 19A and FIG. 19B show representative data illustrating EP-0040180 effects on pS129 signals.

FIG. 20A-C show representative data illustrating that increasing doses of EP-0041161 reduces pS129 α-syn (FIG. 20A), pS129 α-syn (250-12) (FIG. 20B), and pS129 α-syn (monomer) (FIG. 20C) in primary neuron culture.

FIG. 21 shows representative data illustrating that treatment with toxins alter the levels of cleaved caspase-3, but treatment with EP-0041161 does not.

FIG. 22A and FIG. 22B show representative data illustrating EP-0041161 effects on pS129 signals.

FIG. 23A-C show representative data illustrating that increasing doses of EP-0041088 reduces pS129 α-syn (FIG. 23A), pS129 α-syn (250-12) (FIG. 23B), and pS129 α-syn (monomer) (FIG. 23C) in primary neuron culture.

FIG. 24A and FIG. 24B show representative data illustrating EP-0041088 effects on pS129 signals.

FIG. 25A-C show representative data illustrating that increasing doses of EP-0040874 reduces pS129 α-syn (FIG. 25A), pS129 α-syn (250-12) (FIG. 25B), and pS129 α-syn (monomer) (FIG. 25C) in primary neuron culture.

FIG. 26A-C show representative data illustrating that increasing doses of EP-0041668 reduces pS129 α-syn (FIG. 26A), pS129 α-syn (250-12) (FIG. 26B), and pS129 α-syn (monomer) (FIG. 26C) in primary neuron culture.

FIG. 27A-C show representative data illustrating that increasing doses of EP-0041670 reduces pS129 α-syn (FIG. 27A), pS129 α-syn (250-12) (FIG. 27B), and pS129 α-syn (monomer) (FIG. 27C) in primary neuron culture.

FIG. 28 shows representative data demonstrating that PINK1 activators 35985 and 40180 induce mitophagy in a dose dependent manner. ABC123

FIG. 29 shows representative data demonstrating that PINK1 activators 35985 and 40180 accelerate recruitment of Parkin to mitochondria.

FIG. 30A-C show representative data demonstrating that cisplatin induces PINK1 and its direct target pUb. Specifically, FIG. 30A and FIG. 30B shows that cistplatin causes mitochondrial damage in vivo as demonstrated by pS65-Ub increase (FIG. 30A) and induction of PINK1 (FIG. 30B). FIG. 30C shows the correlation between pUb and PINK1.

FIG. 31 shows representative data demonstrating that cisplatin induces reduction in mtDNA/nucDNA ratio.

FIG. 32A and FIG. 32B show representative data demonstrating that cistplatin-induced kidney damage is increased in PINK1 KO mice.

FIG. 33 shows representative data demonstrating that cisplatin does not induce a change in pS65 Ubiquitin in PINK1 KO mice.

FIG. 34 shows representative data demonstrating that cisplatin challenge increases mitochondrial-stress gene expression in PINK1 KO mice.

FIG. 35 shows representative data demonstrating a comparison of mouse plasma pharmacokinetics of 35985 and 40180.

FIG. 36 shows representative data demonstrating that 40180 reduces KIM-1 in cisplatin-challenged mouse.

FIG. 37 show representative data demonstrating that 40180 reduces expression of mitochondrial-stress related genes.

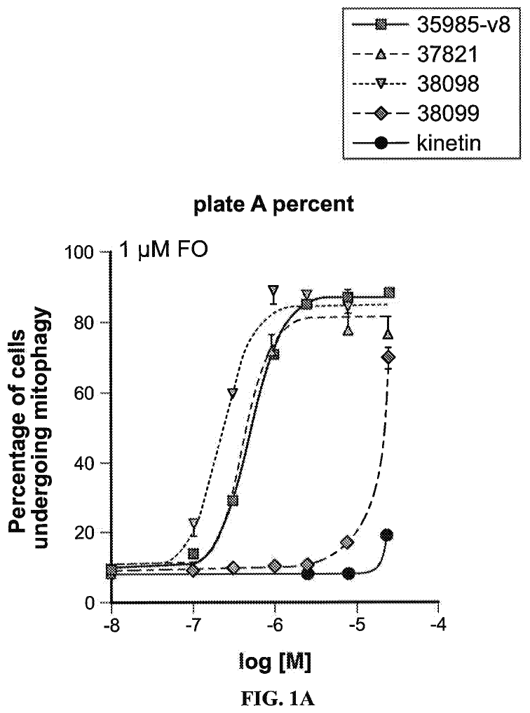

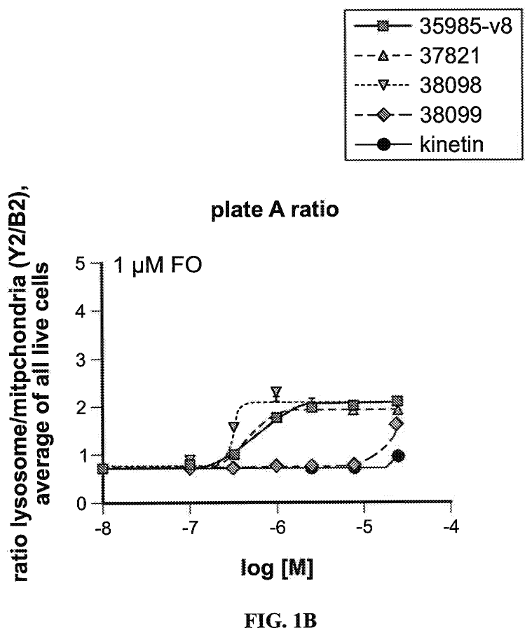

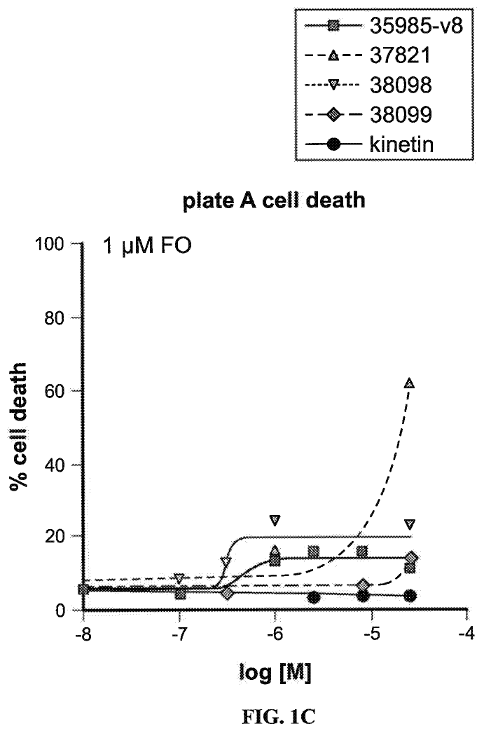

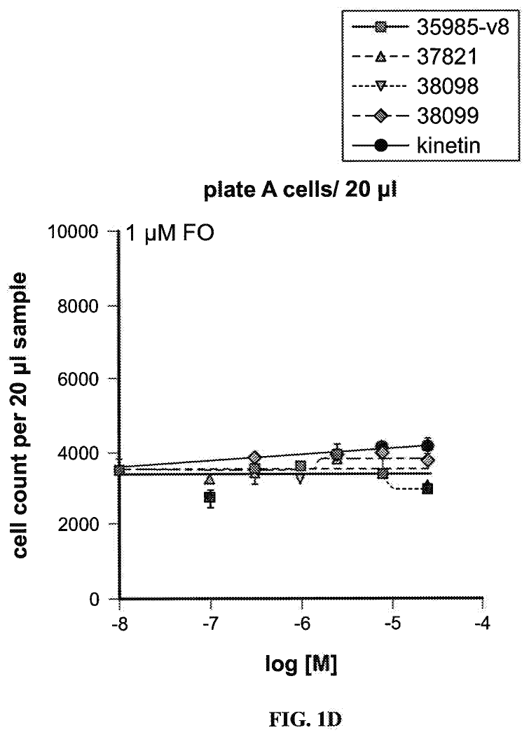

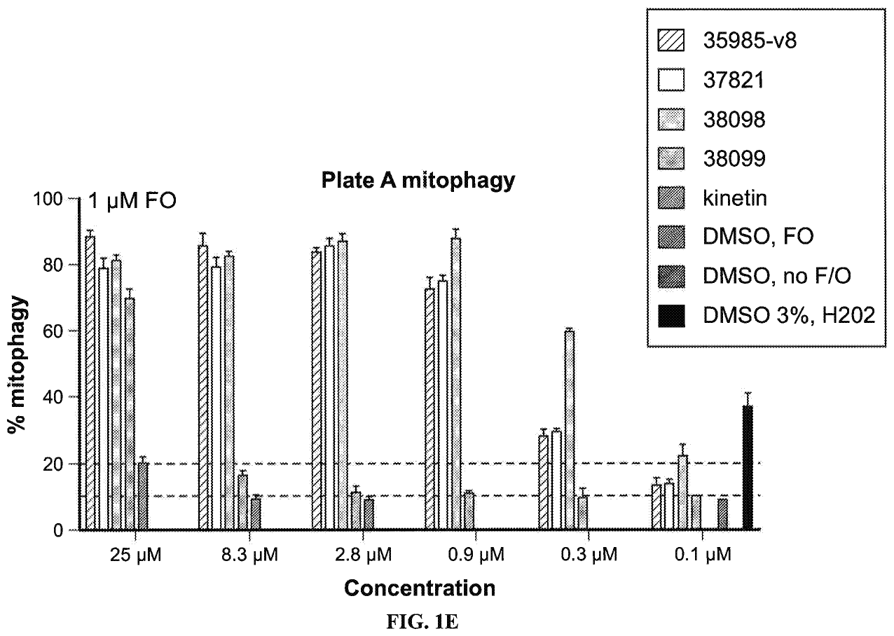

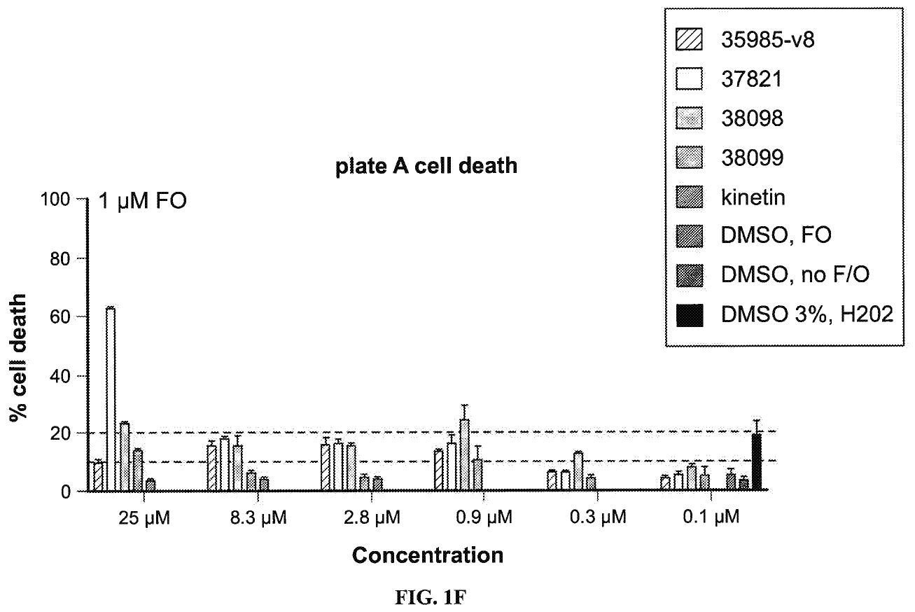

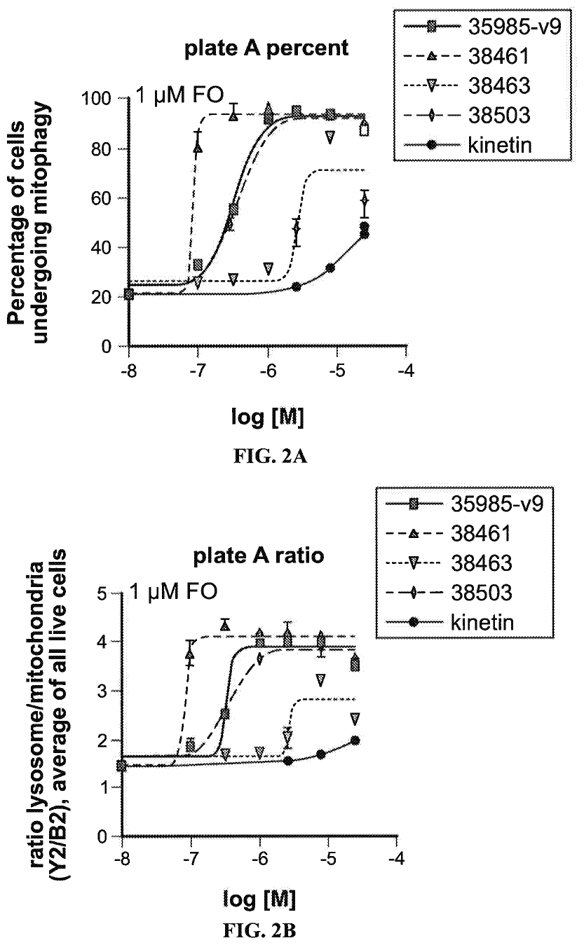

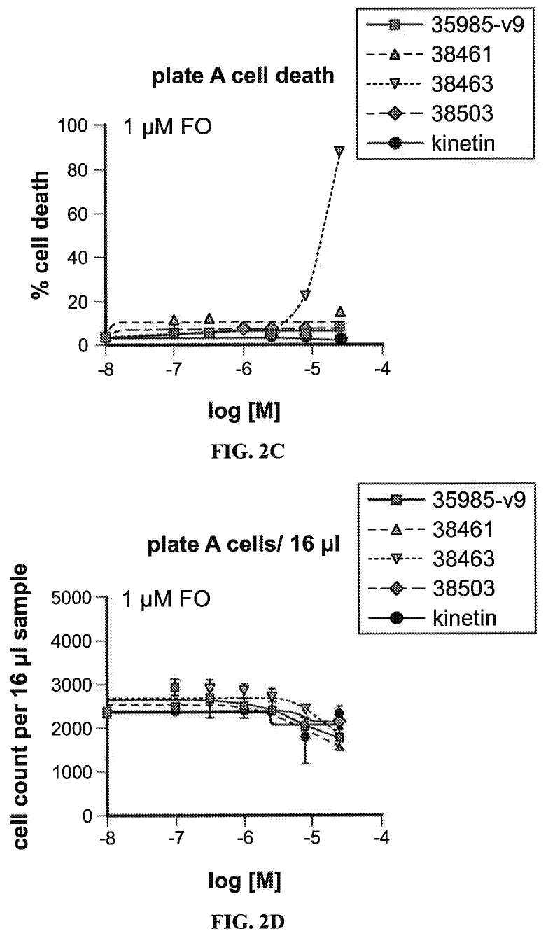

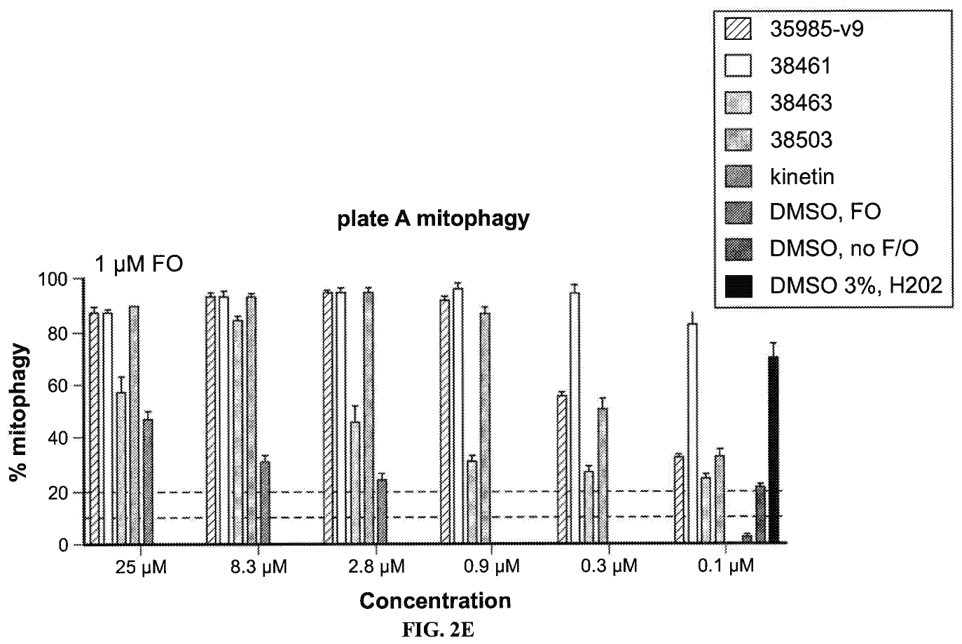

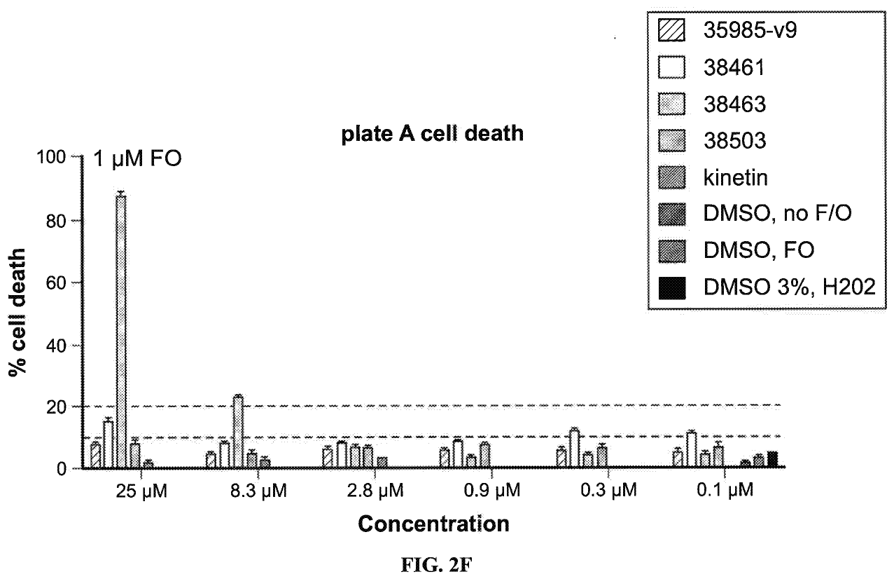

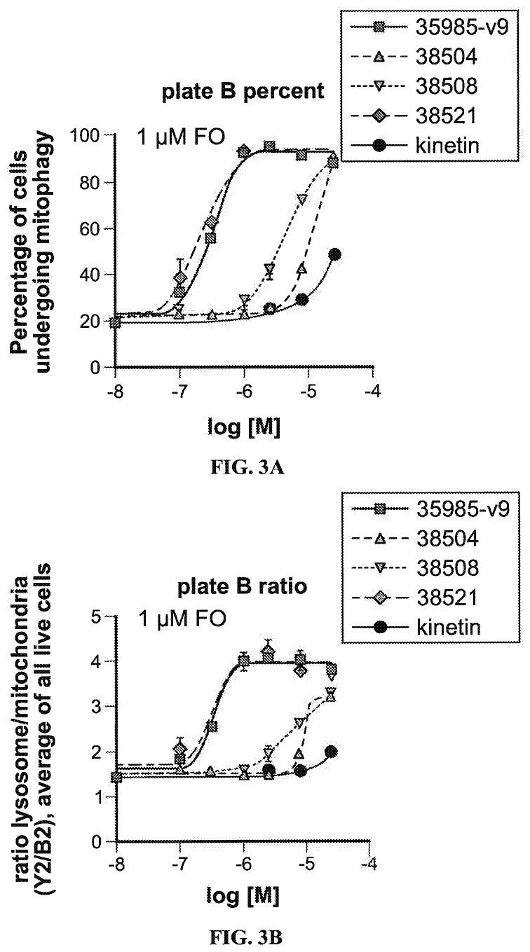

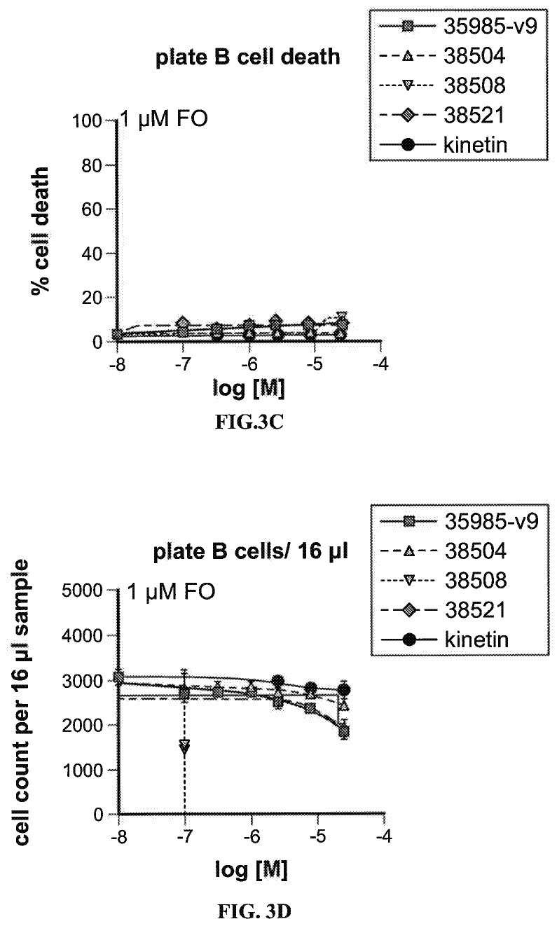

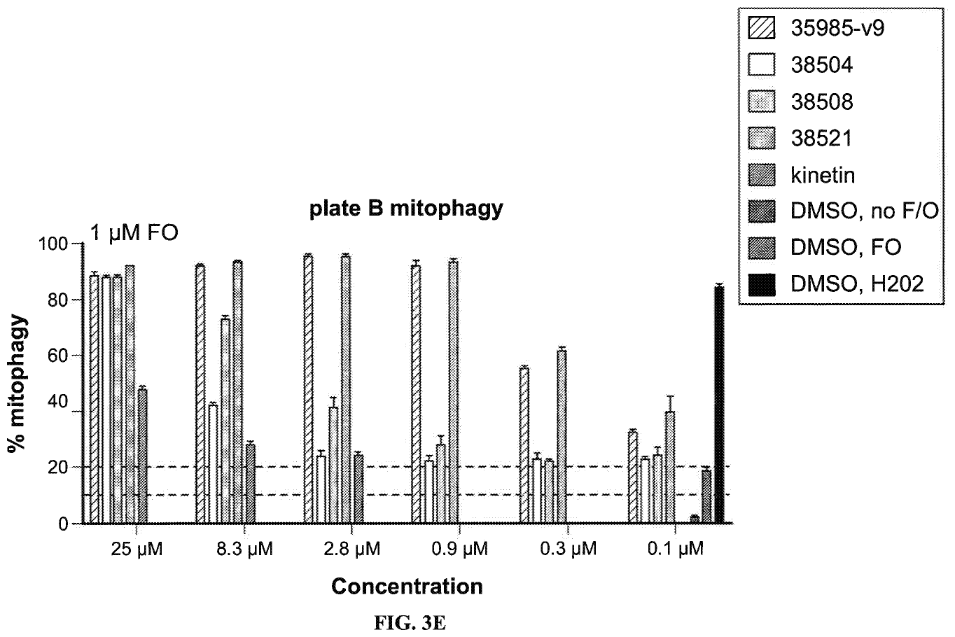

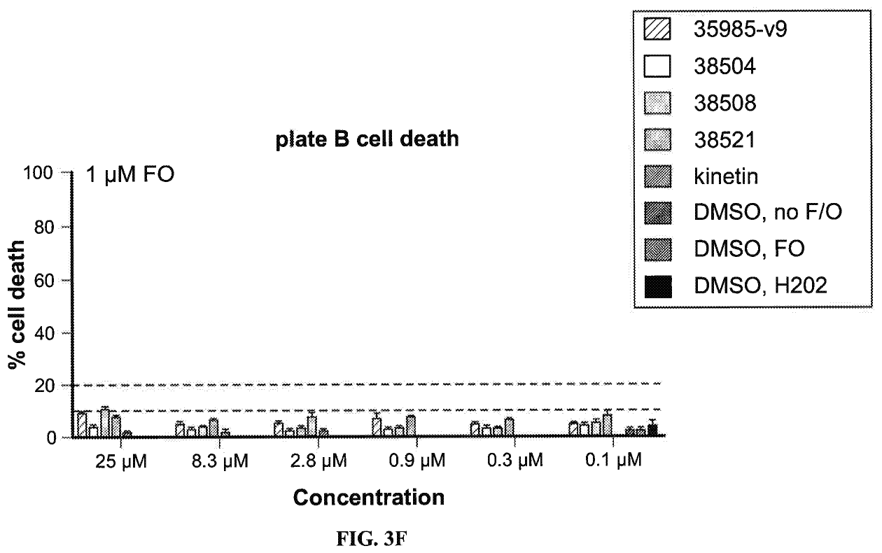

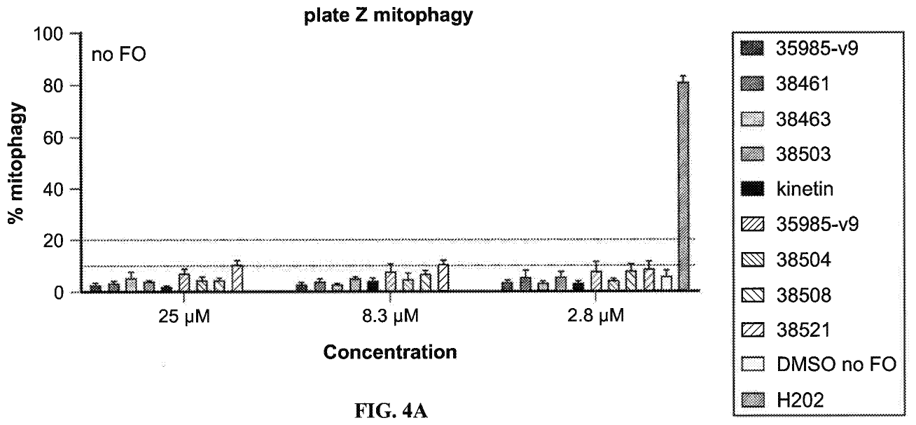

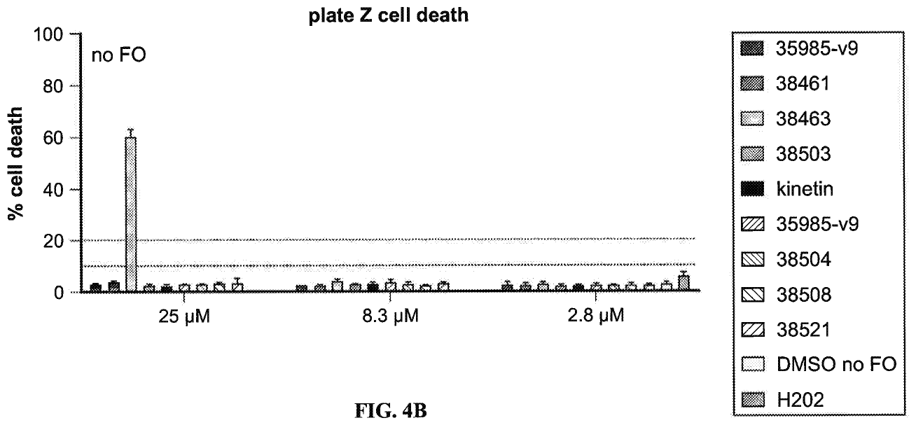

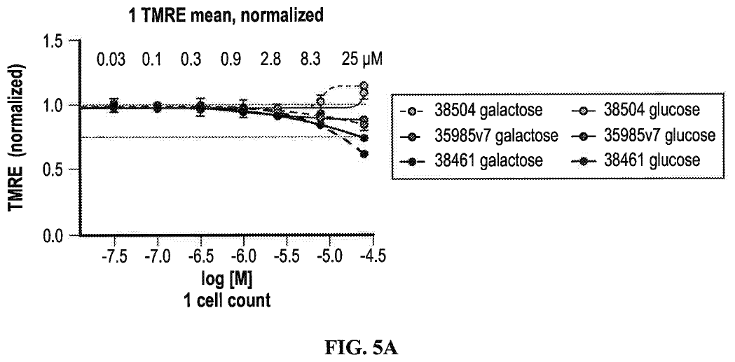

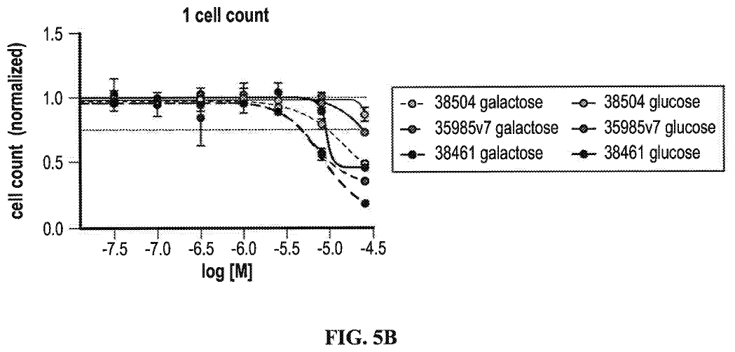

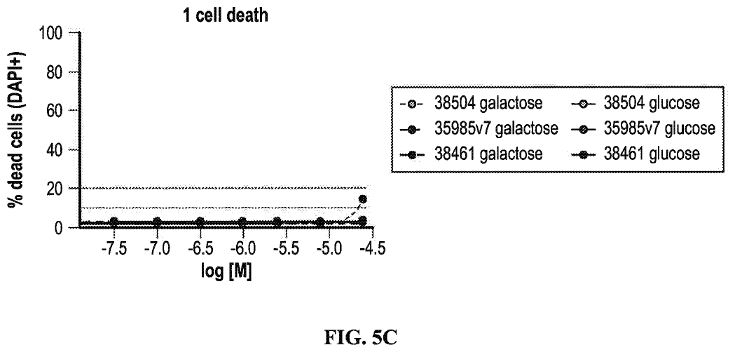

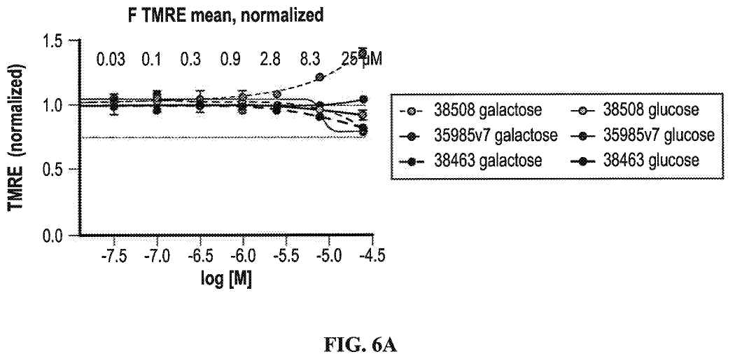

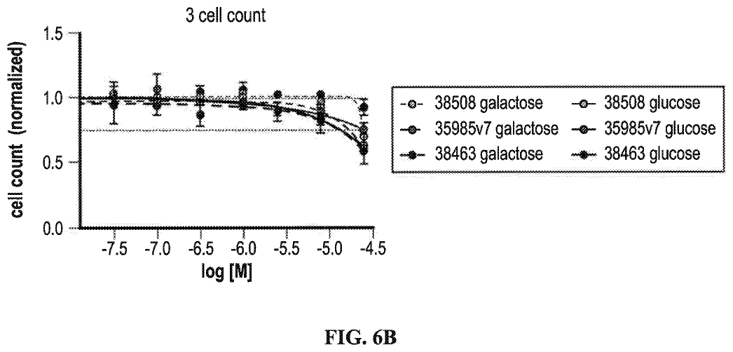

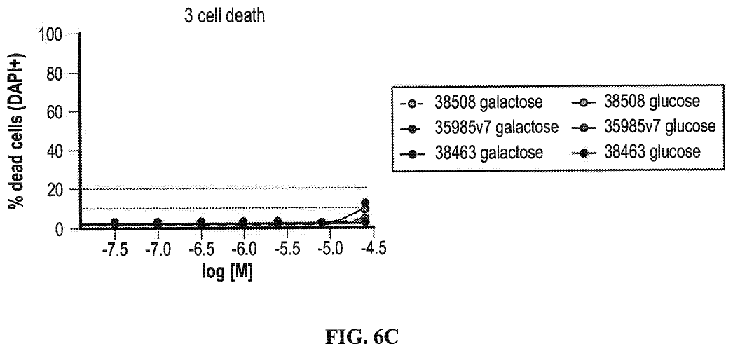

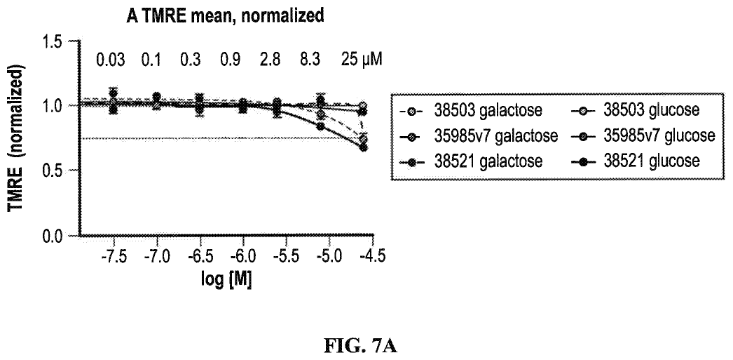

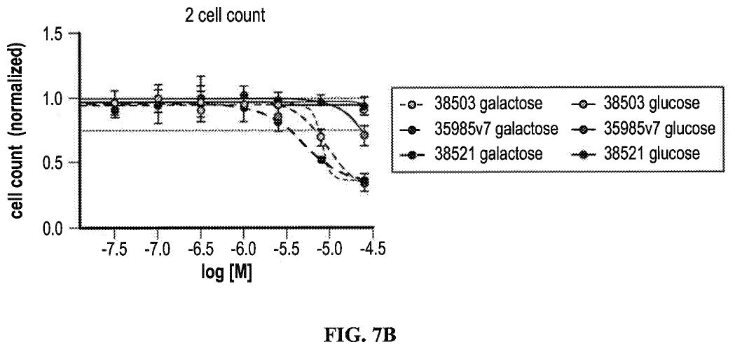

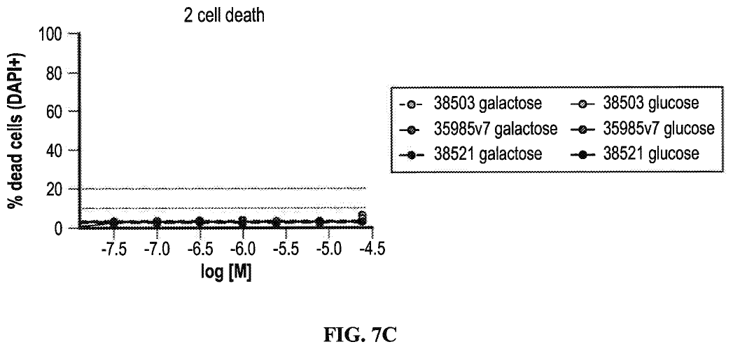

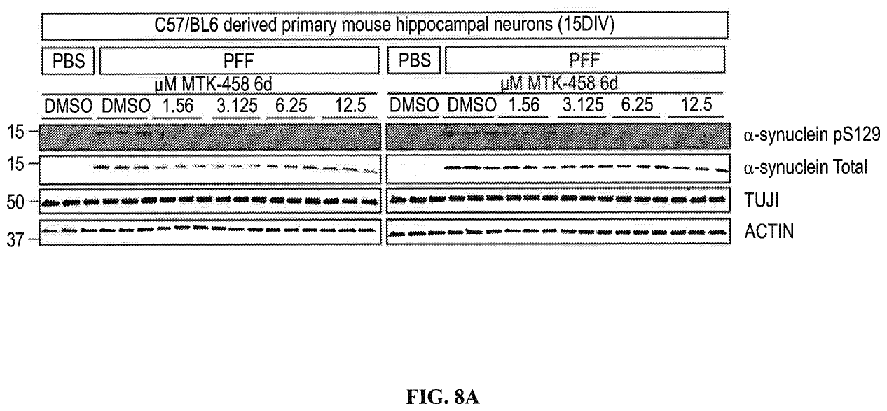

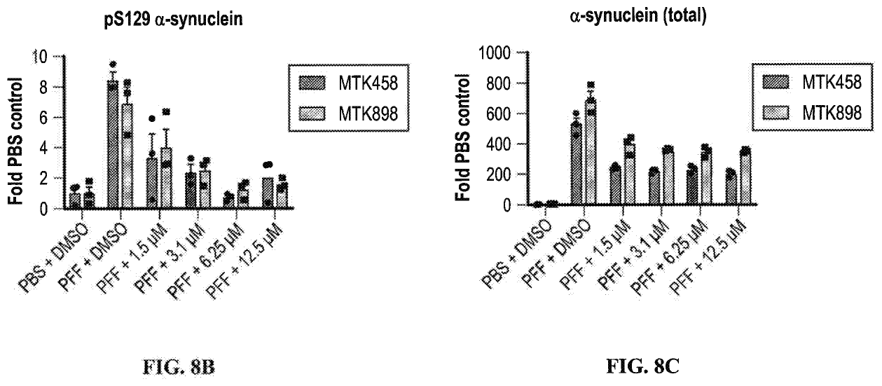

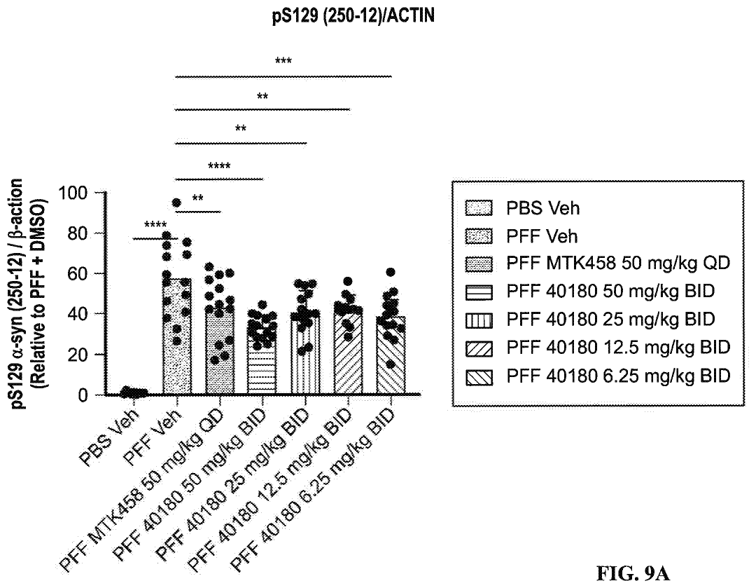

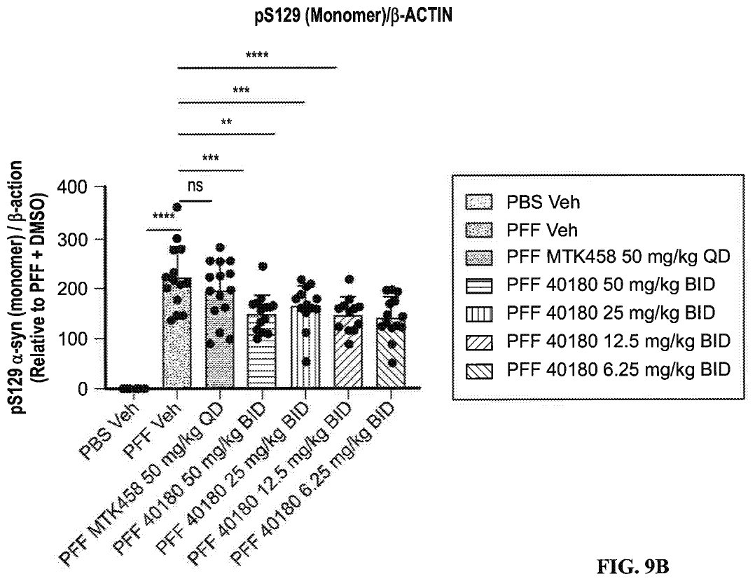

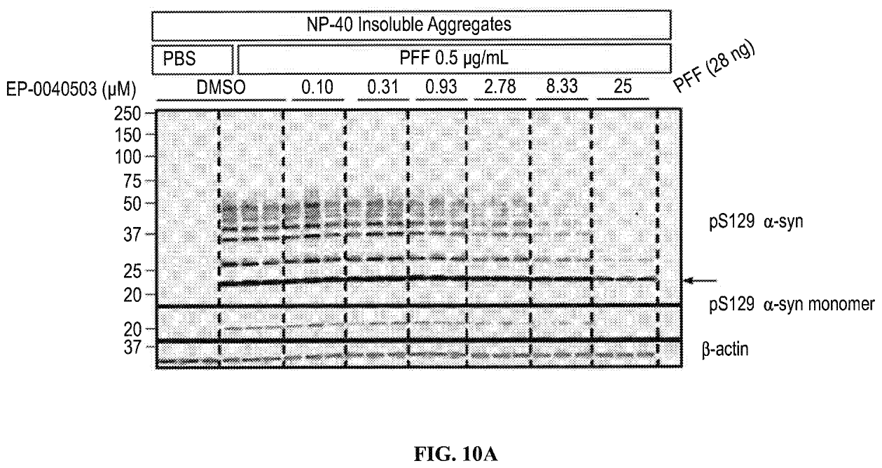

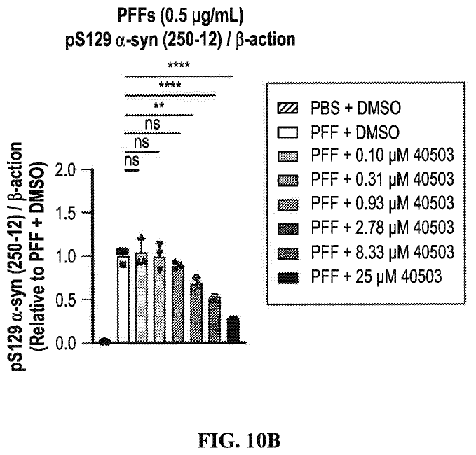

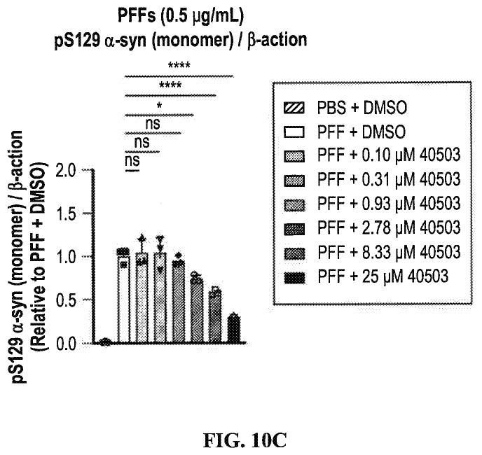

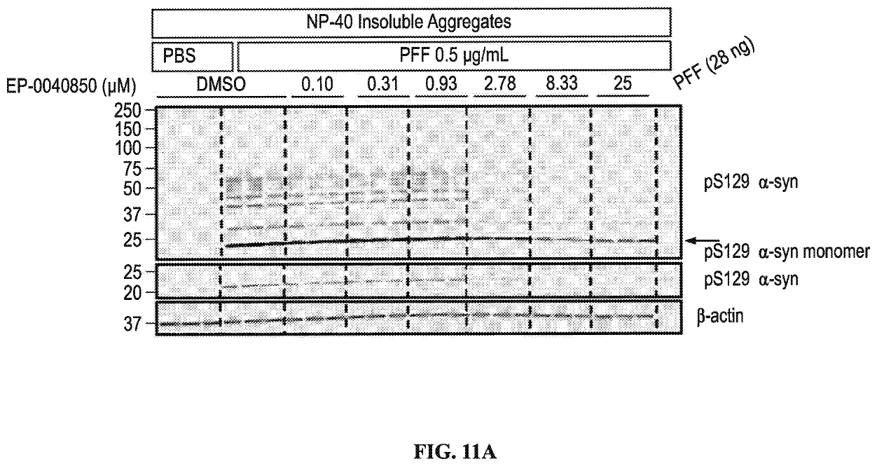

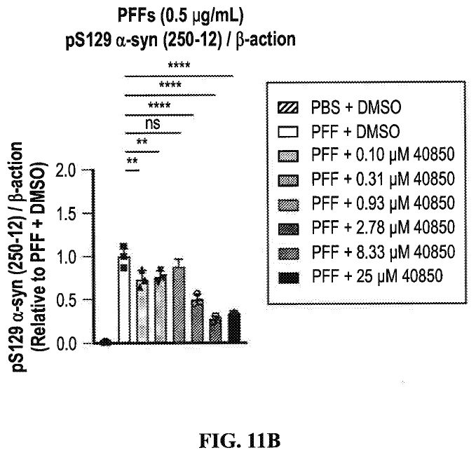

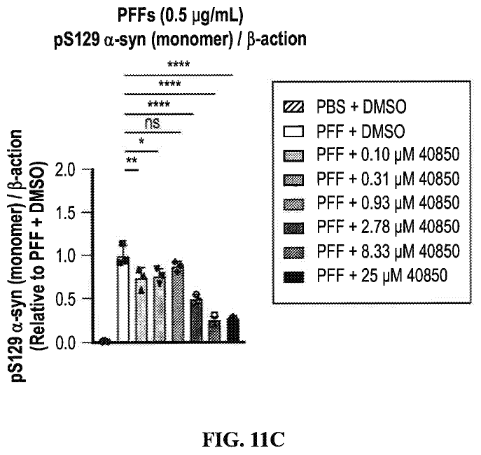

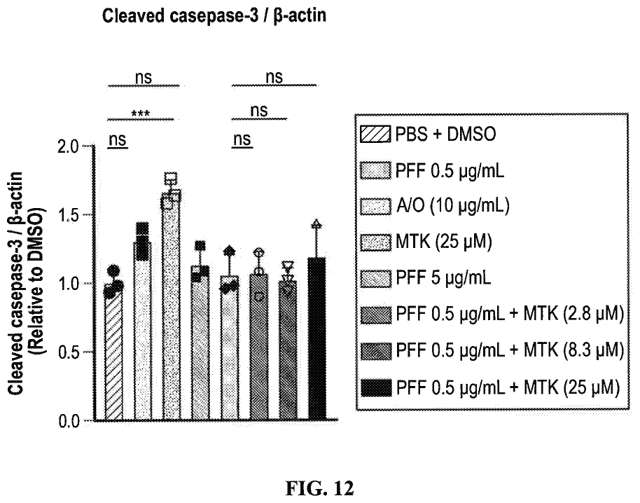

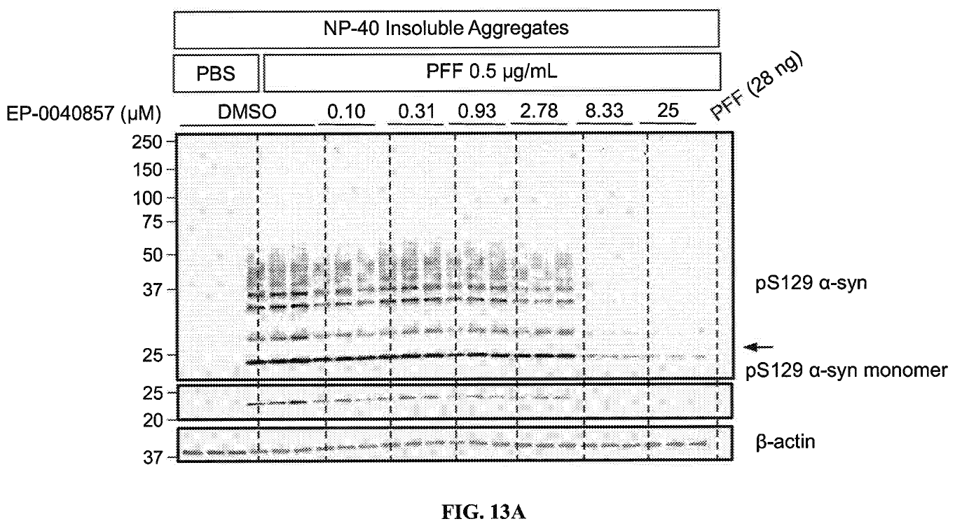

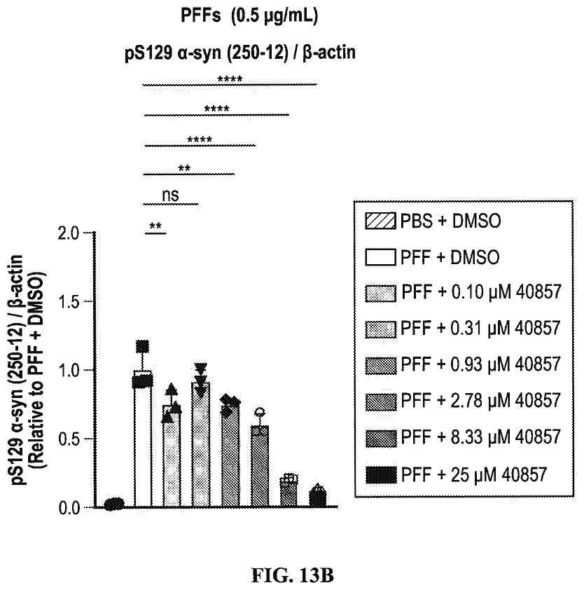

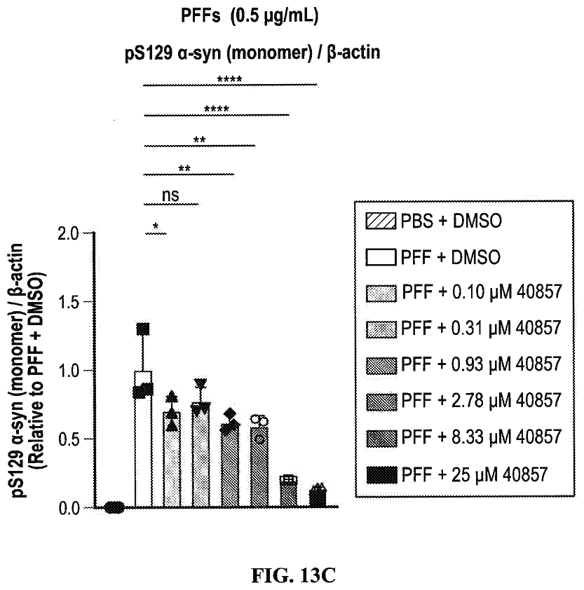

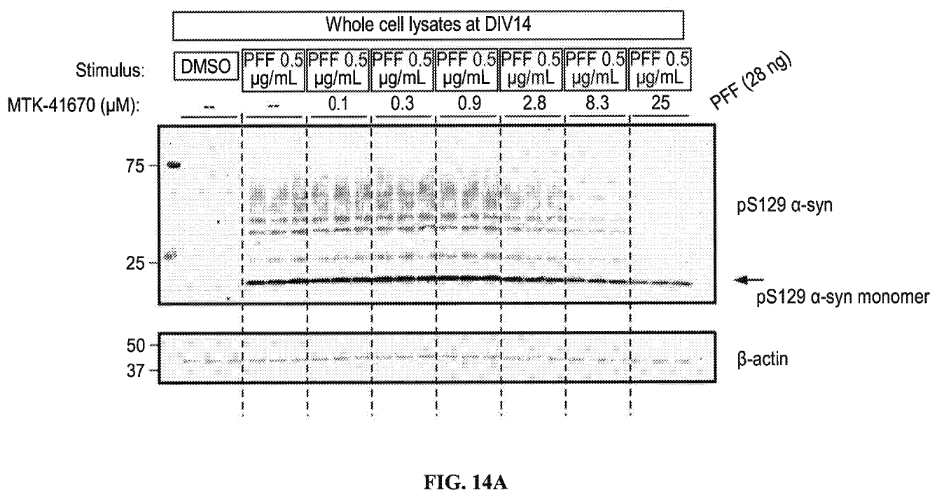

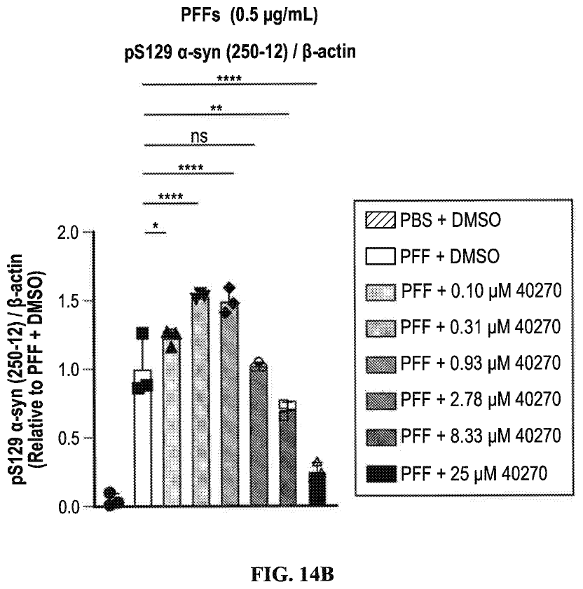

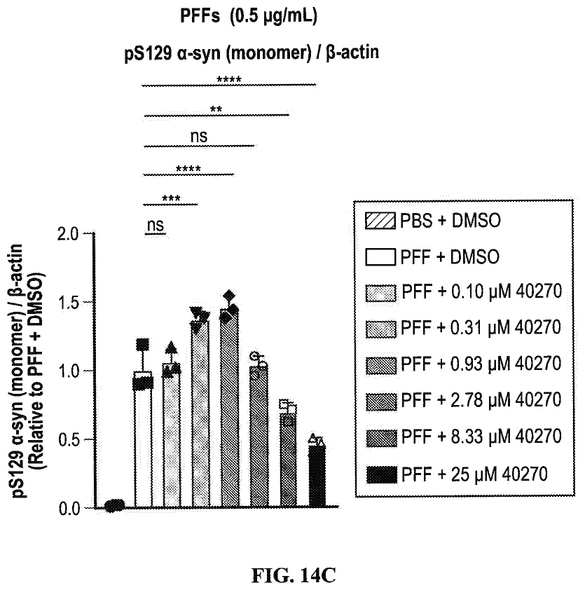

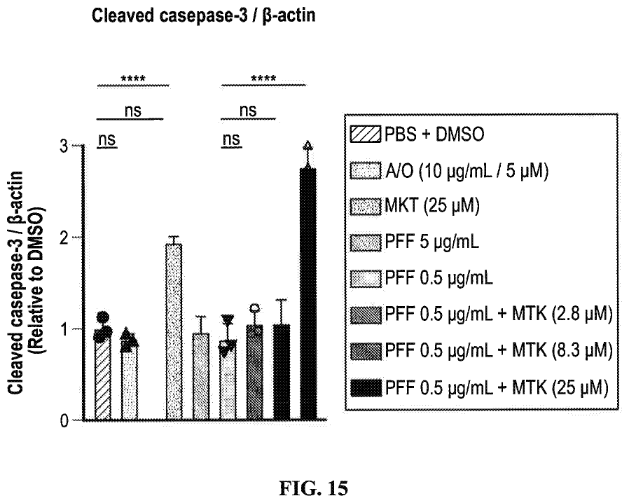

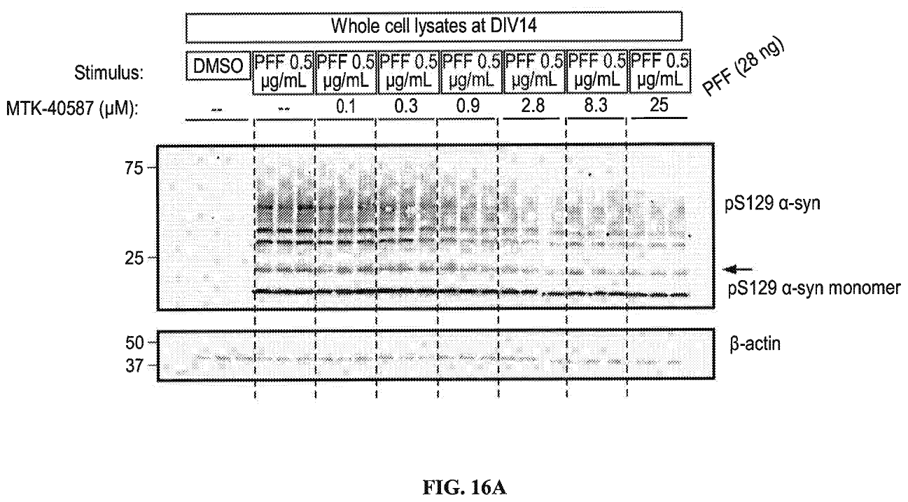

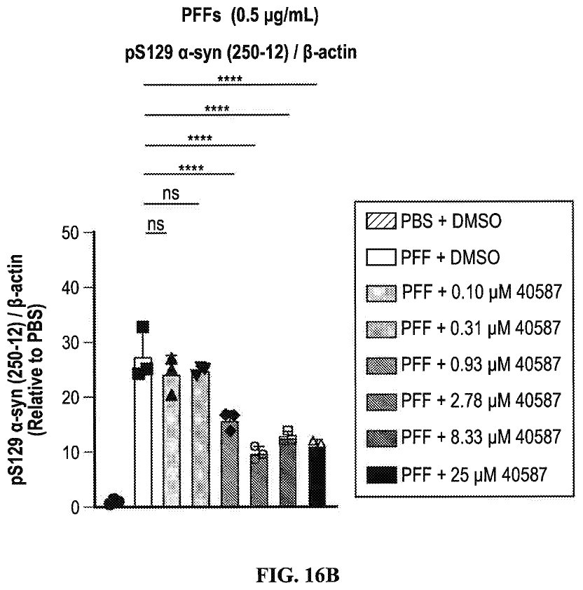

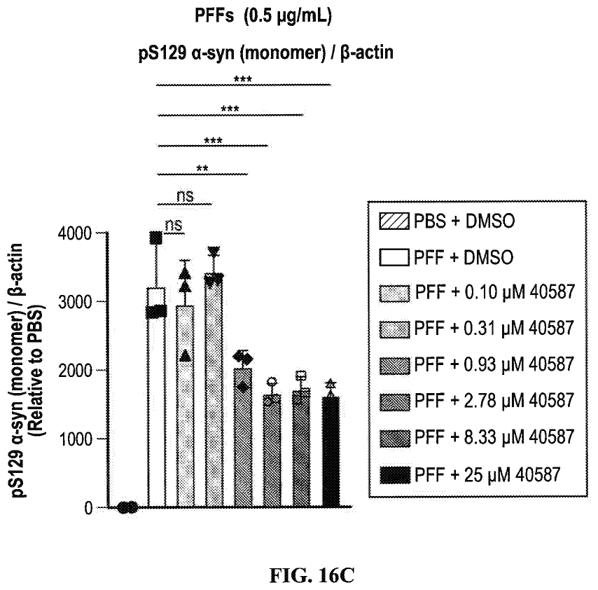

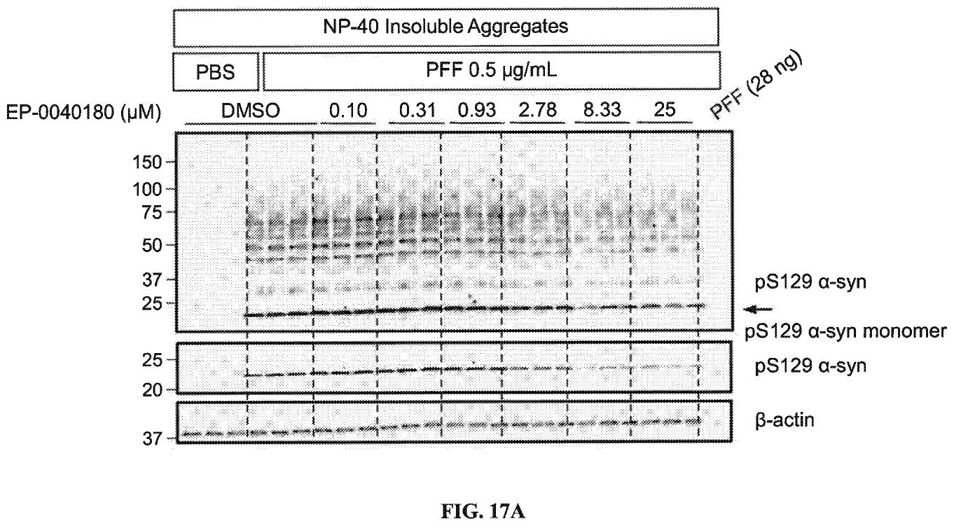

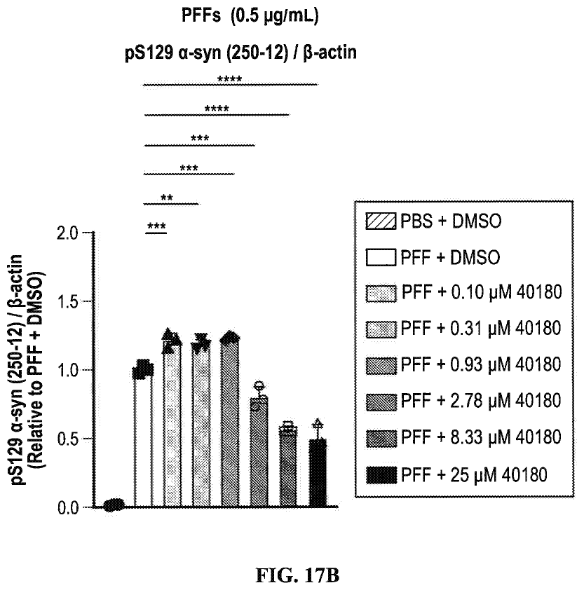

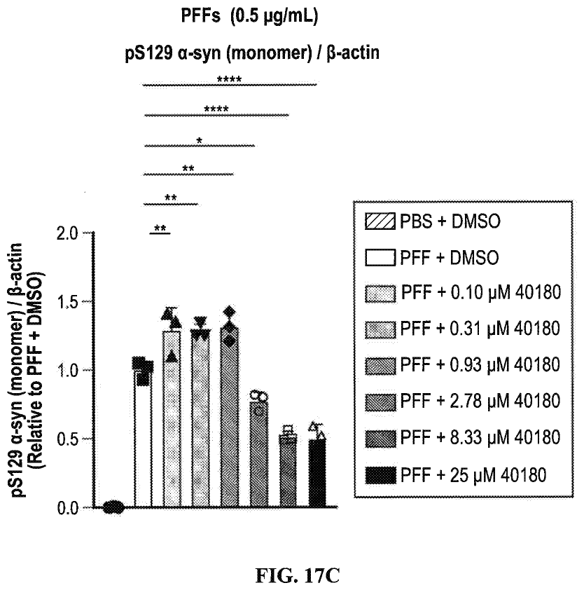

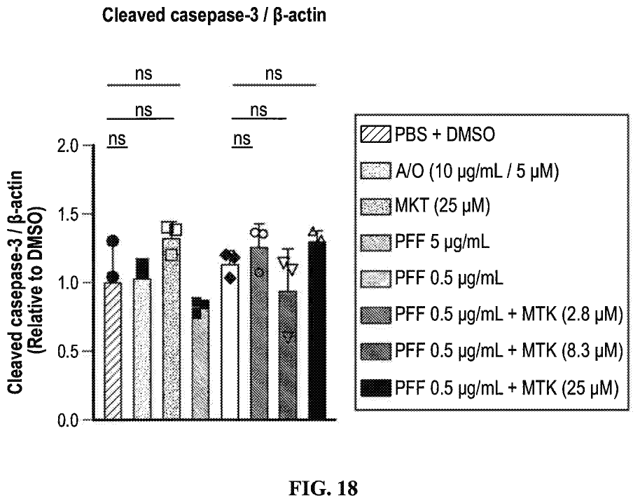

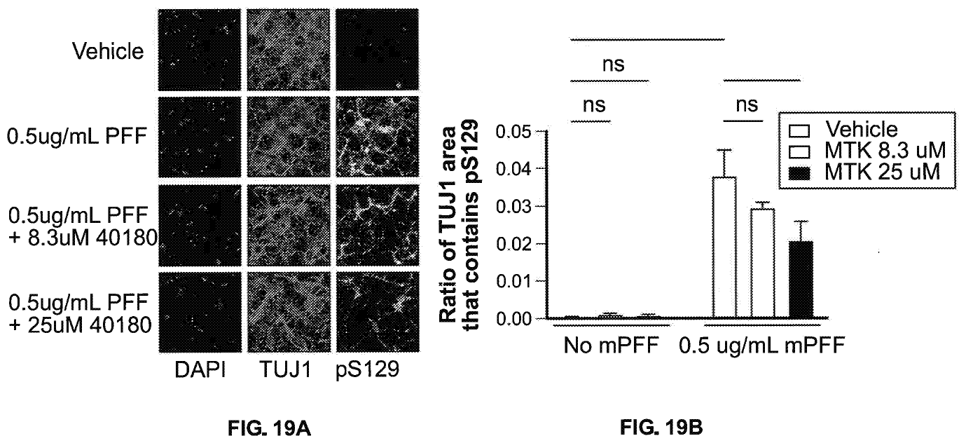

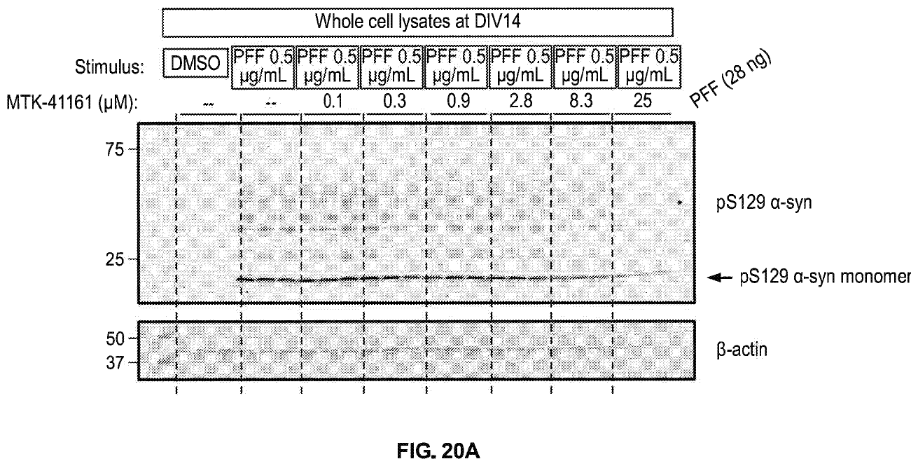

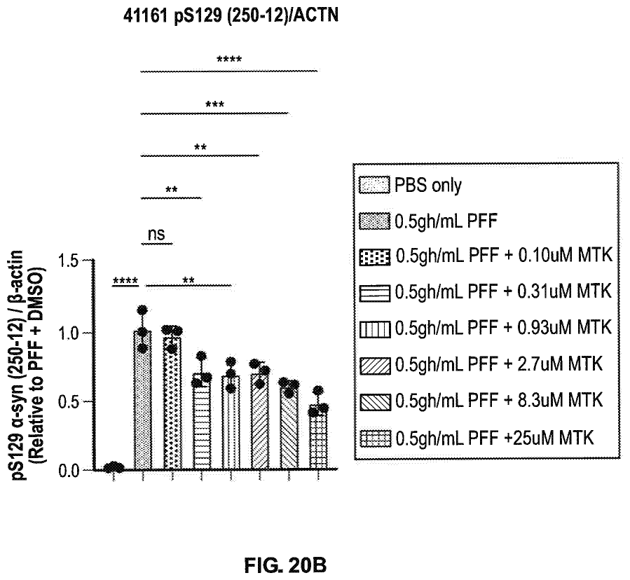

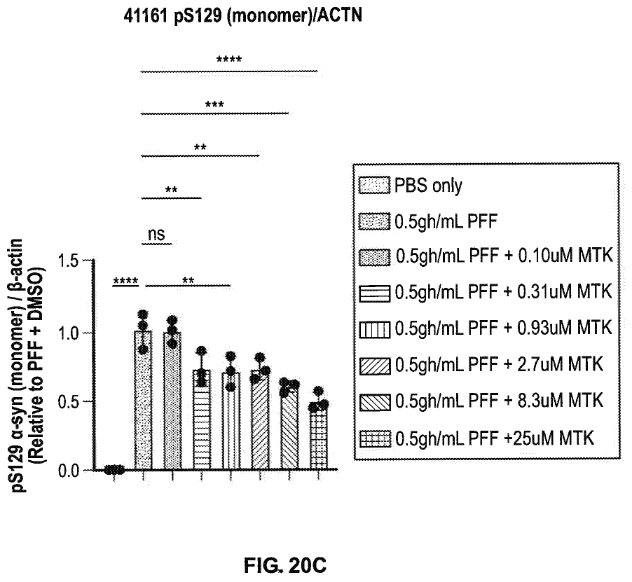

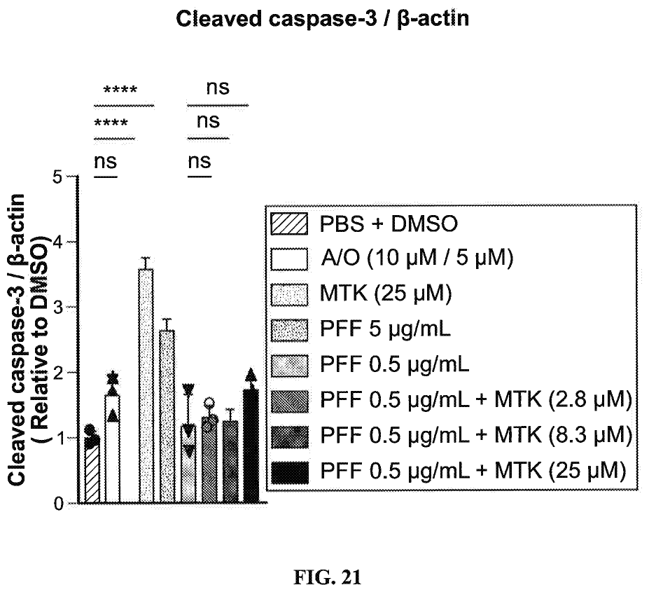

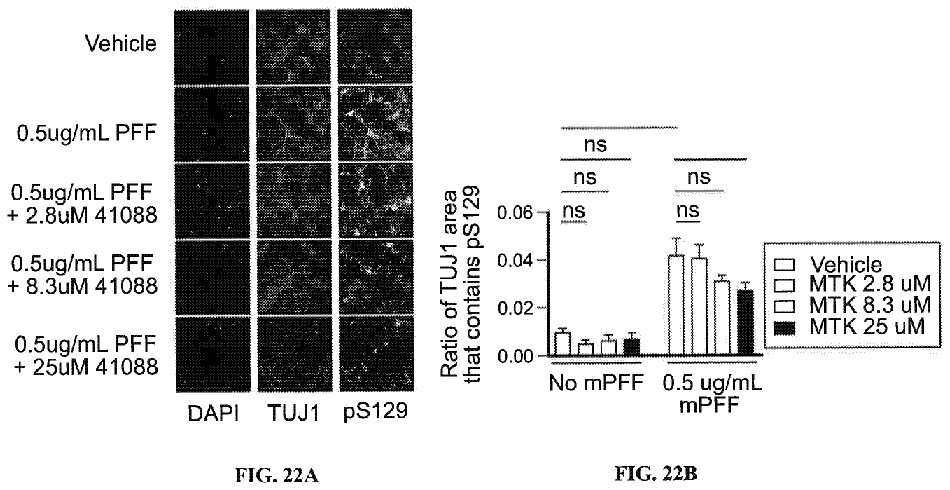

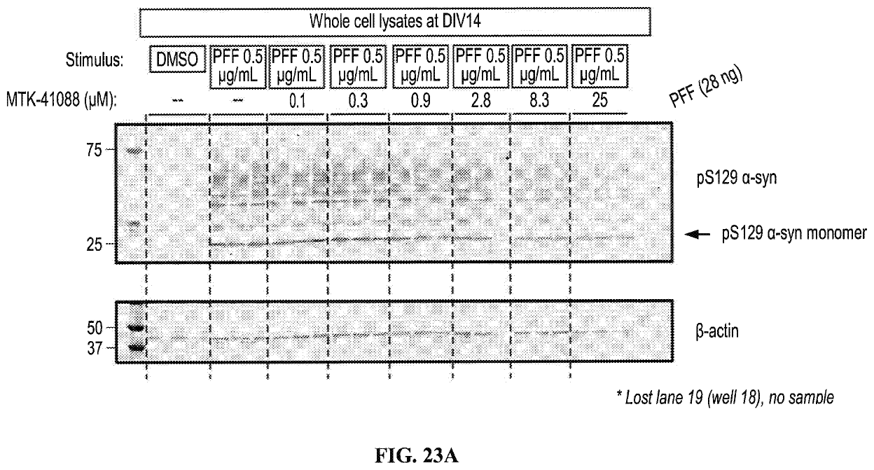

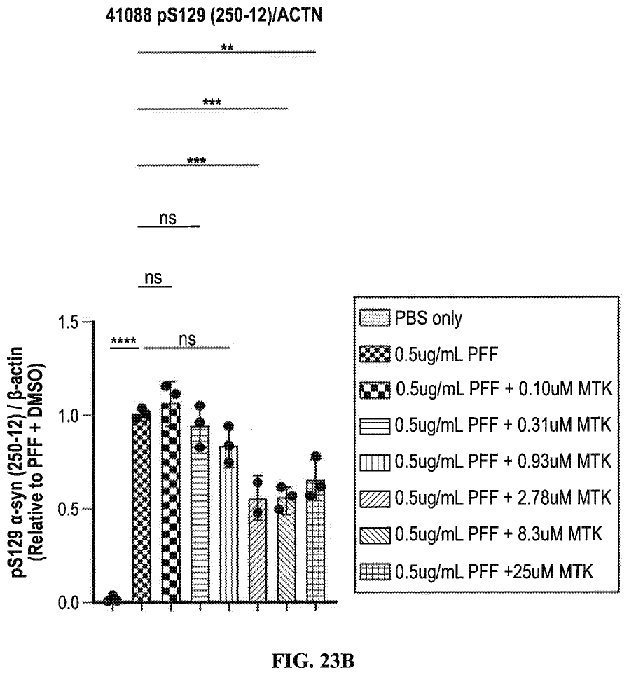

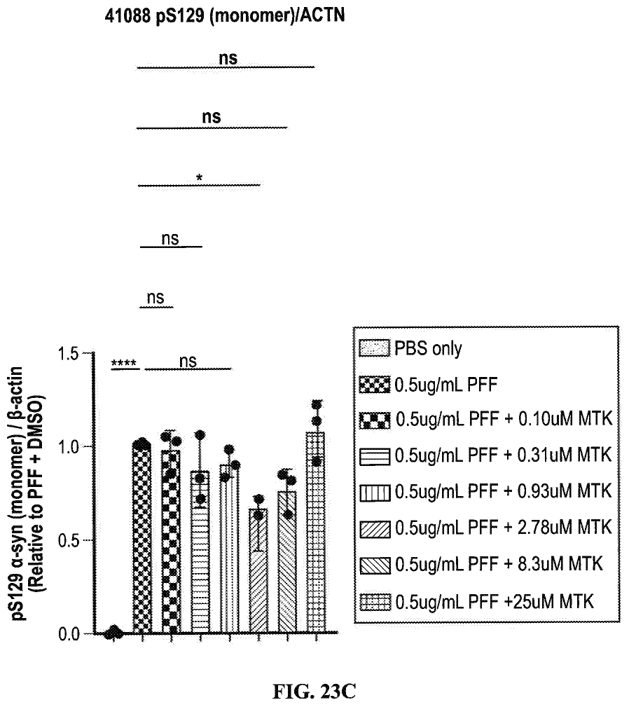

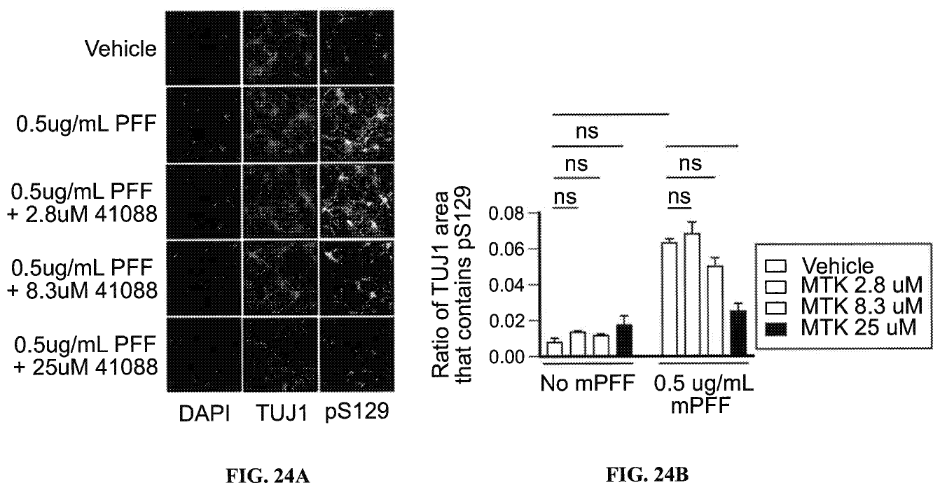

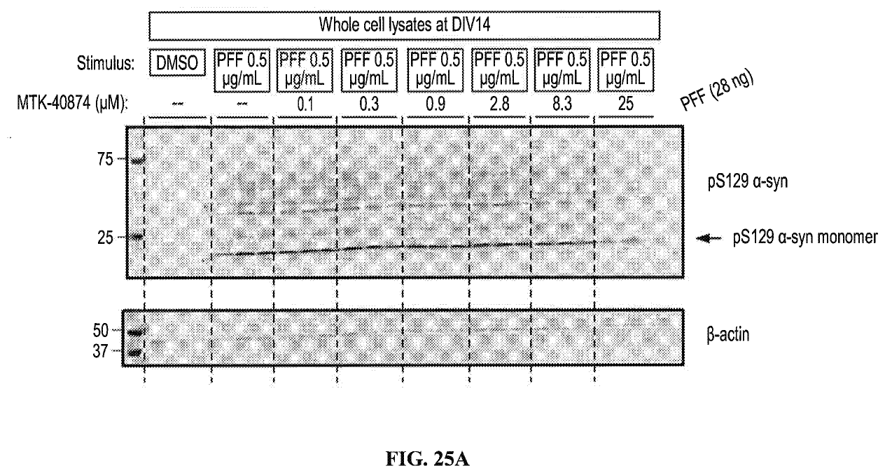

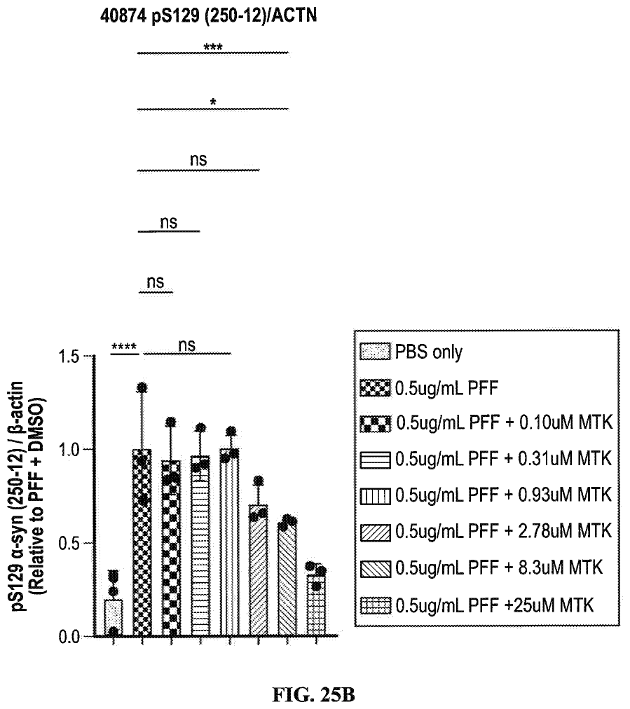

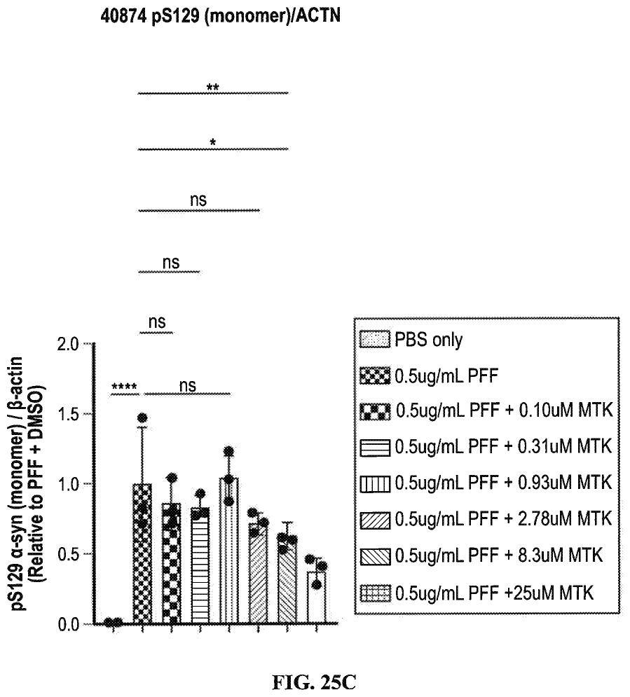

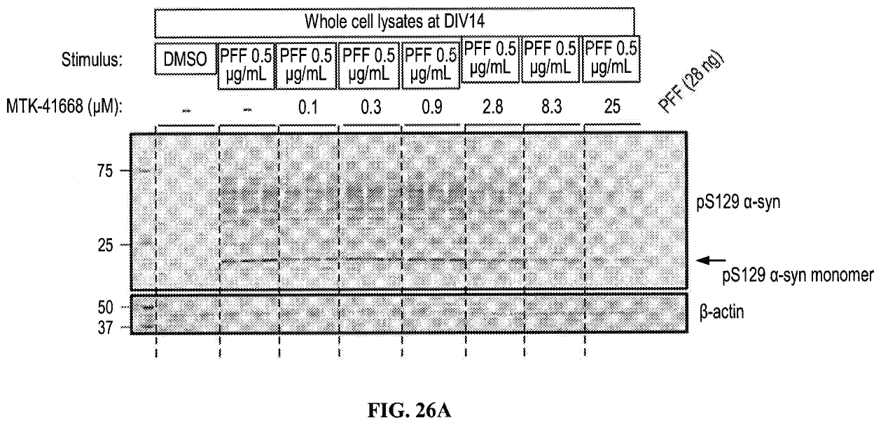

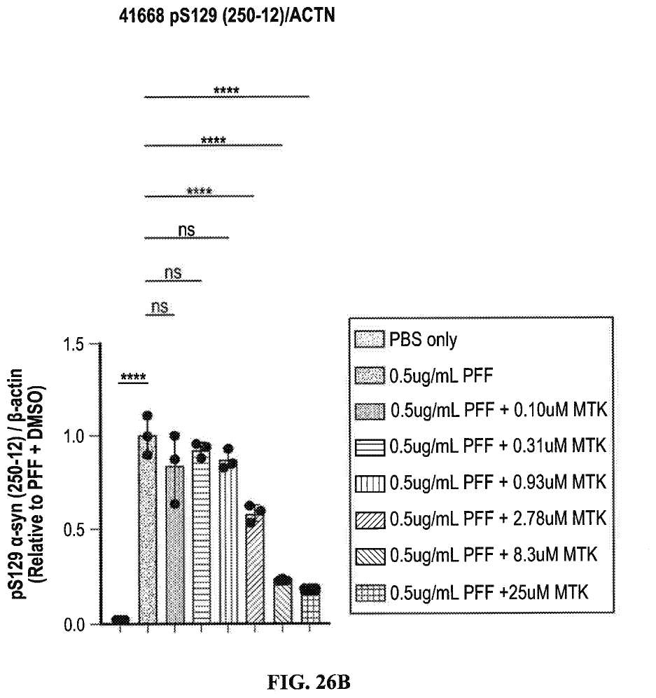

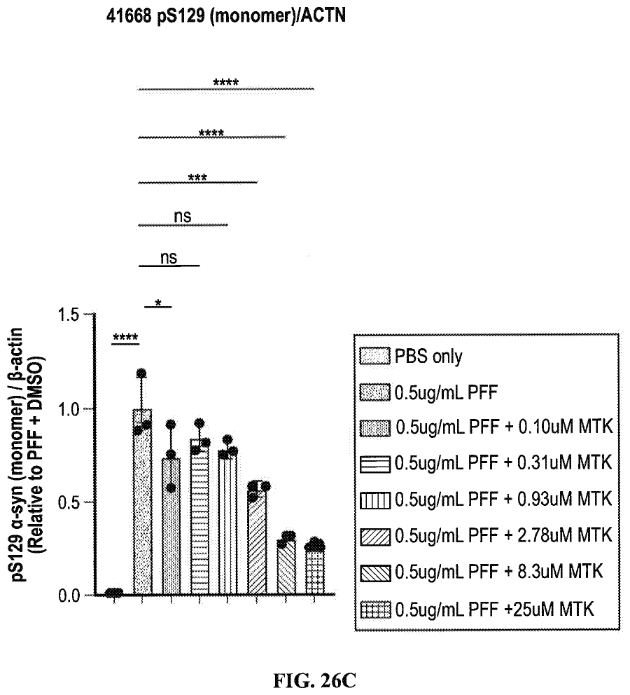

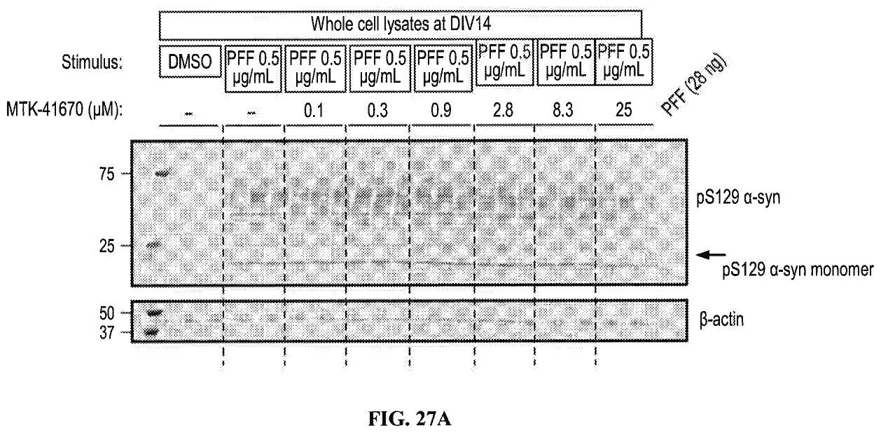

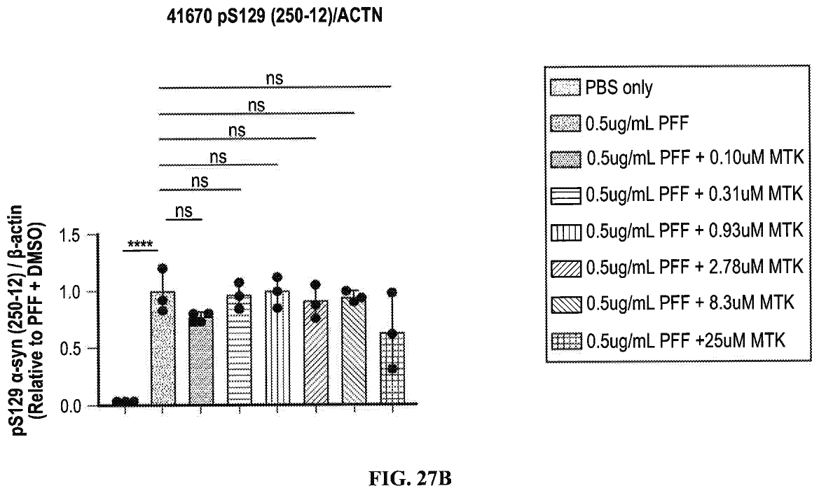

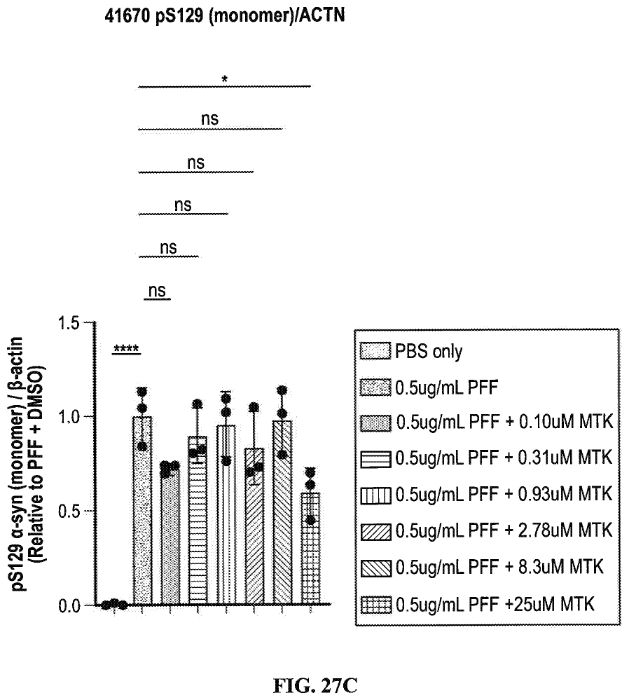

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means that the numerical value is approximate and small variations would not significantly affect the practice of the disclosed embodiments. The term “about” as used herein when referring to a measurable value such as an amount, atemporal duration, and the like, is meant to encompass variations of ±10%, ±5%, ±1%, or ±0.1% from the specified value, as such variations are appropriate to perform the disclosed methods. Where a numerical limitation is used, unless indicated otherwise by the context, “about” means the numerical value can vary by ±10%, ±5%, ±4%, ±3%, ±2%, or ±1% and remain within the scope of the disclosed embodiments.

The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder associated with PINK1 kinase activity such as, for example, a neurodegenerative disease, a mitochondrial disease, fibrosis, and/or cardiomyopathy, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition. The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion. The terms “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. In some embodiments, the compound is administered intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally and intrasternally, by injection, or by infusion.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In some embodiments, an IC₅₀ can refer to the concentration of a substance that is required for 50% inhibition in vivo, as further defined elsewhere herein.

As used herein, “EC₅₀,” is intended to refer to the concentration of a substance (e.g., a compound or a drug) that is results in a half-maximal response (i.e., 50% of the maximum response) of a biological process, or component of a process, including a protein, subunit, organelle, ribonucleoprotein, etc. In some embodiments, an EC₅₀ can refer to the concentration of a substance that is required to achieve 50% of the maximum response in vivo, as further defined elsewhere herein.

The compounds according to this disclosure may form prodrugs at hydroxyl or amino functionalities using alkoxy, amino acids, etc, groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad embodiment, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain embodiments, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl,” as used herein, refers to a monovalent saturated, straight- or branched-chain hydrocarbon radical, having unless otherwise specified, 1-6 carbon atoms. Examples of alkyl radicals include, but are not limited to, methyl, ethyl, n-propyl, isopropyl, n-butyl, iso-butyl, sec-butyl, n-pentyl, tert-pentyl, neopentyl, sec-pentyl, 3-pentyl, sec-isopentyl, hexyl, 2-methylpentane, 3-methylpentane, 2,2-dimethylbutane, 2,3-dimentybutane and the like. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “heteroalkyl,” as used herein refers to an alkyl group containing at least one heteroatom. Suitable heteroatoms include, but are not limited to, 0, N, Si, P, and S, wherein the nitrogen, phosphorous and sulfur atoms are optionally oxidized, and the nitrogen heteroatom is optionally quaternized. Heteroalkyls can be substituted as defined above for alkyl groups.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “9- to 10-membered carbocyclyl” means a 9- or 10-membered monocyclic, bicyclic (e.g., a bridged or spiro bicyclic ring), polycyclic (e.g., tricyclic), or fused hydrocarbon ring system that is saturated or partially unsaturated. The term “9- to 10-membered carbocyclyl” also includes saturated or partially unsaturated hydrocarbon rings that are fused to one or more aromatic or partially saturated hydrocarbon rings (e.g., dihydroindenyl and tetrahydronaphthalenyl). Bridged bicyclic cycloalkyl groups include, without limitation, bicyclo[4.3.1]decanyl and the like. Spiro bicyclic cycloalkyl groups include, e.g., spiro[3.6]decanyl, spiro[4.5]decanyl, spiro [4.4]nonyl and the like. Fused cycloalkyl rings include, e.g., decahydronaphthalenyl, dihydroindenyl, decahydroazulenyl, octahydroazulenyl, tetrahydronaphthalenyl, and the like. It will be understood that when specified, optional substituents on a carbocyclyl (e.g., in the case of an optionally substituted cycloalkyl) may be present on any substitutable position and, include, e.g., the position at which the carbocyclyl group is attached.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, cycloheptyl, cyclooctyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein. In various aspects, the cycloalkyl group and heterocycloalkyl group can be monocyclic, bicyclic (e.g., bridged such as, for example, bicyclo[4.3.1]decanyl or spiro such as, for example, spiro[3.6]decanyl, spiro[4.5]decanyl, spiro [4.4]nonyl), polycyclic (e.g., tricyclic), or a fused hydrocarbon ring system that is saturated or partially unsaturated (e.g., decahydronaphthalenyl, dihydroindenyl, decahydroazulenyl, octahydroazulenyl, tetrahydronaphthalenyl).

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” The heterocycle can be monocyclic, bicyclic (e.g., spiro or bridged), polycyclic, or a fused system that is saturated or partially saturated. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C;2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, Wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “9-membered fused heterocyclyl” means a 9-membered saturated or partially unsaturated fused monocyclic heterocyclic ring comprising at least one oxygen heteroatom and optionally two to four additional heteroatoms independently selected from N, O, and S. The terms “heterocycle,” “heterocyclyl,” “heterocyclyl ring,” “heterocyclic group,” “heterocyclic moiety,” and “heterocyclic radical,” are used interchangeably herein. A heterocyclyl ring can be attached to its pendant group at any heteroatom or carbon atom that results in a stable structure. Examples of fused saturated or partially unsaturated heterocyclic radicals comprising at least one oxygen atom include, without limitation, dihydrobenzofuranyl, dihydrofuropyridinyl, octahydrobenzofuranyl, and the like. Where specified as being optionally substituted, substituents on a heterocyclyl (e.g., in the case of an optionally substituted heterocyclyl) may be present on any substitutable position and include, e.g., the position at which the heterocyclyl group is attached.

The term “aromatic group” as used herein refers to a ring structure having cyclic clouds of delocalized a electrons above and below the plane of the molecule, where the a clouds contain (4n+2) a electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “5- or 6-membered heteroaryl” refers to a 5- or 6-membered aromatic radical containing 1-4 heteroatoms selected from N, O, and S. Nonlimiting examples include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. When specified, optional substituents on a heteroaryl group may be present on any substitutable position and, include, e.g., the position at which the heteroaryl is attached.

The term “aldehyde” as used herein is represented by the formula —C(O)H. Throughout this specification “C(O)” is a short hand notation for a carbonyl group, i.e., C═O,

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain embodiments, unless expressly indicated to the contrary, individual substituents can be further optionally substituted (i.e., further substituted or unsubstituted).

In some embodiments, a structure of a compound can be represented by a formula:

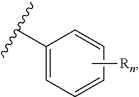

which is understood to be equivalent to a formula:

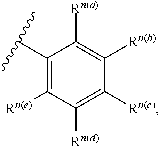

wherein n is typically an integer. That is, Rⁿ is understood to represent five independent substituents, R^{n(a)}, Rⁿ⁽ⁿ⁾, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further embodiments, a structure of a compound can be represented by a formula:

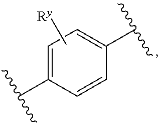

wherein R^{y} represents, for example, 0-2 independent substituents selected from A¹, A², and A³, which is understood to be equivalent to the groups of formulae:

wherein R^{y} represents 0 independent substituents

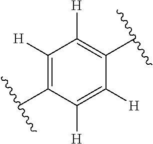

wherein R^{y} represents 1 independent substituent

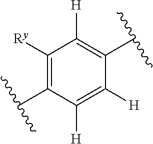

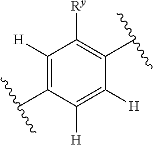

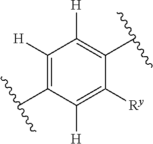

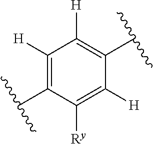

wherein R^{y} represents 2 independent substituents

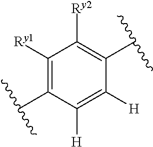

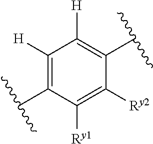

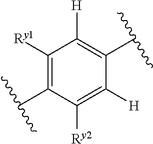

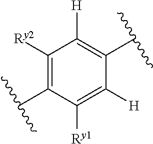

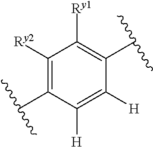

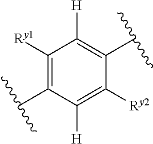

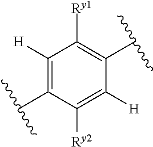

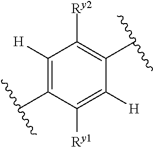

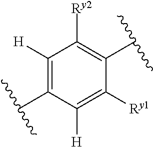

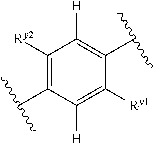

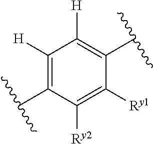

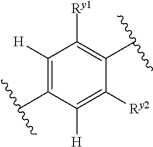

Again, by “independent substituents,” it is meant that each R substituent can be independently defined. For example, if in one instance R^{y1} is A¹, then R^{y2} is not necessarily A¹ in that instance.

In some further embodiments, a structure of a compound can be represented by a formula,

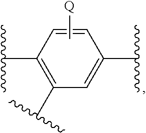

wherein, for example, Q comprises three substituents independently selected from hydrogen and A, which is understood to be equivalent to a formula:

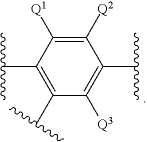

Again, by “independent substituents,” it is meant that each Q substituent is independently defined as hydrogen or A, which is understood to be equivalent to the groups of formulae:

wherein Q comprises three substituents independently
selected from H and A

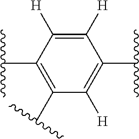 [SMILES: *c1ccc(*)c(*)c1]

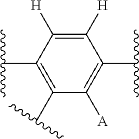

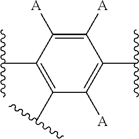

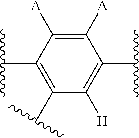

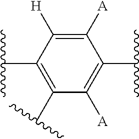

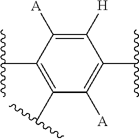

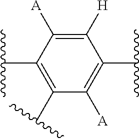

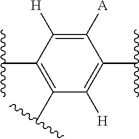

In some embodiment, the disclosed compounds exists as geometric isomers. “Geometric isomer” refers to isomers that differ in the orientation of substituent atoms in relationship to a cycloalkyl ring, i.e., cis or trans isomers. When a disclosed compound is named or depicted by structure without indicating a particular cis or trans geometric isomer form, it is to be understood that the name or structure encompasses one geometric isomer free of other geometric isomers, mixtures of geometric isomers, or mixtures enriched in one geometric isomer relative to its corresponding geometric isomer. When a particular geometric isomer is depicted, i.e., cis or trans, the depicted isomer is at least about 60%, 70%, 80%, 90%, 99%, or 99.9% by weight pure relative to the other geometric isomer.

The compounds described herein may be present in the form of pharmaceutically acceptable salts. For use in medicines, the salts of the compounds described herein refer to non-toxic “pharmaceutically acceptable salts.” As noted above, the compounds of the present invention can be administered, inter alia, as pharmaceutically acceptable salts, esters, amides or prodrugs. The term “salts” refers to inorganic and organic salts of compounds of the present invention. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977). Examples of pharmaceutically acceptable esters of the compounds of the present invention include C1-C8 alkyl esters. Acceptable esters also include C5-C7 cycloalkyl esters, as well as arylalkyl esters such as benzyl. C1-C4 alkyl esters are commonly used. Esters of compounds of the present invention may be prepared according to methods that are well known in the art. Examples of pharmaceutically acceptable amides of the compounds of the present invention include amides derived from ammonia, primary C1-C8 alkyl amines, and secondary C1-C8 dialkyl amines. In the case of secondary amines, the amine may also be in the form of a 5 or 6 membered heterocycloalkyl group containing at least one nitrogen atom. Amides derived from ammonia, C1-C3 primary alkyl amines and C1-C2 dialkyl secondary amines are commonly used. Amides of the compounds of the present invention may be prepared according to methods well known to those skilled in the art.

Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

Disease, disorder, and condition are used interchangeably herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “effective amount” or “therapeutically effective amount” refers to an amount that is sufficient to achieve the desired result (e.g., that will elicit a biological or medical response of a subject e.g., a dosage of between 0.01-100 mg/kg body weight/day) or to have an effect on an undesired condition. For example, a “therapeutically effective amount” refers to an amount that is sufficient to achieve the desired therapeutic result or to have an effect on undesired symptoms. In some embodiments, a “therapeutically effective amount” refers to an amount that is sufficient to achieve the desired therapeutic result or to have an effect on undesired symptoms, but it is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various embodiments, a preparation can be administered in a “prophylactically effective amount”; that is, an amount effective for prevention of a disease or condition.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). In some embodiments, the subject is a human in need of treatment. In some embodiments, “patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In embodiments, a patient is human.

The term “associated” or “associated with” in the context of a substance or substance activity or function associated with a disease (e.g., a protein associated disease, a symptom associated with a cardiomyopathy, neurodegenerative disease, or symptom associated with Parkinson's disease) means that the disease (e.g., cardiomyopathy, neurodegenerative disease or Parkinson's disease) is caused by (in whole or in part), or a symptom of the disease is caused by (in whole or in part) the substance or substance activity or function. For example, a symptom of a disease or condition associated with a reduction in the level of PINK1 activity may be a symptom that results (entirely or partially) from a reduction in the level of PINK1 activity (e.g., loss of function mutation or gene deletion or modulation of PINK1 signal transduction pathway). As used herein, what is described as being associated with a disease, if a causative agent, could be a target for treatment of the disease. For example, a disease associated with PINK1, may be treated with an agent (e.g., compound as described herein) effective for increasing the level of activity of PINK1.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g., PINK1). In embodiments contacting includes allowing a compound described herein to interact with a protein or enzyme that is involved in a signaling pathway.

As defined herein, the term “inhibition,” “inhibit,” “inhibiting.” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The symbol “ [symbol] ” denotes the point of attachment of a chemical moiety to the remainder of a molecule or chemical formula.

As defined herein, the term “activation,” “activate,” “activating” and the like in reference to a protein-activator (e.g., agonist) interaction means positively affecting (e.g., increasing) the activity or function of the protein (e.g., PINK1) relative to the activity or function of the protein in the absence of the activator (e.g., compound described herein). In embodiments, activation refers to an increase in the activity of a signal transduction pathway or signaling pathway (e.g., PINK1 pathway). Thus, activation may include, at least in part, partially or totally increasing stimulation, increasing or enabling activation, or activating, sensitizing, or up-regulating signal transduction or enzymatic activity or the amount of a protein decreased in a disease (e.g., reduction of the level of PINK1 activity or protein associated with a cardiomyopathy or a neurodegenerative disease such as Parkinson's disease). Activation may include, at least in part, partially or totally increasing stimulation, increasing or enabling activation, or activating, sensitizing, or up-regulating signal transduction or enzymatic activity or the amount of a protein (e.g., PINK1) that may modulate the level of another protein or increase cell survival (e.g., increase in PINK1 activity may increase cell survival in cells that may or may not have a reduction in PINK1 activity relative to a non-disease control).

The term “modulator” refers to a composition that increases or decreases the level of a target molecule or the function of a target molecule. In embodiments, the modulator is a modulator of PINK1. In embodiments, the modulator is a modulator of PINK1 and is a compound that reduces the severity of one or more symptoms of a disease associated with PINK1 (e.g., reduction of the level of PINK1 activity or protein associated with a cardiomyopathy, neurodegenerative disease such as Parkinson's disease). In embodiments, a modulator is a compound that reduces the severity of one or more symptoms of a cardiomyopathy or neurodegenerative disease that is not caused or characterized by PINK1 (e.g., loss of PINK1 function) but may benefit from modulation of PINK1 activity (e.g., increase in level of PINK1 or PINK1 activity).

“Disease” or “condition” refer to a state of being or health status of a patient or subject capable of being treated with a compound, pharmaceutical composition, or method provided herein. In embodiments, the disease is a disease related to (e.g., characterized by) a reduction in the level of PINK1. In embodiments, the disease is a disease characterized by loss of dopamine-producing cells (e.g., Parkinson's disease). In embodiments, the disease is a disease characterized by neurodegeneration. In embodiments, the disease is a disease characterized by neural cell death. In embodiments, the disease is a disease characterized by a reduction in the level of PINK1 activity. In embodiments, the disease is Parkinson's disease. In embodiments, the disease is a neurodegenerative disease. In embodiments, the disease is a cardiomyopathy.

As used herein, the term “cardiomyopathy” refers to a disease condition that adversely affects cardiac cell tissue leading to a measurable deterioration in myocardial function (e.g., systolic function, diastolic function). Dilated cardiomyopathy is characterized by ventricular chamber enlargement with systolic dysfunction and no hypertrophy. Hypertrophic cardiomyopathy, is a genetic disease transmitted as an autosomal dominant trait. Hypertrophic cardiomyopathy is morphologically characterized by a hypertrophied and non-dialated left ventricle, Restrictive cardiomyopathy is characterized by nondialated nonhypertrophied morphology with diminished ventricular volume leading to poor ventricular filling. Arrhythmogenic right ventricular cardiomyopathy is an inheritable heart disease characterized by myocardial electric instability. Unclassified cardiomyopathy is a category for cardiomyopathies that do not match the features of any one of the other types. Unclassified cardiomyopathies may have features of multiple types or, for example, have the features of fibroelastosis, noncompacted myocardium, or systolic dysfunction with minimal dilatation.

As used herein, the term “neurodegenerative disease” refers to a disease or condition in which the function of a subject's nervous system becomes impaired. Examples of neurodegenerative diseases that may be treated with a compound or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, epilepsy, Friedreich ataxia, frontotemporal dementia, Gerstmann-Straussler-Scheinker syndrome, Huntington's disease, HIV-associated dementia, Kennedy's disease, Krabbe's disease, kuru, Leigh's disease (Leigh syndrome), Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Shy-Drager syndrome, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4). Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease. In embodiments, dysautonomia is not a neurodegenerative disease.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs.

The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Phar. Pharmacol. 49:669-674, 1997). In embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., PINK1), and/or reducing, eliminating, or slowing the progression of disease symptoms (e.g., symptoms of cardiomyopathy or a neurodegeneration such as symptoms of Parkinson's disease). Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of cardiomyopathy or neurodegeneration such as Parkinson's disease and severity of such symptoms), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a disease associated neurodegeneration (e.g., Parkinson's disease such as levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs), or with adjunctive agents that may not be effective alone, but may contribute to the efficacy of the active agent.

The compounds described herein can be used in combination with one another, with other active agents known to be useful in treating a cardiomyopathy such as Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasixdisease associated neurodegeneration (e.g., Parkinson's disease such as levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs), or with adjunctive agents that may not be effective alone, but may contribute to the efficacy of the active agent.

In embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In embodiments, co-administration can be accomplished by co-formulation, i.e., preparing a single pharmaceutical composition including both active agents. In other embodiments, the active agents can be formulated separately. In embodiments, the active and/or adjunctive agents may be linked or conjugated to one another. In embodiments, the compounds described herein may be combined with treatments for neurodegeneration such as surgery. In embodiments, the compounds described herein may be combined with treatments for cardiomyopathy such as surgery.

“PINK1” is used according to its common, ordinary meaning and refers to proteins of the same or similar names and functional fragments and homologs thereof. The term includes and recombinant or naturally occurring form of PINK1 (e.g., “PTEN induced putative kinase 1”; Entrez Gene 65018, OMIM 608309, UniProtKB Q9BXM7, and/or RefSeq (protein) NP_115785.1). The term includes PINK1 and variants thereof that maintain PINK1 activity (e.g., within at least about 30%, 40%, 50%, 60%, 70%, 80%, 90%, 95%, or 100% activity as compared to PINK1).

The term “neo-substrate” refers to a composition that is structurally similar to a composition that is a substrate for a protein or enzyme during the normal functioning of the protein or enzyme, but that is structurally distinct from the normal substrate of the protein or enzyme. In some embodiments, the composition comprises a neo-substrate. In embodiments, the neo-substrate is a better substrate for the protein or enzyme than the normal substrate (e.g., the reaction kinetics are better (e.g., faster), binding is stronger, turnover rate is higher, reaction is more productive, equilibrium favors product formation). In embodiments, the neo-substrate is a derivative of adenine, adenosine, AMP, ADP, or ATP. In embodiments, the neo-substrate is a substrate for PINK1. In embodiments, the neo-substrate is an N6 substituted adenine, adenosine, AMP, ADP, or ATP.

The term “derivative” as applied to a phosphate containing, monophosphate, diphosphate, or triphosphate group or moiety refers to a chemical modification of such group wherein the modification may include the addition, removal, or substitution of one or more atoms of the phosphate containing, monophosphate, diphosphate, or triphosphate group or moiety. In embodiments, such a derivative is a prodrug of the phosphate containing, monophosphate, diphosphate, or triphosphate group or moiety, which is converted to the phosphate containing, monophosphate, diphosphate, or triphosphate group or moiety from the derivative following administration to a subject, patient, cell, biological sample, or following contact with a subject, patient, cell, biological sample, or protein (e.g., enzyme). In an embodiment, a triphosphate derivative is a gamma-thio triphosphate. In an embodiment, a derivative is a phosphoramidate. In embodiments, the derivative of a phosphate containing, monophosphate, diphosphate, or triphosphate group or moiety is as described in Murakami et al. J. Med Chem., 2011, 54, 5902; Sofia et al., J. Med Chem. 2010, 53, 7202; Lam et al. ACC, 2010, 54, 3187; Chang et al., ACS Med Chem Lett., 2011, 2, 130; Furman et al., Antiviral Res., 2011, 91, 120; Vernachio et al., ACC, 2011, 55, 1843; Zhou et al, AAC, 2011, 44, 76; Reddy et al., BMCL. 2010, 20, 7376; Lam et al., J. Virol., 2011, 85, 12334; Sofia et al., J. Med. Chem., 2012, 55, 2481, Hecker et al., J. Med. Chem., 2008, 51, 2328; or Rautio et al., Nature Rev. Drug. Discov, 2008, 7, 255, all of which are incorporated herein by reference in their entirety for all purposes.

The term “mitochondrial dysfunction” is used in accordance with its ordinary meaning and refers to aberrant activity of function of the mitochondria, including for example aberrant respiratory chain activity, reactive oxygen species levels, calcium homeostasis, programmed cell death mediated by the mitochondria, mitochondrial fusion, mitochondrial fission, mitophagy, lipid concentrations in the mitochondrial membrane, and/or mitochondrial permeability transition.

As used herein, the term “mitochondrial disease” refers to a disease, disorder, or condition in which the function of a subject's mitochondria becomes impaired or dysfunctional. Examples of mitochondrial diseases that may be treated with a compound or method described herein include Alzheimer's disease, amyotrophic lateral sclerosis, Asperger's Disorder, Autistic Disorder, bipolar disorder, cancer, cardiomyopathy, Charcot Marie Tooth disease (CMT, including various subtypes such as CMT type 2b and 2b), Childhood Disintegrative Disorder (CDD), diabetes, diabetic nephropathy, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration, Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease, Peroneal muscular atrophy (PMA), Pervasive Developmental Disorder Not Otherwise Specified (PDD-NOS), renal tubular acidosis, Rett's Disorder, Schizophrenia, and types of stroke.

The term “oxidative stress” is used in accordance with its ordinary meaning and refers to aberrant levels of reactive oxygen species.

As used herein, the term “animal” includes, but is not limited to, humans and non-human vertebrates such as wild, domestic, and farm animals.

As used herein, the term “antagonize” or “antagonizing” means reducing or completely eliminating an effect, such as an activity of GPR109a.

As used herein, the phrase “anti-receptor effective amount” of a compound can be measured by the anti-receptor effectiveness of the compound. In some embodiments, an anti-receptor effective amount inhibits an activity of the receptor by at least 10%, by at least 20%, by at least 30%, by at least 40%, by at least 50%, by at least 60%, by at least 70%, by at least 80%, by at least 90%, or by at least 95%. In some embodiments, an “anti-receptor effective amount” is also a “therapeutically effective amount” whereby the compound reduces or eliminates at least one effect of GPR109a. In some embodiments, the effect is the B-arrestin effect. In some embodiments, the effect is the G-protein mediated effect.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carters can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the phrase “inhibiting activity,” such as enzymatic or receptor activity means reducing by any measurable amount the activity of PINK1.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “prodrug” means a derivative of a known direct acting drug, which derivative has enhanced delivery characteristics and therapeutic value as compared to the drug, and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the invention can optionally exclude one or more embodiment for purposes of claiming the subject matter.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

### B. COMPOUNDS

In various embodiments, the invention relates to compounds useful in treating disorders associated with PINK1 kinase activity such as, for example, a neurodegenerative disease, a mitochondrial disease, fibrosis, and/or cardiomyopathy.

In various embodiments, the compounds are useful in treating a disorder associated with PINK1 kinase activity in a mammal. In a further embodiment, the compounds are useful in treating PINK1 kinase activity in a human.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In some embodiments, provided are compounds having a structure represented by a formula:

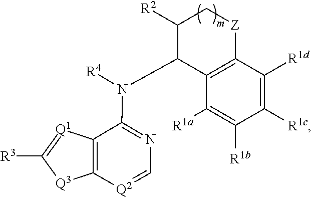

wherein m is 0 or 1; wherein each of Q¹ and Q² is independently N or CH; wherein Q³ is CH₂ or NH: wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —(CH₂)ₙCy¹, —O(CH₂)ₙCy¹, —NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof. In further embodiments, R² is selected from —O(CH₂)ₙCy¹, —NR₁₃(CH₂)ₙCy¹, and Cy¹; Cy¹ is a C3-C9 heterocycle having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and R⁴ is hydrogen.

In some embodiments, provided are compounds having a structure represented by a formula:

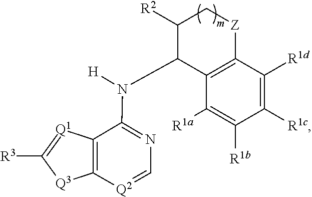

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, NR¹³(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R³ is a 3- to 6-membered cycloalkyl, a C1-C6 haloalkyl, C1-C6 haloalkoxy, or C1-C6 halohydroxyalkyl, or a pharmaceutically acceptable salt thereof.

In some embodiments, provided are compounds having a structure represented by a formula selected from:

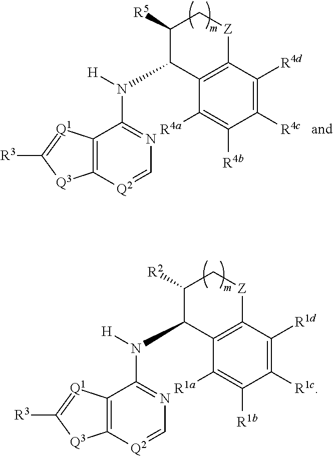

In some embodiments, provided are compounds having a structure represented by a formula selected from:

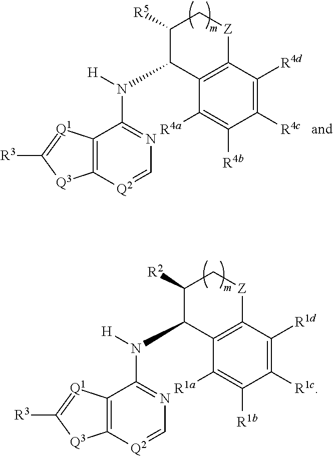

In some embodiments, provided is a compound having a structure:

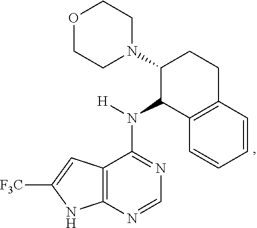

or a pharmaceutically acceptable salt thereof.

In some embodiments, provided is a compound having a structure represented by a formula:

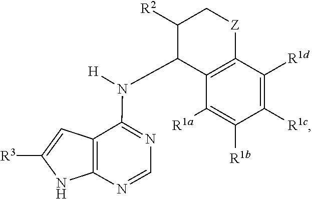

or a pharmaceutically acceptable salt thereof.

In some embodiments, provided is a compound having a structure represented by a formula:

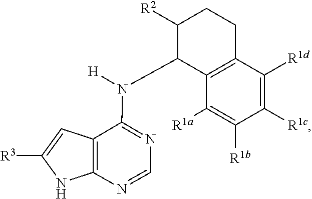

or a pharmaceutically acceptable salt thereof.

In some embodiments, provided is a compound having a structure represented by a formula selected from:

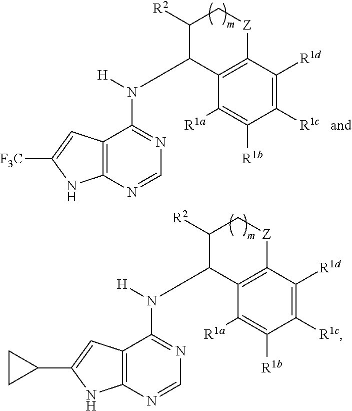

or a pharmaceutically acceptable salt thereof.

In some embodiments, provided is a compound having a structure represented by a formula selected from:

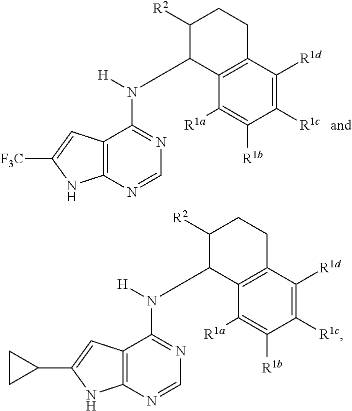

or a pharmaceutically acceptable salt thereof.

In some embodiments, the compound is selected from:

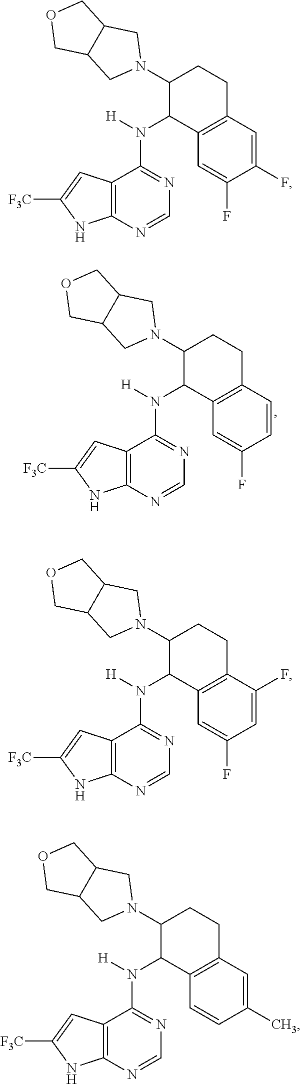 [SMILES: Fc1cc2c(cc1F)C(Nc1ncnc3[nH]c(C(F)(F)F)cc13)C(N1CC3COCC3C1)CC2 ; Fc1ccc2c(c1)C(Nc1ncnc3[nH]c(C(F)(F)F)cc13)C(N1CC3COCC3C1)CC2 ; Fc1cc(F)c2c(c1)C(Nc1ncnc3[nH]c(C(F)(F)F)cc13)C(N1CC3COCC3C1)CC2 ; Cc1ccc2c(c1)CCC(N1CC3COCC3C1)C2Nc1ncnc2[nH]c(C(F)(F)F)cc12]

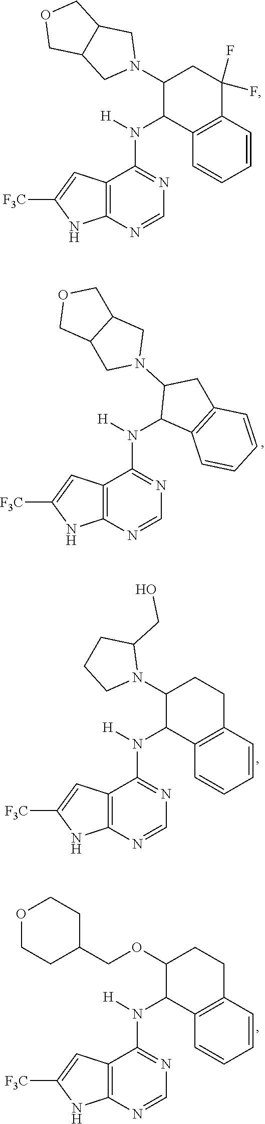 [SMILES: FC(F)(F)c1cc2c(NC3c4ccccc4C(F)(F)CC3N3CC4COCC4C3)ncnc2[nH]1 ; FC(F)(F)c1cc2c(NC3c4ccccc4CC3N3CC4COCC4C3)ncnc2[nH]1 ; OCC1CCCN1C1CCc2ccccc2C1Nc1ncnc2[nH]c(C(F)(F)F)cc12 ; FC(F)(F)c1cc2c(NC3c4ccccc4CCC3OCC3CCOCC3)ncnc2[nH]1]

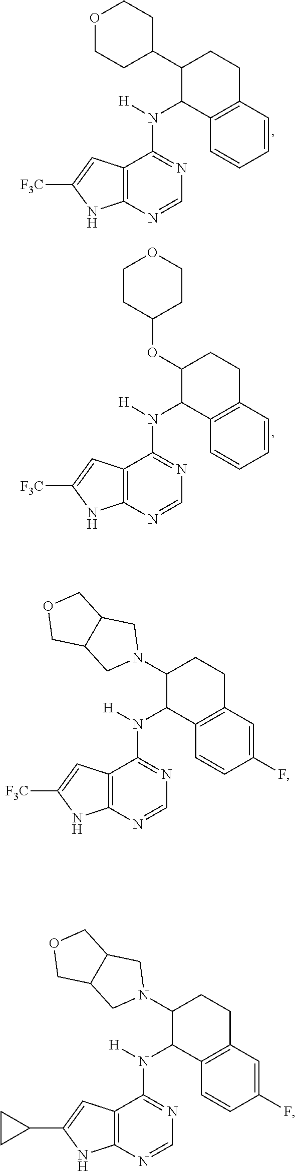

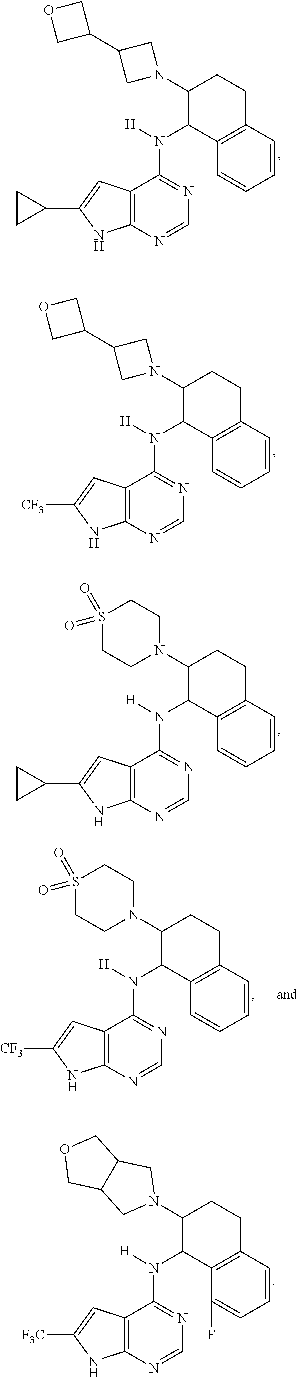 [SMILES: c1ccc2c(c1)CCC(N1CC(C3COC3)C1)C2Nc1ncnc2[nH]c(C3CC3)cc12 ; FC(F)(F)c1cc2c(NC3c4ccccc4CCC3N3CC(C4COC4)C3)ncnc2[nH]1 ; O=S1(=O)CCN(C2CCc3ccccc3C2Nc2ncnc3[nH]c(C4CC4)cc23)CC1 ; O=S1(=O)CCN(C2CCc3ccccc3C2Nc2ncnc3[nH]c(C(F)(F)F)cc23)CC1 ; Fc1cccc2c1C(Nc1ncnc3[nH]c(C(F)(F)F)cc13)C(N1CC3COCC3C1)CC2]

In some embodiments, the compound is selected from:

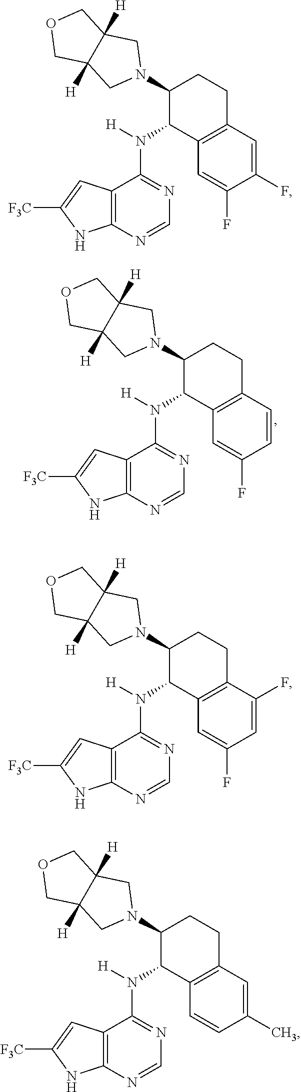

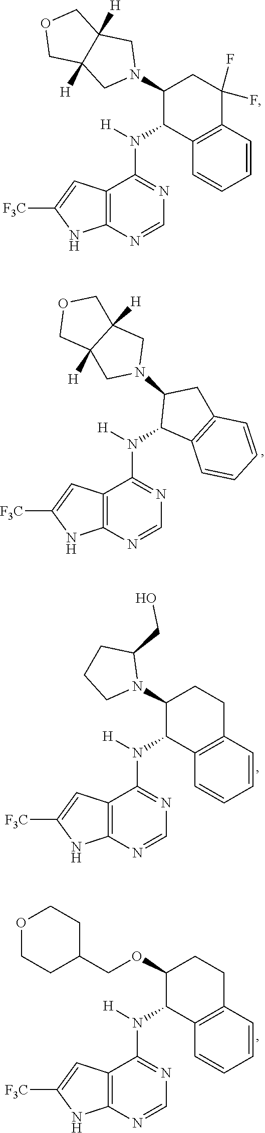 [SMILES: FC(F)(F)c1cc2c(N[C@H]3c4ccccc4C(F)(F)C[C@@H]3N3C[C@H]4COC[C@H]4C3)ncnc2[nH]1 ; FC(F)(F)c1cc2c(N[C@H]3c4ccccc4C[C@@H]3N3C[C@H]4COC[C@H]4C3)ncnc2[nH]1 ; OC[C@@H]1CCCN1[C@H]1CCc2ccccc2[C@@H]1Nc1ncnc2[nH]c(C(F)(F)F)cc12 ; FC(F)(F)c1cc2c(N[C@H]3c4ccccc4CC[C@@H]3OCC3CCOCC3)ncnc2[nH]1]

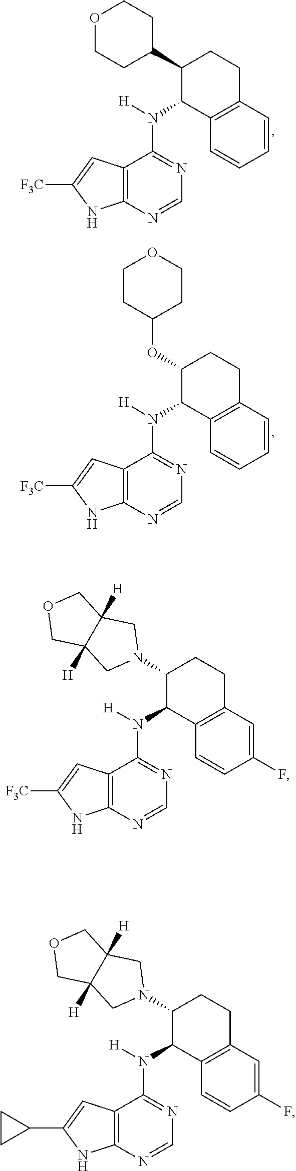

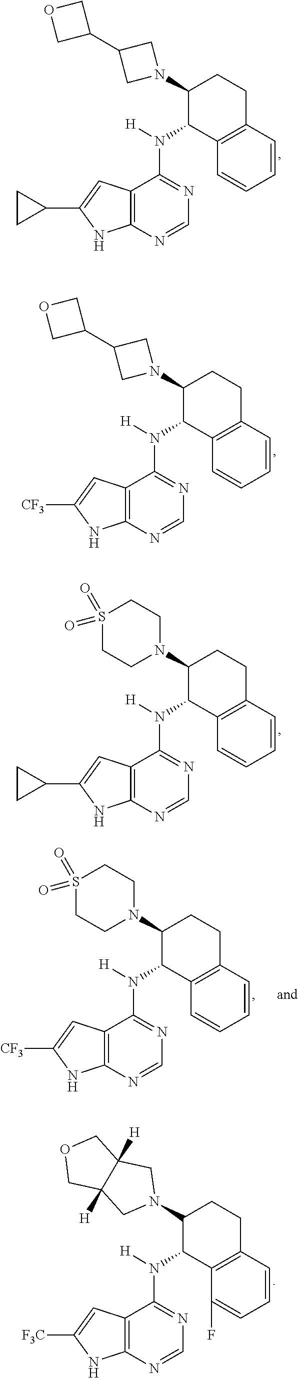

In some embodiments, the compound is selected from:

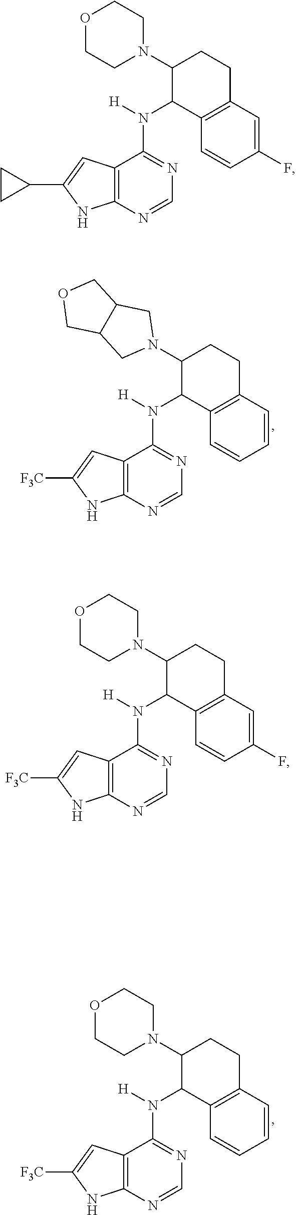 [SMILES: Fc1ccc2c(c1)CCC(N1CCOCC1)C2Nc1ncnc2[nH]c(C3CC3)cc12 ; FC(F)(F)c1cc2c(NC3c4ccccc4CCC3N3CC4COCC4C3)ncnc2[nH]1 ; Fc1ccc2c(c1)CCC(N1CCOCC1)C2Nc1ncnc2[nH]c(C(F)(F)F)cc12 ; FC(F)(F)c1cc2c(NC3c4ccccc4CCC3N3CCOCC3)ncnc2[nH]1]

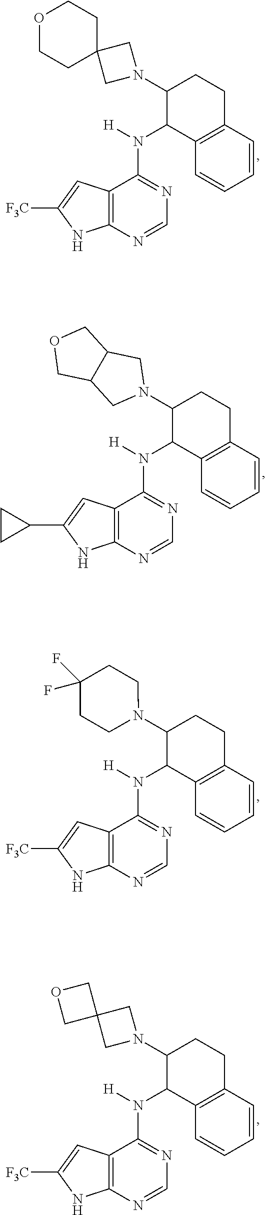 [SMILES: FC(F)(F)c1cc2c(NC3c4ccccc4CCC3N3CC4(CCOCC4)C3)ncnc2[nH]1 ; c1ccc2c(c1)CCC(N1CC3COCC3C1)C2Nc1ncnc2[nH]c(C3CC3)cc12 ; FC1(F)CCN(C2CCc3ccccc3C2Nc2ncnc3[nH]c(C(F)(F)F)cc23)CC1 ; FC(F)(F)c1cc2c(NC3c4ccccc4CCC3N3CC4(COC4)C3)ncnc2[nH]1]

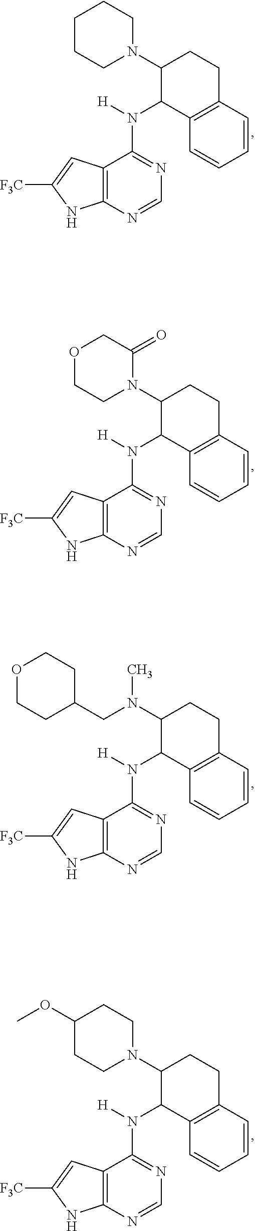 [SMILES: FC(F)(F)c1cc2c(NC3c4ccccc4CCC3N3CCCCC3)ncnc2[nH]1 ; O=C1COCCN1C1CCc2ccccc2C1Nc1ncnc2[nH]c(C(F)(F)F)cc12 ; CN(CC1CCOCC1)C1CCc2ccccc2C1Nc1ncnc2[nH]c(C(F)(F)F)cc12 ; COC1CCN(C2CCc3ccccc3C2Nc2ncnc3[nH]c(C(F)(F)F)cc23)CC1]

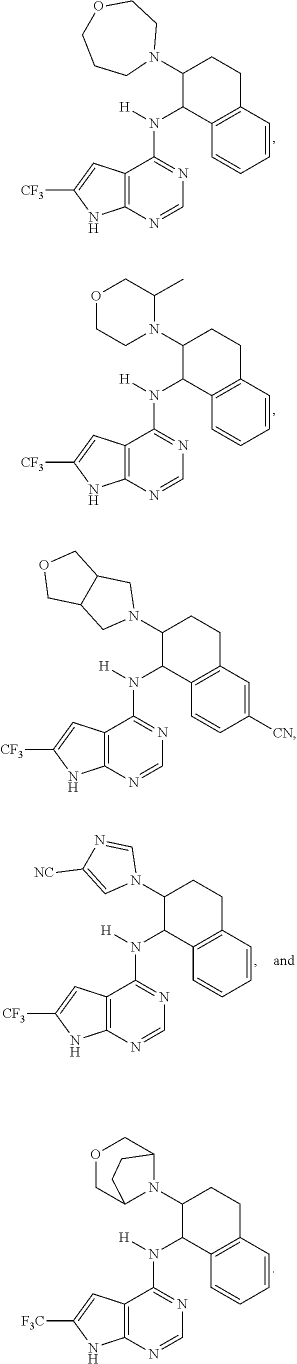 [SMILES: FC(F)(F)c1cc2c(NC3c4ccccc4CCC3N3CCCOCC3)ncnc2[nH]1 ; CC1COCCN1C1CCc2ccccc2C1Nc1ncnc2[nH]c(C(F)(F)F)cc12 ; N#Cc1ccc2c(c1)CCC(N1CC3COCC3C1)C2Nc1ncnc2[nH]c(C(F)(F)F)cc12 ; N#Cc1cn(C2CCc3ccccc3C2Nc2ncnc3[nH]c(C(F)(F)F)cc23)cn1 ; FC(F)(F)c1cc2c(NC3c4ccccc4CCC3N3C4CCC3COC4)ncnc2[nH]1]

In some embodiments, the compound is selected from:

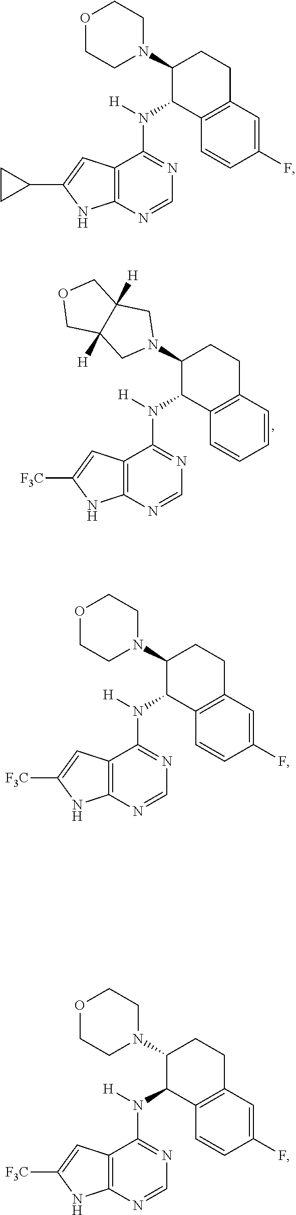 [SMILES: Fc1ccc2c(c1)CC[C@H](N1CCOCC1)[C@H]2Nc1ncnc2[nH]c(C3CC3)cc12 ; FC(F)(F)c1cc2c(N[C@H]3c4ccccc4CC[C@@H]3N3C[C@H]4COC[C@H]4C3)ncnc2[nH]1 ; Fc1ccc2c(c1)CC[C@H](N1CCOCC1)[C@H]2Nc1ncnc2[nH]c(C(F)(F)F)cc12 ; Fc1ccc2c(c1)CC[C@@H](N1CCOCC1)[C@@H]2Nc1ncnc2[nH]c(C(F)(F)F)cc12]

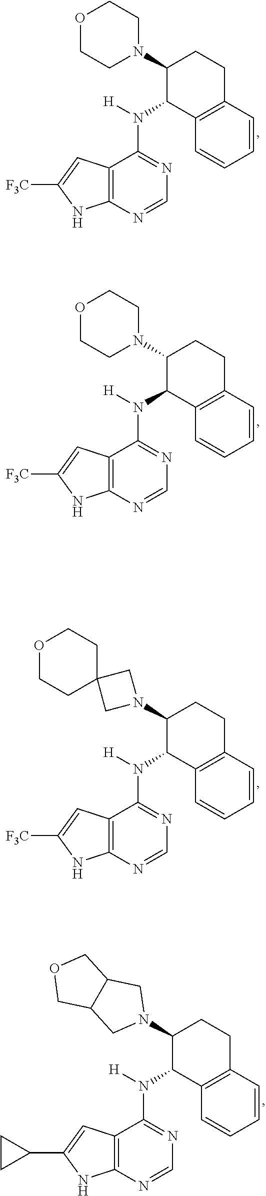 [SMILES: FC(F)(F)c1cc2c(N[C@H]3c4ccccc4CC[C@@H]3N3CCOCC3)ncnc2[nH]1 ; FC(F)(F)c1cc2c(N[C@@H]3c4ccccc4CC[C@H]3N3CCOCC3)ncnc2[nH]1 ; FC(F)(F)c1cc2c(N[C@H]3c4ccccc4CC[C@@H]3N3CC4(CCOCC4)C3)ncnc2[nH]1 ; c1ccc2c(c1)CC[C@H](N1CC3COCC3C1)[C@H]2Nc1ncnc2[nH]c(C3CC3)cc12]

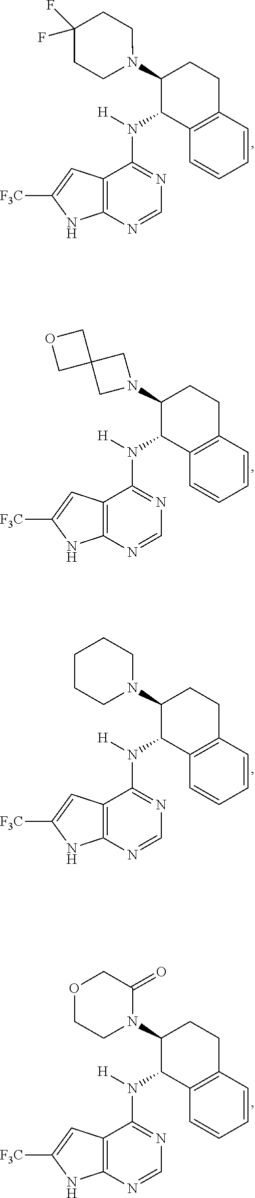 [SMILES: FC1(F)CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CC4(COC4)C3)ncnc2[nH]1 ; FC(F)(F)c1cc2c(N[C@H]3c4ccccc4CC[C@@H]3N3CCCCC3)ncnc2[nH]1 ; O=C1COCCN1[C@H]1CCc2ccccc2[C@@H]1Nc1ncnc2[nH]c(C(F)(F)F)cc12]

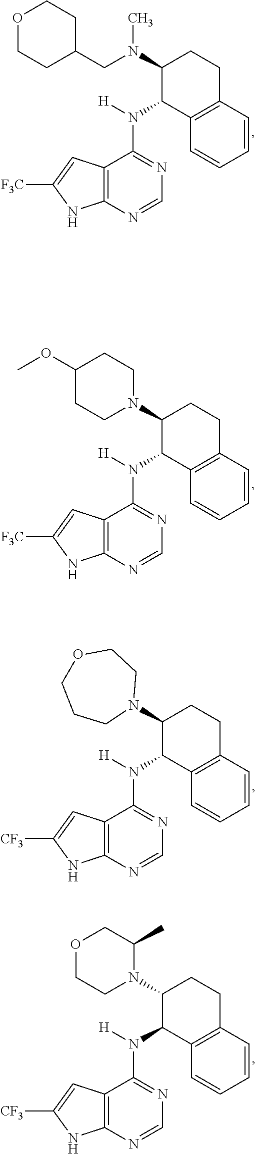 [SMILES: CN(CC1CCOCC1)[C@H]1CCc2ccccc2[C@@H]1Nc1ncnc2[nH]c(C(F)(F)F)cc12 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CCCOCC3)ncnc2[nH]1 ; C[C@@H]1COCCN1[C@@H]1CCc2ccccc2[C@H]1Nc1ncnc2[nH]c(C(F)(F)F)cc12]

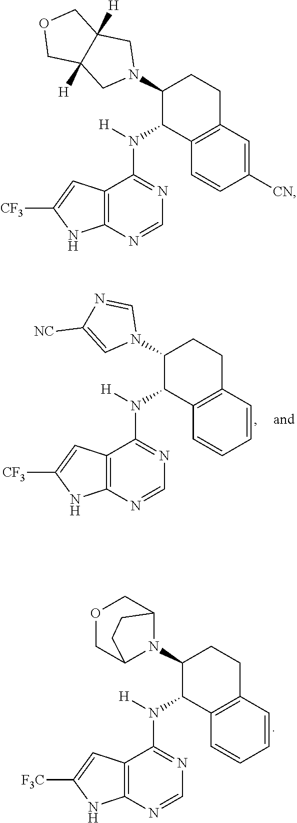 [SMILES: N#Cc1ccc2c(c1)CC[C@H](N1C[C@H]3COC[C@H]3C1)[C@H]2Nc1ncnc2[nH]c(C(F)(F)F)cc12 ; N#Cc1cn([C@@H]2CCc3ccccc3[C@@H]2Nc2ncnc3[nH]c(C(F)(F)F)cc23)cn1 ; FC(F)(F)c1cc2c(N[C@H]3c4ccccc4CC[C@@H]3N3C4CCC3COC4)ncnc2[nH]1]

In some embodiments, Q¹ is CH, Q² is N, and Q³ is NH.

Thus, in some embodiments, m is 0 or 1. In further embodiments, m is 0. In still further embodiments, m is 1.

In some embodiments, n, when present, is 0, 1, or 2. In further embodiments, n, when present, is 0 or 1. In still further embodiments, n, when present, is 1 or 2. In yet further embodiments, n, when present, is 0 or 2. In even further embodiments, n, when present, is 0. In still further embodiments, n, when present, is 0. In yet further embodiments, n, when present, is 1. In even further embodiments, n, when present, is 2.

Specific examples of compounds are provided in the EXAMPLES section and are included herein. Pharmaceutically acceptable salts as well as the neutral forms of these compounds are also included.

a. Q¹ and Q² Groups

In some embodiments, each of Q¹ and Q² is independently N or CH. In further embodiments, each of Q¹ and Q² is CH. In still further embodiments, each of Q¹ and Q² is N.

In yet a further embodiment, Q¹ is N and Q² is CH. In an even further embodiment, Q¹ is CH and Q² is N.

In some embodiments. Q¹ is CH or N. In a further embodiment, Q¹ is N. In a still further embodiment, Q¹ is CH,

In some embodiments, Q² is CH or N. In a further embodiment, Q² is CH. In a still further embodiment, Q² is NH.

b. Q³ Groups

In some embodiments, Q³ is CH₂ or NH. In further embodiments, Q² is CH₂. In still further embodiments, Q² is NH.

c. Z Groups

In some embodiments, Z is CR^{11a}R^{11b}, NR¹², or O. In further embodiments, Z is CR^{11a}R^{11b} or NR¹². In still further embodiments, Z is NR² or 0.

In some embodiments, Z is CR^{11a}R^{11b} or O. In further embodiments, Z is CR^{11a}R^{11b}.

In still further embodiments, Z is CH₂. In yet further embodiments, Z is O.

In some embodiments, Z is NR¹².

d. R^{1A}, R^{1B}, R^{1C}, and R^{1D} Groups (R¹ Groups)

In some embodiments, each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, each of R^{1a}, R^{1b}, R^{1c}, and R^{1d} is independently selected from hydrogen, F, —Cl, —CN, —NH₂, —OH, —NO₂, methyl, ethyl, n-propyl, isopropyl, ethenyl, propenyl, —CHY, —C₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, —CH₂CH₂CH₂CN, —CH₂OH, —CH₂CH₂OH, —CH(CF₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH)CH₂Cl, —OCH₂CH₂CH₂Cl, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{1a}, R^{1b}, R^{1c}, and R^{1d} is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂C, —CH₂CN, —CH₂CH₂CN, —CH₂OH, —CH₂CH₂OH, methoxy, ethoxy, —OCF₃, —OCHF₂, —OCH₂F, —OCH₂CH₂P, —OCCl₃, —OCHCl₂, —OCH₂Cl, —OCH₂CH₂Cl, —NHCH₃. —NHCH₂CH₃, —N(CH₃)₂, and —N(CH₃)CH₂CH₃. In yet further embodiments, each of R^{1a}, R^{1b}, R^{1c}, and R^{1d} is independently selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, each of R^{1a}, R^{1b}, R^{1c}, and R^{1d} is hydrogen.

In various embodiments, each of R^{1a}, R^{1b}, R^{1c}, and R^{1d} is independently selected from hydrogen, halogen, —CN, —NH₂, —OH, —NO₂, C1-C4 hydroxyalkyl, C1-C4 haloalkoxy, C1-C4 alkoxy. In further embodiments, each of R^{1a}, R^{1b}, R^{1c}, and R^{1d} is independently selected from hydrogen, F, —Cl, —CN, —NH₂, —OH, —NO₂, —CH₂OH, —CH₂CH₂OH, —CH(CH₃)CH₂OH, —CH₂CH₂CH₂O, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHC₂, —OCH₂Cl, —OCH₂CH₂Cl, —OCH(CH₃)CH₂Cl, and —OCH₂CH₂CH₂Cl. In still further embodiments, each of R^{1a}, R^{1b}, R^{1c}, and R^{1d} is independently selected from hydrogen, F, —Cl, —CN, —NH₂, —OH, —NO₂, —CH₂OH, —CH₂CH₂OH, methoxy, ethoxy, —OCF₃, —OCHF₂, —OCH₂F. —OCH₂CH₂F, —OCCl₃, —OCHCl₂, —OCH₂C, and —OCH₂CH₂Cl. In yet further embodiments, each of R^{1a}, R^{1b}, R^{1c}, and R^{1d} is independently selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, each of R^{1a}, R^{1b}, R^{1c}, and R^{1d} is independently selected from hydrogen, halogen, —CN, —NH₂, —OH, —NO₂, C1-C4 alkylamino, and (C1-C4)(C1-C4) dialkylamino. In further embodiments, each of R^{1a}, R^{1b}, R^{1c}, and R^{1d} is independently selected from hydrogen, F, —Cl, —CN, —NH₂, —OH, —NO₂, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{1a}, R^{1b}, R^{1c}, and R^{1d} is independently selected from hydrogen, F, —Cl, —CN, —NH₂, —OH, —NO₂, —NHCH₃, —NHCH₂CH₃, —N(CH₃)₂, and —N(CH₃)CH₂CH₃. In yet further embodiments, each of R^{1a}, R^{1b}, R^{1c}, and R^{1d} is independently selected from hydrogen, F, —Cl, —CN, —NH₂, —OH, —NO₂, —NHCH₃, and —N(CH₃)₂.

In various embodiments, each of R^{1a}, R^{1b}, R^{1c}, and R^{1d} is independently selected from hydrogen, halogen, —CN, —NH₂, —OH, —NO₂, C1-C4 haloalkyl, and C1-C4 cyanoalkyl. In further embodiments, each of R^{1a}, R^{1b}, R^{1c}, and R^{1d} is independently selected from hydrogen, F, —Cl, —CN, —NH₂, —OH, —NO₂, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, and —CH₂CH₂CH₂CN. In still further embodiments, each of R^{1a}, R^{1b}, R^{1c}, and R^{1d} is independently selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, each of R^{1a}, R^{1b}, R^{1c}, and R^{1d} is independently selected from hydrogen, F, —Cl, —CN, —NH₂, —OH, —NO₂, —CH₂F, —CH₂Cl, and —CH₂CN.

In various embodiments, each of R^{1a}, R^{1b}, R^{1c}, and R^{1d} is independently selected from hydrogen, halogen, —CN, —NH₂, —OH, —NO₂, C1-C4 alkyl, and C2-C4 alkenyl. In further embodiments, each of R^{1a}, R^{1b}, R^{1c}, and R^{1d} is independently selected from hydrogen, F, —Cl, —CN, —NH₂, —OH, —NO₂, methyl, ethyl, n-propyl, isopropyl, ethenyl, and propenyl. In still further embodiments, each of R^{1a}, R^{1b}, R^{1c}, and R^{1d} is independently selected from hydrogen, F, —Cl, —CN, —NH₂, —OH, —NO₂, methyl, ethyl, and ethenyl. In yet further embodiments, each of R^{1a}, R^{1b}, R^{1c}, and R^{1d} is independently selected from hydrogen, F, —Cl, —CN, —NH₂, —OH, —NO₂, and methyl.

In various embodiments, each of R^{1a}, R^{1b}, R^{1c}, and R^{1d} is independently selected from hydrogen and C1-C4 alkyl. In further embodiments, each of R^{1a}, R^{1b}, R^{1c}, and R^{1d} is independently selected from hydrogen, methyl, ethyl, n-propyl, and isopropyl. In still further embodiments, each of R^{1a}, R^{1b}, R^{1c}, and R^{1d} is independently selected from hydrogen, methyl, and ethyl. In yet further embodiments, each of R^{1a}, R^{1b}, R^{1c}, and R^{1d} is independently selected from hydrogen and methyl.

In various embodiments, each of R^{1a}, R^{1b}, R^{1c}, and R^{1d} is independently selected from hydrogen and halogen. In further embodiments, each of R^{1a}, R^{1b}, R^{1c}, and R^{1d} is independently selected from hydrogen, —F, —Cl, and —Br. In still further embodiments, each of R^{1a}, R^{1b}, R^{1c}, and R^{1d} is independently selected from hydrogen, —F, and —Cl. In yet further embodiments, each of R^{1a}, R^{1b}, R^{1c}, and R^{1d} is independently selected from hydrogen and —Cl. In still further embodiments, each of R^{1a}, R^{1b}, R^{1c}, and R^{1d} is independently selected from hydrogen and —F.

In some embodiments, each of R^{1a}, R^{1b}, R^{1c}, and R^{1d} is independently hydrogen, halogen, or C1-C4 alkyl. In further embodiments, each of R^{1a}, R^{1b}, R^{1c}, and Rid is independently hydrogen, —F, —Cl, —Br, methyl, ethyl, n-propyl, or isopropyl. In still further embodiments, each of R^{1a}, R^{1b}, R^{1c}, and R^{1d} is independently hydrogen, —F, —Cl, methyl, and ethyl. In yet further embodiments, each of R^{1a}, R^{1b}, R^{1c}, and R^{1d} is independently hydrogen, —F, and methyl.

e. R² Groups

In some embodiments, R² is selected from —(CH₂)ₙCy¹, —O(CH₂)ₙCy¹, —NR¹³(CH₂)ₙCy¹, —CH(OH)Cy¹, and Cy¹. In further embodiments, R² is selected from —(CH₂)ₙCy¹ and —CH(OH)Cy¹. In still further embodiments, R² is —(CH₂)ₙCy¹. In yet further embodiments, R² is —CH(OH)Cy¹.

In some embodiments, R² is selected from —O(CH₂)ₙCy¹, —NR³(CH₂)ₙCy¹, and Cy¹. In further embodiments, R² is selected from —OCy¹, —NR¹³Cy¹, —OCH₂Cy¹, —NR¹³CH₂Cy¹, and Cy. In still further embodiments, R² is selected from —OCy¹, —NR¹³Cy¹, and Cy¹.

In some embodiments, R² is selected from —O(CH₂)ₙCy¹ and —NR¹³(CH₂)ₙCy¹. In further embodiments, R² is selected from —OCy¹, —NR³Cy¹, —OCH₂Cy¹, and —NR¹³CH₂Cy¹. In still further embodiments, R² is selected from —OCy¹ and —NR¹³Cy¹.

In some embodiments, R² is Cy¹.

f. R³ Groups

In some embodiments, R³ is a 3- to 6-membered cycloalkyl, CL-C6 haloalkyl, C₁-C₆ haloalkoxy, or C1-C6 halohydroxyalkyl. In further embodiments, R³ is a 3- to 6-membered cycloalkyl, C1-C4 haloalkyl, C1-C4 haloalkoxy, or C1-C4 halohydroxyalkyl. In still further embodiments, R³ is a 3- to 6-membered cycloalkyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R³ is a 3- to 6-membered cycloalkyl, —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl, C1-C6 haloalkoxy, or C1-C6 halohydroxyalkyl. In further embodiments, R³ is C1-C4 haloalkyl, C1-C4 haloalkoxy, or C1-C4 halohydroxyalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R³ is —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl. In further embodiments, R³ is C1-C4 haloalkyl. In still further embodiments, R³ is —CF₃. —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is —CF₃, —CHF₂, —CH₂F, —CCl₃, —CHCl₂, or —CH₂C1.

In some embodiments, R³ is a 3- to 6-membered cycloalkyl. In further embodiments, R³ is a 3- to 5-membered cycloalkyl. In still further embodiments, R³ is a 3- to 4-membered cycloalkyl. In yet further embodiments, R³ is a 3-membered cycloalkyl. In an even further embodiment, R³ is a 4-membered cycloalkyl.

In some embodiments, R³ is hydrogen.

In some embodiments, R³ is hydrogen, halogen, (C1-C4)alkyl, or 3- to 6-membered cycloalkyl. In further embodiments. R³ is hydrogen.

In further embodiments, R³ is hydrogen, —F, —Cl, methyl, ethyl, n-propyl, isopropyl, or 3- to 6-membered cycloalkyl. In still further embodiments, R³ is hydrogen, —F, —Cl, methyl, ethyl, or 3- to 6-membered cycloalkyl. In yet further embodiments, R³ is hydrogen, F, —Cl, methyl, or 3- to 6-membered cycloalkyl.

In further embodiments, R³ is hydrogen or (C1-C4)alkyl. In still further embodiments, R³ is hydrogen, methyl, ethyl, n-propyl, or isopropyl. In yet further embodiments, R³ is hydrogen, methyl, or ethyl. In an even further embodiment, R³ is hydrogen or ethyl. In still further embodiments, R³ is hydrogen or methyl.

In further embodiments, R³ is (C1-C₄)alkyl. In still further embodiments, R³ is methyl, ethyl, n-propyl, or isopropyl. In yet further embodiments, R¹ is methyl or ethyl. In an even further embodiment, R³ is ethyl. In still further embodiments, R³ is methyl.

In further embodiments, R³ is (C1-C4)alkyl. In still further embodiments, R³ is methyl, ethyl, n-propyl, isopropyl, halogenated methyl, halogenated ethyl, halogenated propyl, CF₃, CCl₃, or CBr₃. In yet further embodiments, R³ is methyl or ethyl. In an even further embodiment, R³ is ethyl. In still further embodiments, R³ is methyl. In still further embodiments, R³ is CF₃, CCl₃, or CBr₃.

In further embodiments, R³ is hydrogen or halogen. In still further embodiments, R³ is hydrogen, —F, —Cl, or —Br. In yet further embodiments, R³ is hydrogen, —F, or —Cl. In an even further embodiment, R³ is hydrogen or —F. In still further embodiments, R³ is hydrogen or —Cl.

In further embodiments, R³ is halogen. In still further embodiments, R³ is —F, —Cl, or Br. In yet further embodiments, R³ is —F or —Cl. In an even further embodiment, R³ is —F. In still further embodiments, R³ is —Cl.

In further embodiments, R³ is hydrogen or 3- to 6-membered cycloalkyl. In still further embodiments, R³ is hydrogen, cyclopropyl, cyclobutyl, or cyclopentyl. In yet further embodiments, R³ is hydrogen, cyclopropyl, or cyclobutyl. In an even further embodiment, R³ is hydrogen or cyclopropyl. In some embodiments, R³ is not a methyl, ethyl or butyl. In some embodiments, R³ is not an acyclic alkyl chain comprising from about 1 to about 5 substituted or unsubstituted carbons.

In further embodiments, R³ is 3- to 6-membered cycloalkyl. In still further embodiments, R³ is 3- to 5-membered cycloalkyl. In yet further embodiments, R³ is 3- to 4-membered cycloalkyl. In an even further embodiment, R³ is cyclohexyl. In still further embodiments, R³ is cyclopentyl. In yet further embodiments, 3 is cyclobutyl. In an even further embodiment, R³ is cyclopropyl.

In further embodiments, R³ is a 3- to 6-membered cycloalkyl or a C1-C6 haloalkyl. In still further embodiments, R³ is cyclopropyl, cyclobutyl, cyclopentyl, CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is cyclopropyl, cyclobutyl, CF₃, —CHF₂, —CH₂F, —CH₂CF₃, —CH₂CHF₂, —CH₂CH₂F, —CCl₃, —CHCl₂, —CH₂Cl, or —CH₂CCl₃. In even further embodiments, R³ is cyclopropyl, CF₃, —CHF₂, —CH₂F, —CCl₃, or —CHCl₂.

In further embodiments, R³ is a 3-membered cycloalkyl or —CF₃. In still further embodiments, R³ is a 3-membered cycloalkyl. In yet further embodiments, R³ is —CF₃.

g. R⁴ Groups

In some embodiments, R⁴ is selected from hydrogen and C1-C4 alkyl. In further embodiments, R⁴ is selected from hydrogen, methyl, ethyl, n-propyl, and isopropyl. In still further embodiments, R⁴ is selected from hydrogen, methyl, and ethyl. In yet further embodiments, R⁴ is selected from hydrogen and ethyl. In an even further embodiment, R⁴ is selected from hydrogen and methyl.

In some embodiments, R⁴ is hydrogen.

In some embodiments, R⁴ is C1-C4 alkyl. In further embodiments, R⁴ is selected from methyl, ethyl, n-propyl, and isopropyl. In still further embodiments, R⁴ is selected from methyl and ethyl. In yet further embodiments, R⁴ is ethyl. In an even further embodiment, R⁴ is methyl.

h. R^{11A} and R^{11B} Groups (R¹¹ Groups)

In some embodiments, each of R^{11a} and R^{11b}, when present, is independently selected from hydrogen, halogen, —OH, and C1-C4 alkoxy, or wherein each of R^{11a} and R^{11b} when present, together comprise ═O.

In some embodiments, each of R^{11a} and R^{11b} when present, is independently selected from hydrogen, halogen, —OH, and C1-C4 alkoxy. In further embodiments, each of R^{11a} and R^{11b}, when present, is independently selected from hydrogen, —F, —Cl, —Br, —OH, methoxy, ethoxy, n-propoxy, and isopropoxy. In still further embodiments, each of R^{11a} and R^{11b} when present, is independently selected from hydrogen, —F, —Cl, —OH, methoxy, and ethoxy. In yet further embodiments, each of R^{11a} and R^{11b}, when present, is independently selected from hydrogen, —F, —OH, and methoxy.

In some embodiments, each of R^{11a} and R^{11b}, when present, is independently selected from hydrogen, —OH, and C1-C4 alkoxy. In further embodiments, each of R^{11a} and R^{11b}, when present, is independently selected from hydrogen, —OH, methoxy, ethoxy, n-propoxy, and isopropoxy. In still further embodiments, each of R^{11a} and R^{11b}, when present, is independently selected from hydrogen, —OH, methoxy, and ethoxy. In yet further embodiments, each of R^{11a} and R^{11b}, when present, is independently selected from hydrogen, —OH, and methoxy.

In some embodiments, each of R^{11a} and R^{11b}, when present, is independently selected from hydrogen and C1-C4 alkoxy. In further embodiments, each of R^{11a} and R^{11b}, when present, is independently selected from hydrogen, methoxy, ethoxy, n-propoxy, and isopropoxy. In still further embodiments, each of R^{11a} and R^{11b}, when present, is independently selected from hydrogen, methoxy, and ethoxy. In yet further embodiments, each of R^{11a} and R^{11b}, when present, is independently selected from hydrogen and methoxy.

In some embodiments, each of R^{11a} and R^{11b}, when present, is independently selected from hydrogen and —OH. In further embodiments, each of R^{11a} and R^{11b}, when present, is —OH. In still further embodiments, each of R^{11a} and R^{11b} when present, is hydrogen.

In some embodiments, each of R^{11a} and R^{11b}, when present, is independently selected from hydrogen and halogen. In further embodiments, each of R^{11a} and R^{11b}, when present, is independently selected from hydrogen, —F, —Cl, and —Br. In still further embodiments, each of R^{11a} and R^{11b}, when present, is independently selected from hydrogen, —F, and —Cl. In yet further embodiments, each of R^{11a} and R^{11b}, when present, is independently selected from hydrogen and —F.

In some embodiments, each of R^{11a} and R^{11b}, when present, together comprise ═O.

i. R¹² Groups

In some embodiments, R¹², when present, is hydrogen, C1-C4 alkyl, C3-C6 cycloalkyl, or —(C1-C4 alkyl)(C3-C6 cycloalkyl). In further embodiments, R¹², when present, is hydrogen, methyl, ethyl, n-propyl, isopropyl, cyclopropyl, cyclobutyl, cyclopentyl, CH₂(cyclopropyl), —CH₂CH₂(cyclopropyl), —CH₂CH₂CH₂(cyclopropyl), —CH(CH₃)CH₂(cyclopropyl), —CH₂(cyclobutyl), —CH₂CH₂(cyclobutyl), —CH₂CH₂CH₂(cyclobutyl), —CH(CH₃)CH₂(cyclobutyl), —CH₂(cyclopentyl), —CH₂CH₂(cyclopentyl), —CH₂CH₂CH₂(cyclopentyl), or —CH(CH₃)CH₂(cyclopentyl). In still further embodiments, R¹², when present, is hydrogen, methyl, ethyl, cyclopropyl, cyclobutyl, —CH₂(cyclopropyl), —CH₂CH₂(cyclopropyl), —CH₂(cyclobutyl), —CH₂Cl₂(cyclobutyl), —Cl₂(cyclopentyl), or —CH₂CH₂(cyclopentyl). In yet further embodiments, R¹², when present, is hydrogen, methyl, cyclopropyl, —CH₂(cyclopropyl), —CH₂(cyclobutyl), or —CH₂(cyclopentyl).

In some embodiments, R¹², when present, is hydrogen or C1-C4 alkyl. In further embodiments, R¹², when present, is hydrogen, methyl, ethyl, n-propyl, or isopropyl. In still further embodiments, R¹², when present, is hydrogen, methyl, or ethyl. In yet further embodiments, R¹², when present, is hydrogen or methyl.

In some embodiments, R¹², when present, is C1-C4 alkyl. In further embodiments, R¹², when present, is methyl, ethyl, n-propyl, or isopropyl. In still further embodiments, R¹², when present, is methyl or ethyl. In yet further embodiments, R¹², when present, is methyl.

In some embodiments, R¹², when present, is C3-C6 cycloalkyl or —(C1-C4 alkyl)(C3-C6 cycloalkyl). In further embodiments, R¹², when present, is cyclopropyl, cyclobutyl, cyclopentyl, —CH₂(cyclopropyl), —CH₂CH₂(cyclopropyl), —CH₂CH₂CH₂(cyclopropyl), —CH(CH₃)CH₂(cyclopropyl), —CH₂(cyclobutyl), —CH₂CH₂(cyclobutyl), —CH₂CH₂CH₂(cyclobutyl), —CH(CH₃)CH₂(cyclobutyl), —CH₂(cyclopentyl), —CH₂CH₂(cyclopentyl), —CH₂CH₂CH₂(cyclopentyl), or —CH(CH₃)CH₂(cyclopentyl). In still further embodiments, R¹², when present, is —CH₂(cyclopropyl), —CH₂CH₂(cyclopropyl), —CH₂(cyclobutyl), —CH₂CH₂(cyclobutyl), —CH₂(cyclopentyl), or —CH₂CH₂(cyclopentyl). In yet further embodiments, R¹², when present, is —CH₂(cyclopropyl), —CH₂(cyclobutyl), or —CH₂(cyclopentyl).

In some embodiments, R¹², when present, is hydrogen.

j. R¹³ Groups

In some embodiments, R¹³, when present, is selected from hydrogen and C1-C4 alkyl. In further embodiments, R¹³, when present, is selected from hydrogen, methyl, ethyl, n-propyl, and isopropyl. In still further embodiments, R¹³, when present, is selected from hydrogen, methyl, and ethyl. In yet further embodiments, R¹³, when present, is selected from hydrogen and ethyl. In even further embodiments, R¹³, when present, is selected from hydrogen and methyl.

In some embodiments, R¹³, when present, is C1-C4 alkyl. In further embodiments, R¹³, when present, is selected from methyl, ethyl, n-propyl, and isopropyl. In still further embodiments, R¹³, when present, is selected from methyl and ethyl. In yet further embodiments, R¹³, when present, is ethyl. In even further embodiments, R¹³, when present, is methyl.

In some embodiments, R³, when present, is hydrogen.

k. R¹⁴ Groups

In some embodiments, R¹¹, when present, is selected from —OH, —NH₂, —O(C1-C4 alkyl), —NH(C1-C4 alkyl), and —N(C1-C4 alkyl)(C1-C4 alkyl). In further embodiments, R¹⁴, when present, is selected from —OH, —NH₂, —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃)₂, and —N(CH₃)CH₂CH₂CH₃. In still further embodiments, R¹⁴, when present, is selected from —OH, —NH₂, —OCH₃, —OCH₂CH₃, —NHCH₃, —NHCH₂CH₃, —N(CH₃)₂, and —N(CH₃)CH₂CH₃. In yet further embodiments, R¹⁴, when present, is selected from —OH, —NH₂, —OCH₃, —NHCH₃, and —N(CH₃)₂.

In some embodiments, R¹⁴, when present, is selected from —OH, and —O(C1-C4 alkyl). In further embodiments, R¹⁴, when present, is selected from —OH, —OCH₃, —OCH₂CH₃, —OCH(CH₃)₂, and —OCH₂CH₂CH₃. In still further embodiments, R¹⁴, when present, is selected from —OH, —OCH₃, and —OCH₂CH₃. In yet further embodiments, R¹⁴, when present, is selected from —OH and —OCH₃.

In some embodiments, R¹⁴, when present, is selected from —NH₂, —NH(C1-C4 alkyl), and —N(C1-C4 alkyl)(C1-C4 alkyl). In further embodiments, R¹⁴, when present, is selected from —NIH, —NHCH₃, —NHCH₂CH₃, —NHCH(CH₃)₂, —NHCH₂CH₂CH₃, —N(CH₃)₂, —N(CH₃)CH₂CH₃, —N(CH₃)CH(CH₃)₂, and —N(CH₃)CH₂CH₂CH₃. In still further embodiments, R¹⁴, when present, is selected from —NH₂, —NHCH₃, —NHCH₂CH₃, —N(CH₃)₂, and —N(CH₃)CH₂CH₃. In yet further embodiments, R¹⁴, when present, is selected from —NH₂, —NHCH₃, and —N(CH₃)₂.

In some embodiments, R¹⁴, when present, is selected from —OH and —NH₂. In further embodiments, R¹⁴, when present, is —OH. In still further embodiments, R¹⁴, when present, is —NH₂.

### 1. Cy¹ Groups

In some embodiments. Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino.

In some embodiments, Cy¹ is a C4-C9 cycloalkyl substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C₁-C₄ haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. Examples of C4-C9 cycloalkyls include, but are not limited to, cyclobutyl, cyclopentyl, cyclohexyl, and spiro[2.4]heptane. In further embodiments, Cy¹ is a C4-C9 cycloalkyl substituted with 0, 1, or 2 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, CJ-C4 alkylamino, and (C₁-C₄)(C1-C4) dialkylamino. In still further embodiments, Cy¹ is a C4-C9 cycloalkyl substituted with 0 or 1 group selected from halogen, —CN, —NH₂, —OH, —NO₂, ═O, C₃-C₆ cycloalkyl, C2-C5 heterocycloalkyl, C₁-C₄ alkyl, C2-C4 alkenyl, C1-C4 haloalkyl, C1-C4 cyanoalkyl, C₁-C₄ hydroxyalkyl, C₁-C₄ haloalkoxy, C1-C4 alkoxy, —(C1-C4)-O—(C1-C4 alkyl), —C(O)(C₁-C₄ alkyl), —S(O)R¹⁴, C₁-C₄ alkylamino, and (C1-C4)(C1-C4) dialkylamino. In yet further embodiments, Cy¹ is a C4-C9 cycloalkyl monosubstituted with a group selected from halogen, —CN. NH₂, —OH, —NO₂, ═O, C₃-C₆ cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. In an even further embodiment, Cy¹ is an unsubstituted C4-C9 cycloalkyl.

In some embodiments, Cy¹ is a C3-C9 heterocycle having at least one O, S, or N atom substituted with 0, 1, 2, or 3 groups independently selected from halogen, —CN, —NH₂, —OH, —NO₂, ═O, C₃-C₆ cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. Examples of C3-C9 heterocycles include, but are not limited to, tetrahydrofuran, pyrrolidine, tetrahydrothiophene, piperidine, piperazine, tetrahydropyran, thiane, 1,3-dithiane, 1,4-dithiane, thiomorpholine, dioxane, morpholine, and hexahydro-1H-furo[3,4-c]pyrrole. In further embodiments, Cy¹ is a C3-C9 heterocycle substituted with 0, 1, or 2 groups independently selected from halogen, —CN, —NH₂, —OH, —NO₂, ═O, C₃-C₆ cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. In still further embodiments, Cy¹ is a C3-C9 heterocycle substituted with 0 or 1 group selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. In yet further embodiments, Cy¹ is a C3-C9 heterocycle monosubstituted with a group selected from halogen, —CN, —NH₂, —OH, —NO₂, ═O, C3-C6 cycloalkyl, C2-C5 heterocycloalkyl, C1-C4 alkyl, C2-C4 alkenyl, C₁-C₄ haloalkyl, C1-C4 cyanoalkyl, C1-C4 hydroxyalkyl, C1-C4 haloalkoxy, C1-C4 alkoxy, —(C₁-C₄)—O—(C1-C4 alkyl), —C(O)(C1-C4 alkyl), —S(O)R¹⁴, C1—C4 alkylamino, and (C1-C4)(C1-C4) dialkylamino. In an even further embodiment, Cy¹ is an unsubstituted C3-C9 heterocycle.

In some embodiments, Cy¹ is a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C₁-C₄ haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. Examples of C2-C9 heteroaryls include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, pyrazolopyrimidinyl, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl. In further embodiments, Cy¹ is a C2-C9 heteroaryl substituted with 0, 1, or 2 groups independently selected from halogen, —CN, —NH₂, —OH, —NO₂, ═O, C₃-C₆ cycloalkyl, C2-C5 heterocycloalkyl, C1-C4 alkyl, C2-C4 alkenyl, C1-C4 haloalkyl, C1-C4 cyanoalkyl, C₁-C₄ hydroxyalkyl, C₁-C₄ haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino. In still further embodiments, Cy¹ is a C2-C9 heteroaryl substituted with 0 or 1 group selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C₁-C₄)(C1-C4) dialkylamino. In yet further embodiments, Cy¹ is a C2-C9 heteroaryl monosubstituted with a group selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C₁-C₄ alkyl), —S(O)R¹⁴, C1-C4 alkylamino, and (C1-C4)(C1-C4) dialkylamino. In an even further embodiment, Cy¹ is an unsubstituted C2-C9 heteroaryl.

In some embodiments, Cy¹ is a C3-C9 heterocycle having at least one O, S, or N atom and substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. Examples of C3-C9 heterocycles include, but are not limited to, tetrahydrofuran, pyrrolidine, tetrahydrothiophene, piperidine, piperazine, tetrahydropyran, thiane, 1,3-dithiane, 1,4-dithiane, thiomorpholine, dioxane, morpholine, and hexahydro-1H-furo[3,4-c]pyrrole. In further embodiments, Cy¹ is a C3-C9 heterocycle having at least one 0, S, or N atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹ is a C3-C9 heterocycle having at least one O, S, or N atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹ is a C3-C9 heterocycle having at least one O, S, or N atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹ is a C3-C9 heterocycle having at least one O, S, or N atom and unsubstituted.

In some embodiments, Cy¹ is a C3-C9 heterocycle having at least one O atom and substituted with 0, 1, 2, or 3 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹ is a C3-C9 heterocycle having at least one O atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹ is a C3-C9 heterocycle having at least one O atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹ is a C3-C9 heterocycle having at least one O atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹ is a C3-C9 heterocycle having at least one O atom and unsubstituted.

In some embodiments. Cy¹ is a C3-C9 heterocycle having at least one S atom and substituted with 0, 1, 2, or 3 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹ is a C3-C9 heterocycle having at least one S atom and substituted with 0, 1, or 2 groups independently selected from halogen, —CN, —NH₂, —OH, —N_{O}2, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹ is a C3-C9 heterocycle having at least one S atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4haloalkoxy, C1-C4 alkoxy, C1-C4 alkylamino, and (C1-C4)(C1-C4) dialkylamino. In yet further embodiments, Cy¹ is a C3-C9 heterocycle having at least one S atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C₁-C₄ hydroxyalkyl, C1-C4 haloalkoxy, C1-C4 alkoxy, C1-C4 alkylamino, and (C1-C4)(C1-C4) dialkylamino. In even further embodiments, Cy¹ is a C3-C9 heterocycle having at least one S atom and unsubstituted.

In some embodiments, Cy¹ is a C3-C9 heterocycle having at least one N atom and substituted with 0, 1, 2, or 3 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹ is a C3-C9 heterocycle having at least one N atom and substituted with 0, 1, or 2 groups independently selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹ is a C3-C9 heterocycle having at least one N atom and substituted with 0 or 1 group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1—C4 haloalkoxy, C1-C4 alkoxy, C1-C4 alkylamino, and (C1-C4)(C1-C4) dialkylamino. In yet further embodiments, Cy¹ is a C3-C9 heterocycle having at least one N atom and monosubstituted with a group selected from halogen, —CN, —NH₂, —OH, —NO₂, ═O, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹ is a C3-C9 heterocycle having at least one N atom and unsubstituted.

In some embodiments, Cy¹ is a C3-C9 heterocycle having at least one O, S, or N atom. In further embodiments, the C3-C9 heterocycle is a monocyclic heterocycle. In still further embodiments, the C3-C9 heterocycle is a bicyclic heterocycle. In yet further embodiments, the C3-C9 heterocycle is a spirocyclic heterocycle. In an even further embodiment, the C3-C9 heterocycle is a fused heterocycle.

In some embodiments, Cy¹ is a C2-C9 heteroaryl having at least one O, S, or N atom.

In some embodiments, Cy¹ is a C3-C9 heterocycle having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In some embodiments, Cy¹ is a structure represented by a formula selected from:

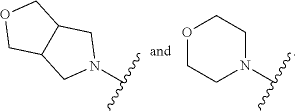 [SMILES: *N1CC2COCC2C1 ; *N1CCOCC1]

In some embodiments, Cy¹ is a structure represented by a formula:

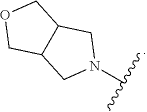 [SMILES: *N1CC2COCC2C1]

In some embodiments, Cy¹ is a structure represented by a formula:

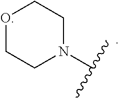 [SMILES: *N1CCOCC1]

### 2. Example Compounds

In some embodiments, a compound can be present as one or more of the following structures:

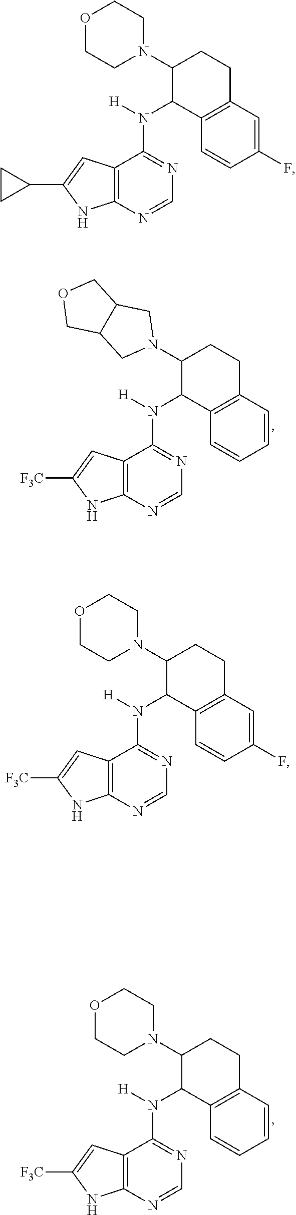 [SMILES: Fc1ccc2c(c1)CCC(N1CCOCC1)C2Nc1ncnc2[nH]c(C3CC3)cc12 ; FC(F)(F)c1cc2c(NC3c4ccccc4CCC3N3CC4COCC4C3)ncnc2[nH]1 ; Fc1ccc2c(c1)CCC(N1CCOCC1)C2Nc1ncnc2[nH]c(C(F)(F)F)cc12 ; FC(F)(F)c1cc2c(NC3c4ccccc4CCC3N3CCOCC3)ncnc2[nH]1]

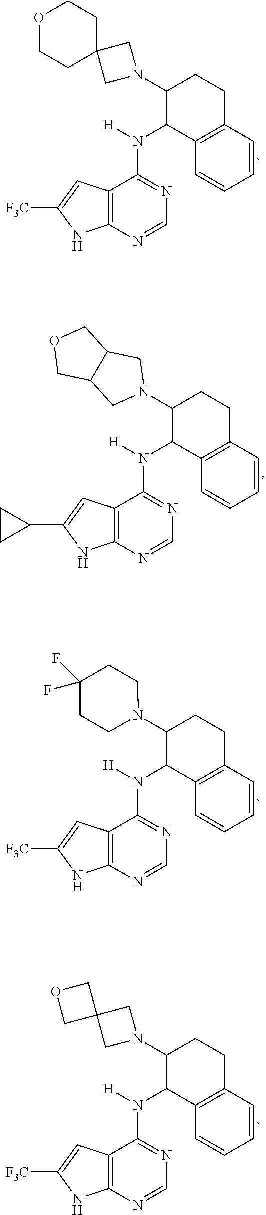 [SMILES: FC(F)(F)c1cc2c(NC3c4ccccc4CCC3N3CC4(CCOCC4)C3)ncnc2[nH]1 ; c1ccc2c(c1)CCC(N1CC3COCC3C1)C2Nc1ncnc2[nH]c(C3CC3)cc12 ; FC1(F)CCN(C2CCc3ccccc3C2Nc2ncnc3[nH]c(C(F)(F)F)cc23)CC1 ; FC(F)(F)c1cc2c(NC3c4ccccc4CCC3N3CC4(COC4)C3)ncnc2[nH]1]

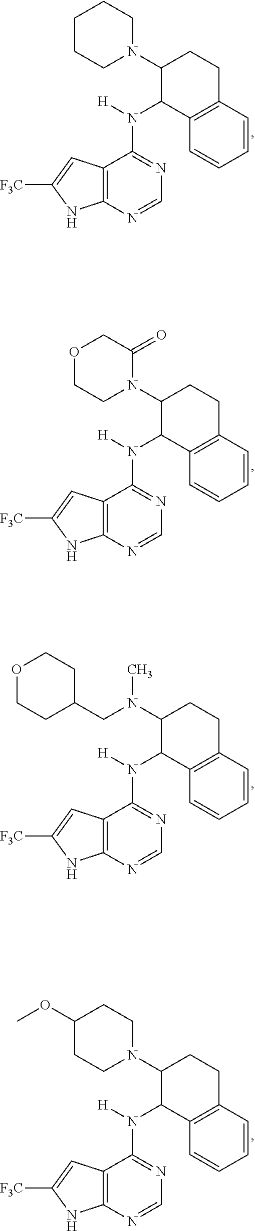 [SMILES: FC(F)(F)c1cc2c(NC3c4ccccc4CCC3N3CCCCC3)ncnc2[nH]1 ; O=C1COCCN1C1CCc2ccccc2C1Nc1ncnc2[nH]c(C(F)(F)F)cc12 ; CN(CC1CCOCC1)C1CCc2ccccc2C1Nc1ncnc2[nH]c(C(F)(F)F)cc12 ; COC1CCN(C2CCc3ccccc3C2Nc2ncnc3[nH]c(C(F)(F)F)cc23)CC1]

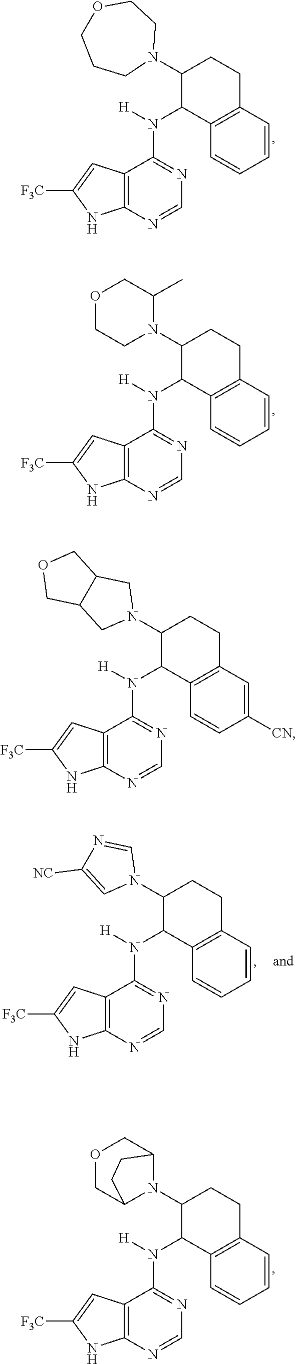 [SMILES: FC(F)(F)c1cc2c(NC3c4ccccc4CCC3N3CCCOCC3)ncnc2[nH]1 ; CC1COCCN1C1CCc2ccccc2C1Nc1ncnc2[nH]c(C(F)(F)F)cc12 ; N#Cc1ccc2c(c1)CCC(N1CC3COCC3C1)C2Nc1ncnc2[nH]c(C(F)(F)F)cc12 ; N#Cc1cn(C2CCc3ccccc3C2Nc2ncnc3[nH]c(C(F)(F)F)cc23)cn1 ; FC(F)(F)c1cc2c(NC3c4ccccc4CCC3N3C4CCC3COC4)ncnc2[nH]1]

or a pharmaceutically acceptable salt thereof.

In some embodiments, a compound can be present as one or more of the following structures:

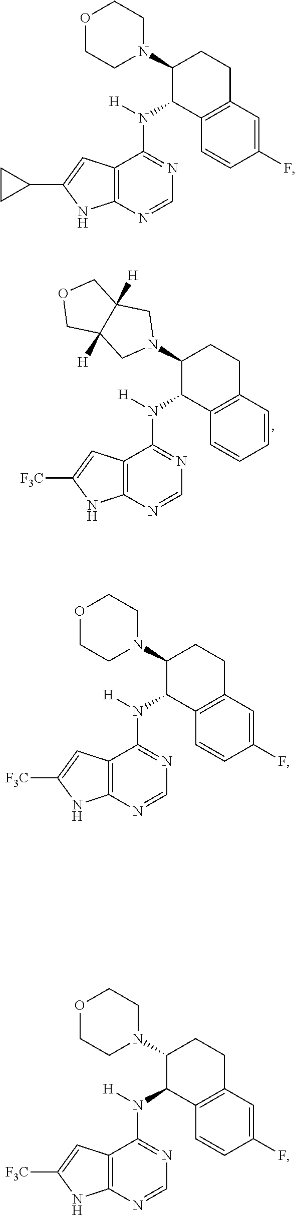 [SMILES: Fc1ccc2c(c1)CC[C@H](N1CCOCC1)[C@H]2Nc1ncnc2[nH]c(C3CC3)cc12 ; FC(F)(F)c1cc2c(N[C@H]3c4ccccc4CC[C@@H]3N3C[C@H]4COC[C@H]4C3)ncnc2[nH]1 ; Fc1ccc2c(c1)CC[C@H](N1CCOCC1)[C@H]2Nc1ncnc2[nH]c(C(F)(F)F)cc12 ; Fc1ccc2c(c1)CC[C@@H](N1CCOCC1)[C@@H]2Nc1ncnc2[nH]c(C(F)(F)F)cc12]

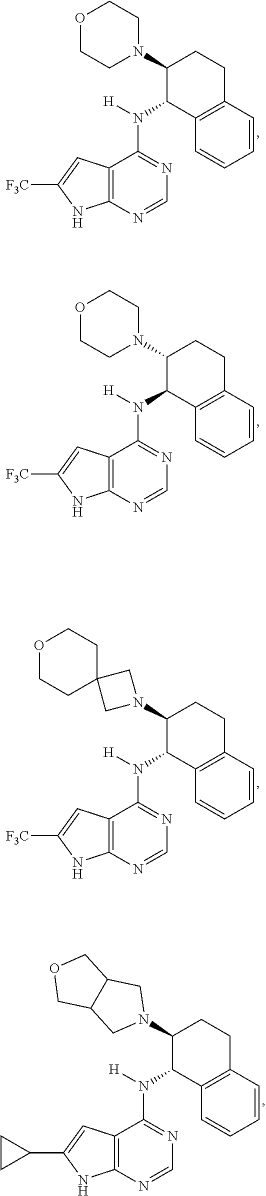 [SMILES: FC(F)(F)c1cc2c(N[C@H]3c4ccccc4CC[C@@H]3N3CCOCC3)ncnc2[nH]1 ; FC(F)(F)c1cc2c(N[C@@H]3c4ccccc4CC[C@H]3N3CCOCC3)ncnc2[nH]1 ; FC(F)(F)c1cc2c(N[C@H]3c4ccccc4CC[C@@H]3N3CC4(CCOCC4)C3)ncnc2[nH]1 ; c1ccc2c(c1)CC[C@H](N1CC3COCC3C1)[C@H]2Nc1ncnc2[nH]c(C3CC3)cc12]

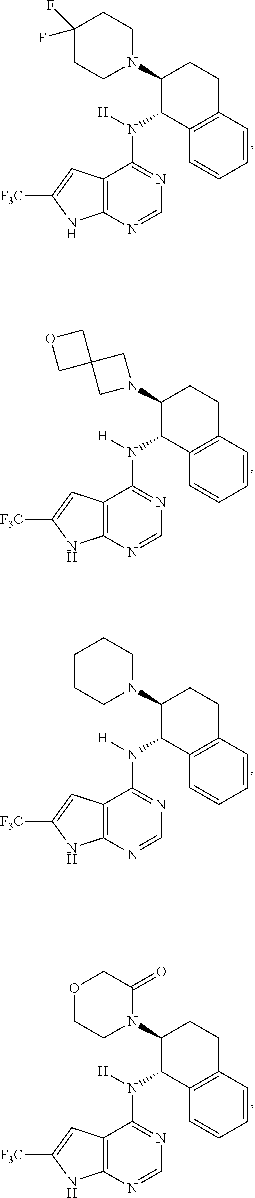 [SMILES: FC1(F)CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CC4(COC4)C3)ncnc2[nH]1 ; FC(F)(F)c1cc2c(N[C@H]3c4ccccc4CC[C@@H]3N3CCCCC3)ncnc2[nH]1 ; O=C1COCCN1[C@H]1CCc2ccccc2[C@@H]1Nc1ncnc2[nH]c(C(F)(F)F)cc12]

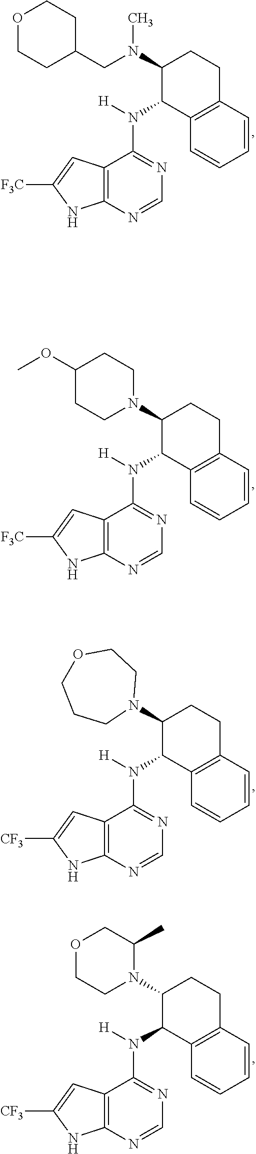 [SMILES: CN(CC1CCOCC1)[C@H]1CCc2ccccc2[C@@H]1Nc1ncnc2[nH]c(C(F)(F)F)cc12 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CCCOCC3)ncnc2[nH]1 ; C[C@@H]1COCCN1[C@@H]1CCc2ccccc2[C@H]1Nc1ncnc2[nH]c(C(F)(F)F)cc12]

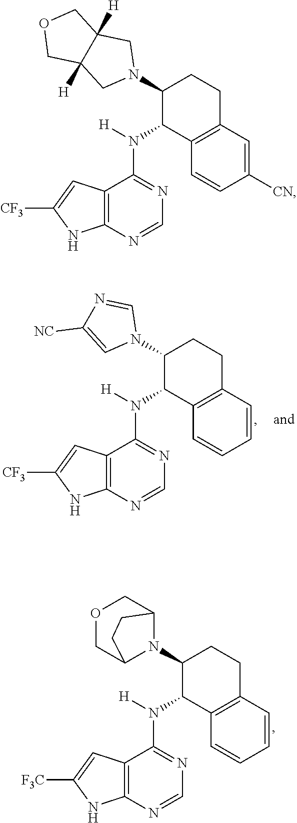 [SMILES: N#Cc1ccc2c(c1)CC[C@H](N1C[C@H]3COC[C@H]3C1)[C@H]2Nc1ncnc2[nH]c(C(F)(F)F)cc12 ; N#Cc1cn([C@@H]2CCc3ccccc3[C@@H]2Nc2ncnc3[nH]c(C(F)(F)F)cc23)cn1 ; FC(F)(F)c1cc2c(N[C@H]3c4ccccc4CC[C@@H]3N3C4CCC3COC4)ncnc2[nH]1]

or a pharmaceutically acceptable salt thereof.

In some embodiments, a compound can be present as one or more of the following structures:

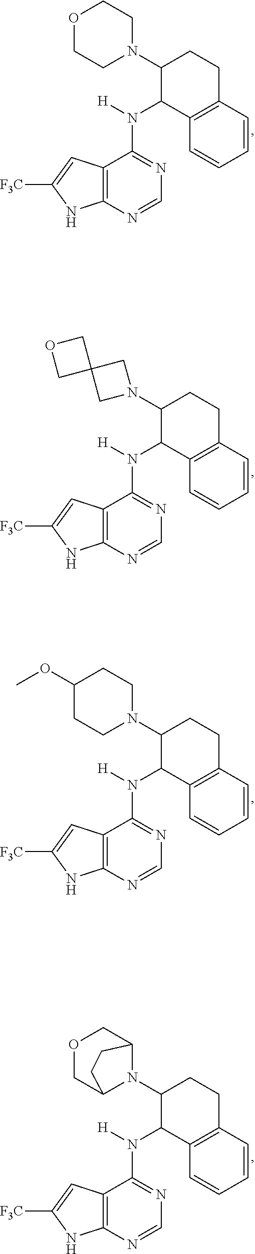 [SMILES: FC(F)(F)c1cc2c(NC3c4ccccc4CCC3N3CCOCC3)ncnc2[nH]1 ; FC(F)(F)c1cc2c(NC3c4ccccc4CCC3N3CC4(COC4)C3)ncnc2[nH]1 ; COC1CCN(C2CCc3ccccc3C2Nc2ncnc3[nH]c(C(F)(F)F)cc23)CC1 ; FC(F)(F)c1cc2c(NC3c4ccccc4CCC3N3C4CCC3COC4)ncnc2[nH]1]

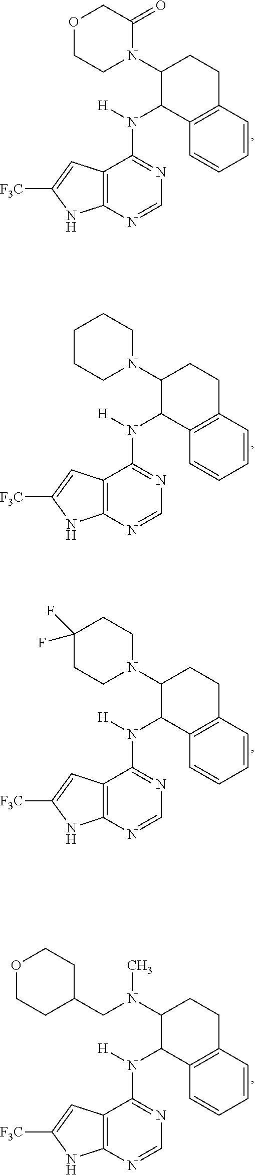 [SMILES: O=C1COCCN1C1CCc2ccccc2C1Nc1ncnc2[nH]c(C(F)(F)F)cc12 ; FC(F)(F)c1cc2c(NC3c4ccccc4CCC3N3CCCCC3)ncnc2[nH]1 ; FC1(F)CCN(C2CCc3ccccc3C2Nc2ncnc3[nH]c(C(F)(F)F)cc23)CC1 ; CN(CC1CCOCC1)C1CCc2ccccc2C1Nc1ncnc2[nH]c(C(F)(F)F)cc12]

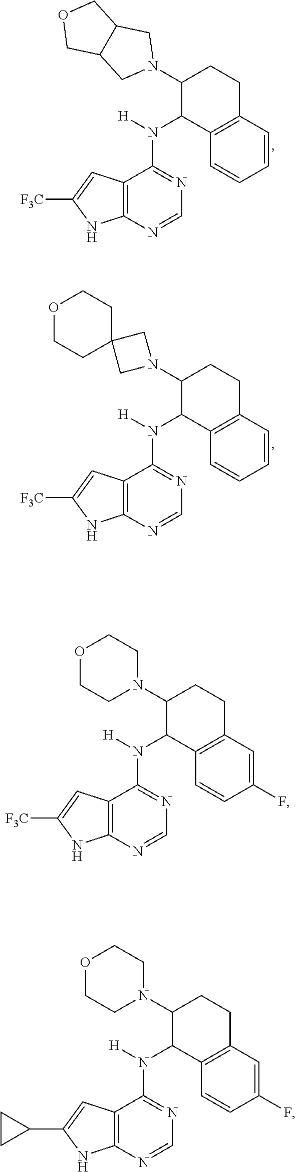 [SMILES: FC(F)(F)c1cc2c(NC3c4ccccc4CCC3N3CC4COCC4C3)ncnc2[nH]1 ; FC(F)(F)c1cc2c(NC3c4ccccc4CCC3N3CC4(CCOCC4)C3)ncnc2[nH]1 ; Fc1ccc2c(c1)CCC(N1CCOCC1)C2Nc1ncnc2[nH]c(C(F)(F)F)cc12 ; Fc1ccc2c(c1)CCC(N1CCOCC1)C2Nc1ncnc2[nH]c(C3CC3)cc12]

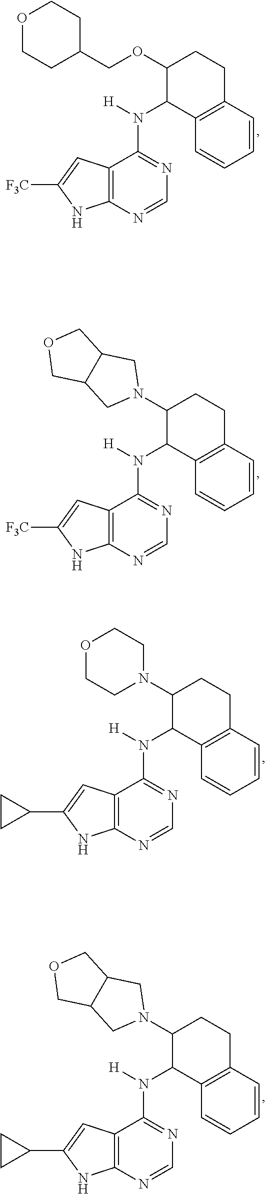 [SMILES: FC(F)(F)c1cc2c(NC3c4ccccc4CCC3OCC3CCOCC3)ncnc2[nH]1 ; FC(F)(F)c1cc2c(NC3c4ccccc4CCC3N3CC4COCC4C3)ncnc2[nH]1 ; c1ccc2c(c1)CCC(N1CCOCC1)C2Nc1ncnc2[nH]c(C3CC3)cc12 ; c1ccc2c(c1)CCC(N1CC3COCC3C1)C2Nc1ncnc2[nH]c(C3CC3)cc12]

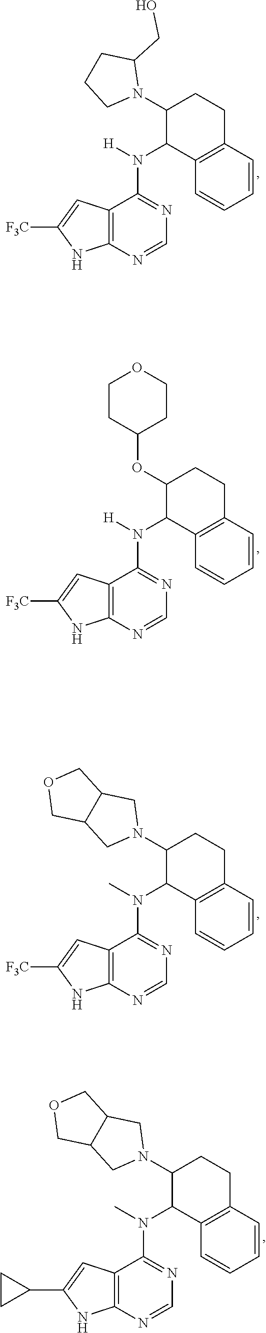 [SMILES: OCC1CCCN1C1CCc2ccccc2C1Nc1ncnc2[nH]c(C(F)(F)F)cc12 ; FC(F)(F)c1cc2c(NC3c4ccccc4CCC3OC3CCOCC3)ncnc2[nH]1 ; CN(c1ncnc2[nH]c(C(F)(F)F)cc12)C1c2ccccc2CCC1N1CC2COCC2C1 ; CN(c1ncnc2[nH]c(C3CC3)cc12)C1c2ccccc2CCC1N1CC2COCC2C1]

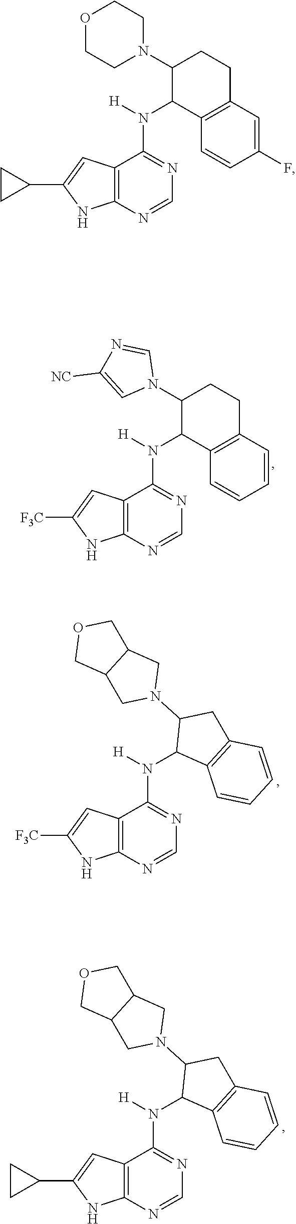 [SMILES: Fc1ccc2c(c1)CCC(N1CCOCC1)C2Nc1ncnc2[nH]c(C3CC3)cc12 ; N#Cc1cn(C2CCc3ccccc3C2Nc2ncnc3[nH]c(C(F)(F)F)cc23)cn1 ; FC(F)(F)c1cc2c(NC3c4ccccc4CC3N3CC4COCC4C3)ncnc2[nH]1 ; c1ccc2c(c1)CC(N1CC3COCC3C1)C2Nc1ncnc2[nH]c(C3CC3)cc12]

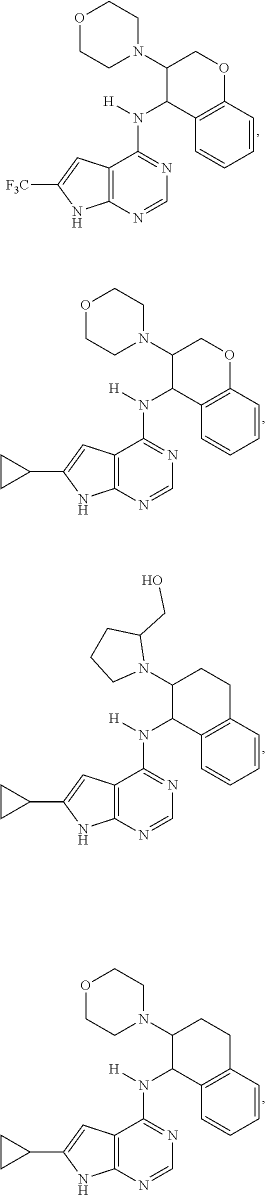 [SMILES: FC(F)(F)c1cc2c(NC3c4ccccc4OCC3N3CCOCC3)ncnc2[nH]1 ; c1ccc2c(c1)OCC(N1CCOCC1)C2Nc1ncnc2[nH]c(C3CC3)cc12 ; OCC1CCCN1C1CCc2ccccc2C1Nc1ncnc2[nH]c(C3CC3)cc12 ; c1ccc2c(c1)CCC(N1CCOCC1)C2Nc1ncnc2[nH]c(C3CC3)cc12]

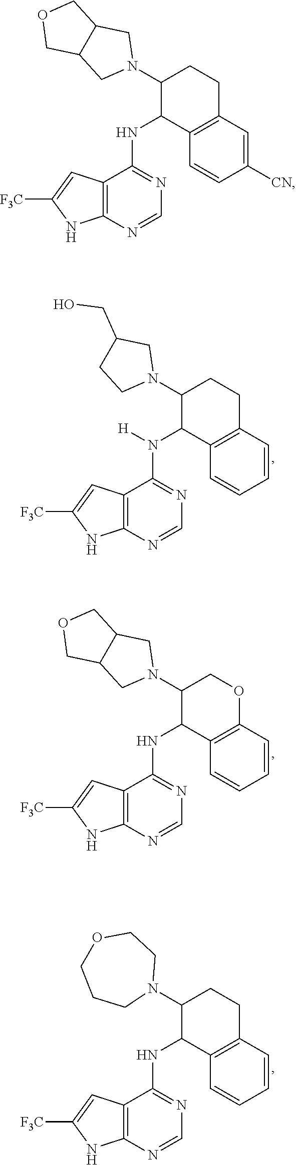 [SMILES: N#Cc1ccc2c(c1)CCC(N1CC3COCC3C1)C2Nc1ncnc2[nH]c(C(F)(F)F)cc12 ; OCC1CCN(C2CCc3ccccc3C2Nc2ncnc3[nH]c(C(F)(F)F)cc23)C1 ; FC(F)(F)c1cc2c(NC3c4ccccc4OCC3N3CC4COCC4C3)ncnc2[nH]1 ; FC(F)(F)c1cc2c(NC3c4ccccc4CCC3N3CCCOCC3)ncnc2[nH]1]

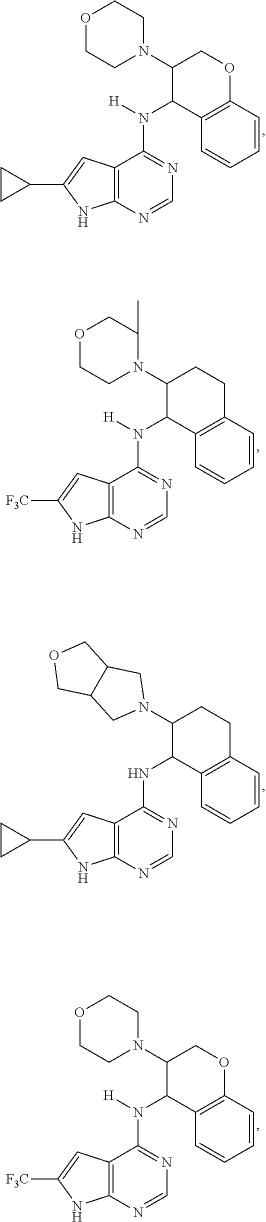 [SMILES: c1ccc2c(c1)OCC(N1CCOCC1)C2Nc1ncnc2[nH]c(C3CC3)cc12 ; CC1COCCN1C1CCc2ccccc2C1Nc1ncnc2[nH]c(C(F)(F)F)cc12 ; c1ccc2c(c1)CCC(N1CC3COCC3C1)C2Nc1ncnc2[nH]c(C3CC3)cc12 ; FC(F)(F)c1cc2c(NC3c4ccccc4OCC3N3CCOCC3)ncnc2[nH]1]

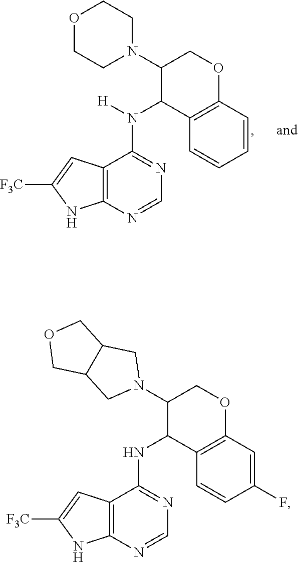 [SMILES: FC(F)(F)c1cc2c(NC3c4ccccc4OCC3N3CCOCC3)ncnc2[nH]1 ; Fc1ccc2c(c1)OCC(N1CC3COCC3C1)C2Nc1ncnc2[nH]c(C(F)(F)F)cc12]

or a pharmaceutically acceptable salt thereof.

In some embodiments, a compound can be present as one or more of the following structures:

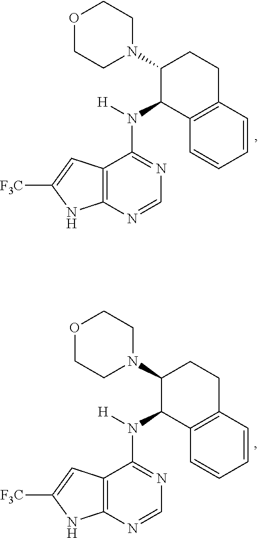 [SMILES: FC(F)(F)c1cc2c(N[C@@H]3c4ccccc4CC[C@H]3N3CCOCC3)ncnc2[nH]1 ; FC(F)(F)c1cc2c(N[C@@H]3c4ccccc4CC[C@@H]3N3CCOCC3)ncnc2[nH]1]

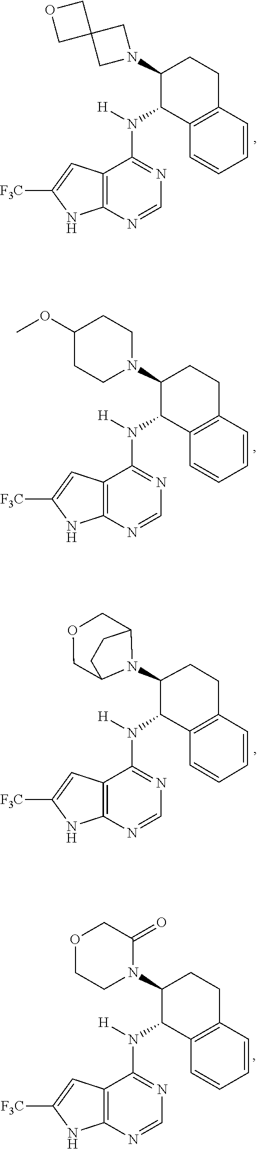 [SMILES: FC(F)(F)c1cc2c(N[C@H]3c4ccccc4CC[C@@H]3N3CC4(COC4)C3)ncnc2[nH]1 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3C4CCC3COC4)ncnc2[nH]1 ; O=C1COCCN1[C@H]1CCc2ccccc2[C@@H]1Nc1ncnc2[nH]c(C(F)(F)F)cc12]

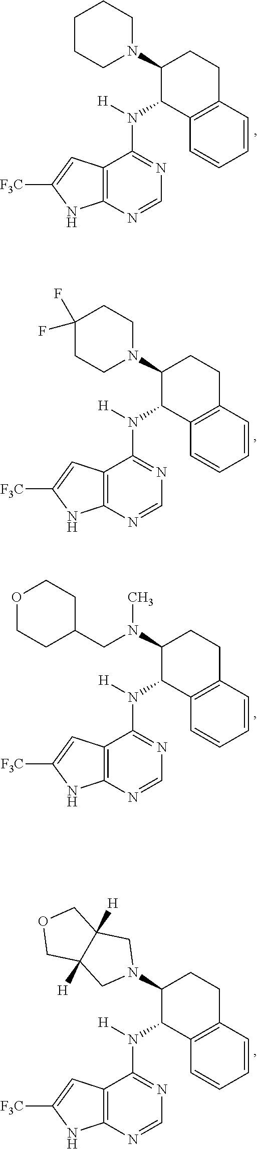 [SMILES: FC(F)(F)c1cc2c(N[C@H]3c4ccccc4CC[C@@H]3N3CCCCC3)ncnc2[nH]1 ; FC1(F)CCN([C@H]2CCc3ccccc3[C@@H]2Nc2ncnc3[nH]c(C(F)(F)F)cc23)CC1 ; CN(CC1CCOCC1)[C@H]1CCc2ccccc2[C@@H]1Nc1ncnc2[nH]c(C(F)(F)F)cc12 ; FC(F)(F)c1cc2c(N[C@H]3c4ccccc4CC[C@@H]3N3C[C@H]4COC[C@H]4C3)ncnc2[nH]1]

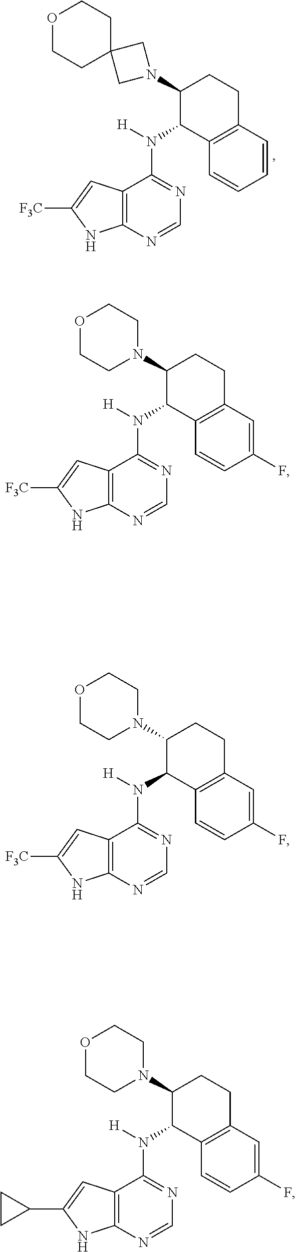 [SMILES: FC(F)(F)c1cc2c(N[C@H]3c4ccccc4CC[C@@H]3N3CC4(CCOCC4)C3)ncnc2[nH]1 ; Fc1ccc2c(c1)CC[C@H](N1CCOCC1)[C@H]2Nc1ncnc2[nH]c(C(F)(F)F)cc12 ; Fc1ccc2c(c1)CC[C@@H](N1CCOCC1)[C@@H]2Nc1ncnc2[nH]c(C(F)(F)F)cc12 ; Fc1ccc2c(c1)CC[C@H](N1CCOCC1)[C@H]2Nc1ncnc2[nH]c(C3CC3)cc12]

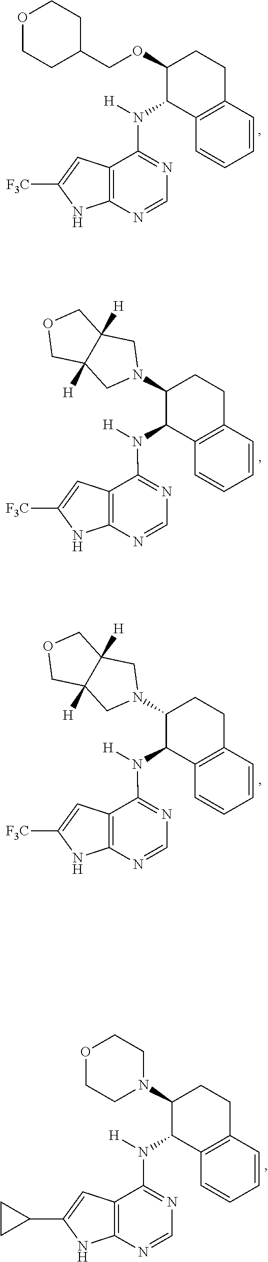 [SMILES: FC(F)(F)c1cc2c(N[C@H]3c4ccccc4CC[C@@H]3OCC3CCOCC3)ncnc2[nH]1 ; FC(F)(F)c1cc2c(N[C@@H]3c4ccccc4CC[C@@H]3N3C[C@H]4COC[C@H]4C3)ncnc2[nH]1 ; FC(F)(F)c1cc2c(N[C@@H]3c4ccccc4CC[C@H]3N3C[C@H]4COC[C@H]4C3)ncnc2[nH]1 ; c1ccc2c(c1)CC[C@H](N1CCOCC1)[C@H]2Nc1ncnc2[nH]c(C3CC3)cc12]

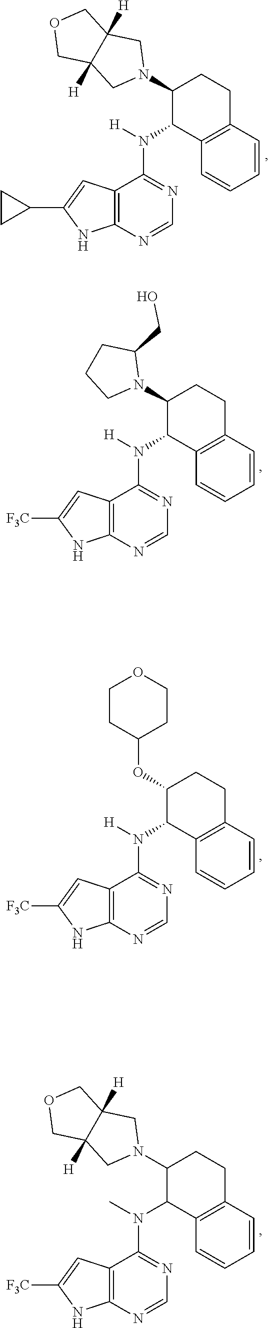 [SMILES: c1ccc2c(c1)CC[C@H](N1C[C@H]3COC[C@H]3C1)[C@H]2Nc1ncnc2[nH]c(C3CC3)cc12 ; OC[C@@H]1CCCN1[C@H]1CCc2ccccc2[C@@H]1Nc1ncnc2[nH]c(C(F)(F)F)cc12 ; FC(F)(F)c1cc2c(N[C@H]3c4ccccc4CC[C@H]3OC3CCOCC3)ncnc2[nH]1 ; CN(c1ncnc2[nH]c(C(F)(F)F)cc12)C1c2ccccc2CCC1N1C[C@H]2COC[C@H]2C1]

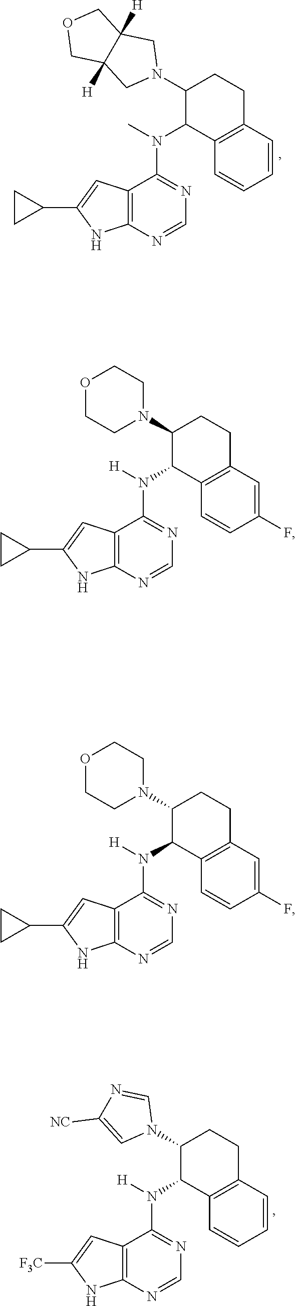 [SMILES: CN(c1ncnc2[nH]c(C3CC3)cc12)C1c2ccccc2CCC1N1C[C@H]2COC[C@H]2C1 ; Fc1ccc2c(c1)CC[C@H](N1CCOCC1)[C@H]2Nc1ncnc2[nH]c(C3CC3)cc12 ; Fc1ccc2c(c1)CC[C@@H](N1CCOCC1)[C@@H]2Nc1ncnc2[nH]c(C3CC3)cc12 ; N#Cc1cn([C@@H]2CCc3ccccc3[C@@H]2Nc2ncnc3[nH]c(C(F)(F)F)cc23)cn1]

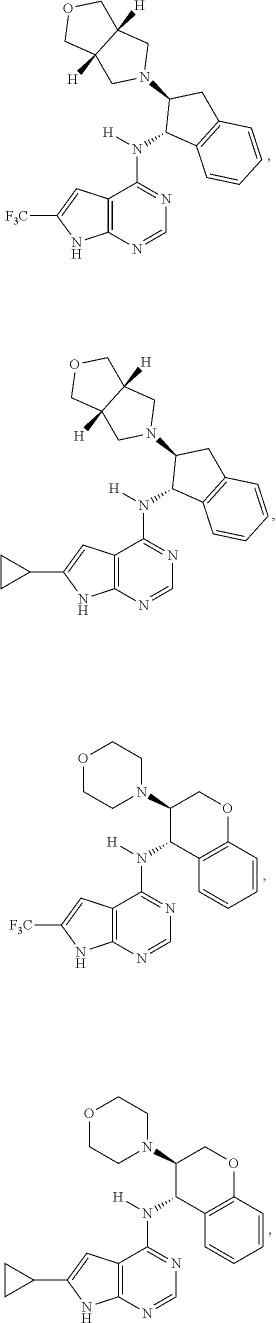 [SMILES: FC(F)(F)c1cc2c(N[C@H]3c4ccccc4C[C@@H]3N3C[C@H]4COC[C@H]4C3)ncnc2[nH]1 ; c1ccc2c(c1)C[C@H](N1C[C@H]3COC[C@H]3C1)[C@H]2Nc1ncnc2[nH]c(C3CC3)cc12 ; FC(F)(F)c1cc2c(N[C@H]3c4ccccc4OC[C@@H]3N3CCOCC3)ncnc2[nH]1 ; c1ccc2c(c1)OC[C@H](N1CCOCC1)[C@H]2Nc1ncnc2[nH]c(C3CC3)cc12]

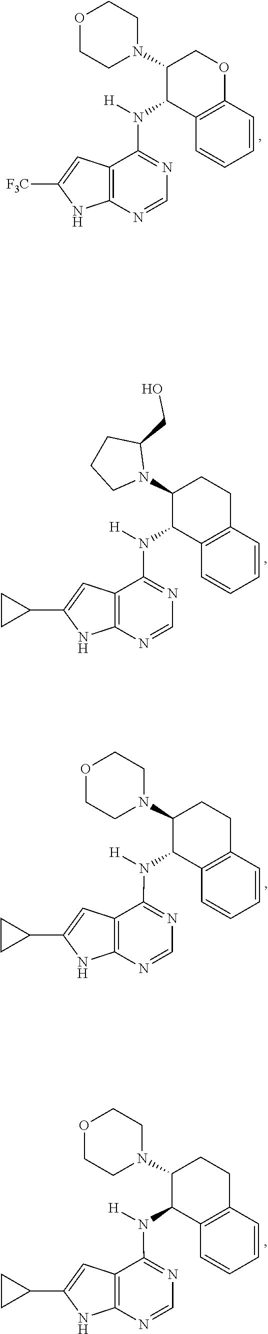 [SMILES: FC(F)(F)c1cc2c(N[C@H]3c4ccccc4OC[C@H]3N3CCOCC3)ncnc2[nH]1 ; OC[C@@H]1CCCN1[C@H]1CCc2ccccc2[C@@H]1Nc1ncnc2[nH]c(C3CC3)cc12 ; c1ccc2c(c1)CC[C@H](N1CCOCC1)[C@H]2Nc1ncnc2[nH]c(C3CC3)cc12 ; c1ccc2c(c1)CC[C@@H](N1CCOCC1)[C@@H]2Nc1ncnc2[nH]c(C3CC3)cc12]

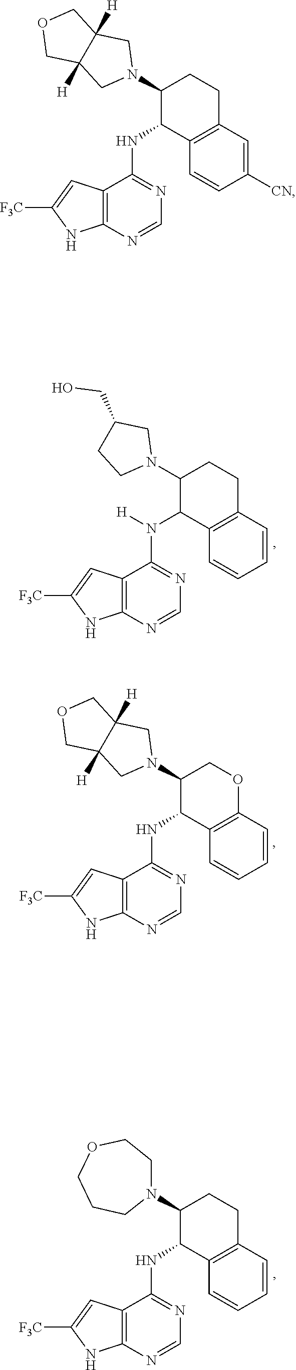 [SMILES: N#Cc1ccc2c(c1)CC[C@H](N1C[C@H]3COC[C@H]3C1)[C@H]2Nc1ncnc2[nH]c(C(F)(F)F)cc12 ; OC[C@H]1CCN(C2CCc3ccccc3C2Nc2ncnc3[nH]c(C(F)(F)F)cc23)C1 ; FC(F)(F)c1cc2c(N[C@H]3c4ccccc4OC[C@@H]3N3C[C@H]4COC[C@H]4C3)ncnc2[nH]1 ; FC(F)(F)c1cc2c(N[C@H]3c4ccccc4CC[C@@H]3N3CCCOCC3)ncnc2[nH]1]

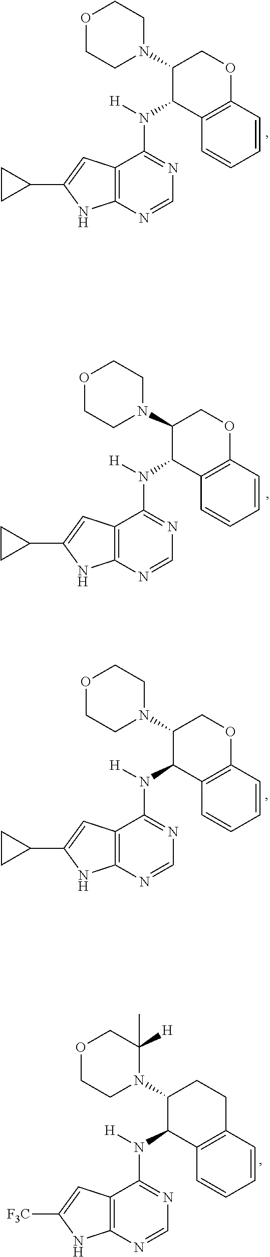 [SMILES: c1ccc2c(c1)OC[C@@H](N1CCOCC1)[C@H]2Nc1ncnc2[nH]c(C3CC3)cc12 ; c1ccc2c(c1)OC[C@H](N1CCOCC1)[C@H]2Nc1ncnc2[nH]c(C3CC3)cc12 ; c1ccc2c(c1)OC[C@@H](N1CCOCC1)[C@@H]2Nc1ncnc2[nH]c(C3CC3)cc12 ; C[C@H]1COCCN1[C@@H]1CCc2ccccc2[C@H]1Nc1ncnc2[nH]c(C(F)(F)F)cc12]

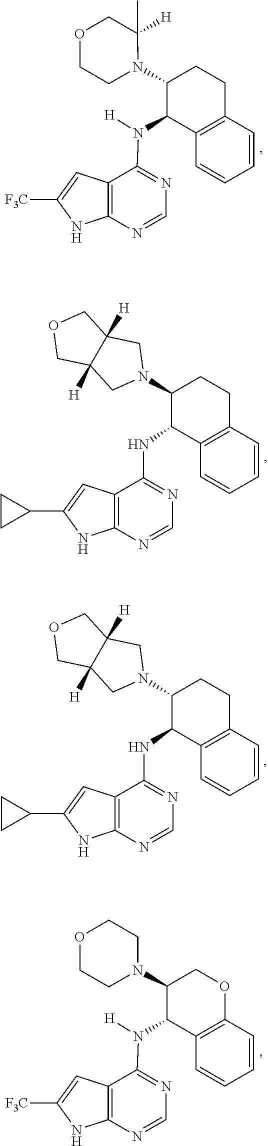

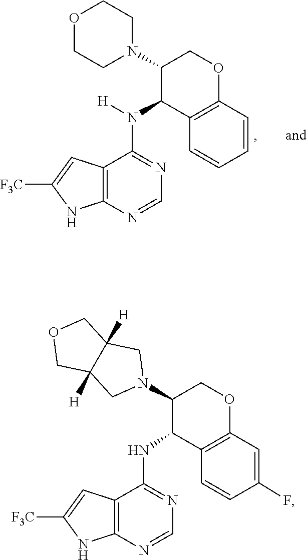 [SMILES: FC(F)(F)c1cc2c(N[C@@H]3c4ccccc4OC[C@H]3N3CCOCC3)ncnc2[nH]1 ; Fc1ccc2c(c1)OC[C@H](N1C[C@H]3COC[C@H]3C1)[C@H]2Nc1ncnc2[nH]c(C(F)(F)F)cc12]

or a pharmaceutically acceptable salt thereof.

In some embodiments, a compound can be present as one or more of the following structures:

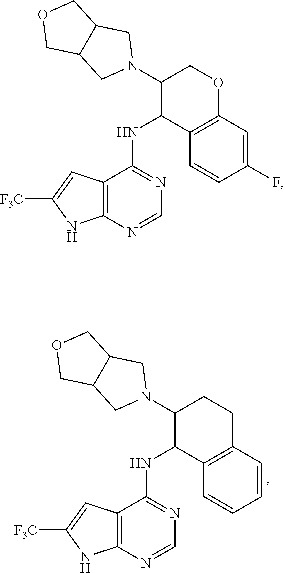 [SMILES: Fc1ccc2c(c1)OCC(N1CC3COCC3C1)C2Nc1ncnc2[nH]c(C(F)(F)F)cc12 ; FC(F)(F)c1cc2c(NC3c4ccccc4CCC3N3CC4COCC4C3)ncnc2[nH]1]

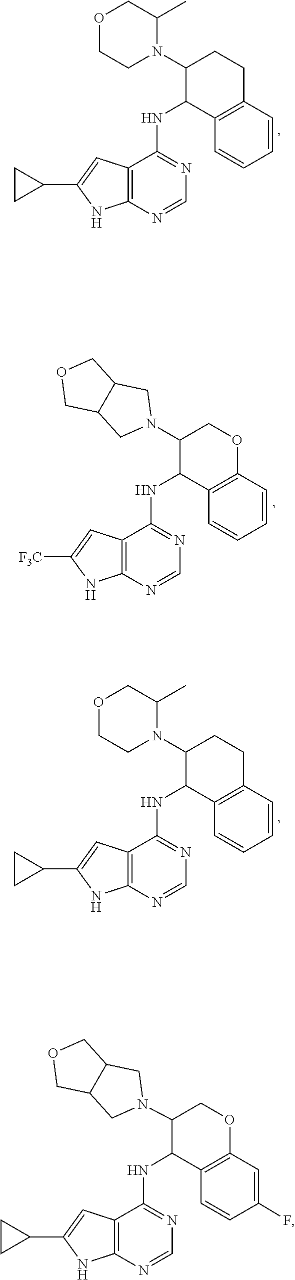 [SMILES: CC1COCCN1C1CCc2ccccc2C1Nc1ncnc2[nH]c(C3CC3)cc12 ; FC(F)(F)c1cc2c(NC3c4ccccc4OCC3N3CC4COCC4C3)ncnc2[nH]1 ; CC1COCCN1C1CCc2ccccc2C1Nc1ncnc2[nH]c(C3CC3)cc12 ; Fc1ccc2c(c1)OCC(N1CC3COCC3C1)C2Nc1ncnc2[nH]c(C3CC3)cc12]

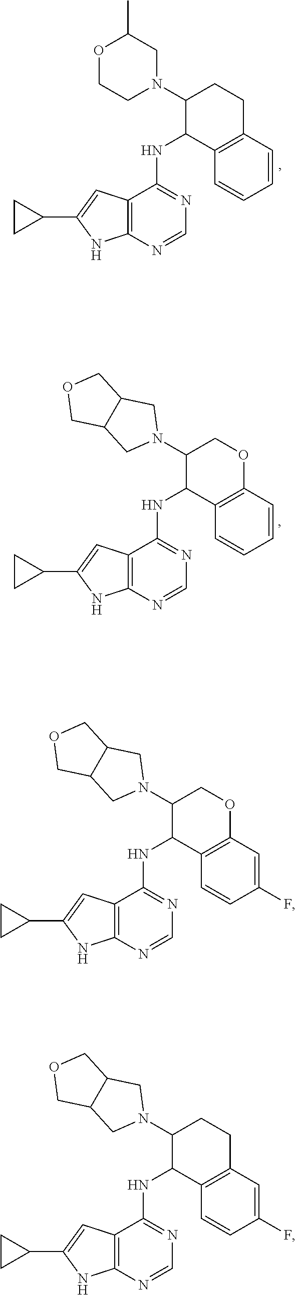 [SMILES: CC1CN(C2CCc3ccccc3C2Nc2ncnc3[nH]c(C4CC4)cc23)CCO1 ; c1ccc2c(c1)OCC(N1CC3COCC3C1)C2Nc1ncnc2[nH]c(C3CC3)cc12 ; Fc1ccc2c(c1)OCC(N1CC3COCC3C1)C2Nc1ncnc2[nH]c(C3CC3)cc12 ; Fc1ccc2c(c1)CCC(N1CC3COCC3C1)C2Nc1ncnc2[nH]c(C3CC3)cc12]

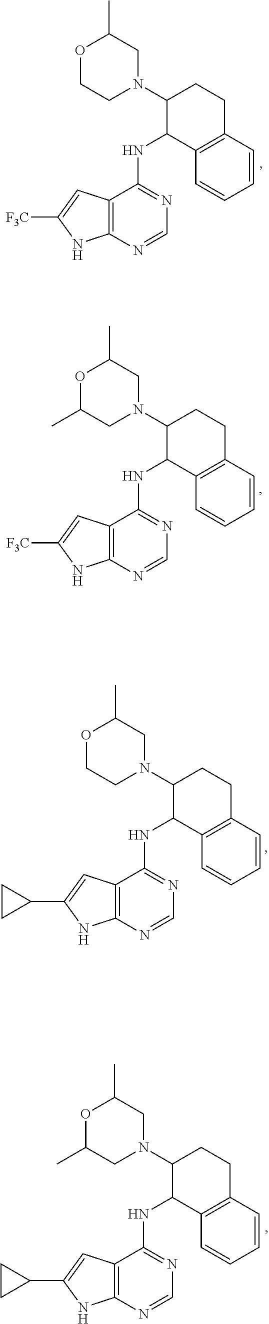 [SMILES: CC1CN(C2CCc3ccccc3C2Nc2ncnc3[nH]c(C(F)(F)F)cc23)CCO1 ; CC1CN(C2CCc3ccccc3C2Nc2ncnc3[nH]c(C(F)(F)F)cc23)CC(C)O1 ; CC1CN(C2CCc3ccccc3C2Nc2ncnc3[nH]c(C4CC4)cc23)CCO1 ; CC1CN(C2CCc3ccccc3C2Nc2ncnc3[nH]c(C4CC4)cc23)CC(C)O1]

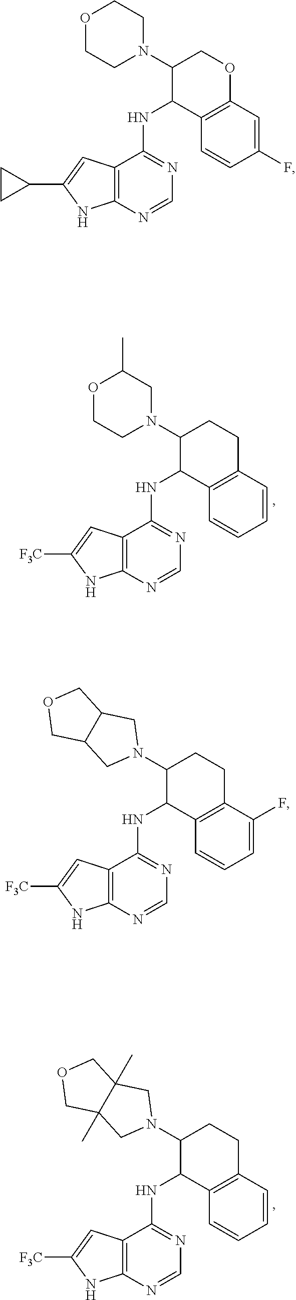 [SMILES: Fc1ccc2c(c1)OCC(N1CCOCC1)C2Nc1ncnc2[nH]c(C3CC3)cc12 ; CC1CN(C2CCc3ccccc3C2Nc2ncnc3[nH]c(C(F)(F)F)cc23)CCO1 ; Fc1cccc2c1CCC(N1CC3COCC3C1)C2Nc1ncnc2[nH]c(C(F)(F)F)cc12 ; CC12COCC1(C)CN(C1CCc3ccccc3C1Nc1ncnc3[nH]c(C(F)(F)F)cc13)C2]

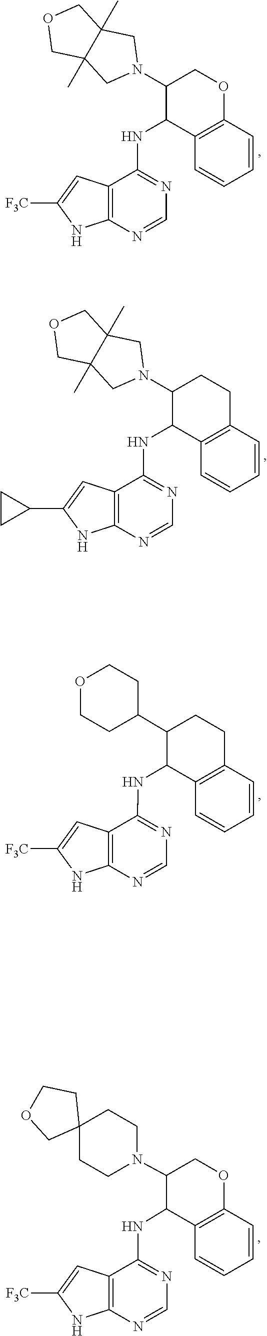 [SMILES: CC12COCC1(C)CN(C1COc3ccccc3C1Nc1ncnc3[nH]c(C(F)(F)F)cc13)C2 ; CC12COCC1(C)CN(C1CCc3ccccc3C1Nc1ncnc3[nH]c(C4CC4)cc13)C2 ; FC(F)(F)c1cc2c(NC3c4ccccc4CCC3C3CCOCC3)ncnc2[nH]1 ; FC(F)(F)c1cc2c(NC3c4ccccc4OCC3N3CCC4(CCOC4)CC3)ncnc2[nH]1]

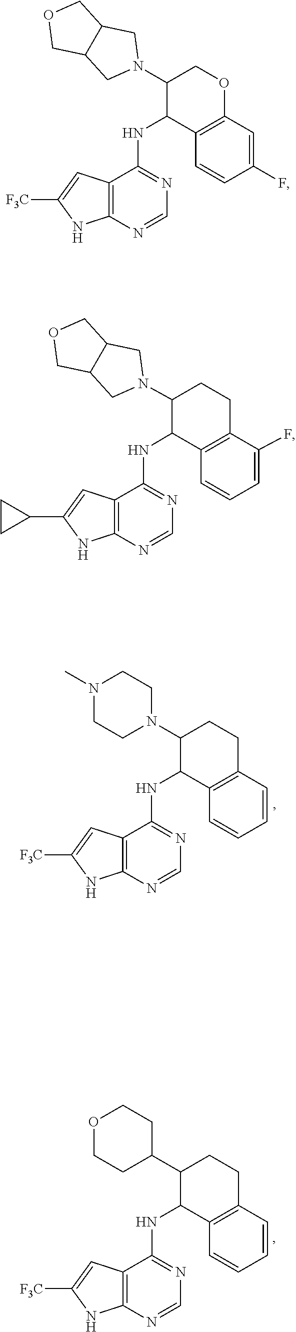 [SMILES: Fc1ccc2c(c1)OCC(N1CC3COCC3C1)C2Nc1ncnc2[nH]c(C(F)(F)F)cc12 ; Fc1cccc2c1CCC(N1CC3COCC3C1)C2Nc1ncnc2[nH]c(C3CC3)cc12 ; CN1CCN(C2CCc3ccccc3C2Nc2ncnc3[nH]c(C(F)(F)F)cc23)CC1 ; FC(F)(F)c1cc2c(NC3c4ccccc4CCC3C3CCOCC3)ncnc2[nH]1]

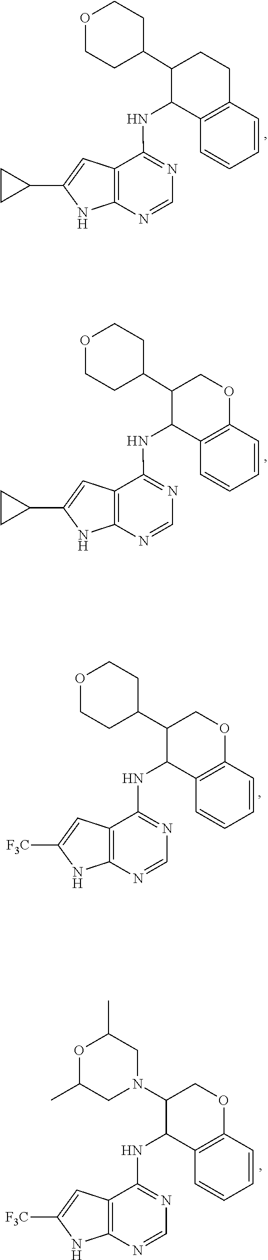 [SMILES: c1ccc2c(c1)CCC(C1CCOCC1)C2Nc1ncnc2[nH]c(C3CC3)cc12 ; c1ccc2c(c1)OCC(C1CCOCC1)C2Nc1ncnc2[nH]c(C3CC3)cc12 ; FC(F)(F)c1cc2c(NC3c4ccccc4OCC3C3CCOCC3)ncnc2[nH]1 ; CC1CN(C2COc3ccccc3C2Nc2ncnc3[nH]c(C(F)(F)F)cc23)CC(C)O1]

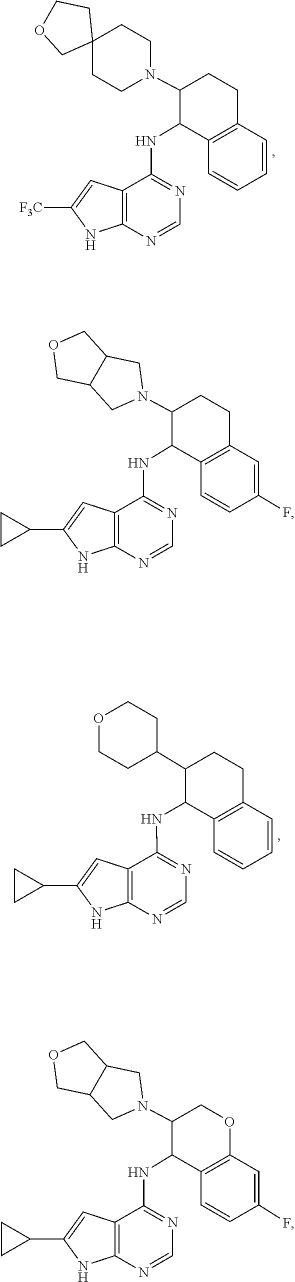 [SMILES: FC(F)(F)c1cc2c(NC3c4ccccc4CCC3N3CCC4(CCOC4)CC3)ncnc2[nH]1 ; Fc1ccc2c(c1)CCC(N1CC3COCC3C1)C2Nc1ncnc2[nH]c(C3CC3)cc12 ; c1ccc2c(c1)CCC(C1CCOCC1)C2Nc1ncnc2[nH]c(C3CC3)cc12 ; Fc1ccc2c(c1)OCC(N1CC3COCC3C1)C2Nc1ncnc2[nH]c(C3CC3)cc12]

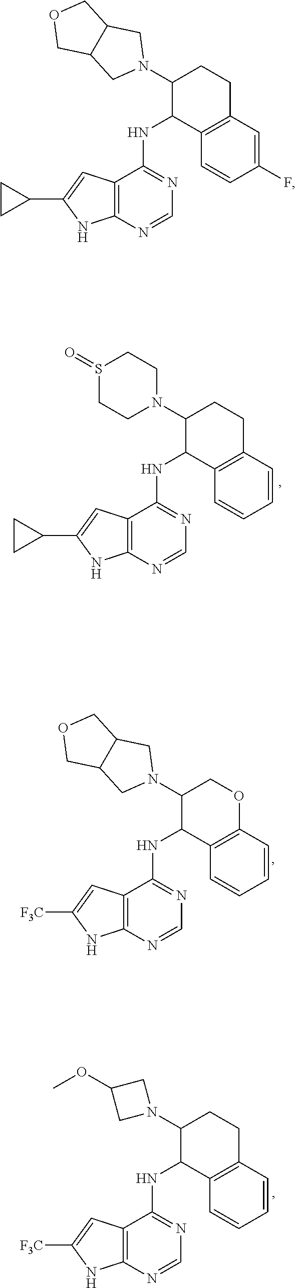 [SMILES: Fc1ccc2c(c1)CCC(N1CC3COCC3C1)C2Nc1ncnc2[nH]c(C3CC3)cc12 ; O=S1CCN(C2CCc3ccccc3C2Nc2ncnc3[nH]c(C4CC4)cc23)CC1 ; FC(F)(F)c1cc2c(NC3c4ccccc4OCC3N3CC4COCC4C3)ncnc2[nH]1 ; COC1CN(C2CCc3ccccc3C2Nc2ncnc3[nH]c(C(F)(F)F)cc23)C1]

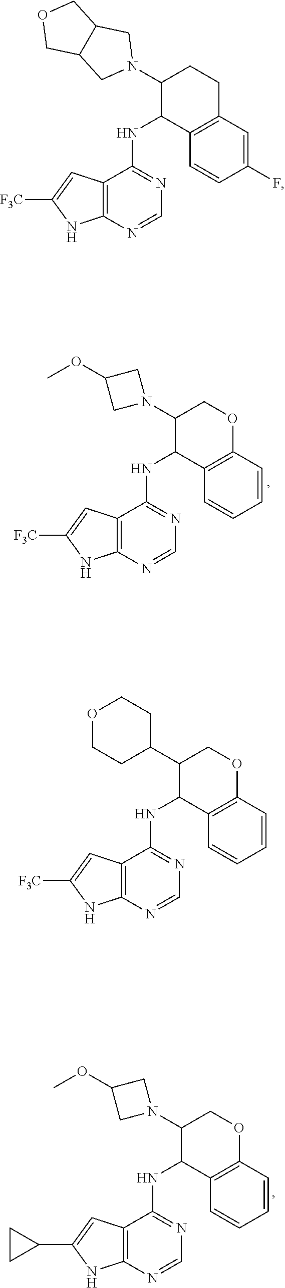 [SMILES: Fc1ccc2c(c1)CCC(N1CC3COCC3C1)C2Nc1ncnc2[nH]c(C(F)(F)F)cc12 ; COC1CN(C2COc3ccccc3C2Nc2ncnc3[nH]c(C(F)(F)F)cc23)C1 ; FC(F)(F)c1cc2c(NC3c4ccccc4OCC3C3CCOCC3)ncnc2[nH]1 ; COC1CN(C2COc3ccccc3C2Nc2ncnc3[nH]c(C4CC4)cc23)C1]

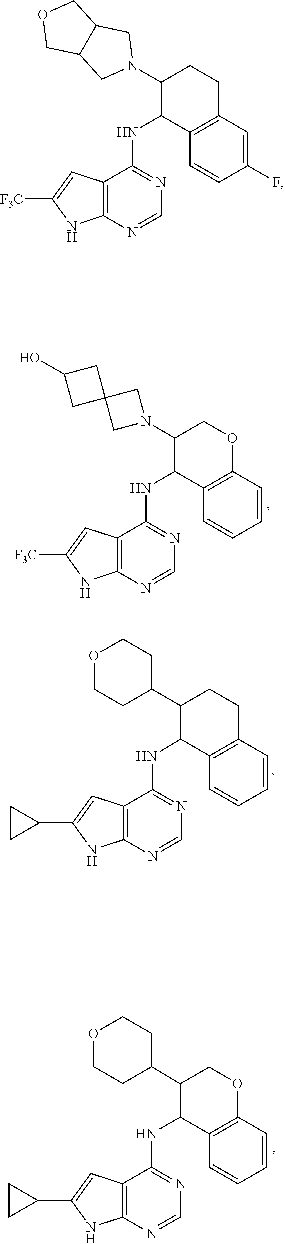 [SMILES: Fc1ccc2c(c1)CCC(N1CC3COCC3C1)C2Nc1ncnc2[nH]c(C(F)(F)F)cc12 ; OC1CC2(C1)CN(C1COc3ccccc3C1Nc1ncnc3[nH]c(C(F)(F)F)cc13)C2 ; c1ccc2c(c1)CCC(C1CCOCC1)C2Nc1ncnc2[nH]c(C3CC3)cc12 ; c1ccc2c(c1)OCC(C1CCOCC1)C2Nc1ncnc2[nH]c(C3CC3)cc12]

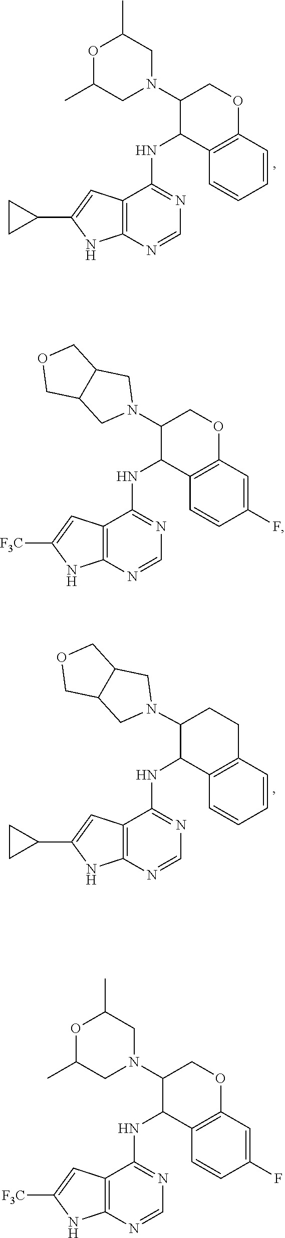 [SMILES: CC1CN(C2COc3ccccc3C2Nc2ncnc3[nH]c(C4CC4)cc23)CC(C)O1 ; Fc1ccc2c(c1)OCC(N1CC3COCC3C1)C2Nc1ncnc2[nH]c(C(F)(F)F)cc12 ; c1ccc2c(c1)CCC(N1CC3COCC3C1)C2Nc1ncnc2[nH]c(C3CC3)cc12 ; CC1CN(C2COc3cc(F)ccc3C2Nc2ncnc3[nH]c(C(F)(F)F)cc23)CC(C)O1]

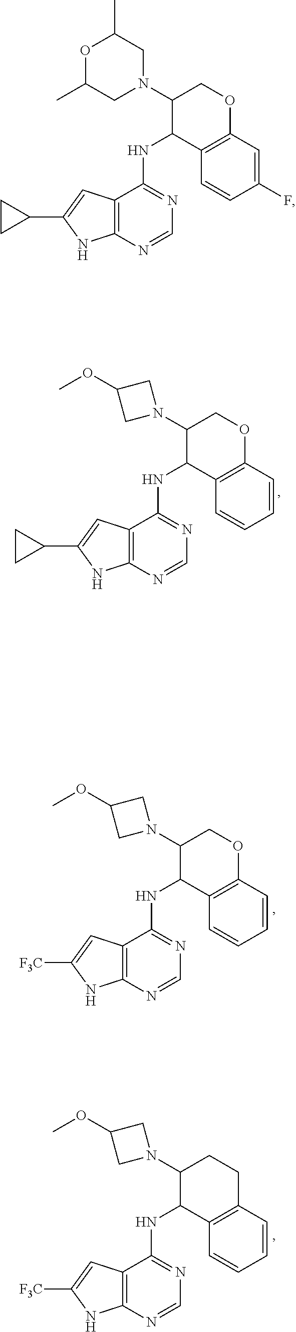 [SMILES: CC1CN(C2COc3cc(F)ccc3C2Nc2ncnc3[nH]c(C4CC4)cc23)CC(C)O1 ; COC1CN(C2COc3ccccc3C2Nc2ncnc3[nH]c(C4CC4)cc23)C1 ; COC1CN(C2COc3ccccc3C2Nc2ncnc3[nH]c(C(F)(F)F)cc23)C1 ; COC1CN(C2CCc3ccccc3C2Nc2ncnc3[nH]c(C(F)(F)F)cc23)C1]

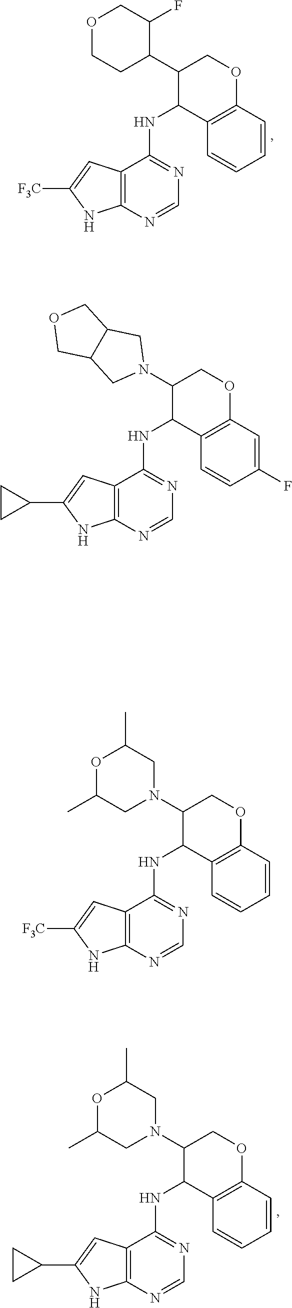 [SMILES: FC1COCCC1C1COc2ccccc2C1Nc1ncnc2[nH]c(C(F)(F)F)cc12 ; Fc1ccc2c(c1)OCC(N1CC3COCC3C1)C2Nc1ncnc2[nH]c(C3CC3)cc12 ; CC1CN(C2COc3ccccc3C2Nc2ncnc3[nH]c(C(F)(F)F)cc23)CC(C)O1 ; CC1CN(C2COc3ccccc3C2Nc2ncnc3[nH]c(C4CC4)cc23)CC(C)O1]

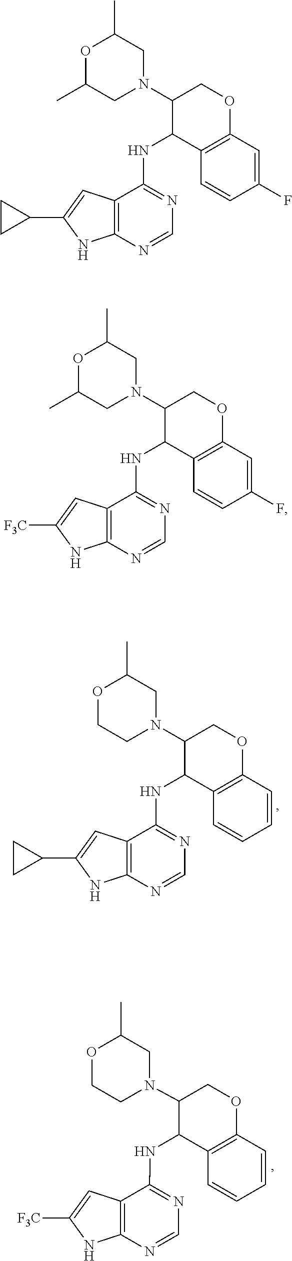 [SMILES: CC1CN(C2COc3cc(F)ccc3C2Nc2ncnc3[nH]c(C4CC4)cc23)CC(C)O1 ; CC1CN(C2COc3cc(F)ccc3C2Nc2ncnc3[nH]c(C(F)(F)F)cc23)CC(C)O1 ; CC1CN(C2COc3ccccc3C2Nc2ncnc3[nH]c(C4CC4)cc23)CCO1 ; CC1CN(C2COc3ccccc3C2Nc2ncnc3[nH]c(C(F)(F)F)cc23)CCO1]

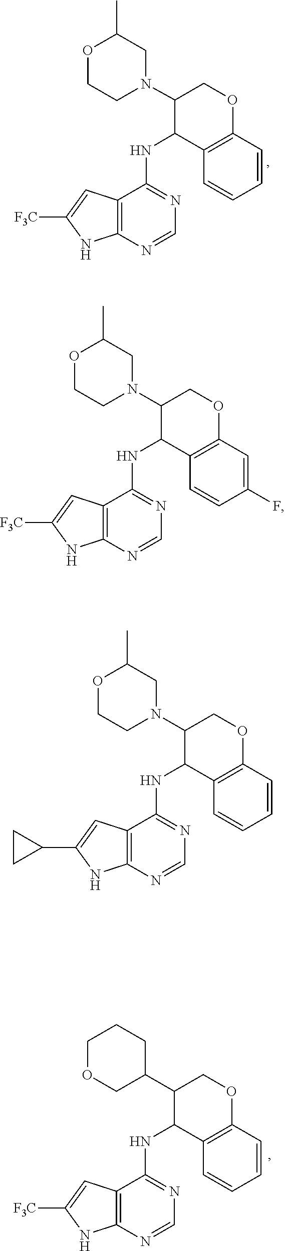 [SMILES: CC1CN(C2COc3ccccc3C2Nc2ncnc3[nH]c(C(F)(F)F)cc23)CCO1 ; CC1CN(C2COc3cc(F)ccc3C2Nc2ncnc3[nH]c(C(F)(F)F)cc23)CCO1 ; CC1CN(C2COc3ccccc3C2Nc2ncnc3[nH]c(C4CC4)cc23)CCO1 ; FC(F)(F)c1cc2c(NC3c4ccccc4OCC3C3CCCOC3)ncnc2[nH]1]

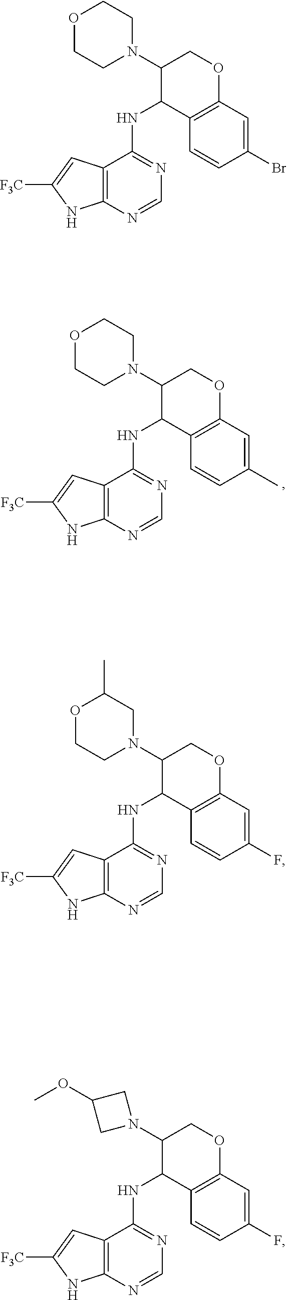 [SMILES: FC(F)(F)c1cc2c(NC3c4ccc(Br)cc4OCC3N3CCOCC3)ncnc2[nH]1 ; Cc1ccc2c(c1)OCC(N1CCOCC1)C2Nc1ncnc2[nH]c(C(F)(F)F)cc12 ; CC1CN(C2COc3cc(F)ccc3C2Nc2ncnc3[nH]c(C(F)(F)F)cc23)CCO1 ; COC1CN(C2COc3cc(F)ccc3C2Nc2ncnc3[nH]c(C(F)(F)F)cc23)C1]

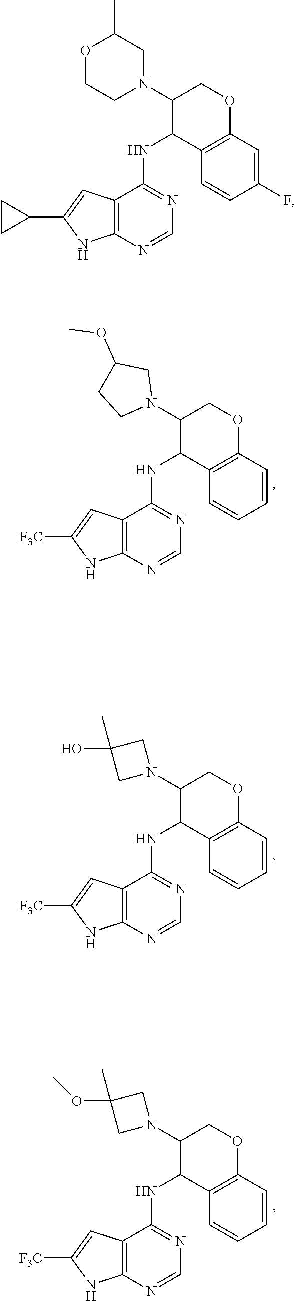 [SMILES: CC1CN(C2COc3cc(F)ccc3C2Nc2ncnc3[nH]c(C4CC4)cc23)CCO1 ; COC1CCN(C2COc3ccccc3C2Nc2ncnc3[nH]c(C(F)(F)F)cc23)C1 ; CC1(O)CN(C2COc3ccccc3C2Nc2ncnc3[nH]c(C(F)(F)F)cc23)C1 ; COC1(C)CN(C2COc3ccccc3C2Nc2ncnc3[nH]c(C(F)(F)F)cc23)C1]

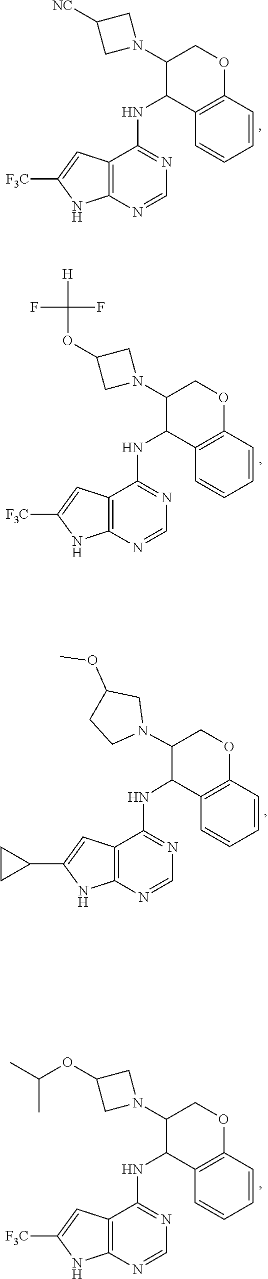 [SMILES: N#CC1CN(C2COc3ccccc3C2Nc2ncnc3[nH]c(C(F)(F)F)cc23)C1 ; FC(F)OC1CN(C2COc3ccccc3C2Nc2ncnc3[nH]c(C(F)(F)F)cc23)C1 ; COC1CCN(C2COc3ccccc3C2Nc2ncnc3[nH]c(C4CC4)cc23)C1 ; CC(C)OC1CN(C2COc3ccccc3C2Nc2ncnc3[nH]c(C(F)(F)F)cc23)C1]

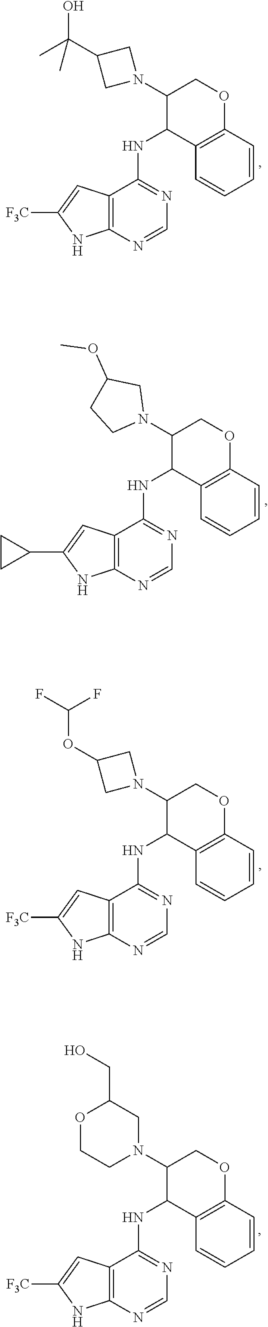 [SMILES: CC(C)(O)C1CN(C2COc3ccccc3C2Nc2ncnc3[nH]c(C(F)(F)F)cc23)C1 ; COC1CCN(C2COc3ccccc3C2Nc2ncnc3[nH]c(C4CC4)cc23)C1 ; FC(F)OC1CN(C2COc3ccccc3C2Nc2ncnc3[nH]c(C(F)(F)F)cc23)C1 ; OCC1CN(C2COc3ccccc3C2Nc2ncnc3[nH]c(C(F)(F)F)cc23)CCO1]

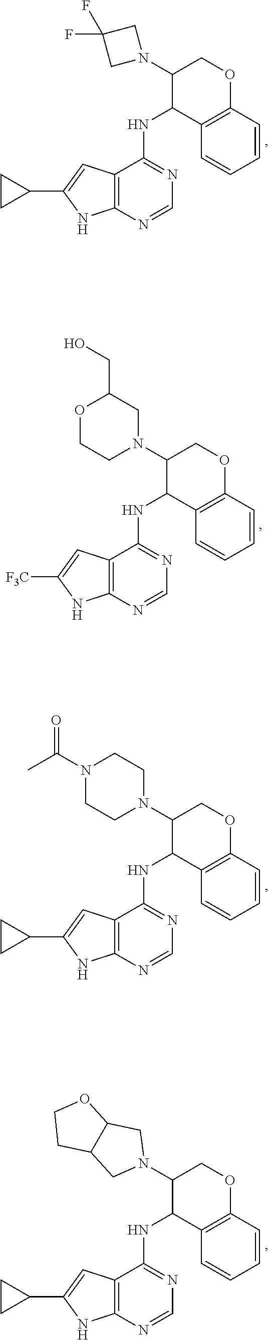 [SMILES: FC1(F)CN(C2COc3ccccc3C2Nc2ncnc3[nH]c(C4CC4)cc23)C1 ; OCC1CN(C2COc3ccccc3C2Nc2ncnc3[nH]c(C(F)(F)F)cc23)CCO1 ; CC(=O)N1CCN(C2COc3ccccc3C2Nc2ncnc3[nH]c(C4CC4)cc23)CC1 ; c1ccc2c(c1)OCC(N1CC3CCOC3C1)C2Nc1ncnc2[nH]c(C3CC3)cc12]

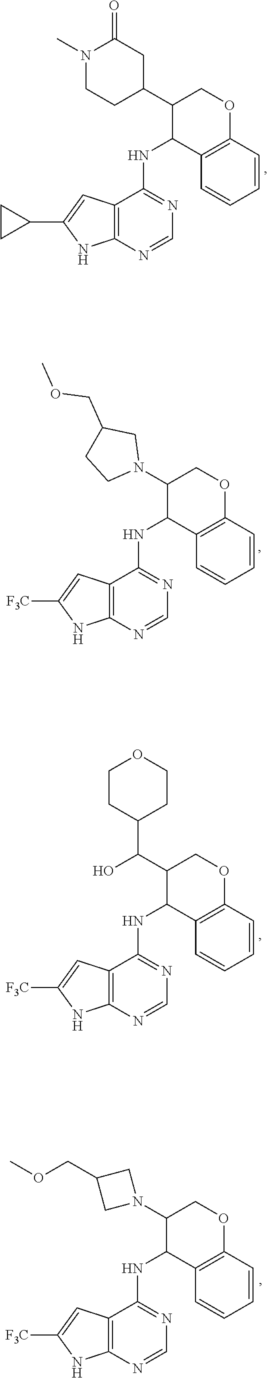 [SMILES: CN1CCC(C2COc3ccccc3C2Nc2ncnc3[nH]c(C4CC4)cc23)CC1=O ; COCC1CCN(C2COc3ccccc3C2Nc2ncnc3[nH]c(C(F)(F)F)cc23)C1 ; OC(C1CCOCC1)C1COc2ccccc2C1Nc1ncnc2[nH]c(C(F)(F)F)cc12 ; COCC1CN(C2COc3ccccc3C2Nc2ncnc3[nH]c(C(F)(F)F)cc23)C1]

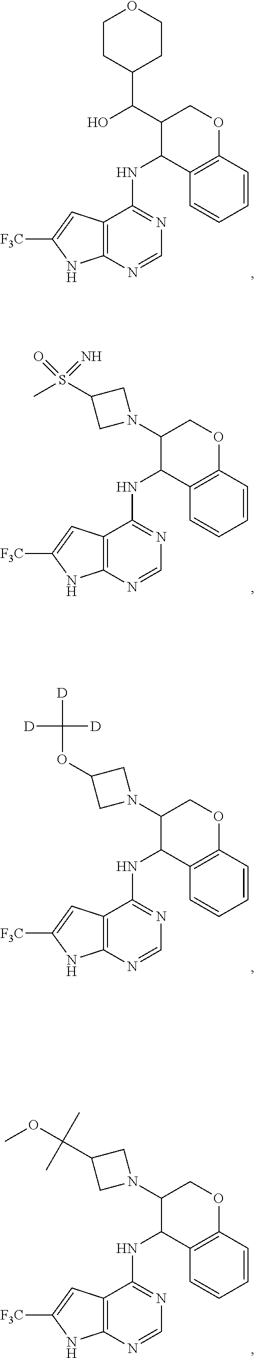 [SMILES: OC(C1CCOCC1)C1COc2ccccc2C1Nc1ncnc2[nH]c(C(F)(F)F)cc12 ; CS(=N)(=O)C1CN(C2COc3ccccc3C2Nc2ncnc3[nH]c(C(F)(F)F)cc23)C1 ; [2H]C([2H])([2H])OC1CN(C2COc3ccccc3C2Nc2ncnc3[nH]c(C(F)(F)F)cc23)C1 ; COC(C)(C)C1CN(C2COc3ccccc3C2Nc2ncnc3[nH]c(C(F)(F)F)cc23)C1]

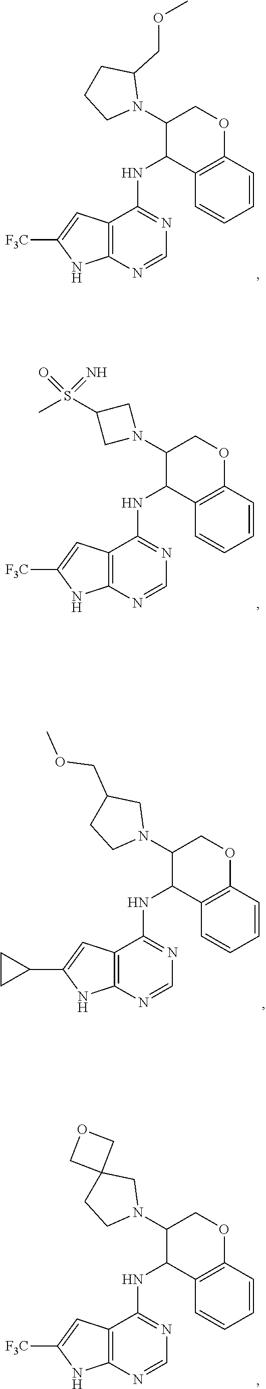 [SMILES: COCC1CCCN1C1COc2ccccc2C1Nc1ncnc2[nH]c(C(F)(F)F)cc12 ; CS(=N)(=O)C1CN(C2COc3ccccc3C2Nc2ncnc3[nH]c(C(F)(F)F)cc23)C1 ; COCC1CCN(C2COc3ccccc3C2Nc2ncnc3[nH]c(C4CC4)cc23)C1 ; FC(F)(F)c1cc2c(NC3c4ccccc4OCC3N3CCC4(COC4)C3)ncnc2[nH]1]

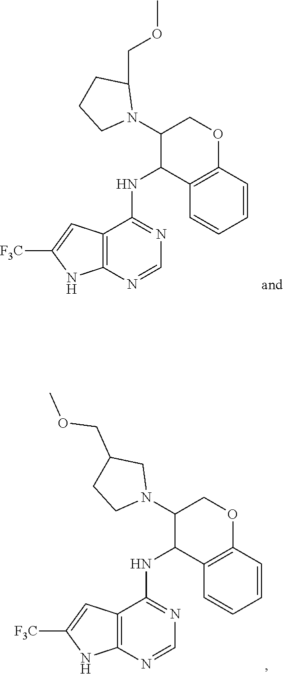 [SMILES: COCC1CCCN1C1COc2ccccc2C1Nc1ncnc2[nH]c(C(F)(F)F)cc12 ; COCC1CCN(C2COc3ccccc3C2Nc2ncnc3[nH]c(C(F)(F)F)cc23)C1]

or a pharmaceutically acceptable salt thereof.

In some embodiments, a compound can be present as one or more of the following structures:

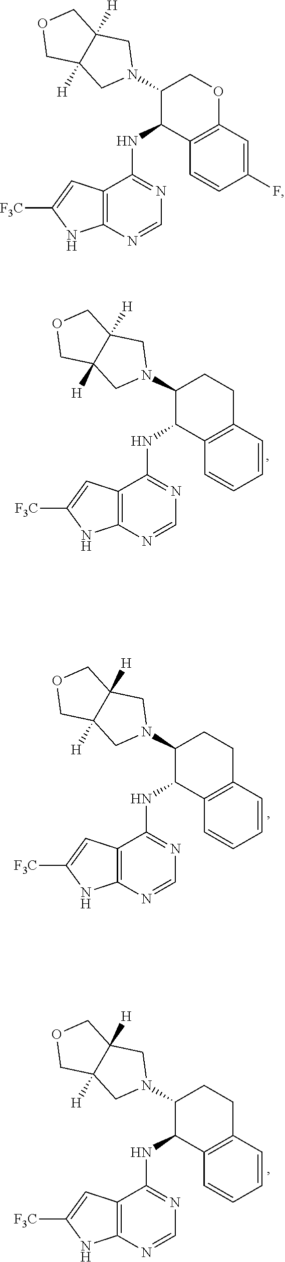 [SMILES: Fc1ccc2c(c1)OC[C@@H](N1C[C@H]3COC[C@H]3C1)[C@@H]2Nc1ncnc2[nH]c(C(F)(F)F)cc12 ; FC(F)(F)c1cc2c(N[C@H]3c4ccccc4CC[C@@H]3N3C[C@@H]4COC[C@H]4C3)ncnc2[nH]1 ; FC(F)(F)c1cc2c(N[C@H]3c4ccccc4CC[C@@H]3N3C[C@H]4COC[C@@H]4C3)ncnc2[nH]1 ; FC(F)(F)c1cc2c(N[C@@H]3c4ccccc4CC[C@H]3N3C[C@H]4COC[C@@H]4C3)ncnc2[nH]1]

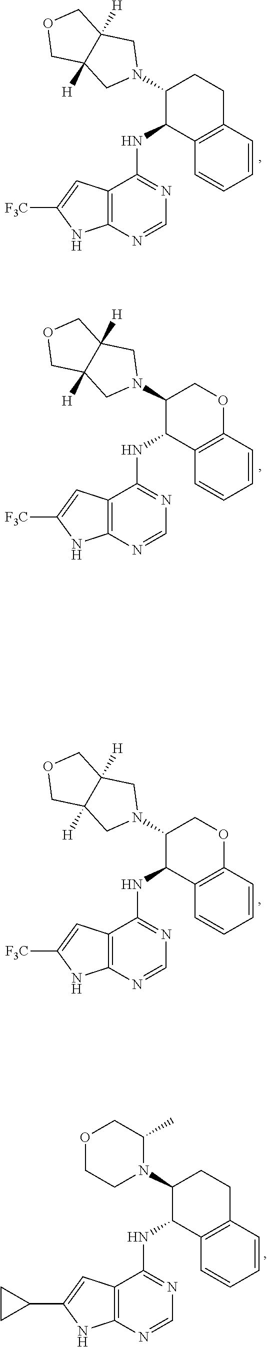 [SMILES: FC(F)(F)c1cc2c(N[C@@H]3c4ccccc4CC[C@H]3N3C[C@@H]4COC[C@H]4C3)ncnc2[nH]1 ; FC(F)(F)c1cc2c(N[C@H]3c4ccccc4OC[C@@H]3N3C[C@H]4COC[C@H]4C3)ncnc2[nH]1 ; FC(F)(F)c1cc2c(N[C@@H]3c4ccccc4OC[C@H]3N3C[C@H]4COC[C@H]4C3)ncnc2[nH]1 ; C[C@H]1COCCN1[C@H]1CCc2ccccc2[C@@H]1Nc1ncnc2[nH]c(C3CC3)cc12]

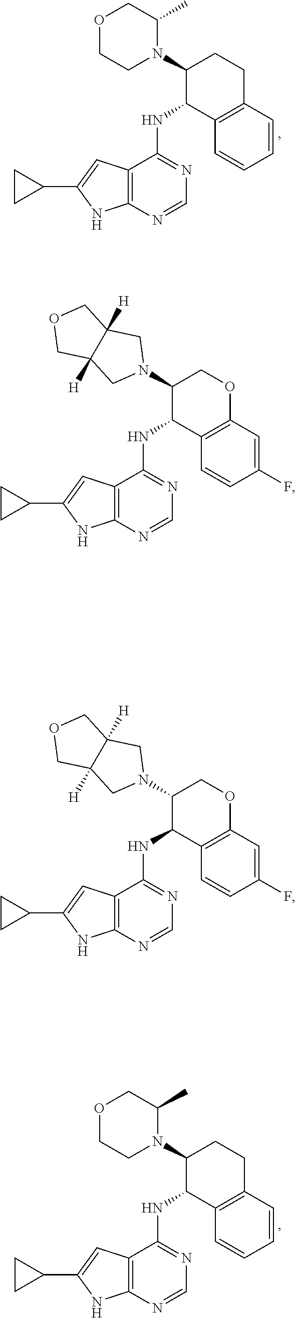 [SMILES: C[C@H]1COCCN1[C@H]1CCc2ccccc2[C@@H]1Nc1ncnc2[nH]c(C3CC3)cc12 ; Fc1ccc2c(c1)OC[C@H](N1C[C@H]3COC[C@H]3C1)[C@H]2Nc1ncnc2[nH]c(C3CC3)cc12 ; Fc1ccc2c(c1)OC[C@@H](N1C[C@H]3COC[C@H]3C1)[C@@H]2Nc1ncnc2[nH]c(C3CC3)cc12 ; C[C@@H]1COCCN1[C@H]1CCc2ccccc2[C@@H]1Nc1ncnc2[nH]c(C3CC3)cc12]

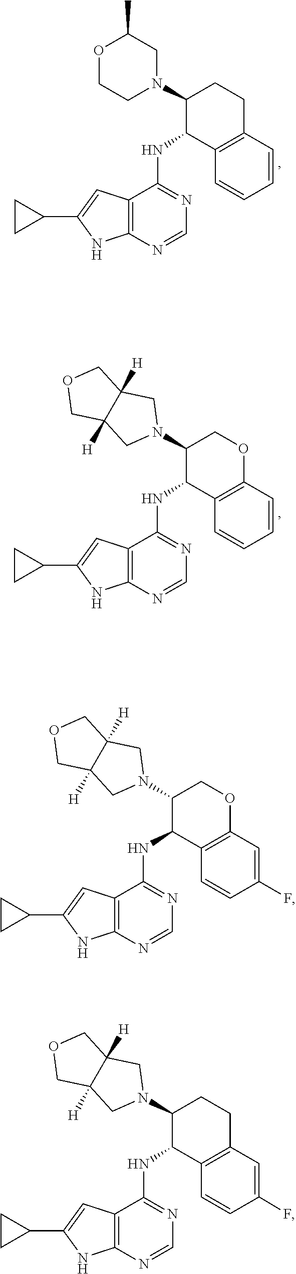 [SMILES: C[C@H]1CN([C@H]2CCc3ccccc3[C@@H]2Nc2ncnc3[nH]c(C4CC4)cc23)CCO1 ; c1ccc2c(c1)OC[C@H](N1C[C@H]3COC[C@H]3C1)[C@H]2Nc1ncnc2[nH]c(C3CC3)cc12 ; Fc1ccc2c(c1)OC[C@@H](N1C[C@H]3COC[C@H]3C1)[C@@H]2Nc1ncnc2[nH]c(C3CC3)cc12 ; Fc1ccc2c(c1)CC[C@H](N1C[C@H]3COC[C@@H]3C1)[C@H]2Nc1ncnc2[nH]c(C3CC3)cc12]

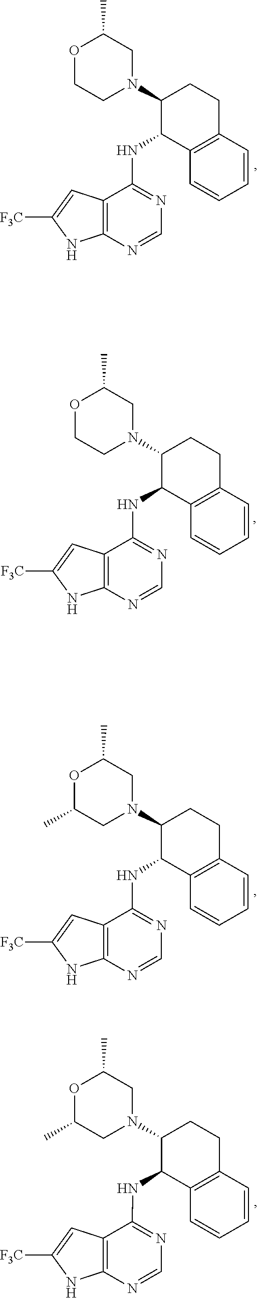 [SMILES: C[C@@H]1CN([C@H]2CCc3ccccc3[C@@H]2Nc2ncnc3[nH]c(C(F)(F)F)cc23)CCO1 ; C[C@@H]1CN([C@@H]2CCc3ccccc3[C@H]2Nc2ncnc3[nH]c(C(F)(F)F)cc23)CCO1 ; C[C@@H]1CN([C@H]2CCc3ccccc3[C@@H]2Nc2ncnc3[nH]c(C(F)(F)F)cc23)C[C@H](C)O1 ; C[C@@H]1CN([C@@H]2CCc3ccccc3[C@H]2Nc2ncnc3[nH]c(C(F)(F)F)cc23)C[C@H](C)O1]

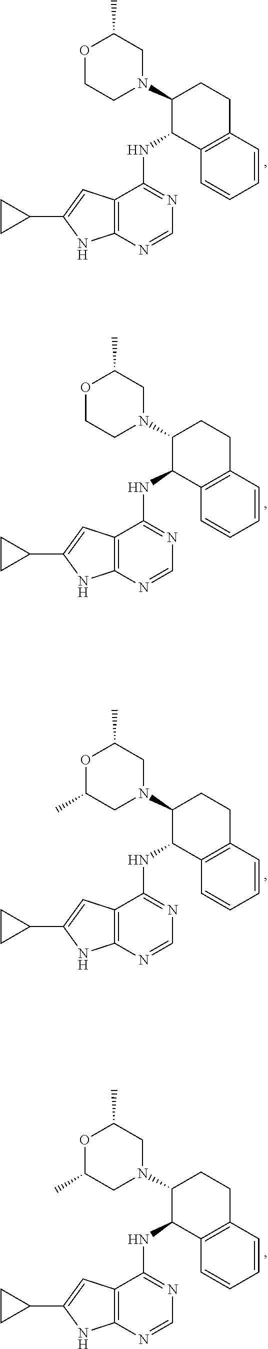 [SMILES: C[C@@H]1CN([C@H]2CCc3ccccc3[C@@H]2Nc2ncnc3[nH]c(C4CC4)cc23)CCO1 ; C[C@@H]1CN([C@@H]2CCc3ccccc3[C@H]2Nc2ncnc3[nH]c(C4CC4)cc23)CCO1 ; C[C@@H]1CN([C@H]2CCc3ccccc3[C@@H]2Nc2ncnc3[nH]c(C4CC4)cc23)C[C@H](C)O1 ; C[C@@H]1CN([C@@H]2CCc3ccccc3[C@H]2Nc2ncnc3[nH]c(C4CC4)cc23)C[C@H](C)O1]

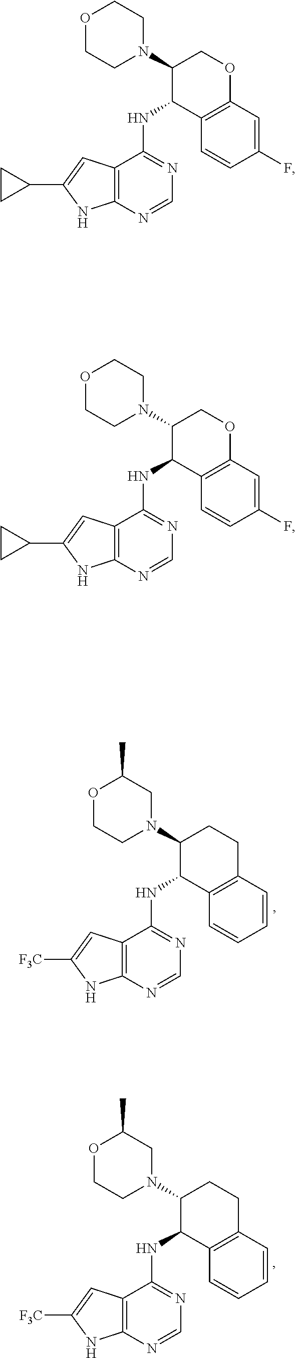 [SMILES: Fc1ccc2c(c1)OC[C@H](N1CCOCC1)[C@H]2Nc1ncnc2[nH]c(C3CC3)cc12 ; Fc1ccc2c(c1)OC[C@@H](N1CCOCC1)[C@@H]2Nc1ncnc2[nH]c(C3CC3)cc12 ; C[C@H]1CN([C@H]2CCc3ccccc3[C@@H]2Nc2ncnc3[nH]c(C(F)(F)F)cc23)CCO1 ; C[C@H]1CN([C@@H]2CCc3ccccc3[C@H]2Nc2ncnc3[nH]c(C(F)(F)F)cc23)CCO1]

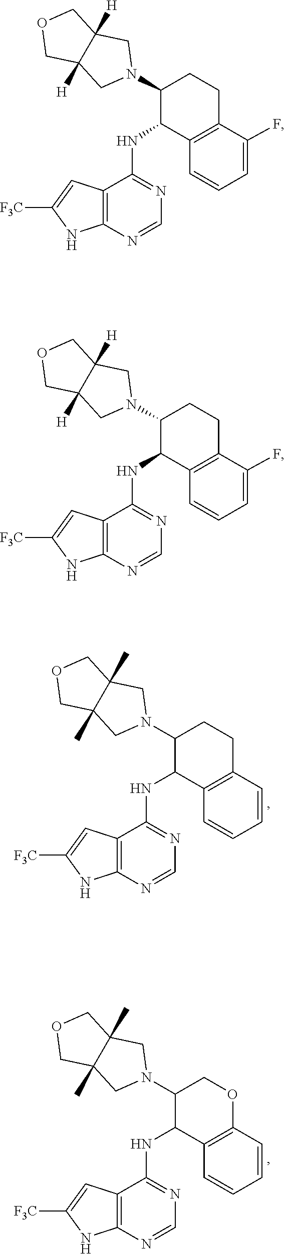 [SMILES: Fc1cccc2c1CC[C@H](N1C[C@H]3COC[C@H]3C1)[C@H]2Nc1ncnc2[nH]c(C(F)(F)F)cc12 ; Fc1cccc2c1CC[C@@H](N1C[C@H]3COC[C@H]3C1)[C@@H]2Nc1ncnc2[nH]c(C(F)(F)F)cc12 ; C[C@@]12COC[C@]1(C)CN(C1CCc3ccccc3C1Nc1ncnc3[nH]c(C(F)(F)F)cc13)C2 ; C[C@@]12COC[C@]1(C)CN(C1COc3ccccc3C1Nc1ncnc3[nH]c(C(F)(F)F)cc13)C2]

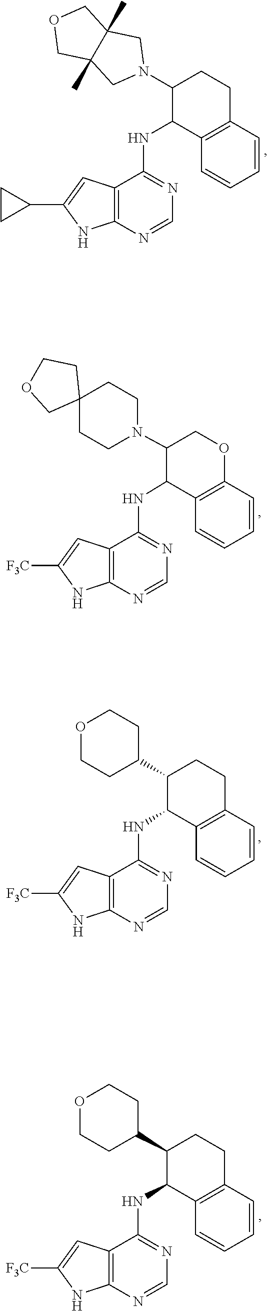 [SMILES: C[C@@]12COC[C@]1(C)CN(C1CCc3ccccc3C1Nc1ncnc3[nH]c(C4CC4)cc13)C2 ; FC(F)(F)c1cc2c(NC3c4ccccc4OCC3N3CCC4(CCOC4)CC3)ncnc2[nH]1 ; FC(F)(F)c1cc2c(N[C@H]3c4ccccc4CC[C@H]3C3CCOCC3)ncnc2[nH]1 ; FC(F)(F)c1cc2c(N[C@@H]3c4ccccc4CC[C@@H]3C3CCOCC3)ncnc2[nH]1]

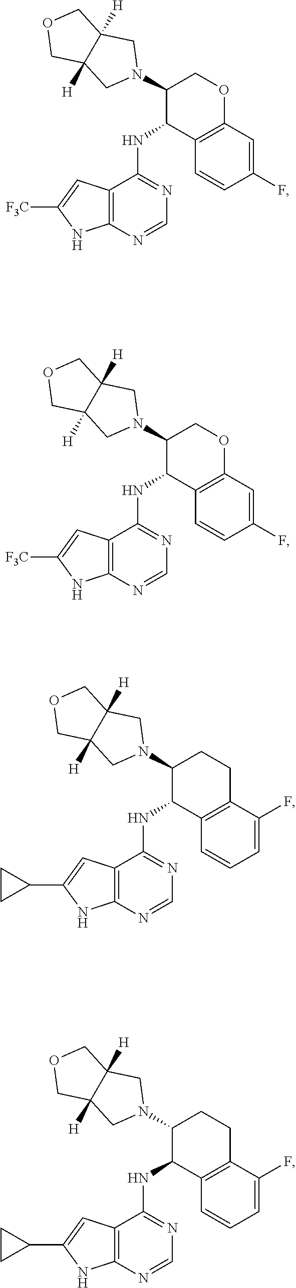 [SMILES: Fc1ccc2c(c1)OC[C@H](N1C[C@@H]3COC[C@H]3C1)[C@H]2Nc1ncnc2[nH]c(C(F)(F)F)cc12 ; Fc1ccc2c(c1)OC[C@H](N1C[C@H]3COC[C@@H]3C1)[C@H]2Nc1ncnc2[nH]c(C(F)(F)F)cc12 ; Fc1cccc2c1CC[C@H](N1C[C@H]3COC[C@H]3C1)[C@H]2Nc1ncnc2[nH]c(C3CC3)cc12 ; Fc1cccc2c1CC[C@@H](N1C[C@H]3COC[C@H]3C1)[C@@H]2Nc1ncnc2[nH]c(C3CC3)cc12]

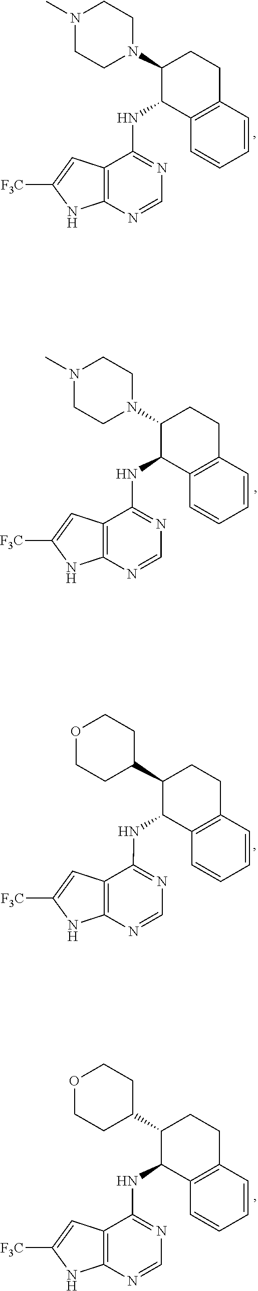 [SMILES: CN1CCN([C@H]2CCc3ccccc3[C@@H]2Nc2ncnc3[nH]c(C(F)(F)F)cc23)CC1 ; CN1CCN([C@@H]2CCc3ccccc3[C@H]2Nc2ncnc3[nH]c(C(F)(F)F)cc23)CC1 ; FC(F)(F)c1cc2c(N[C@H]3c4ccccc4CC[C@@H]3C3CCOCC3)ncnc2[nH]1 ; FC(F)(F)c1cc2c(N[C@@H]3c4ccccc4CC[C@H]3C3CCOCC3)ncnc2[nH]1]

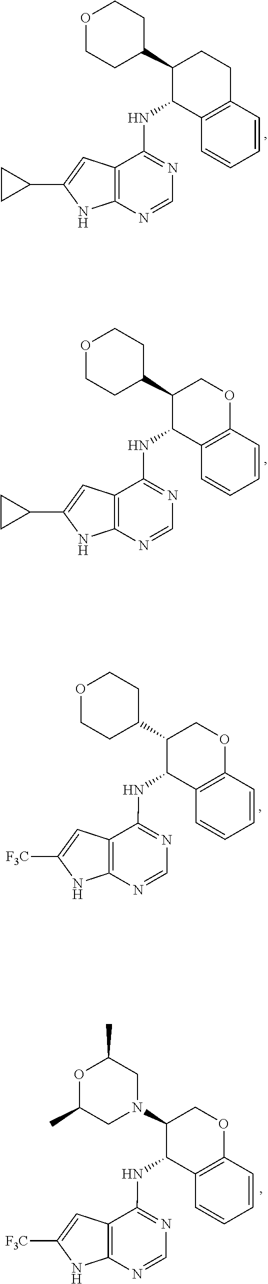 [SMILES: c1ccc2c(c1)CC[C@H](C1CCOCC1)[C@H]2Nc1ncnc2[nH]c(C3CC3)cc12 ; c1ccc2c(c1)OC[C@H](C1CCOCC1)[C@H]2Nc1ncnc2[nH]c(C3CC3)cc12 ; FC(F)(F)c1cc2c(N[C@H]3c4ccccc4OC[C@H]3C3CCOCC3)ncnc2[nH]1 ; C[C@@H]1CN([C@H]2COc3ccccc3[C@@H]2Nc2ncnc3[nH]c(C(F)(F)F)cc23)C[C@H](C)O1]

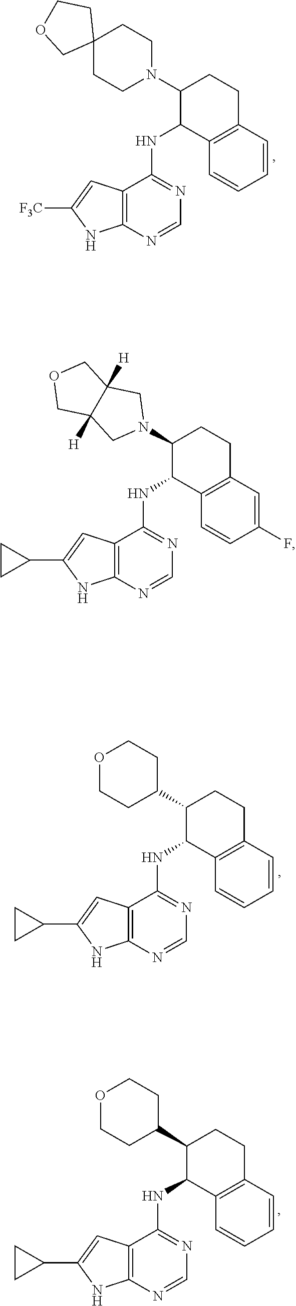 [SMILES: FC(F)(F)c1cc2c(NC3c4ccccc4CCC3N3CCC4(CCOC4)CC3)ncnc2[nH]1 ; Fc1ccc2c(c1)CC[C@H](N1C[C@H]3COC[C@H]3C1)[C@H]2Nc1ncnc2[nH]c(C3CC3)cc12 ; c1ccc2c(c1)CC[C@@H](C1CCOCC1)[C@H]2Nc1ncnc2[nH]c(C3CC3)cc12 ; c1ccc2c(c1)CC[C@H](C1CCOCC1)[C@@H]2Nc1ncnc2[nH]c(C3CC3)cc12]

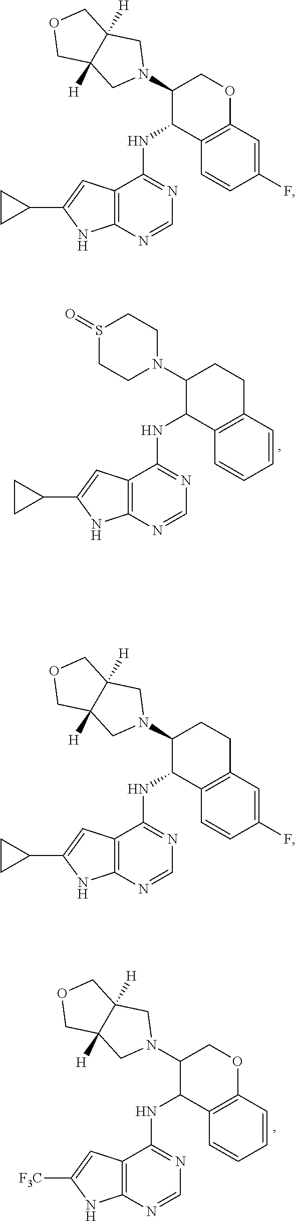 [SMILES: Fc1ccc2c(c1)OC[C@H](N1C[C@@H]3COC[C@H]3C1)[C@H]2Nc1ncnc2[nH]c(C3CC3)cc12 ; O=S1CCN(C2CCc3ccccc3C2Nc2ncnc3[nH]c(C4CC4)cc23)CC1 ; Fc1ccc2c(c1)CC[C@H](N1C[C@@H]3COC[C@H]3C1)[C@H]2Nc1ncnc2[nH]c(C3CC3)cc12 ; FC(F)(F)c1cc2c(NC3c4ccccc4OCC3N3C[C@@H]4COC[C@H]4C3)ncnc2[nH]1]

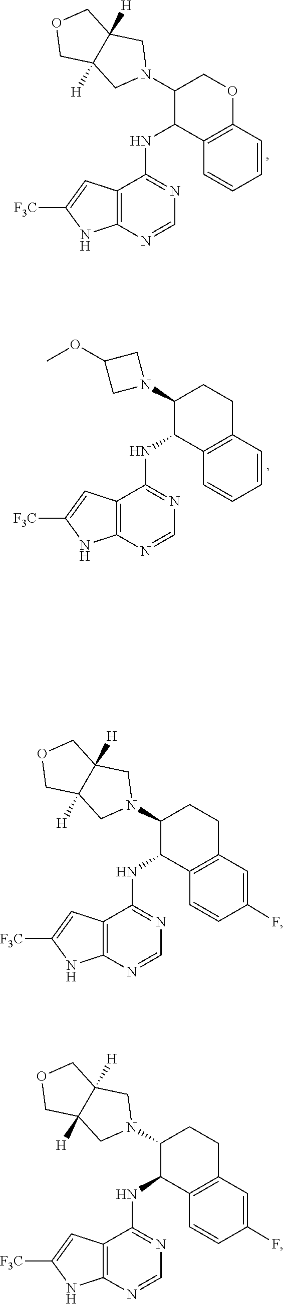 [SMILES: FC(F)(F)c1cc2c(NC3c4ccccc4OCC3N3C[C@H]4COC[C@@H]4C3)ncnc2[nH]1 ; COC1CN([C@H]2CCc3ccccc3[C@@H]2Nc2ncnc3[nH]c(C(F)(F)F)cc23)C1 ; Fc1ccc2c(c1)CC[C@H](N1C[C@H]3COC[C@@H]3C1)[C@H]2Nc1ncnc2[nH]c(C(F)(F)F)cc12 ; Fc1ccc2c(c1)CC[C@@H](N1C[C@@H]3COC[C@H]3C1)[C@@H]2Nc1ncnc2[nH]c(C(F)(F)F)cc12]

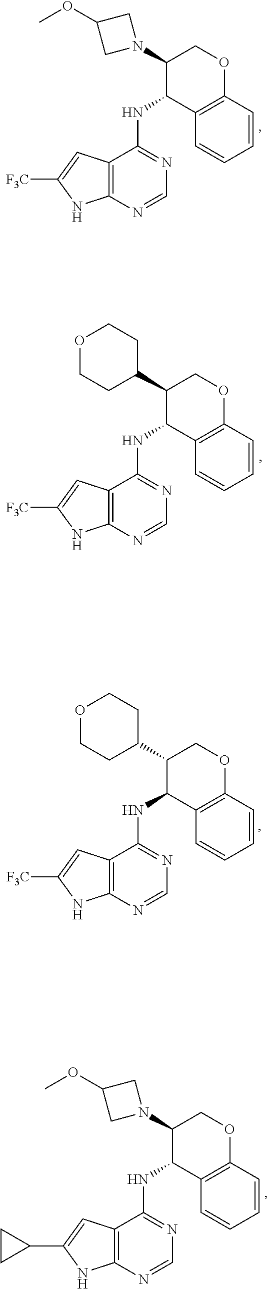 [SMILES: COC1CN([C@H]2COc3ccccc3[C@@H]2Nc2ncnc3[nH]c(C(F)(F)F)cc23)C1 ; FC(F)(F)c1cc2c(N[C@H]3c4ccccc4OC[C@@H]3C3CCOCC3)ncnc2[nH]1 ; FC(F)(F)c1cc2c(N[C@@H]3c4ccccc4OC[C@H]3C3CCOCC3)ncnc2[nH]1 ; COC1CN([C@H]2COc3ccccc3[C@@H]2Nc2ncnc3[nH]c(C4CC4)cc23)C1]

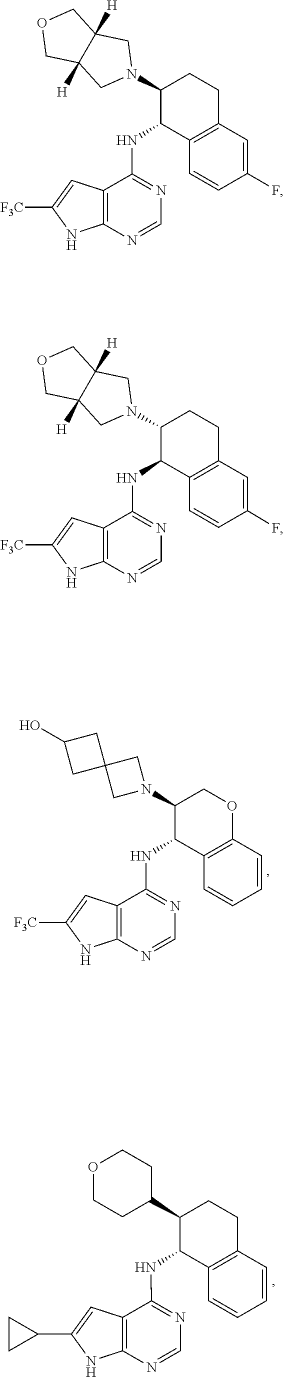 [SMILES: Fc1ccc2c(c1)CC[C@H](N1C[C@H]3COC[C@H]3C1)[C@H]2Nc1ncnc2[nH]c(C(F)(F)F)cc12 ; Fc1ccc2c(c1)CC[C@@H](N1C[C@H]3COC[C@H]3C1)[C@@H]2Nc1ncnc2[nH]c(C(F)(F)F)cc12 ; OC1CC2(C1)CN([C@H]1COc3ccccc3[C@@H]1Nc1ncnc3[nH]c(C(F)(F)F)cc13)C2 ; c1ccc2c(c1)CC[C@H](C1CCOCC1)[C@H]2Nc1ncnc2[nH]c(C3CC3)cc12]

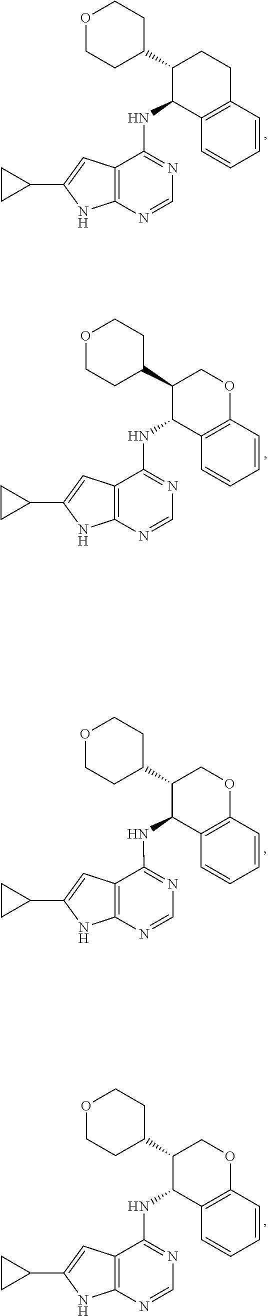 [SMILES: c1ccc2c(c1)CC[C@@H](C1CCOCC1)[C@@H]2Nc1ncnc2[nH]c(C3CC3)cc12 ; c1ccc2c(c1)OC[C@H](C1CCOCC1)[C@H]2Nc1ncnc2[nH]c(C3CC3)cc12 ; c1ccc2c(c1)OC[C@@H](C1CCOCC1)[C@@H]2Nc1ncnc2[nH]c(C3CC3)cc12 ; c1ccc2c(c1)OC[C@@H](C1CCOCC1)[C@H]2Nc1ncnc2[nH]c(C3CC3)cc12]

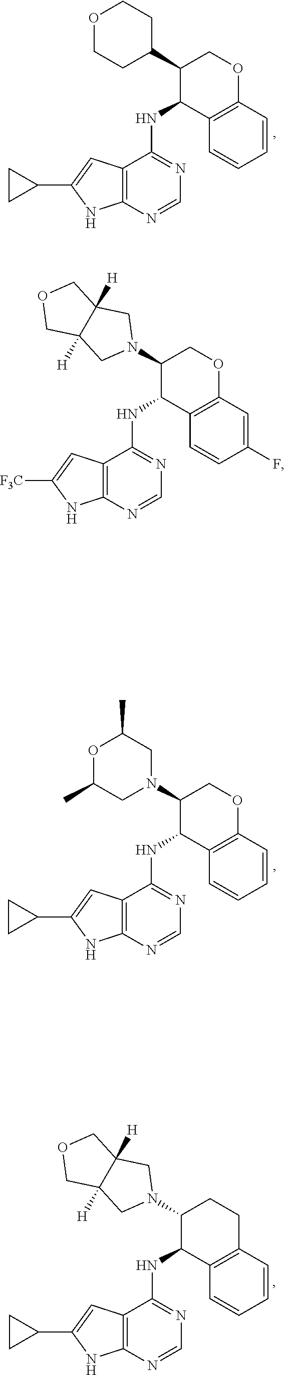 [SMILES: c1ccc2c(c1)OC[C@H](C1CCOCC1)[C@@H]2Nc1ncnc2[nH]c(C3CC3)cc12 ; Fc1ccc2c(c1)OC[C@H](N1C[C@H]3COC[C@@H]3C1)[C@H]2Nc1ncnc2[nH]c(C(F)(F)F)cc12 ; C[C@@H]1CN([C@H]2COc3ccccc3[C@@H]2Nc2ncnc3[nH]c(C4CC4)cc23)C[C@H](C)O1 ; c1ccc2c(c1)CC[C@@H](N1C[C@H]3COC[C@@H]3C1)[C@@H]2Nc1ncnc2[nH]c(C3CC3)cc12]

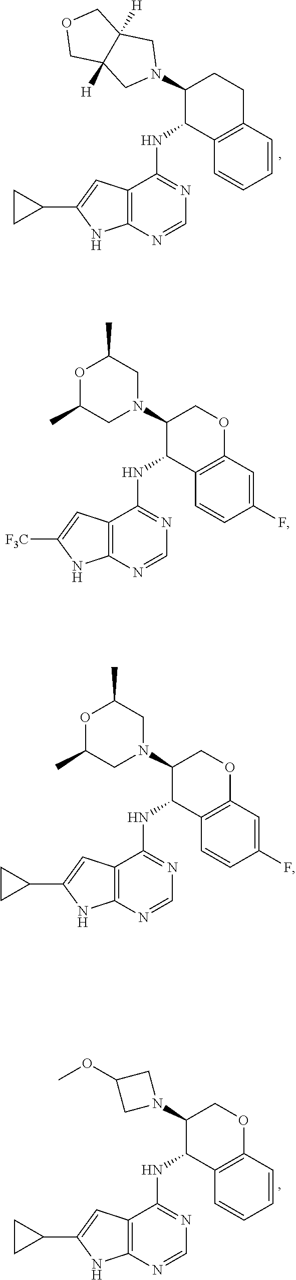 [SMILES: c1ccc2c(c1)CC[C@H](N1C[C@@H]3COC[C@H]3C1)[C@H]2Nc1ncnc2[nH]c(C3CC3)cc12 ; C[C@@H]1CN([C@H]2COc3cc(F)ccc3[C@@H]2Nc2ncnc3[nH]c(C(F)(F)F)cc23)C[C@H](C)O1 ; C[C@@H]1CN([C@H]2COc3cc(F)ccc3[C@@H]2Nc2ncnc3[nH]c(C4CC4)cc23)C[C@H](C)O1 ; COC1CN([C@H]2COc3ccccc3[C@@H]2Nc2ncnc3[nH]c(C4CC4)cc23)C1]

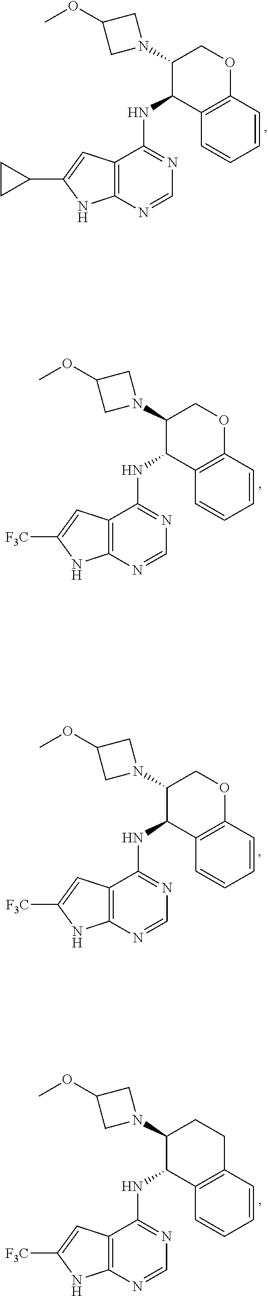 [SMILES: COC1CN([C@@H]2COc3ccccc3[C@H]2Nc2ncnc3[nH]c(C4CC4)cc23)C1 ; COC1CN([C@H]2COc3ccccc3[C@@H]2Nc2ncnc3[nH]c(C(F)(F)F)cc23)C1 ; COC1CN([C@@H]2COc3ccccc3[C@H]2Nc2ncnc3[nH]c(C(F)(F)F)cc23)C1 ; COC1CN([C@H]2CCc3ccccc3[C@@H]2Nc2ncnc3[nH]c(C(F)(F)F)cc23)C1]

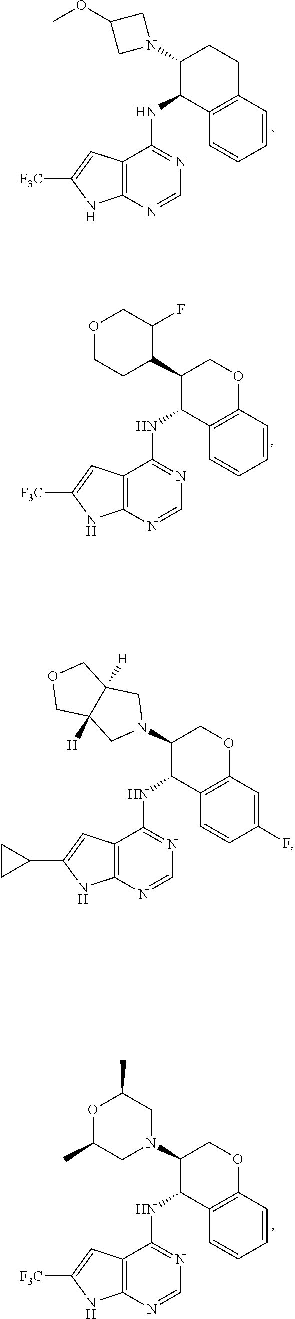 [SMILES: COC1CN([C@@H]2CCc3ccccc3[C@H]2Nc2ncnc3[nH]c(C(F)(F)F)cc23)C1 ; FC1COCCC1[C@H]1COc2ccccc2[C@@H]1Nc1ncnc2[nH]c(C(F)(F)F)cc12 ; Fc1ccc2c(c1)OC[C@H](N1C[C@@H]3COC[C@H]3C1)[C@H]2Nc1ncnc2[nH]c(C3CC3)cc12 ; C[C@@H]1CN([C@H]2COc3ccccc3[C@@H]2Nc2ncnc3[nH]c(C(F)(F)F)cc23)C[C@H](C)O1]

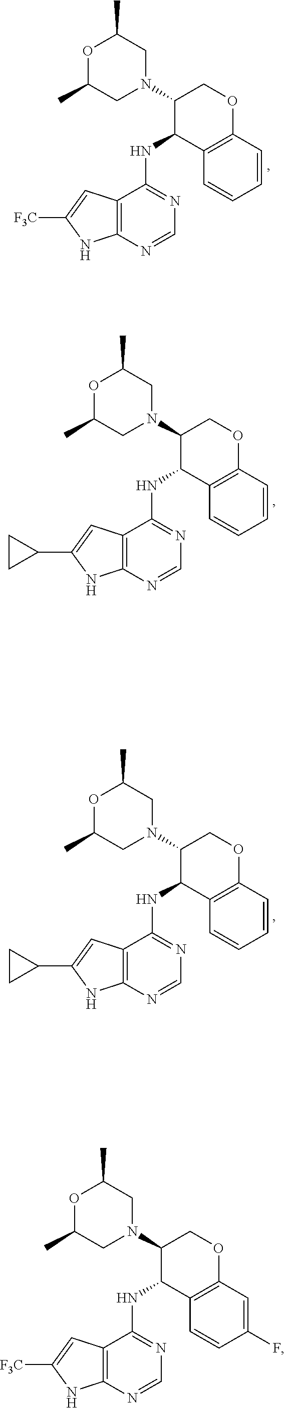 [SMILES: C[C@@H]1CN([C@@H]2COc3ccccc3[C@H]2Nc2ncnc3[nH]c(C(F)(F)F)cc23)C[C@H](C)O1 ; C[C@@H]1CN([C@H]2COc3ccccc3[C@@H]2Nc2ncnc3[nH]c(C4CC4)cc23)C[C@H](C)O1 ; C[C@@H]1CN([C@@H]2COc3ccccc3[C@H]2Nc2ncnc3[nH]c(C4CC4)cc23)C[C@H](C)O1 ; C[C@@H]1CN([C@H]2COc3cc(F)ccc3[C@@H]2Nc2ncnc3[nH]c(C(F)(F)F)cc23)C[C@H](C)O1]

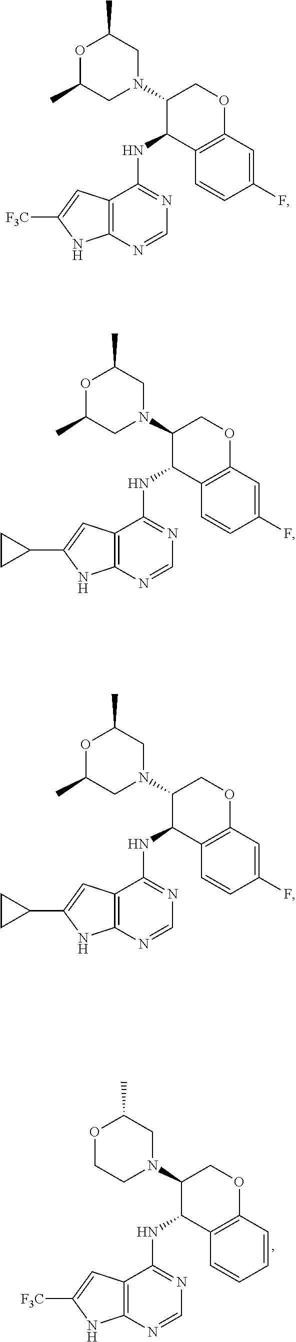 [SMILES: C[C@@H]1CN([C@@H]2COc3cc(F)ccc3[C@H]2Nc2ncnc3[nH]c(C(F)(F)F)cc23)C[C@H](C)O1 ; C[C@@H]1CN([C@H]2COc3cc(F)ccc3[C@@H]2Nc2ncnc3[nH]c(C4CC4)cc23)C[C@H](C)O1 ; C[C@@H]1CN([C@@H]2COc3cc(F)ccc3[C@H]2Nc2ncnc3[nH]c(C4CC4)cc23)C[C@H](C)O1 ; C[C@@H]1CN([C@H]2COc3ccccc3[C@@H]2Nc2ncnc3[nH]c(C(F)(F)F)cc23)CCO1]

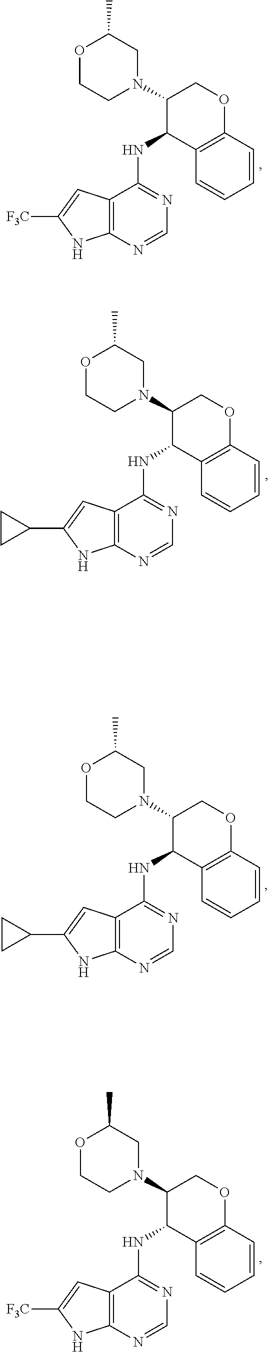 [SMILES: C[C@@H]1CN([C@@H]2COc3ccccc3[C@H]2Nc2ncnc3[nH]c(C(F)(F)F)cc23)CCO1 ; C[C@@H]1CN([C@H]2COc3ccccc3[C@@H]2Nc2ncnc3[nH]c(C4CC4)cc23)CCO1 ; C[C@@H]1CN([C@@H]2COc3ccccc3[C@H]2Nc2ncnc3[nH]c(C4CC4)cc23)CCO1 ; C[C@H]1CN([C@H]2COc3ccccc3[C@@H]2Nc2ncnc3[nH]c(C(F)(F)F)cc23)CCO1]

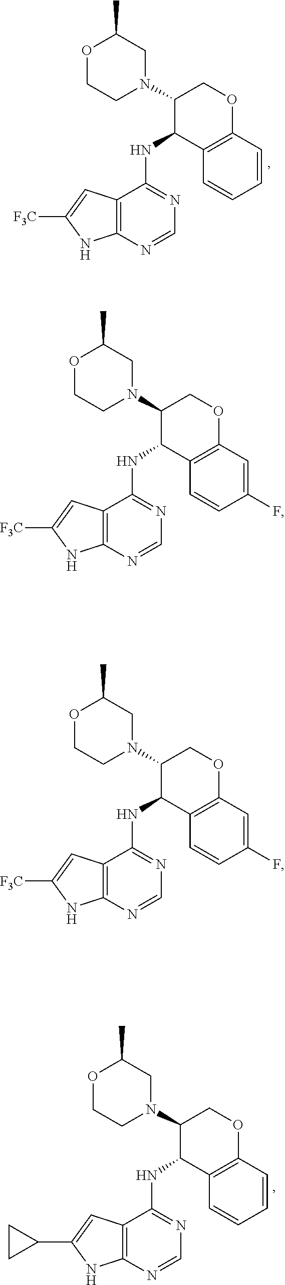 [SMILES: C[C@H]1CN([C@@H]2COc3ccccc3[C@H]2Nc2ncnc3[nH]c(C(F)(F)F)cc23)CCO1 ; C[C@H]1CN([C@H]2COc3cc(F)ccc3[C@@H]2Nc2ncnc3[nH]c(C(F)(F)F)cc23)CCO1 ; C[C@H]1CN([C@@H]2COc3cc(F)ccc3[C@H]2Nc2ncnc3[nH]c(C(F)(F)F)cc23)CCO1 ; C[C@H]1CN([C@H]2COc3ccccc3[C@@H]2Nc2ncnc3[nH]c(C4CC4)cc23)CCO1]

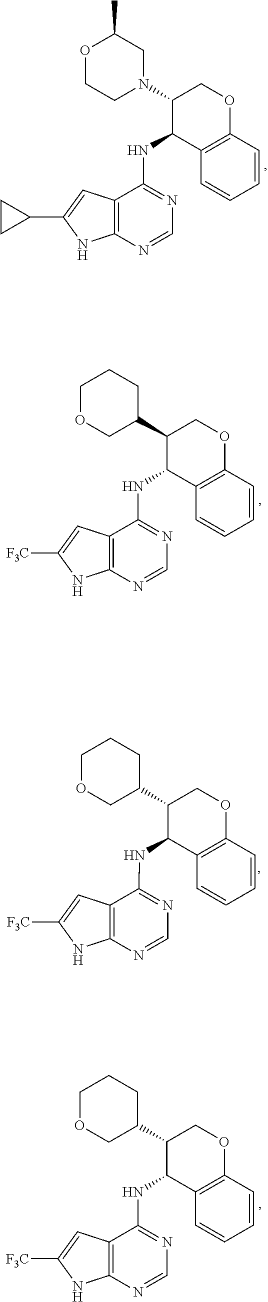 [SMILES: C[C@H]1CN([C@@H]2COc3ccccc3[C@H]2Nc2ncnc3[nH]c(C4CC4)cc23)CCO1 ; FC(F)(F)c1cc2c(N[C@H]3c4ccccc4OC[C@@H]3C3CCCOC3)ncnc2[nH]1 ; FC(F)(F)c1cc2c(N[C@@H]3c4ccccc4OC[C@H]3C3CCCOC3)ncnc2[nH]1 ; FC(F)(F)c1cc2c(N[C@H]3c4ccccc4OC[C@H]3C3CCCOC3)ncnc2[nH]1]

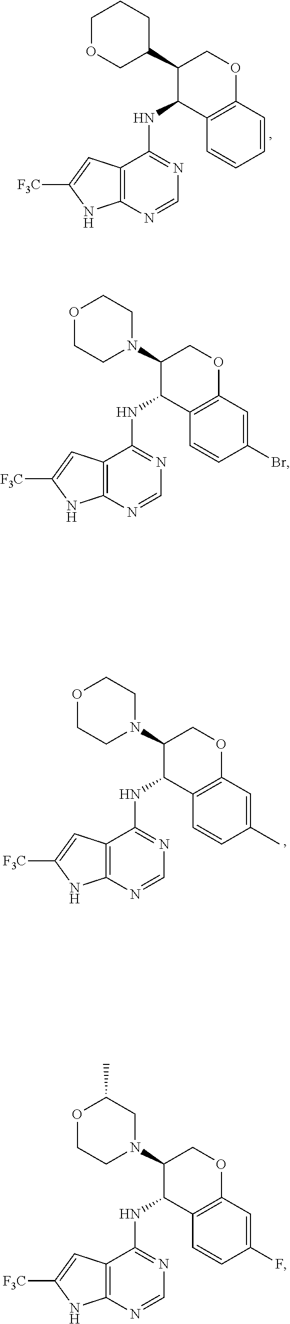 [SMILES: FC(F)(F)c1cc2c(N[C@@H]3c4ccccc4OC[C@@H]3C3CCCOC3)ncnc2[nH]1 ; FC(F)(F)c1cc2c(N[C@H]3c4ccc(Br)cc4OC[C@@H]3N3CCOCC3)ncnc2[nH]1 ; Cc1ccc2c(c1)OC[C@H](N1CCOCC1)[C@H]2Nc1ncnc2[nH]c(C(F)(F)F)cc12 ; C[C@@H]1CN([C@H]2COc3cc(F)ccc3[C@@H]2Nc2ncnc3[nH]c(C(F)(F)F)cc23)CCO1]

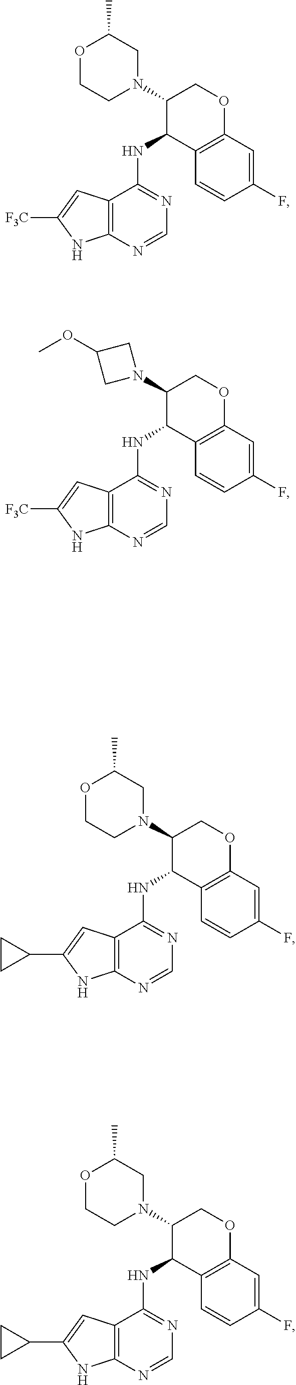 [SMILES: C[C@@H]1CN([C@@H]2COc3cc(F)ccc3[C@H]2Nc2ncnc3[nH]c(C(F)(F)F)cc23)CCO1 ; COC1CN([C@H]2COc3cc(F)ccc3[C@@H]2Nc2ncnc3[nH]c(C(F)(F)F)cc23)C1 ; C[C@@H]1CN([C@H]2COc3cc(F)ccc3[C@@H]2Nc2ncnc3[nH]c(C4CC4)cc23)CCO1 ; C[C@@H]1CN([C@@H]2COc3cc(F)ccc3[C@H]2Nc2ncnc3[nH]c(C4CC4)cc23)CCO1]

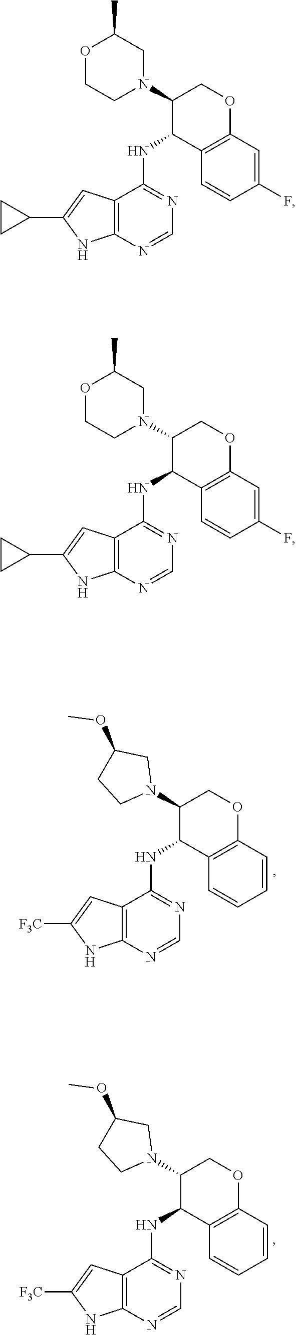 [SMILES: C[C@H]1CN([C@H]2COc3cc(F)ccc3[C@@H]2Nc2ncnc3[nH]c(C4CC4)cc23)CCO1 ; C[C@H]1CN([C@@H]2COc3cc(F)ccc3[C@H]2Nc2ncnc3[nH]c(C4CC4)cc23)CCO1 ; CO[C@@H]1CCN([C@H]2COc3ccccc3[C@@H]2Nc2ncnc3[nH]c(C(F)(F)F)cc23)C1 ; CO[C@@H]1CCN([C@@H]2COc3ccccc3[C@H]2Nc2ncnc3[nH]c(C(F)(F)F)cc23)C1]

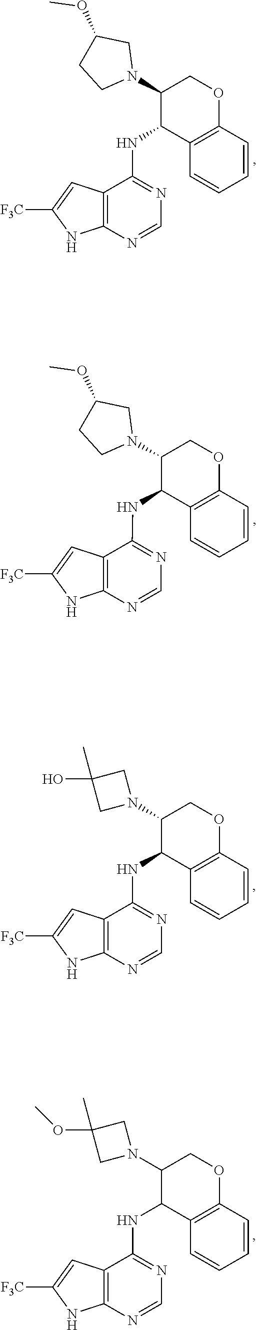 [SMILES: CO[C@H]1CCN([C@H]2COc3ccccc3[C@@H]2Nc2ncnc3[nH]c(C(F)(F)F)cc23)C1 ; CO[C@H]1CCN([C@@H]2COc3ccccc3[C@H]2Nc2ncnc3[nH]c(C(F)(F)F)cc23)C1 ; CC1(O)CN([C@@H]2COc3ccccc3[C@H]2Nc2ncnc3[nH]c(C(F)(F)F)cc23)C1 ; COC1(C)CN(C2COc3ccccc3C2Nc2ncnc3[nH]c(C(F)(F)F)cc23)C1]

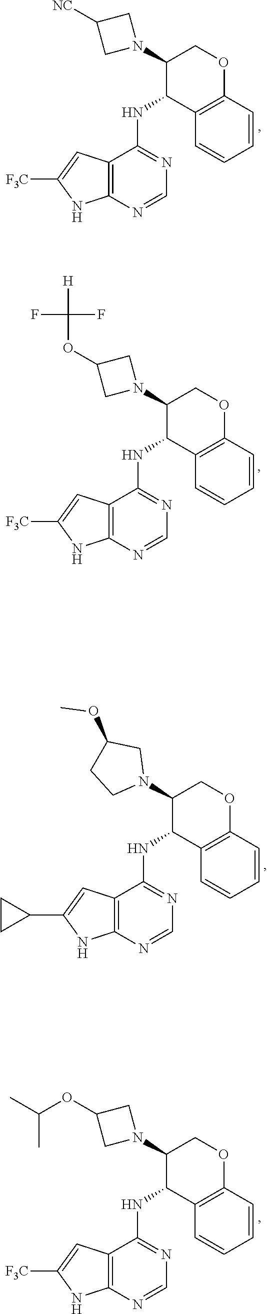 [SMILES: N#CC1CN([C@H]2COc3ccccc3[C@@H]2Nc2ncnc3[nH]c(C(F)(F)F)cc23)C1 ; FC(F)OC1CN([C@H]2COc3ccccc3[C@@H]2Nc2ncnc3[nH]c(C(F)(F)F)cc23)C1 ; CO[C@@H]1CCN([C@H]2COc3ccccc3[C@@H]2Nc2ncnc3[nH]c(C4CC4)cc23)C1 ; CC(C)OC1CN([C@H]2COc3ccccc3[C@@H]2Nc2ncnc3[nH]c(C(F)(F)F)cc23)C1]

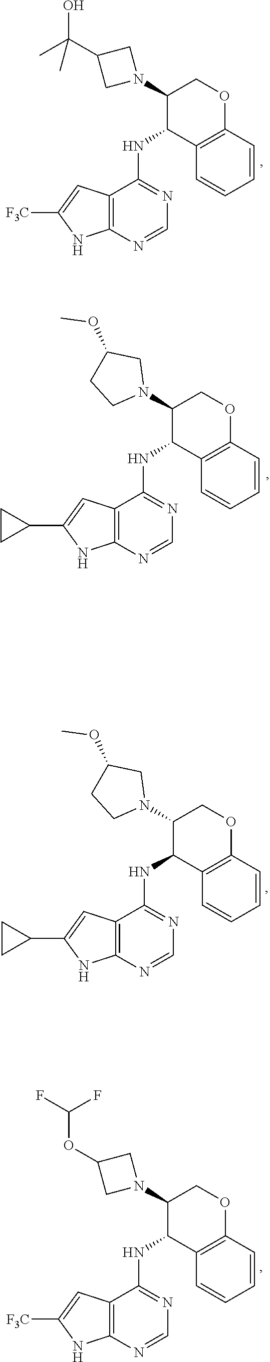 [SMILES: CC(C)(O)C1CN([C@H]2COc3ccccc3[C@@H]2Nc2ncnc3[nH]c(C(F)(F)F)cc23)C1 ; CO[C@H]1CCN([C@H]2COc3ccccc3[C@@H]2Nc2ncnc3[nH]c(C4CC4)cc23)C1 ; CO[C@H]1CCN([C@@H]2COc3ccccc3[C@H]2Nc2ncnc3[nH]c(C4CC4)cc23)C1 ; FC(F)OC1CN([C@H]2COc3ccccc3[C@@H]2Nc2ncnc3[nH]c(C(F)(F)F)cc23)C1]

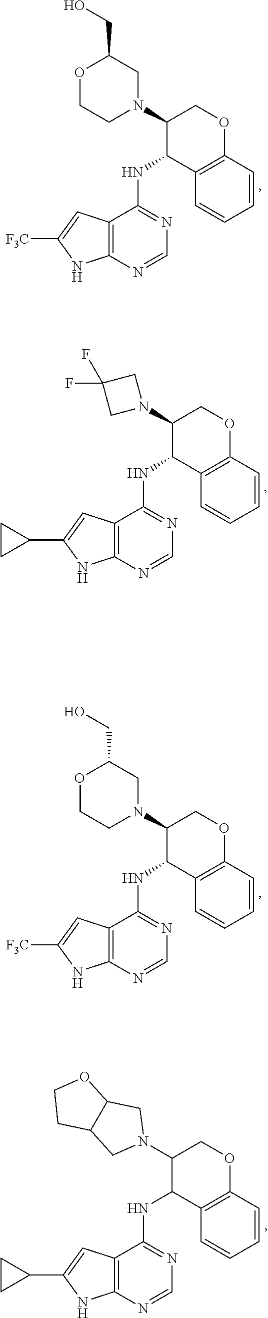 [SMILES: OC[C@H]1CN([C@H]2COc3ccccc3[C@@H]2Nc2ncnc3[nH]c(C(F)(F)F)cc23)CCO1 ; FC1(F)CN([C@H]2COc3ccccc3[C@@H]2Nc2ncnc3[nH]c(C4CC4)cc23)C1 ; OC[C@@H]1CN([C@H]2COc3ccccc3[C@@H]2Nc2ncnc3[nH]c(C(F)(F)F)cc23)CCO1 ; c1ccc2c(c1)OCC(N1CC3CCOC3C1)C2Nc1ncnc2[nH]c(C3CC3)cc12]

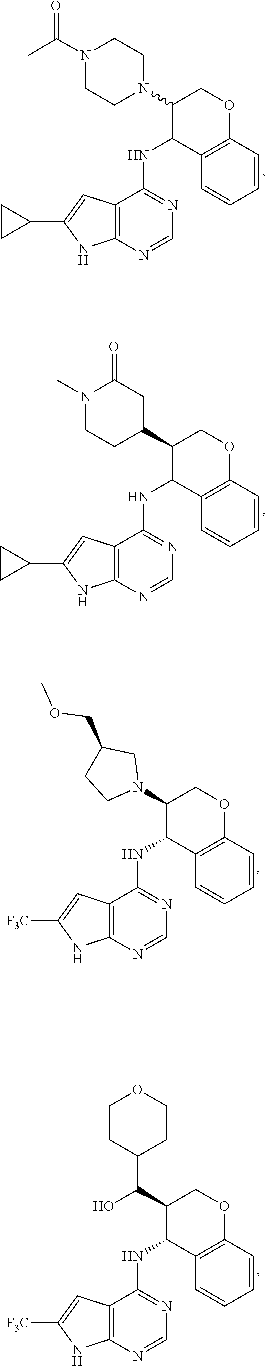 [SMILES: CC(=O)N1CCN(C2COc3ccccc3C2Nc2ncnc3[nH]c(C4CC4)cc23)CC1 ; CN1CCC([C@H]2COc3ccccc3C2Nc2ncnc3[nH]c(C4CC4)cc23)CC1=O ; COC[C@@H]1CCN([C@H]2COc3ccccc3[C@@H]2Nc2ncnc3[nH]c(C(F)(F)F)cc23)C1 ; OC(C1CCOCC1)[C@H]1COc2ccccc2[C@@H]1Nc1ncnc2[nH]c(C(F)(F)F)cc12]

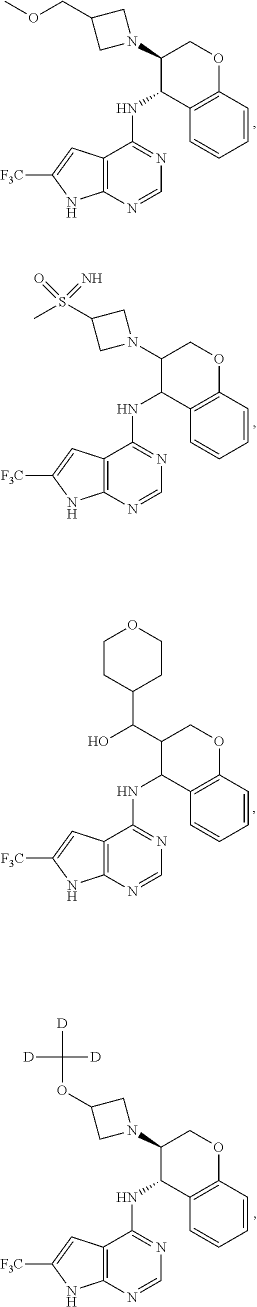 [SMILES: COCC1CN([C@H]2COc3ccccc3[C@@H]2Nc2ncnc3[nH]c(C(F)(F)F)cc23)C1 ; CS(=N)(=O)C1CN(C2COc3ccccc3C2Nc2ncnc3[nH]c(C(F)(F)F)cc23)C1 ; OC(C1CCOCC1)C1COc2ccccc2C1Nc1ncnc2[nH]c(C(F)(F)F)cc12 ; [2H]C([2H])([2H])OC1CN([C@H]2COc3ccccc3[C@@H]2Nc2ncnc3[nH]c(C(F)(F)F)cc23)C1]

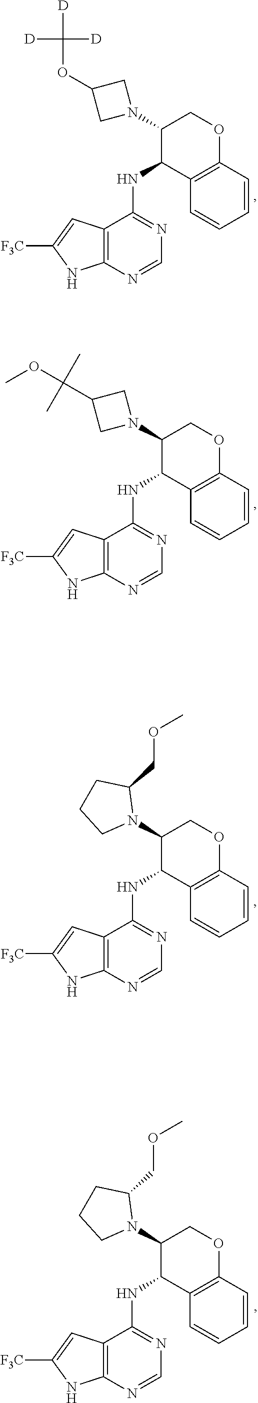 [SMILES: [2H]C([2H])([2H])OC1CN([C@@H]2COc3ccccc3[C@H]2Nc2ncnc3[nH]c(C(F)(F)F)cc23)C1 ; COC(C)(C)C1CN([C@H]2COc3ccccc3[C@@H]2Nc2ncnc3[nH]c(C(F)(F)F)cc23)C1 ; COC[C@@H]1CCCN1[C@H]1COc2ccccc2[C@@H]1Nc1ncnc2[nH]c(C(F)(F)F)cc12 ; COC[C@H]1CCCN1[C@H]1COc2ccccc2[C@@H]1Nc1ncnc2[nH]c(C(F)(F)F)cc12]

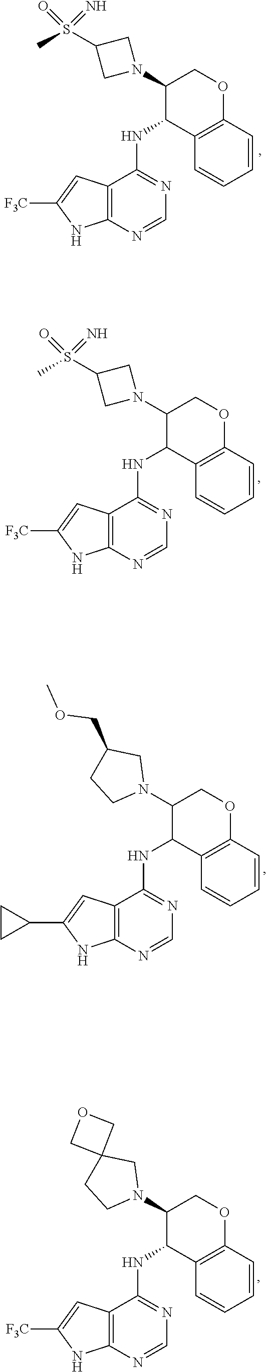 [SMILES: C[S@](=N)(=O)C1CN([C@H]2COc3ccccc3[C@@H]2Nc2ncnc3[nH]c(C(F)(F)F)cc23)C1 ; C[S@@](=N)(=O)C1CN(C2COc3ccccc3C2Nc2ncnc3[nH]c(C(F)(F)F)cc23)C1 ; COC[C@@H]1CCN(C2COc3ccccc3C2Nc2ncnc3[nH]c(C4CC4)cc23)C1 ; FC(F)(F)c1cc2c(N[C@H]3c4ccccc4OC[C@@H]3N3CCC4(COC4)C3)ncnc2[nH]1]

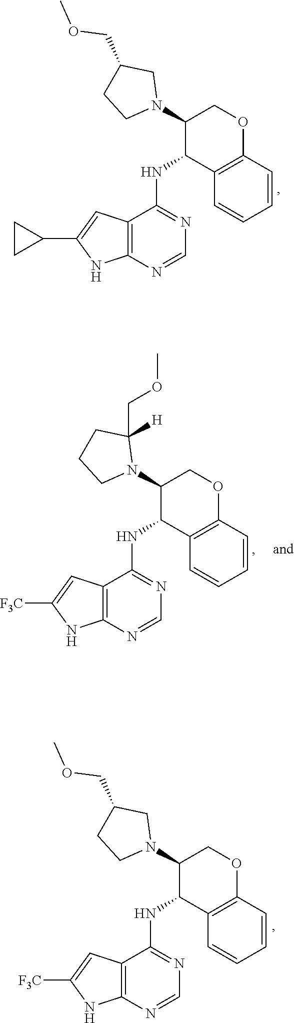 [SMILES: COC[C@H]1CCN([C@H]2COc3ccccc3[C@@H]2Nc2ncnc3[nH]c(C4CC4)cc23)C1 ; COC[C@H]1CCCN1[C@H]1COc2ccccc2[C@@H]1Nc1ncnc2[nH]c(C(F)(F)F)cc12 ; COC[C@H]1CCN([C@H]2COc3ccccc3[C@@H]2Nc2ncnc3[nH]c(C(F)(F)F)cc23)C1]

or a pharmaceutically acceptable salt thereof

### 3. Prophetic Compound Examples

The following compound examples are prophetic, and can be prepared using the synthesis methods described herein above and other general methods as needed as would be known to one skilled in the art. It is anticipated that the prophetic compounds would be active as modulators of RNA polymerase-I signaling, and such activity can be determined using the assay methods described herein below.

In one aspect, a compound is selected from:

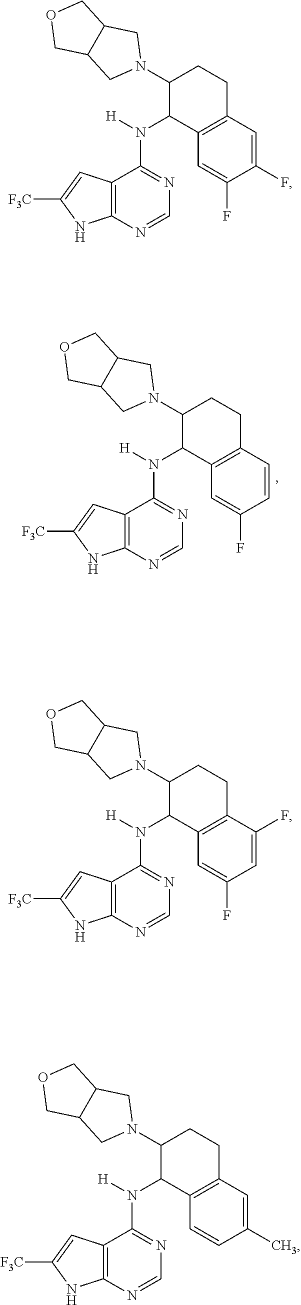 [SMILES: Fc1cc2c(cc1F)C(Nc1ncnc3[nH]c(C(F)(F)F)cc13)C(N1CC3COCC3C1)CC2 ; Fc1ccc2c(c1)C(Nc1ncnc3[nH]c(C(F)(F)F)cc13)C(N1CC3COCC3C1)CC2 ; Fc1cc(F)c2c(c1)C(Nc1ncnc3[nH]c(C(F)(F)F)cc13)C(N1CC3COCC3C1)CC2 ; Cc1ccc2c(c1)CCC(N1CC3COCC3C1)C2Nc1ncnc2[nH]c(C(F)(F)F)cc12]

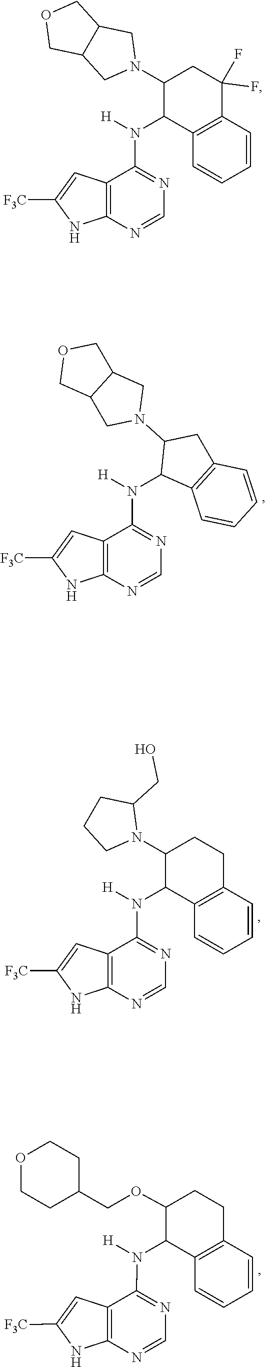 [SMILES: FC(F)(F)c1cc2c(NC3c4ccccc4C(F)(F)CC3N3CC4COCC4C3)ncnc2[nH]1 ; FC(F)(F)c1cc2c(NC3c4ccccc4CC3N3CC4COCC4C3)ncnc2[nH]1 ; OCC1CCCN1C1CCc2ccccc2C1Nc1ncnc2[nH]c(C(F)(F)F)cc12 ; FC(F)(F)c1cc2c(NC3c4ccccc4CCC3OCC3CCOCC3)ncnc2[nH]1]

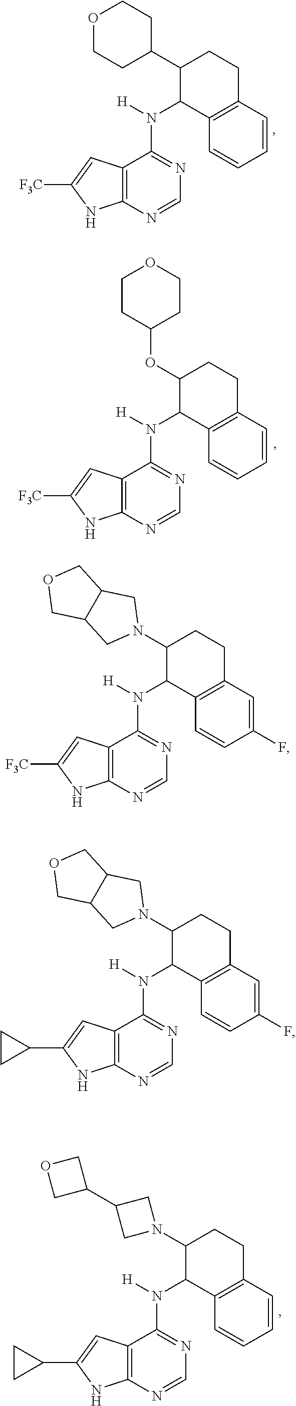

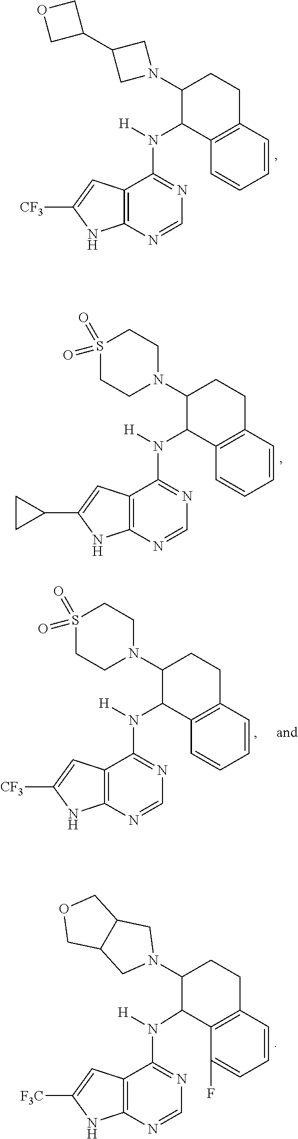 [SMILES: FC(F)(F)c1cc2c(NC3c4ccccc4CCC3N3CC(C4COC4)C3)ncnc2[nH]1 ; O=S1(=O)CCN(C2CCc3ccccc3C2Nc2ncnc3[nH]c(C4CC4)cc23)CC1 ; O=S1(=O)CCN(C2CCc3ccccc3C2Nc2ncnc3[nH]c(C(F)(F)F)cc23)CC1 ; Fc1cccc2c1C(Nc1ncnc3[nH]c(C(F)(F)F)cc13)C(N1CC3COCC3C1)CC2]

In one aspect, a compound is selected from:

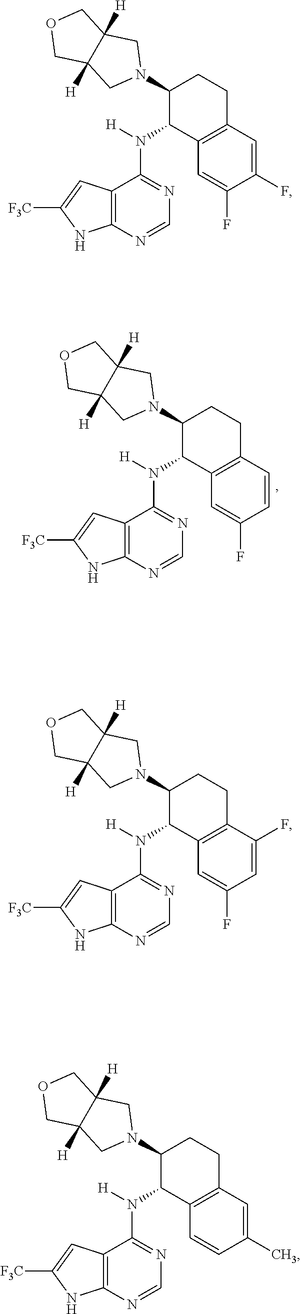 [SMILES: Fc1cc2c(cc1F)[C@H](Nc1ncnc3[nH]c(C(F)(F)F)cc13)[C@@H](N1C[C@H]3COC[C@H]3C1)CC2 ; Fc1ccc2c(c1)[C@H](Nc1ncnc3[nH]c(C(F)(F)F)cc13)[C@@H](N1C[C@H]3COC[C@H]3C1)CC2 ; Fc1cc(F)c2c(c1)[C@H](Nc1ncnc3[nH]c(C(F)(F)F)cc13)[C@@H](N1C[C@H]3COC[C@H]3C1)CC2 ; Cc1ccc2c(c1)CC[C@H](N1C[C@H]3COC[C@H]3C1)[C@H]2Nc1ncnc2[nH]c(C(F)(F)F)cc12]

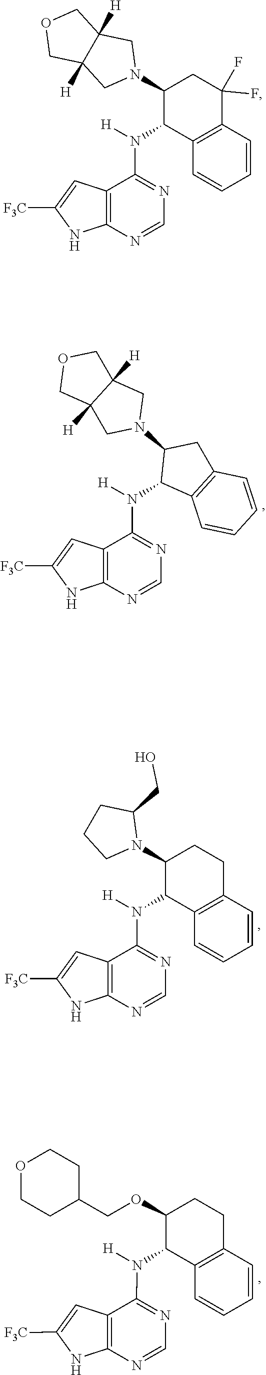

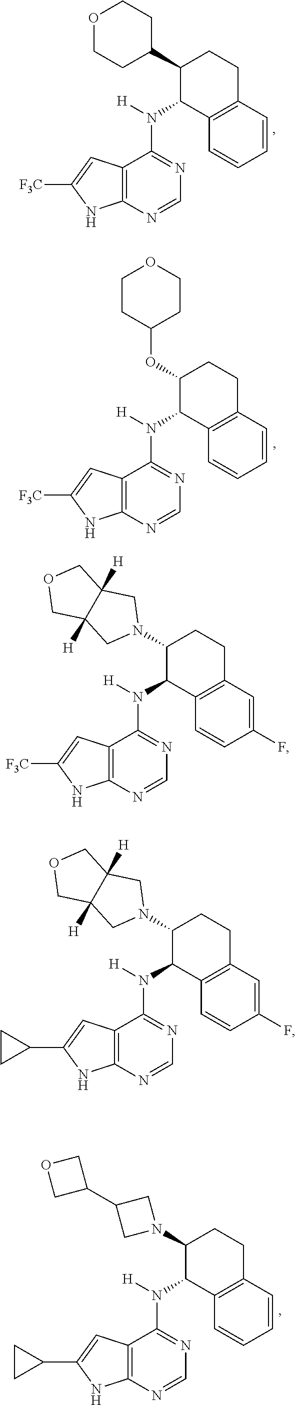

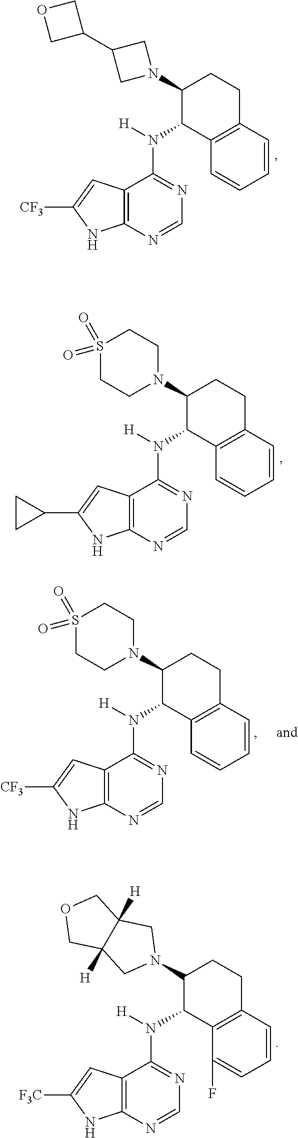

In one aspect, a compound is selected from:

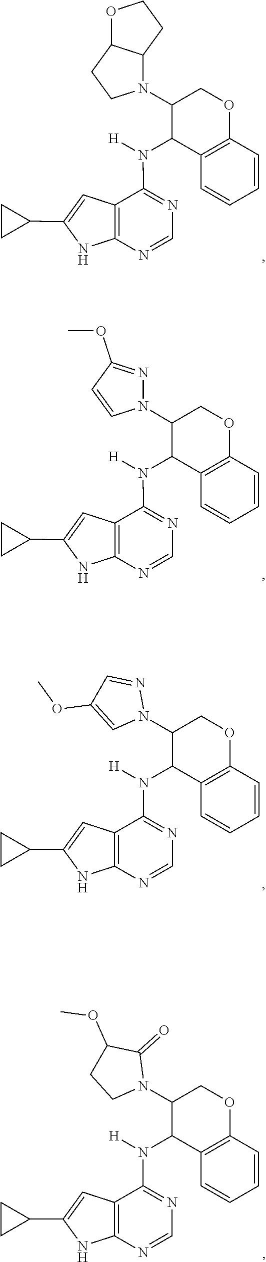 [SMILES: c1ccc2c(c1)OCC(N1CCC3OCCC31)C2Nc1ncnc2[nH]c(C3CC3)cc12 ; COc1ccn(C2COc3ccccc3C2Nc2ncnc3[nH]c(C4CC4)cc23)n1 ; COc1cnn(C2COc3ccccc3C2Nc2ncnc3[nH]c(C4CC4)cc23)c1 ; COC1CCN(C2COc3ccccc3C2Nc2ncnc3[nH]c(C4CC4)cc23)C1=O]

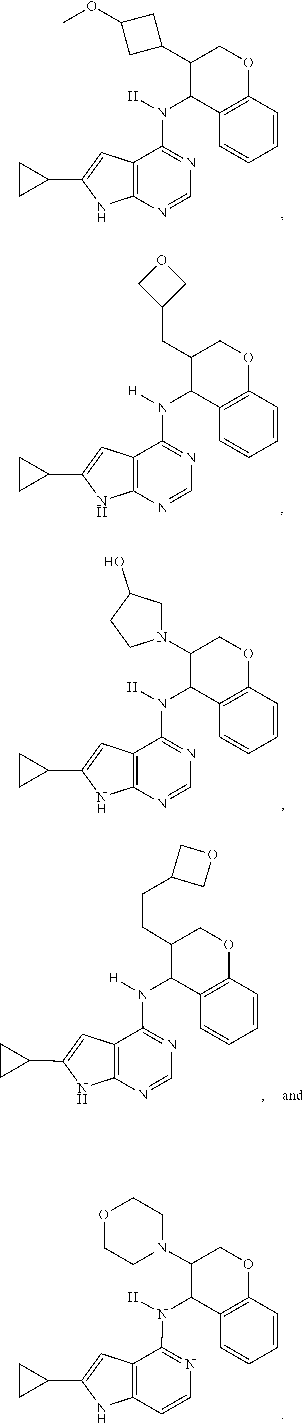

In one aspect, a compound is selected from:

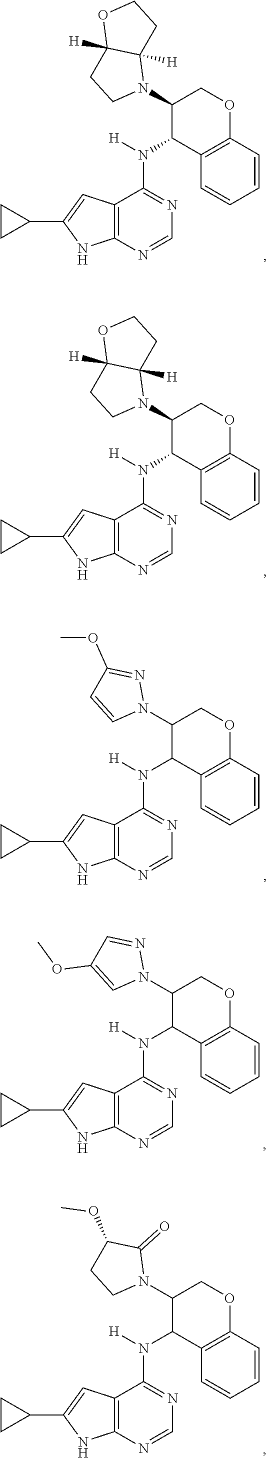 [SMILES: c1ccc2c(c1)OC[C@H](N1CC[C@@H]3OCC[C@H]31)[C@H]2Nc1ncnc2[nH]c(C3CC3)cc12 ; c1ccc2c(c1)OC[C@H](N1CC[C@@H]3OCC[C@@H]31)[C@H]2Nc1ncnc2[nH]c(C3CC3)cc12 ; COc1ccn(C2COc3ccccc3C2Nc2ncnc3[nH]c(C4CC4)cc23)n1 ; COc1cnn(C2COc3ccccc3C2Nc2ncnc3[nH]c(C4CC4)cc23)c1 ; CO[C@H]1CCN(C2COc3ccccc3C2Nc2ncnc3[nH]c(C4CC4)cc23)C1=O]

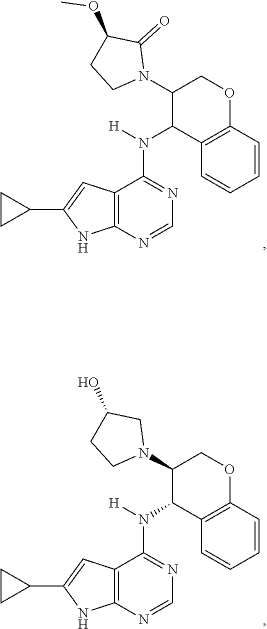 [SMILES: CO[C@@H]1CCN(C2COc3ccccc3C2Nc2ncnc3[nH]c(C4CC4)cc23)C1=O ; O[C@H]1CCN([C@H]2COc3ccccc3[C@@H]2Nc2ncnc3[nH]c(C4CC4)cc23)C1]

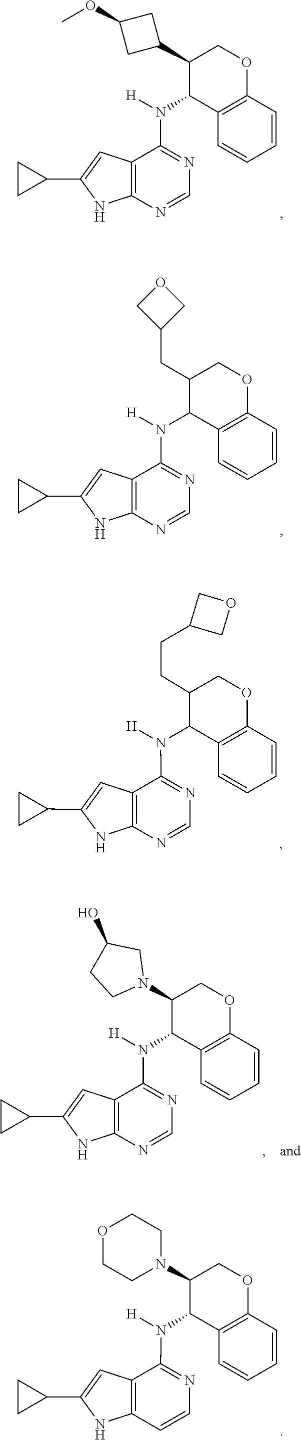

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. PHARMACEUTICAL COMPOSITIONS

Also provided herein are pharmaceutical compositions comprising a disclosed compound, or pharmaceutically acceptable salt thereof; and a pharmaceutically acceptable carrier. Thus, in various embodiments, disclosed are pharmaceutical compositions comprising a therapeutically effective amount at least one disclosed compound and a pharmaceutically acceptable carrier. In a further embodiment, a pharmaceutical composition can be provided comprising a therapeutically effective amount of at least one disclosed compound. In a still further embodiment, a pharmaceutical composition can be provided comprising a prophylactically effective amount of at least one disclosed compound. In yet a further embodiment, the invention relates to pharmaceutical compositions comprising a pharmaceutically acceptable carrier and a compound, wherein the compound is present in an effective amount.

Thus, in various embodiments, provided herein are pharmaceutical compositions comprising a therapeutically effective amount of a compound having a structure represented by a formula:

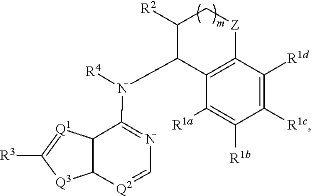

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b} when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —(CH₂)Cy¹, —O(CH₂)ₙCy¹, —NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof, and a pharmaceutically acceptable carrier.

In various embodiments, provided herein are pharmaceutical compositions comprising a therapeutically effective amount of a compound having a structure represented by a formula:

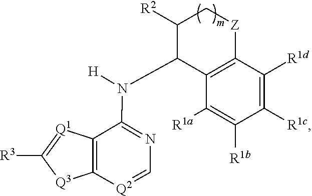

wherein m is 0 or 1; wherein each of Q¹ and Q² is independently N or CH; wherein Q³ is CH₂ or NH; wherein Z is CR^{11a}R^{11b} NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, —NR¹³(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R³ is a 3- to 6-membered cycloalkyl, a C1-C6 haloalkyl, C1-C6 haloalkoxy, or C1-C6 halohydroxyalkyl, or a pharmaceutically acceptable salt thereof, and a pharmaceutically acceptable carrier.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent. Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present disclosure may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agaragar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hardfilled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, catanionic vesicles, and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate. Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

In further embodiments, the pharmaceutical composition is administered to a mammal. In still further embodiments, the mammal is a human. In an even further embodiment, the human is a patient.

In further embodiments, the pharmaceutical composition is administered following identification of the mammal in need of treatment of a disorder associated with PINK1 kinase activity. In still further embodiments, the mammal has been diagnosed with a need for treatment of a disorder associated with PINK1 kinase activity prior to the administering step.

In various embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard-or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, tale, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1, 3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldiallylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, PA, Banker and Chalmers, Eds., 238-250 (1982) and, ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the treatment (e.g., prophylactic or therapeutic) of a disorder associated with PINK1 kinase activity. The method also includes the administration of a therapeutically effect amount of the compound for the treatment of patient having a predisposition for being afflicted with a disorder associated with PINK1 kinase activity. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal, the body weight of the animal, as well as the severity and stage of the disorder.

The total amount of the compound of the present disclosure administered in a typical treatment is preferably from about 1 mg/kg to about 100 mg/kg of body weight for mice, and from about 10 mg/kg to about 50 mg/kg of body weight, and from about 20 mg/kg to about 40 mg/kg of body weight for humans per daily dose. This total amount is typically, but not necessarily, administered as a series of smaller doses over a period of about one time per day to about three times per day for about 24 months, and over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In certain embodiments, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

A compound described herein can be administered alone or can be coadministered with an additional therapeutic agent. Thus, the preparations can also be combined, when desired, with other active substances (e.g., to reduce metabolic degradation). Additional therapeutic agents include, but are not limited to, other active agents known to be useful in treating a disease associated neurodegeneration (e.g., Parkinson's disease such as levodopa), dopamine agonists (e.g., bromocriptine, pergolide, pramipexole, ropinirole, piribedil, cabergoline, apomorphine, lisuride), MAO-B inhibitors (e.g., selegiline or rasagiline), arnantadine, anticholinergics, antipsychotics (e.g., clozapine), cholinesterase inhibitors, modafinil, or non-steroidal anti-inflammatory drugs), Argiolensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics.

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, 1.2% saline, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, tosylate, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences. A.R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present disclosure is in the form of a liquid wherein the active agent is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact Solid articles suitable for implantation in the eye in such fashion are generally composed primarily of polymers and can be bioerodible or non-bioerodible. Bioerodible polymers that can be used in the preparation of ocular implants carrying one or more of the compounds described herein in accordance with the present disclosure include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

In some embodiments, the compound or pharmaceutical composition comprising the compounds disclosed herein, or the pharmaceutically acceptable salts herein, are neo-substrates of PINK1. In some embodiments, the neo-substrate is not kinetin. In some embodiments, the neo-substrate is not kinetin riboside. In some embodiments, the neo-substrate is not kinetin riboside 5′ monophosphate. In some embodiments, the neo-substrate is not kinetin riboside 5′ diphosphate. In some embodiments, the neo-substrate is not kinetin riboside 5′ triphosphate. In some embodiments, the neo-substrate is not a derivative (e.g., prodrug) of kinetin, kinetin riboside, kinetin riboside 5′ monophosphate, kinetin riboside 5′ diphosphate, or kinetin riboside 5′ triphosphate. In some embodiments, the neo-substrate is not N6-(delta 2-Isopentenyl)-adenine. In some embodiments, the neo-substrate is not N6-(delta 2-Isopentenyl)-adenosine, N6-(delta 2-Isopentenyl)-adenosine 5′ monophosphate, N6-(delta 2-Isopentenyl)-adenosine 5′ diphosphate, N6-(delta 2-Isopentenyl)-adenosine 5′ triphosphate, or a derivative (e.g., prodrug) thereof. In some embodiments, the neo-substrate is not a cytokinin. In some embodiments, the neo-substrate is not a cytokinin riboside, cytokinin riboside 5′ monophosphate, cytokinin riboside 5′ diphosphate, cytokinin riboside 5′ triphosphate, or a derivative (e.g., prodrug) thereof.

It is understood that the disclosed compositions can be prepared from the disclosed compounds. It is also understood that the disclosed compositions can be employed in the disclosed methods of using.

### D. METHODS OF MAKING THE COMPOUNDS

In various embodiments, the inventions relates to methods of making compounds useful to treat a disorder associated with PINK1 kinase activity. Thus, in some embodiments, disclosed are methods of making a disclosed compound.

Compounds according to the present disclosure can, for example, be prepared by the several methods outlined below. A practitioner skilled in the art will understand the appropriate use of protecting groups [see: Greene and Wuts, Protective Groups in Organic Synthesis] and the preparation of known compounds found in the literature using the standard methods of organic synthesis. There may come from time to time the need to rearrange the order of the recommended synthetic steps, however this will be apparent to the judgment of a chemist skilled in the art of organic synthesis. The following examples are provided so that the invention might be more fully understood, are illustrative only, and should not be construed as limiting.

In some embodiments, the disclosed compounds comprise the products of the synthetic methods described herein. In further embodiments, the disclosed compounds comprise a compound produced by a synthetic method described herein. In still further embodiments, the invention comprises a pharmaceutical composition comprising a therapeutically effective amount of the product of the disclosed methods and a pharmaceutically acceptable carrier. In still further embodiments, the invention comprises a method for manufacturing a medicament comprising combining at least one compound of any of disclosed compounds or at least one product of the disclosed methods with a pharmaceutically acceptable carrier or diluent.

### 1. Route 1

In some embodiments, compounds can be prepared as shown below,

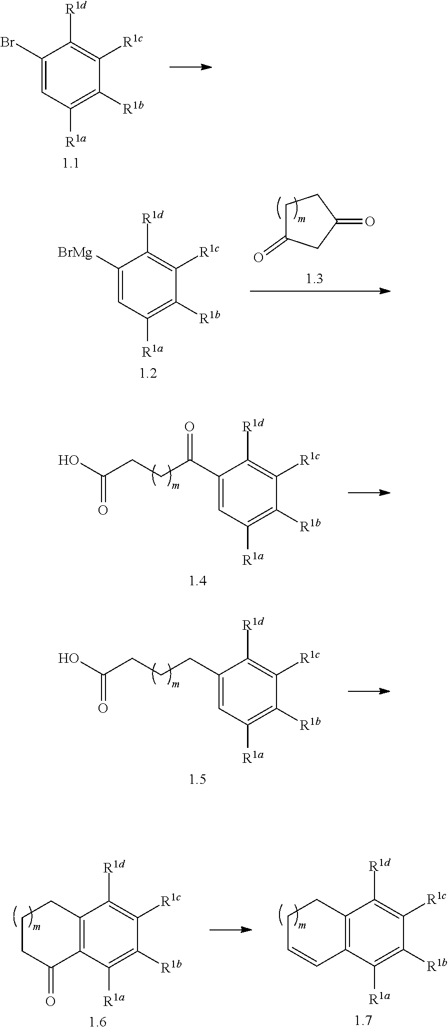

Compounds are represented in generic form, with substituents as noted in compound descriptions elsewhere herein. A more specific example is set forth below.

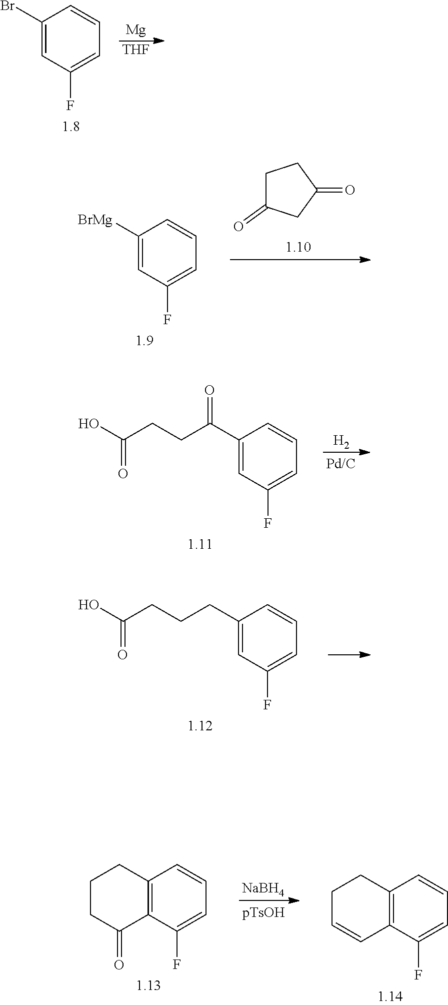

In some embodiments, compounds of type 1.14, and similar compounds, can be prepared according to reaction Scheme 1B above. Thus, compounds of type 1.11 can be prepared by a Grignard reaction of an appropriate aryl bromine, e.g., 1.8 as shown above. Appropriate aryl bromines are commercially available or prepared by methods known to one skilled in the art. The Grignard reaction is carried out in the presence of an appropriate metal source, e.g., magnesium metal, in an appropriate solvent, e.g., tetrahydrofuran (THF), followed by reaction with an appropriate carbonyl analog, e.g., 1.10 as shown above. Appropriate carbonyl analogs are commercially available or prepared by methods known to one skilled in the art. Compounds of type 1.12 can be prepared by reduction of an appropriate ketone, e.g., 1.11 as shown above. The reduction is carried out in the presence of an appropriate reducing agent, e.g., hydrogen gas, and an appropriate catalyst, e.g., palladium on carbon. Compounds of type 1.13 can be prepared by cyclization of an appropriate aryl carboxylic acid analog, e.g., 1,12 as shown above. The cyclization is carried out in the presence of strong acid (like trifluorosulfonic acid) and heat. Compounds of type 1.14 can be prepared by reduction of an appropriate ketone, e.g., 1.13 as shown above. The reduction is carried out in the presence of an appropriate activating agent, e.g., para-toluenesulfonic acid, and an appropriate reducing agent, e.g., sodium borohydride. As can be appreciated by one skilled in the art, the above reaction provides an example of a generalized approach wherein compounds similar in structure to the specific reactants above (compounds similar to compounds of type 1.1, 1.2, 1.3, 1.4, 1.5, and 1.6), can be substituted in the reaction to provide compounds similar to Formula 1.7.

### 2. Route II

In some embodiments, compounds can be prepared as shown below.

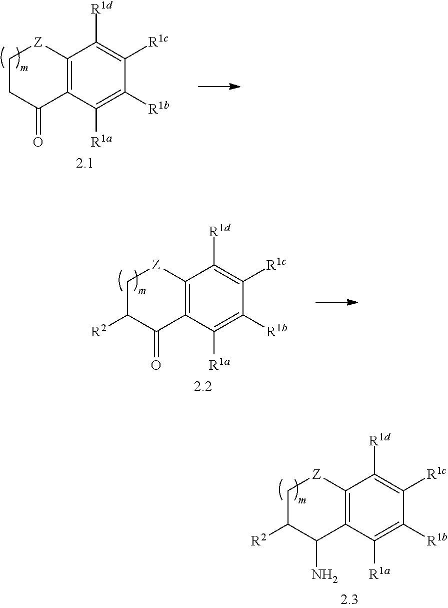

Compounds are represented in generic form, with substituents as noted in compound descriptions elsewhere herein. A more specific example is set forth below.

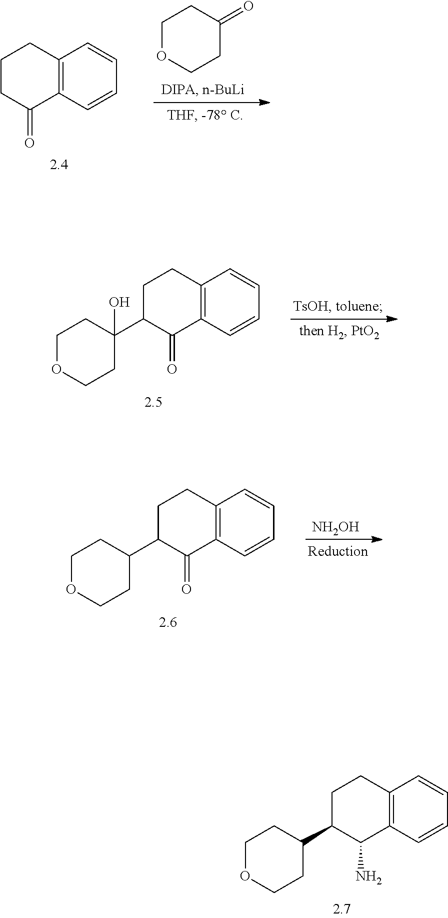

In some embodiments, compounds of type 2.7, and similar compounds, can be prepared according to reaction Scheme 2B above. Thus, compounds of type 2.5 can be prepared by reaction of an appropriate aryl ketone, e.g., 1.8 as shown above, and an appropriate ketone, e.g., tetrahydro-4H-pyran4-one as shown above. Appropriate aryl ketones and appropriate ketones are commercially available or prepared by methods known to one skilled in the art. The reaction is carried out in the presence of an appropriate amine, e.g., diisopropylamine (DIPA), and an appropriate base, e.g., n-butyl lithium, in an appropriate solvent, e.g., THF1, at an appropriate temperature, e.g., −78 °C. Compounds of type 2.6 can be prepared by reduction of an appropriate alcohol, e.g., 2.5 as shown above. The reduction is carried out in the presence of an appropriate activating agent, e.g., toluenesulfonic acid, in an appropriate solvent, e.g., toluene, followed by reaction with an appropriate reducing agent, e.g., hydrogen gas, and an appropriate catalyst, e.g., platinum oxide. Compounds of type 2.7 can be prepared by reductive amination of an appropriate ketone, e.g., 2.6 as shown above. The reductive amination is carried out in the presence of an appropriate agent, e.g., hydroxylamine. As can be appreciated by one skilled in the art, the above reaction provides an example of a generalized approach wherein compounds similar in structure to the specific reactants above (compounds similar to compounds of type 2.1 and 2.2), can be substituted in the reaction to provide compounds similar to Formula 2.3.

### 3. Route III

In some embodiments, compounds can be prepared as shown below.

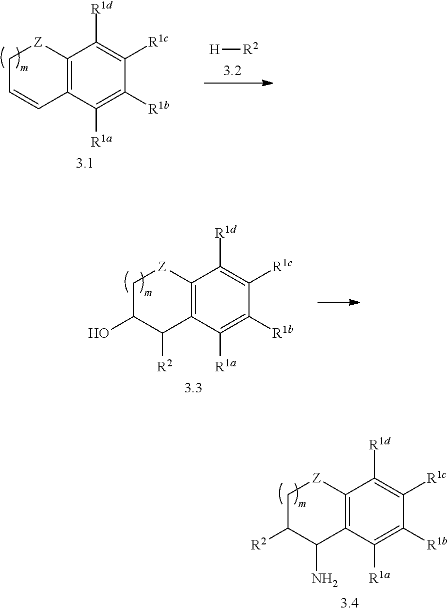

Compounds are represented in generic form, with substituents as noted in compound descriptions elsewhere herein. A more specific example is set forth below.

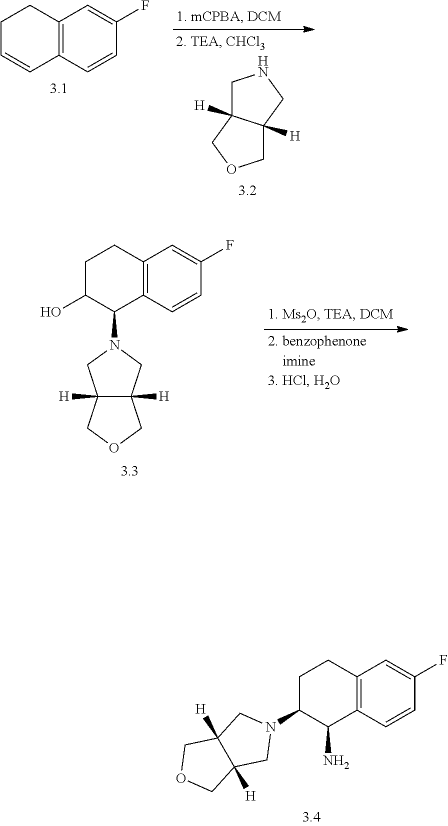 [SMILES: Fc1ccc2c(c1)CCC=C2 ; C1NC[C@H]2COC[C@@H]12 ; OC1CCc2cc(F)ccc2[C@H]1N1C[C@H]2COC[C@H]2C1 ; N[C@@H]1c2ccc(F)cc2CC[C@@H]1N1C[C@H]2COC[C@H]2C1]

In some embodiments, compounds of type 3.4, and similar compounds, can be prepared according to reaction Scheme 3B above. Thus, compounds of type 3.3 can be prepared by epoxidation of an appropriate alkene, e.g., 3.1 as shown above. The epoxodation is carried out in the presence of an appropriate oxidizing agent, e.g., meta-chloroperoxybenzoic acid (mCPBA), in an appropriate solvent, e.g., dichloromethane (DCM), followed by ring opening in the presence of an appropriate amine, e.g., 3.2 as shown above. Appropriate amines are commercially available or prepared by methods known to those skilled in the art. The ring opening is carried out in the presence of an appropriate base, e.g., triethylamine (TEA), in an appropriate solvent, e.g., chloroform (CHCl₃). Compounds of type 3.4 can be prepared by rearrangement of an appropriate alcohol, e.g., 3.3 as shown above. The rearrangement is carried out in the presence of an appropriate activating agent, e.g., methanesulfonic anhydride, an appropriate base, e.g., TEA, in an appropriate solvent, e.g., DCM, followed by reaction with an appropriate imine, e.g., benzophenone imine. As can be appreciated by one skilled in the art, the above reaction provides an example of a generalized approach wherein compounds similar in structure to the specific reactants above (compounds similar to compounds of type 3.1, 3.2, and 3.3), can be substituted in the reaction to provide compounds similar to Formula 3.4.

### 4. Route IV

In some embodiments, compounds can be prepared as shown below.

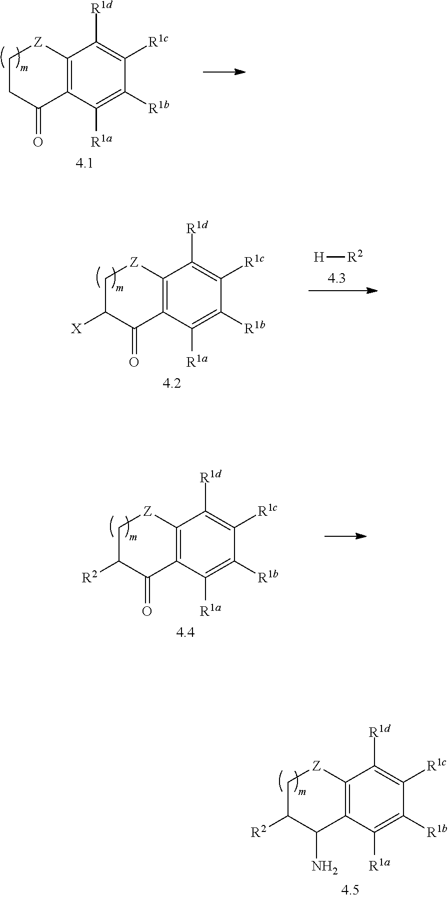

Compounds are represented in generic form, with substituents as noted in compound descriptions elsewhere herein. A more specific example is set forth below.

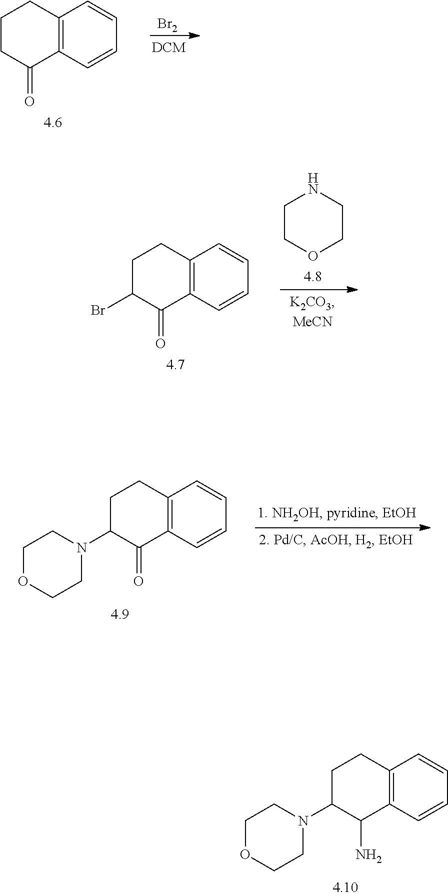

In some embodiments, compounds of type 4.10, and similar compounds, can be prepared according to reaction Scheme 4B above. Thus, compounds of type 4.7 can be prepared by halogenation of an appropriate ketone, e.g., 4.6 as shown above. Appropriate ketones are commercially available or prepared by methods known to one skilled in the art The halogenation is carried out in the presence of an appropriate halide source, e.g., bromine, in an appropriate solvent, e.g., DCM. Compounds of type 4.9 can be prepared by displacement of an appropriate halide, e.g., 4.7 as shown above. The displacement reaction is carried out in the presence of an appropriate nucleophilic agent, e.g., 4.8 as shown above, and an appropriate base, e.g., potassium carbonate, in an appropriate solvent, e.g., acetonitrile. Appropriate nucleophilic agents are commercially available or prepared by methods known to one skilled in the art. Compounds of type 4.10 can be prepared by reductive amination of an appropriate ketone, e.g., 4.9 as shown above. The reductive amination is carried out in the presence of an appropriate amine, e.g., hydroxylamine, and an appropriate base, e.g., pyridine, in an appropriate solvent, e.g., ethanol, followed by reaction with an appropriate reducing agent, e.g., hydrogen gas, an appropriate catalyst, e.g., palladium on carbon, and an appropriate acid, e.g., acetic acid, in an appropriate solvent, e.g., ethanol. As can be appreciated by one skilled in the art, the above reaction provides an example of a generalized approach wherein compounds similar in structure to the specific reactants above (compounds similar to compounds of type 4.1, 4.2, 4.3, and 4.4), can be substituted in the reaction to provide compounds similar to Formula 4.5.

### 5. Route V

In some embodiments, adenine analogs can be prepared as shown below.

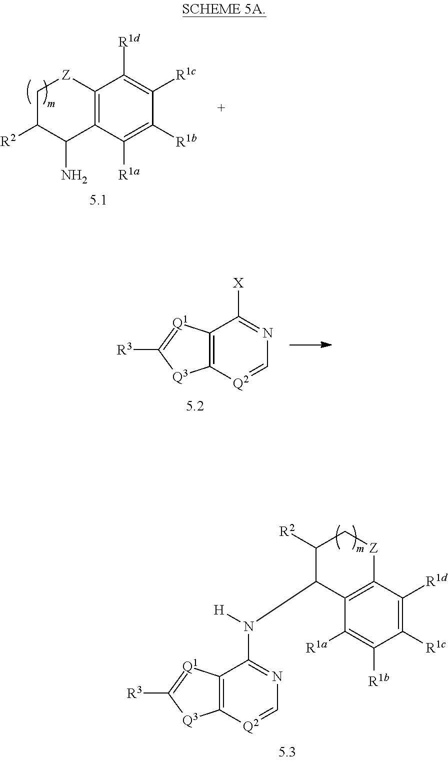

Compounds are represented in generic form, wherein X is halogen and with other substituents as noted in compound descriptions elsewhere herein. A more specific example is set forth below.

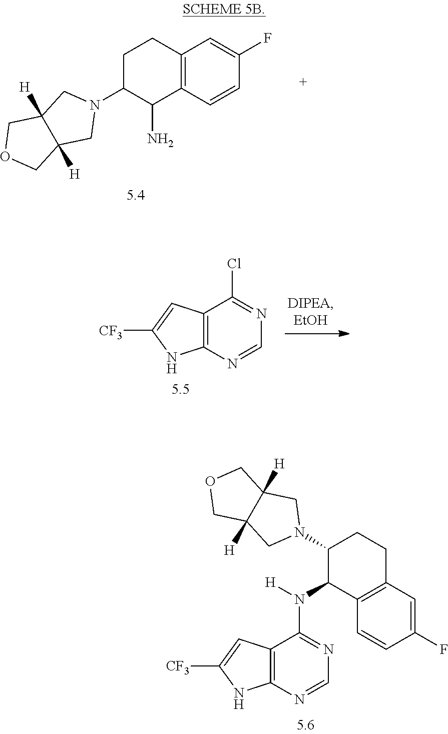 [SMILES: NC1c2ccc(F)cc2CCC1N1C[C@H]2COC[C@H]2C1 ; FC(F)(F)c1cc2c(Cl)ncnc2[nH]1 ; Fc1ccc2c(c1)CC[C@@H](N1C[C@H]3COC[C@H]3C1)[C@@H]2Nc1ncnc2[nH]c(C(F)(F)F)cc12]

In some embodiments, compounds of type 5.3, and similar compounds, can be prepared according to reaction Scheme 5B above. Thus, compounds of type 5.3 can be prepared by arylation of an appropriate amine, e.g., 5.1 as shown above. The arylation is carried out in the presence of an appropriate halide, e.g., 5.2 as shown above, and an appropriate base, e.g., diisopropylethyl amine (DIPEA), in an appropriate solvent, e.g., ethanol (EtOH). Appropriate halides are commercially available or prepared by methods known to those skilled in the art. As can be appreciated by one skilled in the art, the above reaction provides an example of a generalized approach wherein compounds similar in structure to the specific reactants above (compounds similar to compounds of type 5.1 and 5.2), can be substituted in the reaction to provide adenine analogs similar to Formula 5.3.

Compounds and compositions described herein are generally useful for modulating the activity of PINK1. In some embodiments, the compounds and compositions described herein inhibit the activity of PINK1.

### E. METHODS OF USING THE COMPOUNDS

The compounds and pharmaceutical compositions of the invention are useful in treating or controlling disorders associated with PINK1 kinase activity. To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of the disorder associated with PINK1 kinase activity.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disease or condition.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Treatment Methods

The compounds disclosed herein are useful for treating or controlling disorders associated with PINK1 kinase activity. Thus, provided is a method comprising administering a therapeutically effective amount of a composition comprising a disclosed compound to a subject.

Accordingly, in some embodiments, the present disclosure provides methods of treating or preventing a neurodegenerative disease (e.g., Parkinson's disease, Leigh's disease) in a subject comprising administering to the subject one or more compounds, or a pharmaceutically acceptable salt thereof, of any one of the compounds described herein or a pharmaceutical composition comprising one or more of the compounds described herein, or pharmaceutically acceptable salt thereof. In some embodiments, the treating of the neurodegenerative disease comprises ameliorating symptoms by stimulating PINK1 or a mutated PINK1.

In some embodiments, the present disclosure provides methods of treating or preventing a mitochondrial disease in a subject comprising administering to the subject one or more compounds, or a pharmaceutically acceptable salt thereof, of any one of the compounds described herein or a pharmaceutical composition comprising one or more of the compounds described herein, or pharmaceutically acceptable salt thereof. In some embodiments, the treating of the mitochondrial disease comprises ameliorating symptoms by stimulating PINK1 or a mutated PINK1.

In some embodiments, the present disclosure provides methods of treating or preventing fibrosis in a subject comprising administering to the subject one or more compounds, or a pharmaceutically acceptable salt thereof, of any one of the compounds described herein or a pharmaceutical composition comprising one or more of the compounds described herein, or pharmaceutically acceptable salt thereof. In some embodiments, the treating of the fibrosis comprises ameliorating symptoms by stimulating PINK1 or a mutated PINK1.

In some embodiments, the present disclosure provides methods of treating or preventing cardiomyopathy in a subject comprising administering to the subject one or more compounds, or a pharmaceutically acceptable salt thereof, of any one of the compounds described herein or a pharmaceutical composition comprising one or more of the compounds described herein, or pharmaceutically acceptable salt thereof. In some embodiments, the treating of the cardiomyopathy comprises ameliorating symptoms by stimulating PINK1 or a mutated PINK1.

In some embodiments, a method of treating one or more of the following mitochondrial diseases in a subject is provided: LHON, MELAS, and Charcot Marie Tooth. In some embodiments, the method comprises administering to a subject one or more compounds described herein, or a pharmaceutically acceptable salt thereof, or a pharmaceutical composition comprising one or more compounds described herein, or pharmaceutically acceptable salt thereof. In some embodiments, the method comprises administering to a subject a compound or pharmaceutically acceptable salt thereof that acts as a PINK1 substrate with one or more compounds described herein, or a pharmaceutically acceptable salt thereof, or a pharmaceutical composition comprising one or more compounds described herein, or pharmaceutically acceptable salt thereof. In some embodiments, the cholesterol therapeutic is niacin or acifran. In some embodiments, the subject is a subject in need thereof.

a. Treating a Disorder Associated with Pink1 Activity

In some embodiments, compounds and compositions described herein are useful in treating a disorder associated with PINK1 function. Thus, provided herein are methods of treating a disorder associated with PINK1 function, comprising administering to a subject in need thereof, a therapeutically effective amount of a compound described herein, or a pharmaceutically acceptable salt thereof, or a composition comprising a disclosed compound or pharmaceutically acceptable salt thereof. Disorders treatable by the present compounds and compositions include, e.g., a neurodegenerative disease, a mitochondrial disease, fibrosis, or cardiomyopathy.

Thus, in various embodiments, disclosed are methods of treating a disorder in a subject in need thereof, the method comprising administering to the subject in need thereof an effective amount of a compound having a structure represented by a formula:

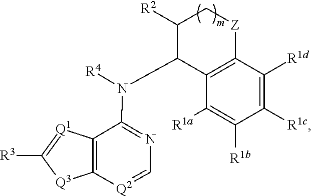

wherein in is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c} and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —(CH₂)ₙCy¹, —O(CH₂)ₙCy¹, —NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one 0, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof, wherein the disorder is a neurodegenerative disorder, a mitochondrial disorder, a fibrosis, or cardiomyopathy.

In various embodiments, disclosed are methods of treating a disorder in a subject in need thereof, the method comprising administering to the subject in need thereof an effective amount of a compound having a structure represented by a formula:

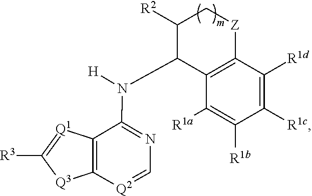

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b} when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, —NR^{II}(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, CL-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R³ is a 3- to 6-membered cycloalkyl, a C1-C6 haloalkyl, CJ-C6 haloalkoxy, or C1-C6 halohydroxyalkyl, or a pharmaceutically acceptable salt thereof, wherein the disorder is a neurodegenerative disorder, a mitochondrial disorder, a fibrosis, or cardiomyopathy.

Examples of neurodegenerative diseases that may be treated with a compound or composition described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, dysautonomia, epilepsy, Friedreich ataxia, frontotemporal dementia, Gerstmann-Straussler-Scheinker syndrome, Huntington's disease, HIV-associated dementia, Kennedy's disease, Krabbe's disease, kuru, Leigh's disease (Leigh syndrome), Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Shy-Drager syndrome, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease. In some embodiments, dysautonomia is not a neurodegenerative disease.

Examples of mitochondrial diseases that may be treated with a compound or composition described herein include Alzheimer's disease, amyotrophic lateral sclerosis, Asperger's Disorder, Autistic Disorder, bipolar disorder, cancer, cardiomyopathy, Charcot Marie Tooth disease (CMT, including various subtypes such as CMT type 2b and 2b), Childhood Disintegrative Disorder (CDD), diabetes, diabetic nephropathy, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration. Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease. Peroneal muscular atrophy (PMA), Pervasive Developmental Disorder Not Otherwise Specified (PDD-NOS), renal tubular acidosis, Rett's Disorder, Schizophrenia, and types of stroke.

Cardiomyopathy refers to a disease condition that adversely affects cardiac cell tissue leading to a measurable deterioration in myocardial function (e.g., systolic function, diastolic function). Dilated cardiomyopathy is characterized by ventricular chamber enlargement with systolic dysfunction and no hypertrophy. Hypertrophic cardiomyopathy, is a genetic disease transmitted as an autosomal dominant trait. Hypertrophic cardiomyopathy is morphologically characterized by a hypertrophied and non-dialated left ventricle. Restrictive cardiomyopathy is characterized by nondialated nonhypertrophied morphology with diminished ventricular volume leading to poor ventricular filling. Arrhythmogenic right ventricular cardiomyopathy is an inheritable heart disease characterized by myocardial electric instability. Unclassified cardiomyopathy is a category for cardiomyopathies that do not match the features of any one of the other types. Unclassified cardiomyopathies may have features of multiple types or, for example, have the features of fibroelastosis, noncompacted myocardium, or systolic dysfunction with minimal dilatation.

In certain embodiments, the compounds and compositions described herein can be used to treat Parkinson's disease by decreasing the production of Lewy bodies, decreasing the accumulation of alpha-synuclein, decreasing cell death, decreasing loss of dopamine-generating cells, decreasing loss of cells in the substantia nigra, decreasing loss of dopamine production, decreasing a symptom of Parkinson's disease, decreasing loss of motor function, decreasing shaking or slowing an increase in shaking (tremor), decreasing rigidity or an increase in rigidity, decreasing slowness (bradykinesia) of movement or a slowing of movement, decreasing sensory symptoms, decreasing insomnia, decreasing sleepiness, increasing mental wellbeing, increasing mental function, slowing the decrease of mental function, decreasing dementia, delaying the onset of dementia, improving cognitive skills, decreasing the loss of cognitive skills, improving memory, decreasing the degradation of memory, or extending survival. In certain embodiments, the compounds and compositions described herein can be used to treat cardiomyopathy by increasing cardiac performance, improving exercise tolerance, preventing heart failure, increasing blood oxygen content, or improving respiratory function.

In certain embodiments, the disease treated by a disclosed compound or composition is one that is characterized by a reduction in the level of PINK1. In certain embodiments, the disease is one characterized by loss of dopamine-producing cells (e.g., Parkinson's disease). In certain embodiments, the disease is one characterized by neurodegeneration. In certain embodiments, the disease is one characterized by neural cell death. In certain embodiments, the disease is one characterized by a reduction in the level of PINK1 activity. In certain embodiments, the disease is Parkinson's disease. In certain embodiments, the disease is a neurodegenerative disease. In certain embodiments, the disease is a cardiomyopathy.

In further embodiments, the neurodegenerative disorder is Parkinson's disease, Huntington's disease, or amyotrophic lateral sclerosis.

In further embodiments, the subject has been diagnosed with a need for treatment of a disorder associated with PINK1 kinase activity prior to the administering step.

In further embodiments, the subject is a mammal. In still further embodiments, the mammal is a human.

In further embodiments, the method further comprises the step of identifying a subject in need of treatment of a disorder associated with PINK1 kinase activity.

In further embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

### 2. Methods of Modulating Pink1 Kinase Activity in a Mammal

In some embodiments, disclosed are methods of modulating PINK1 kinase activity in a mammal, the method comprising the step of administering to the mammal a therapeutically effective amount of at least one disclosed compound, or a pharmaceutically acceptable salt thereof.

Thus, in various embodiments, disclosed are methods of modulating PINK1 kinase activity in a subject in need thereof, the method comprising administering to the subject in need thereof an effective amount of a compound having a structure represented by a formula:

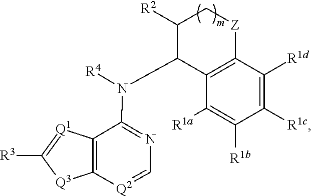

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, CP—C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —(CH₂)ₙCy¹, —O(CH₂)Cy¹, —NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof

In various embodiments, disclosed are methods of modulating PINK1 kinase activity in a subject in need thereof, the method comprising administering to the subject in need thereof an effective amount of a compound having a structure represented by a formula:

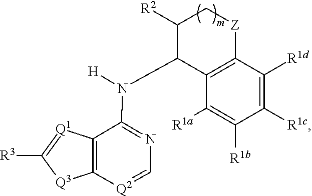

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, —NR¹³(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R³ is a 3- to 6-membered cycloalkyl, a C1-C6 haloalkyl, C1-C6 haloalkoxy, or C1-C6 halohydroxyalkyl, or a pharmaceutically acceptable salt thereof.

As used herein, “modulation” can refer to either inhibition or enhancement of a specific activity. For example, the modulation of PINK1 activity can refer to the inhibition and/or activation of PINK1 dependent activities, such as a decrease in Parkin recruitment. In some embodiments, the modulation refers to the inhibition or activation of Parkin recruitment. In some embodiments, the compounds described herein activate PINK1 activity by a factor from about 1% to about 50%. The activity of PINK1 can be measured by any method including but not limited to the methods described herein.

The compounds described herein are neo-substrates of PINK1. The ability of the compounds to stimulate or inhibit PINK1 activity may be measured using any assay known in the art used to detect Parkin recruitment or PINK1 phosphorylation, or the absence of such signaling/activity. “PINK1 activity” refers to the ability of PINK1 to phosphorylate any substrate. Such activity can be measured, e.g., in a cell(s), by expressing mutant PINK1, administering the compounds disclosed herein and measuring the degree to which cells expressing the mutant PINK1 were able to phosphorylate an enzymatically active substrate as compared to a cell(s) expressing wild-type PINK1.

PINK1 activity can be measured by changes in the time necessary to recruit 50% of a substrate (“R₅₀”). In some embodiments, the compounds reduce a R50 by a factor of about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. In some embodiments, the compounds reduce a R₅₀ by a factor from about 1% to about 50%. In some embodiments, the compounds reduce a R₅₀ by a factor from about 2% to about 50%. In some embodiments, the compounds reduce a R₅₀ by a factor from about 3% to about 50%. In some embodiments, the compounds reduce a R₅₀ by a factor from about 4% to about 50%. In some embodiments, the compounds reduce a R₅₀ by a factor from about 5% to about 50%. In some embodiments, the compounds reduce a R₅₀ by a factor from about 6% to about 50%. In some embodiments, the compounds reduce a R₅₀ by a factor from about 7% to about 50%. In some embodiments, the compounds reduce a R₅₀ by a factor from about 8% to about 50%. In some embodiments, the compounds reduce a R₅₀ by a factor from about 9% to about 50%. In some embodiments, the compounds reduce a R₅₀ by a factor from about 10% to about 50%. In some embodiments, the compounds reduce a R₅₀ by a factor from about 15% to about 50%. In some embodiments, the compounds reduce a R₅₀ by a factor from about 20% to about 50%. In some embodiments, the compounds reduce a R₅₀ by a factor from about 25% to about 50%. In some embodiments, the compounds reduce a R₅₀ by a factor from about 30% to about 50%. In some embodiments, the compounds reduce a R₅₀ by a factor from about 35% to about 50%. In some embodiments, the compounds reduce a R₅₀ by a factor from about 40% to about 50%. In some embodiments, the compounds reduce a R₅₀ by a factor from about 45% to about 50%. In some embodiments, the compounds reduce a R₅₀ by a factor from about 10% to about 40%. In some embodiments, the compounds reduce a R₅₀ by a factor from about 10% to about 30%. In some embodiments, the compounds reduce a R₅₀ by a factor from about 10% to about 200%.

Plasmids expressing PINK1 can be transfected into an isolated cell and expressed in an isolated cell, expressed in a membrane derived from a cell, expressed in tissue or in an animal. For example, neuronal cells, cells of the immune system, transformed cells, or membranes can be used to test the PINK1 activity described above. Modulation is tested using one of the in vitro or in vivo assays described herein. Other assays generally known can also be used to test the compounds. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

In some embodiments, a compound's effect on the modulation of PINK1 will be measured using cells expressing mutant and wild-type versions of PINK1. PINK1 is generally known. In some embodiments, the enzymatic rescue is measured. Enzymatic rescue experiments are experiments in which cells expressing mutated forms of the PINK1 with reduced or deficient enzymatic activity are contacted with compounds of the present invention and are able to re-activate the mutated PINK1 enzymatic activity. PINK1 molecules are known. In some embodiments, the compounds of the present invention are able to enzymatically rescue human PINK1 (accession number NM_032409.3, which is incorporated by reference in its entirety) having the following amino acid sequence:

(SEQ ID NO: 1)
MAVRQALGRGLQLGRALLLRFTGKPGRAYGLGRPGPAAGCVRGERPGW
AAGPGAEPRRVGLGLPNRLRFFRQSVAGLAARLQRQFVVRAWGCAGPCG
RAVFLAFGLGLGLIEEKQAESRRAVSACQEIQAIFTQKSKPGPDPLDTR
RLQGFRLEEYLIGQSIGKGCSAAVYEATMPTLPQNLEVTKSTGLLPGRG
PGTSAPGEGQERAPGAPAFPLAIKMMWNISAGSSSEAILNTMSQELVPA
SRVALAGEYGAVTYRKSKRGPKQLAPHPNIIRVLRAFTSSVPLLPGALV
DYPDVLPSRLHPEGLGHGRTLFLVMKNYPCTLRQYLCVNTPSPRLAAMM
LLQLLEGVDHLVQQGIAHRDLKSDNILVELDPDGCPWLVIADFGCCLAD
ESIGLQLPFSSWYVDRGGNGCLMAPEVSTARPGPRAVIDYSKADAWAVG
AIAYEIFGLVNPFYGQGKAHLESRSYQEAQLPALPESVPPDVRQLVRAL
LQREASKRPSARVAANVLHLSLWGEHILALKNLKLDKMVGWLLQQSAAT
LLANRLTEKCCVETKMKMLFLANLECETLCQAALLLCSWRAAL.

In some embodiment, the compounds of the present invention are able to enzymatically rescue mouse PINK1 (accession number XM 924521, which is incorporated by reference in its entirety) having the following amino acid sequence:

(SEQ ID NO: 2)
MAVRQALGRGLQLGRALLLRFAPKPGPLFGWGKPGPAAAWGRGERPGQ
VVSPGAQPRPVGLPLPDRYRFFRQSVAGLAARIQRQFMVRARGGAGPCG
RAVFLAFGLGLGLIEEKQAEGRRAASACQEIQAIFTQKTKRVSDPLDTR
CWQGFRLEDYLIGQAIGKGCNAAVYEATMPTLPQHLEKAKHLGLIGKGP
DVVLKGADGEQAPGTPTFPFAIKMMWNISAGSSSEAILSKMSQELVPAS
RVALAGEYGAVTYRRSRDGPKQLAPHPNIIRVFRAFTSSVPLLPGALAD
YPDMLPPHYYPEGLGHGRTLFLVMKNYPCTLRQYLEEQTPSSRLATMMT
LQLLEGVDHLVQQGIAHRDLKSDNILVEWDSDGCPWLVISDFGCCLADQ
HVGLRLPFNSSSVERGGNGSLMAPEVSTAHSGPSAVIDYSKADTWAVGA
IAYEIFGLANPFYGQGSAHLESRSYQEAQLPEMPESVPPEARRLVRSLL
QREASKRPSARLAANVLHLSLWGEHLLALKNLKLDKMIAWLLQQSAATL
LADRLREKSCVETKLQMLFLANLECEALCQAALLLSSWRAAP.

In some embodiments, the compounds of the present invention are able to enzymatically rescue rat PINK1 (accession number XM 216565, which is incorporated by reference in its entirety) having the following amino acid sequence:

(SEQ ID NO: 3)
MAVRQALGRGLQLGRALLLRFAPKPGPVSGWGKPGPGAAWGRGERPGR
VSSPGAQPRPLGLPLPDRYRFFRQSVAGLAARIQRQFVVRARGGAGPCG
RAVFLAFGLGLGLIEEKQAESRRAASACQEIQAIFTQKNKQVSDPLDTR
RWQGFRLEDYLIGQAIGKGCNAAVYEATMPTLPQHLEKAKHLGLLGKGP
DVVSKGADGEQAPGAPAFPFAIKMMWNISAGSSSEAILSKMSQELVPAS
RMALDGEYGAVTYRRSRDGPKQLAPHPNIIRVFRAFTSSVPLLPGALAD
YPDMLPPHYYPEGLGHGRTLFLVMKNYPCTLRQYLEEQTPSSRLATMMT
LQLLEGVDHLVQQGIAHRDLKSDNILVEWDSDGCPWLVISDFGCCLADE
RVGLQLPFNSSSVERGGNGSLMAPEVSTAHSGPHAVIDYSKADTWAVGA
IAYEIFGLANPFYGQGSAHLESRSYQEAQLPEMPKSVPPETRQLVRSLL
QREANKRPSARIAANVLHLSLWGEHLLALKNLKLDKMIAWLLQQSAATL
LADRLREKSCVETKLQMLFLANLECEALCQAALLLSSWRAAP.

In further embodiments, modulating is inhibiting. In still further embodiments, modulating is decreasing.

In further embodiments, the compound exhibits inhibition of PINK1 kinase activity with an IC₅₀ of less than about 30 μM. In still further embodiments, the compound exhibits inhibition of PINK1 kinase activity with an IC₅₀ of less than about 25 μM. In yet further embodiments, the compound exhibits inhibition of PINK1 kinase activity with an IC₅₀ of less than about 20 μM. In an even further embodiment, the compound exhibits inhibition of PINK1 kinase activity with an IC₅₀ of less than about 15 μM. In still further embodiments, the compound exhibits inhibition of PINK1 kinase activity with an IC₅₀ of less than about 10 μM. In yet further embodiments, the compound exhibits inhibition of PINK1 kinase activity with an IC₅₀ of less than about 5 μM. In an even further embodiment, the compound exhibits inhibition of PINK1 kinase activity with an IC₅₀ of less than about 1 μM. In still further embodiments, the compound exhibits inhibition of PINK1 kinase activity with an IC₅₀ of less than about 0.5 μM.

In further embodiments, the subject is a mammal. In still further embodiments, the subject is a human.

In further embodiments, the subject has been diagnosed with a need for treatment of an disorder associated with PINK1 kinase dysfunction prior to the administering step. In still further embodiments, the method further comprises the step of identifying a subject at risk of becoming infected with a disorder associated with PINK1 kinase dysfunction prior to treatment of the disorder.

### 3. Methods of Modulating Pink1 Kinase Activity in at Least One Cell

In some embodiments, disclosed are methods for modulating PINK1 kinase activity in at least one cell, the method comprising the step of contacting the at least one cell with an effective amount of at least one disclosed compound, or a pharmaceutically acceptable salt thereof.

Thus, in various embodiments, disclosed are methods for modulating PINK1 kinase activity in at least one cell, the method comprising contacting the cell with an effective amount of a compound having a structure represented by a formula:

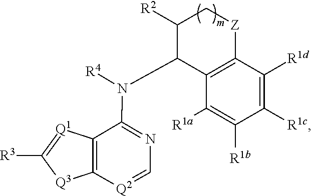

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —(CH₂)ₙCy¹, —O(CH₂)ₙCy¹, —NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof.

In various embodiments, disclosed are methods for modulating PINK1 kinase activity in at least one cell, the method comprising contacting the cell with an effective amount of a compound having a structure represented by a formula:

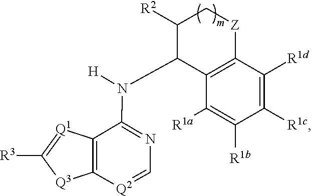

wherein m is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, —NR¹(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R³ is a 3- to 6-membered cycloalkyl, a C1-C6 haloalkyl, C1-C6 haloalkoxy, or C1-C6 halohydroxyalkyl, or a pharmaceutically acceptable salt thereof.

In further embodiments, the cell is mammalian. In still further embodiments, the cell is human. In yet further embodiments, the cell has been isolated from a mammal prior to the contacting step.

In further embodiments, modulating is inhibiting. In still further embodiments, modulating is decreasing.

In further embodiments, contacting is via administration to a mammal.

In further embodiments, the step of contacting is performed in vitro.

### 4. Use of Compounds

Also provided herein is the use of a compound described herein, or a pharmaceutically acceptable salt thereof, or a composition comprising a disclosed compound or pharmaceutically acceptable salt thereof, for the manufacture of a medicament for treating a disorder described herein. Also provided is a compound described herein, or a pharmaceutically acceptable salt thereof, or a composition comprising a disclosed compound or pharmaceutically acceptable salt thereof, for use in treating a disorder described herein.

Thus, in some embodiments, the invention relates to the use of a disclosed compound or a product of a disclosed method. In further embodiments, a use relates to the manufacture of a medicament for the treatment of a disorder associated with PINK1 kinase activity in a mammal.

Also provided are the uses of the disclosed compounds and products. In some embodiments, the invention relates to use of at least one disclosed compound; or a pharmaceutically acceptable salt, hydrate, solvate, or polymorph thereof. In further embodiments, the compound used is a product of a disclosed method of making.

In further embodiments, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In further embodiments, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various embodiments, the use relates to a treatment of a disorder associated with PINK1 kinase activity in a mammal. In some embodiments, the use is characterized in that the mammal is a human. In some embodiments, the use is characterized in that the disorder associated with PINK1 kinase activity is a neurodegenerative disease, a mitochondrial disease, fibrosis, and/or cardiomyopathy.

In further embodiments, the use relates to the manufacture of a medicament for the treatment of a disorder associated with PINK1 kinase activity in a mammal.

It is understood that the disclosed uses can be employed in connection with the disclosed compounds, products of disclosed methods of making, methods, compositions, and kits. In further embodiments, the invention relates to the use of a disclosed compound or a disclosed product in the manufacture of a medicament for the treatment of a disorder associated with PINK1 kinase activity in a mammal.

### 5. Manufacture of a Medicament

In some embodiments, the invention relates to a method for the manufacture of a medicament for treating a disorder associated with PINK1 kinase activity in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in treatment of a disorder associated with PINK1 kinase activity. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the compound of the present disclosure administered in atypical treatment is preferably between about 1 mg/kg and about 100 mg/kg of body weight for mice, and between about 10 mg/kg and about 50 mg/kg of body weight, and more preferably between 20 mg/kg and about 40 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Any medicament having utility in an application described herein can be used in co-therapy, co-administration or co-formulation with a composition as described above. Such additional medicaments include, medicines for cholesterol, such as but not limited to niacin, acifran, a statin, such as, but not limited to, lovastatin, atorvastatin, fluvastatin, pitavastatin, rosuvastatin, simvastatin, and the like. Other additional medicaments include, but are not limited to, ezetimibe, Trilipix (fenofibric acid), and the like. Other medicaments and compositions include, but are not limited to, fish oil, red yeast rice, omega fatty acids, and the like.

The additional medicament can be administered in co-therapy (including co-formulation) with the one or more of the compounds described herein.

In some embodiments, the response of the disease or disorder to the treatment is monitored and the treatment regimen is adjusted if necessary in light of such monitoring.

Frequency of administration is typically such that the dosing interval, for example, the period of time between one dose and the next, during waking hours is from about 2 to about 12 hours, from about 3 to about 8 hours, or from about 4 to about 6 hours. It will be understood by those of skill in the art that an appropriate dosing interval is dependent to some degree on the length of time for which the selected composition is capable of maintaining a concentration of the compound(s) in the subject and/or in the target tissue (e.g., above the EC₅₀ (the minimum concentration of the compound which modulates the receptor's activity by 90%). Ideally the concentration remains above the EC₅₀ for at least 100% of the dosing interval. Where this is not achievable it is desired that the concentration should remain above 5% of the EC₅₀, above 10% of the EC₅₀, above 25% of the EC₅₀, or above 50% of the EC₅₀ for the dosing period.

Thus, in some embodiments, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 6. Kits

In some embodiments, disclosed are kits comprising a compound having a structure represented by a formula:

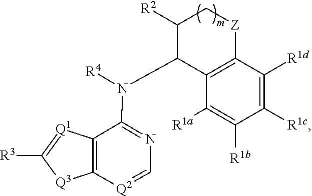

wherein in is 0 or 1; wherein each of Q¹ and Q² is independently N or CH; wherein Q³ is CH₂ or NH; wherein Z is CR^{11a}R^{11b}, NR¹², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —(CH₂)ₙCy¹, —O(CH₂)ₙCy¹, NR¹³(CH₂)ₙCy¹, —CH(OH)Cy¹, and Cy¹; wherein n, when present, is 0, 1, or 2; wherein R¹³, when present, is selected from hydrogen and C1-C4 alkyl; wherein Cy¹ is a C4-C9 cycloalkyl, a C3-C9 heterocycle having at least one O, S, or N atom, or a C2-C9 heteroaryl having at least one 0, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C₁-C₄ hydroxyalkyl, C1-C4 haloalkoxy, C1-C4 alkoxy, —(C₁-C₄)—O—(C1-C4 alkyl), —C(O)(C1-C4 alkyl), —S(O)R¹⁴, C1-C4 alkylamino, and (C1-C4)(C1-C4) dialkylamino; wherein R¹⁴, when present, is selected from —OH, —NH₂, —O(C1-C4 alkyl), —NH(C1-C4 alkyl), and —N(C1-C4 alkyl)(C1-C4 alkyl); wherein R³ is a 3- to 6-membered cycloalkyl, a C1-C6 haloalkyl, C1-C6 haloalkoxy, or C1-C6 halohydroxyalkyl; and wherein R⁴ is selected from hydrogen and C1-C4 alkyl, or a pharmaceutically acceptable salt thereof, and one or more of: (a) at least one agent known for the treatment of a neurodegenerative disease, a mitochondrial disease, fibrosis, and/or cardiomyopathy; (b) instructions for administering the compound in connection with treating a neurodegenerative disease, a mitochondrial disease, fibrosis, and/or cardiomyopathy; and (c) instructions for treating a neurodegenerative disease, a mitochondrial disease, fibrosis, and/or cardiomyopathy.

In some embodiments, disclosed are kits comprising a compound having a structure represented by a formula:

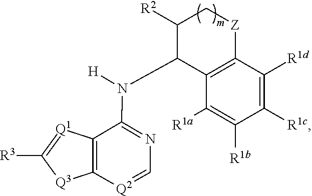

wherein m is 0 or 1; wherein each of Q¹ and Q² is independently N or CH; wherein Q³ is CH₂ or NH; wherein Z is CR^{11a}R^{11b}, NR², or O; wherein each of R^{11a} and R^{11b}, when present, is independently selected from hydrogen, halogen, —OH, and C1-C4 alkyloxy, or wherein each of R^{11a} and R^{11b}, when present, together comprise ═O; wherein R¹², when present, is hydrogen, C1-C4 alkyl, C3-C6 cycloalkyl, or —(C1-C4 alkyl)(C3-C6 cycloalkyl); wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R² is selected from —O(CH₂)ₙCy¹, —NR¹³(CH₂)ₙCy¹, and Cy¹; wherein n, when present, is 0, 1, or 2; wherein R¹³, when present, is selected from hydrogen and C1-C4 alkyl; wherein Cy¹ is a C3-C9 heterocycle having at least one O, S, or N atom and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R is a 3- to 6-membered cycloalkyl, a C1-C6 haloalkyl, C1-C6 haloalkoxy, or C1-C6 halohydroxyalkyl, or a pharmaceutically acceptable salt thereof, and one or more of: (a) at least one agent known for the treatment of a neurodegenerative disease, a mitochondrial disease, fibrosis, and/or cardiomyopathy; (b) instructions for administering the compound in connection with treating a neurodegenerative disease, a mitochondrial disease, fibrosis, and/or cardiomyopathy; and (c) instructions for treating a neurodegenerative disease, a mitochondrial disease, fibrosis, and/or cardiomyopathy.

In further embodiments, the agent is known for the treatment of a neurodegenerative disorder. Examples of agents known for the treatment of neurodegenerative disorders include, but are not limited to, cholinesterase inhibitor, an antidepressant, memantine, rilutek, radicava, levodopa, carbidopa, a dopamine agonist, a MAO-B inhibitor, a catechol-O-methyltransferase inhibitor, an anticholinergic, spinraza, tetrabenazine, an antipsychotic agent, levetiracetam, clonazepam, an antipsychotic agent, a mood-stabilizing agent, and amantadine.

In further embodiments, the agent is known for the treatment of a mitochondrial disease. Examples of agents known for the treatment of mitochondrial diseases include, but are not limited to, vitamins and supplements such as coenzyme Q10, B complex vitamins (e.g., thiamine (B1) and riboflavin (B2)), alpha lipoic acid, L-carnitine (Carnitor), creatine, and L-arginine.

In further embodiments, the agent is known for the treatment of fibrosis such as, for example, idiopathic pulmonary fibrosis (IPF), non-alcoholic fatty liver disease (NASH), liver fibrosis, heart fibrosis, mediastinal fibrosis, bone marrow fibrosis, retroperitoneal cavity fibrosis, and renal fibrosis. Examples of agents known for the treatment of fibrosis include, but are not limited to, pirfenidone, nintedanib, a prostaglandin such as latanoprost and bimaotoprost, a beta blocker such as timolo1 and betaxolol, an alpha-adrenergic agonist such as apraclonidine and brimonidine, a carbonic anhydrase inhibitor such as dorzolamide and brinzolamide, a moitic or cholinergic agent such as pilocarpine, a diuretic, an angiotenisin-converting enzyme (ACE) inhibitor, an angiotensin II receptor blocker, an anti-inflammatory agent, and an anti-fibrotic agent.

In further embodiments, the agent is known for the treatment of cardiomyopathy. Examples of agents known for the treatment of cardiomyopathy include, but are not limited to, ACE inhibitors, angiotensin II receptor blockers, beta blockers, calcium channel blockers, digoxin, and antiarrhythmics. In various embodiments, the agent known for the treatment of cardiomyopathy is a medical device such as, for example, an implantable cardioverter-defibrillator (ICD), a ventricular assist device (VAD), or a pacemaker.

In further embodiments, the at least one compound and the at least one agent are co-formulated. In further embodiments, the at least one compound and the at least one agent are co-packaged.

In further embodiments, the compound and the agent are administered sequentially. In still further embodiments, the compound and the agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. EXAMPLES

Representative examples of the disclosed compounds are illustrated in the following non-limiting methods, schemes, and examples.

### 1. General Experimental Method

General starting materials used were obtained from commercial sources or prepared in other examples, unless otherwise noted. All temperatures are in degrees Celsius (°C) and are uncorrected. Reagent grade chemicals and anhydrous solvent were purchased from commercial sources and unless otherwise mentioned, were used without further purification. The names of the products were determined using the naming software included in Biovia electronic lab notebook. Silica gel chromatography was performed on Teledyne Isco instruments using pre-packaged disposable SiO₂ stationary phase columns with eluent flow rate range of 15 to 200 mL/min, UV detection (254 and 280 nm), Reverse phase preparative HPLC was carried out using C18 columns, UV detection (214 and 254 nim) eluting with gradients of MeCN in H₂O (0.03% (NH₄)₂CO₃/ 0.375% NH₄OH, high pH) or MeCN in H₂O (0.1% HCOOH, low pH). The analytical HPLC chromatograms were performed using an Agilent 1100 series instrument with DAD detector (190 nm to 300 un). The mass spectra were recorded with a Waters Micromass ZQ detector at 130 °C. The mass spectrometer was equipped with an electrospray ion source (ESI) operated in a positive ion mode and was set to scan between m/z 150-750 with a scan time of 0.3 s. Products and intermediates were analyzed by HPLC/MS on a Gemini-NX (5 μM, 2.0×30 mm) using a high pH buffer gradient of 5% to 100% of MeCN in H₂O (0.03% (NH₄)₂CO₃/0.375% NH₄OH) over 2.5 min at 1.8 mL/min for a 3.5 min run (B05) and EVO C18 (5 μM, 3.0×50 mm) using a low pH buffer gradient of 5% to 100% of MeCN in H₂O (0.1% HCOOH) over 2.5 min at 2.2 mL/min for a 3.5 min run (A05). The ¹H NMR spectra were recorded on a Bruker UltraShield 500 MHz/54 mm instrument (BZH 43/500/70B, D221/54-3209). The chemical shifts are referenced to solvent peaks, which in ¹H NMR appear at 7.26 ppm for CDCl₃, 2.50 for DMSO-d6, and 3.31 ppm for CD₃OD.

The following abbreviations have the indicated meanings:
aq aqueous;
(Bpin)₂ bis(pinacolato)diboron;
Comins' reagent N-bis(trifluoromethanesulfonimide);
DBDMI 1,3-dibromo-5,5-dimethylhydantoin
DMF N,N-dimethyl formamide;
DMSO dimethyl sulfoxide;
Et₂O diethyl ether;
EtOAc ethyl acetate;
EtOH ethanol;
eq. or equiv. equivalent
h hour(s);
HPLC high performance liquid chromatography;
LCMS liquid chromatography mass spectrometry
LiHMDS lithium bis(trimethylsilyl)amide
MeOH methanol;
m minute(s);
MS mass spectrometry
NaHMDS sodium bis(trimethylsilyl)amide
NMP N-methylpyrrolidone
NMR nuclear magnetic resonance:
23 °C room temperature;
sat. saturated;
SFC supercritical fluid chromatography;
THF tetrahydrofuran;
OTf trifluoromethanesulfonate;

### 2. Synthesis of Adenine Analogs

The synthetic protocols used to access the exemplary compounds disclosed herein are illustrated in Schemes 1-5 below,

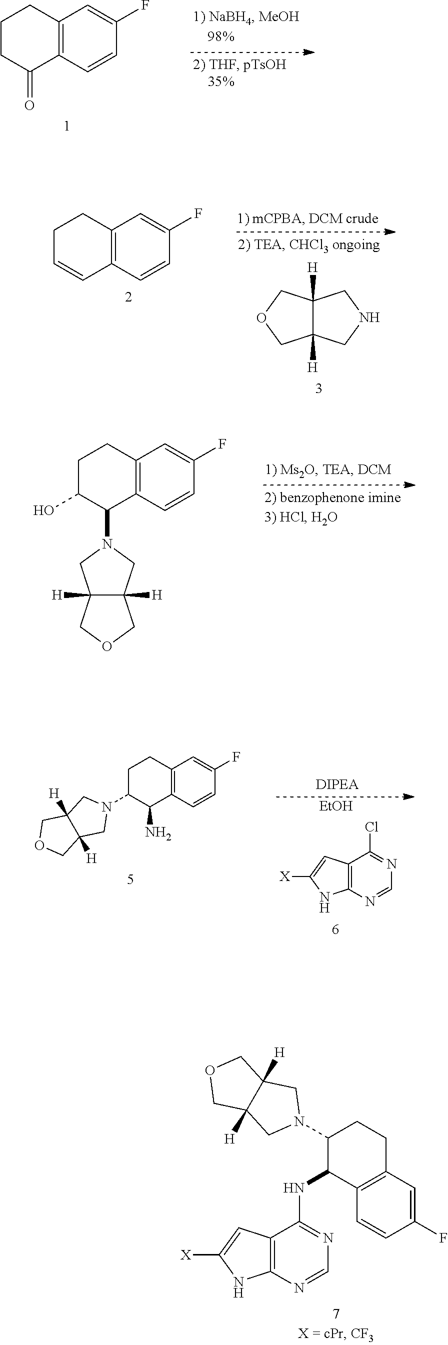

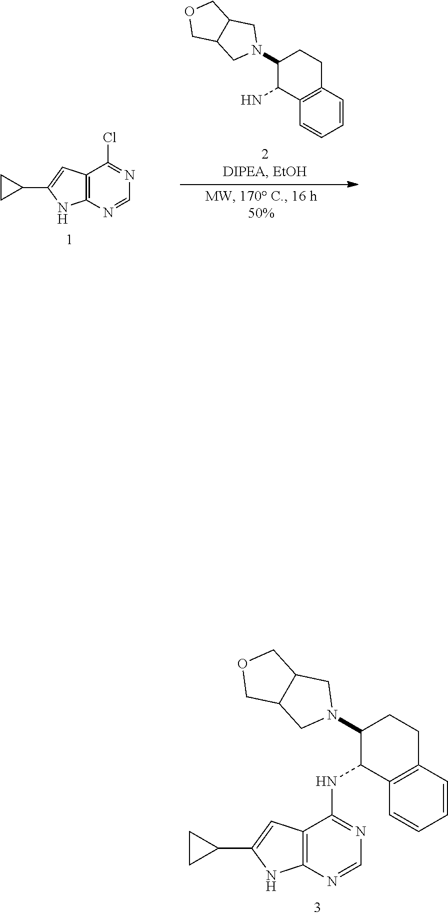

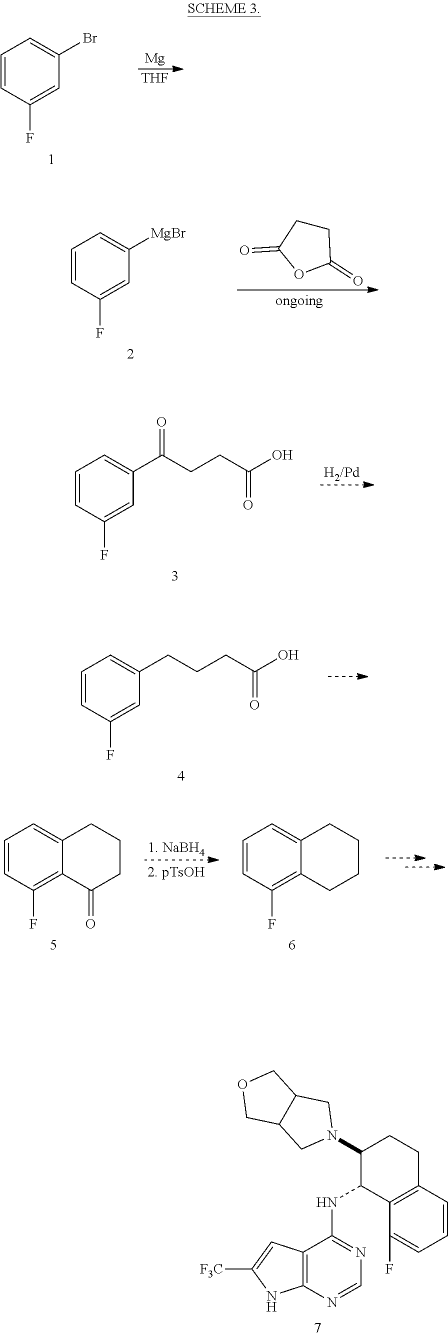

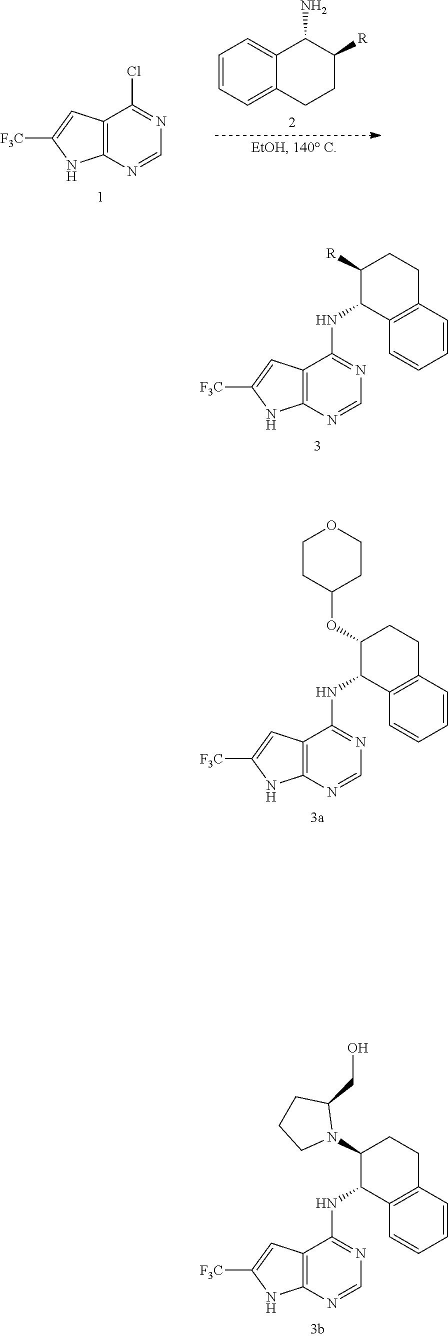

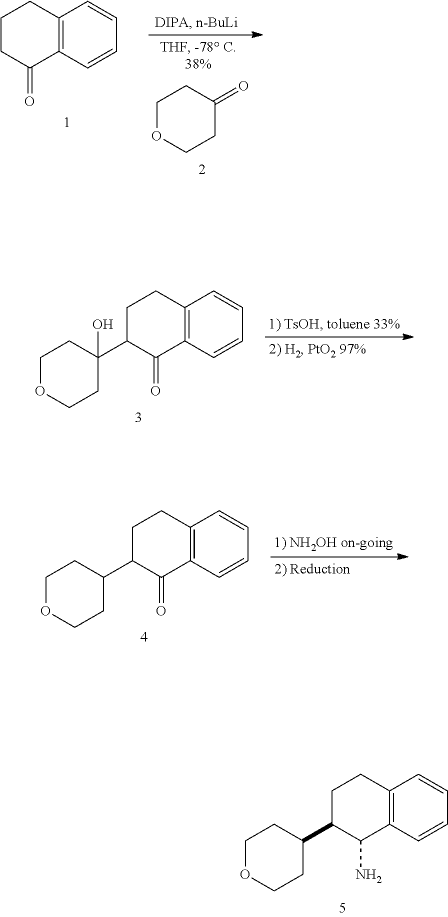 [SMILES: O=C1CCCc2ccccc21 ; O=C1CCOCC1 ; O=C1c2ccccc2CCC1C1(O)CCOCC1 ; O=C1c2ccccc2CCC1C1CCOCC1 ; N[C@H]1c2ccccc2CC[C@@H]1C1CCOCC1]

### 3. Evaluation OF ADENINE ANALOGS FOR PINK1 KINASE ACTIVITY

A list of compounds evaluated and their corresponding activity is shown in Tables 1 and 2 below.

TABLE 1*

| No. | Structure |
|---|---|
| kinetin | 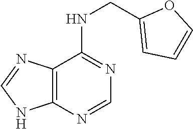 [SMILES: c1coc(CNc2ncnc3[nH]cnc23)c1] |
| EP-0035985 | 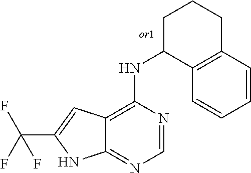 [SMILES: FC(F)(F)c1cc2c(NC3CCCc4ccccc43)ncnc2[nH]1] |
| EP-0037820 | 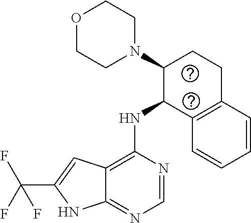 [SMILES: FC(F)(F)c1cc2c(N[C@@H]3c4ccccc4CC[C@@H]3N3CCOCC3)ncnc2[nH]1] |
| EP-0037821 (Racemic mixture) | 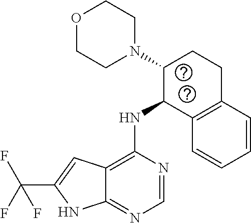 [SMILES: FC(F)(F)c1cc2c(N[C@@H]3c4ccccc4CC[C@H]3N3CCOCC3)ncnc2[nH]1] |
| EP-0038098 | 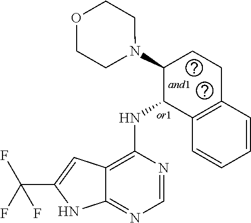 [SMILES: FC(F)(F)c1cc2c(N[C@H]3c4ccccc4CC[C@@H]3N3CCOCC3)ncnc2[nH]1] |
| EP-0038099 | 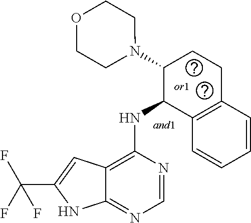 [SMILES: FC(F)(F)c1cc2c(N[C@@H]3c4ccccc4CC[C@H]3N3CCOCC3)ncnc2[nH]1] |
| EP-0038205 | 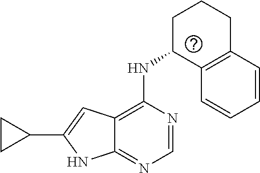 [SMILES: c1ccc2c(c1)CCC[C@H]2Nc1ncnc2[nH]c(C3CC3)cc12] |
| EP-0038249 | 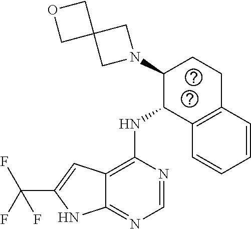 [SMILES: FC(F)(F)c1cc2c(N[C@H]3c4ccccc4CC[C@@H]3N3CC4(COC4)C3)ncnc2[nH]1] |
| EP-0038282 | 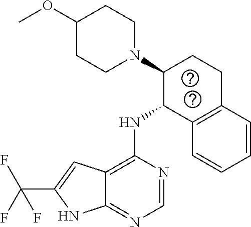 [SMILES: COC1CCN([C@H]2CCc3ccccc3[C@@H]2Nc2ncnc3[nH]c(C(F)(F)F)cc23)CC1] |
| EP-0038283 | 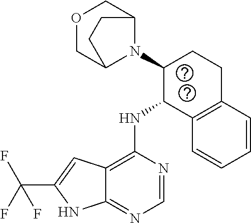 [SMILES: FC(F)(F)c1cc2c(N[C@H]3c4ccccc4CC[C@@H]3N3C4CCC3COC4)ncnc2[nH]1] |
| EP-0038378 | 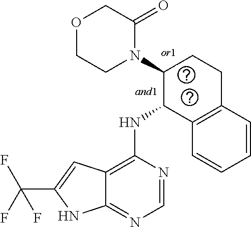 [SMILES: O=C1COCCN1[C@H]1CCc2ccccc2[C@@H]1Nc1ncnc2[nH]c(C(F)(F)F)cc12] |
| EP-0038392 | 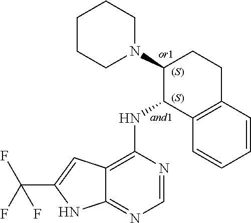 |
| EP-0038393 | 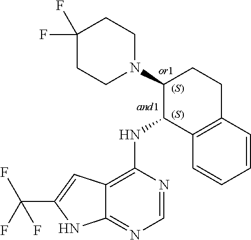 |
| EP-0038394 | 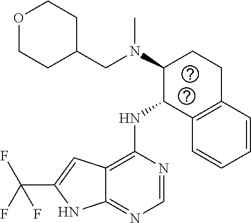 [SMILES: CN(CC1CCOCC1)[C@H]1CCc2ccccc2[C@@H]1Nc1ncnc2[nH]c(C(F)(F)F)cc12] |
| EP-0038461 | 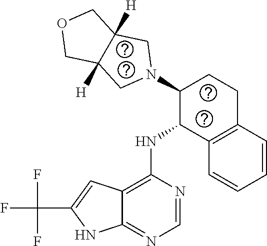 [SMILES: FC(F)(F)c1cc2c(N[C@H]3c4ccccc4CC[C@@H]3N3C[C@H]4COC[C@H]4C3)ncnc2[nH]1] |
| EP-0038463 | 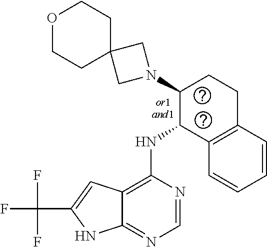 [SMILES: FC(F)(F)c1cc2c(N[C@H]3c4ccccc4CC[C@@H]3N3CC4(CCOCC4)C3)ncnc2[nH]1] |
| EP-0038503 | 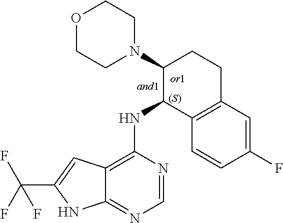 |
| EP-0038504 | 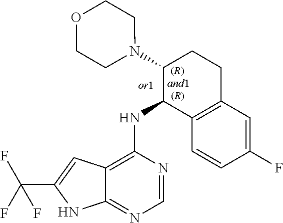 |
| EP-0038508 | 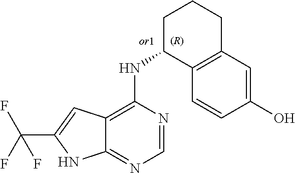 |
| EP-0038521 | 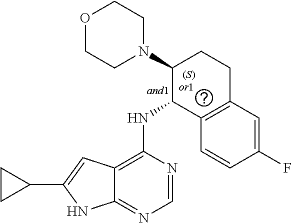 |
| EP-0038582 | 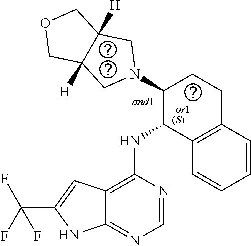 |
| EP-0038583 | 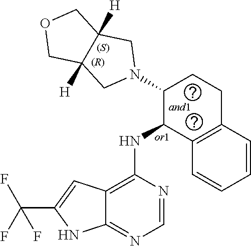 |
| EP-0039677 | 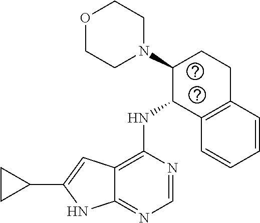 [SMILES: c1ccc2c(c1)CC[C@H](N1CCOCC1)[C@H]2Nc1ncnc2[nH]c(C3CC3)cc12] |
| EP-0039678 | 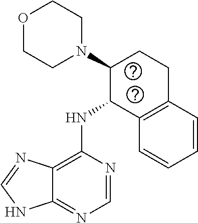 [SMILES: c1ccc2c(c1)CC[C@H](N1CCOCC1)[C@H]2Nc1ncnc2[nH]cnc12] |
| EP-0039713 | 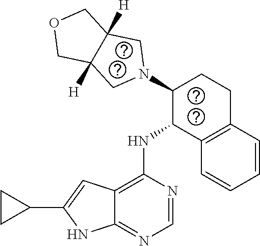 [SMILES: c1ccc2c(c1)CC[C@H](N1C[C@H]3COC[C@H]3C1)[C@H]2Nc1ncnc2[nH]c(C3CC3)cc12] |
| EP-0039742 | 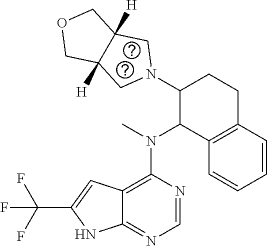 [SMILES: CN(c1ncnc2[nH]c(C(F)(F)F)cc12)C1c2ccccc2CCC1N1C[C@H]2COC[C@H]2C1] |
| EP-0039743 | 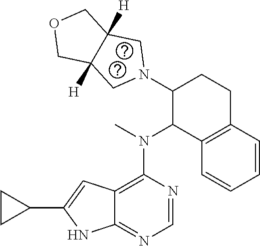 [SMILES: CN(c1ncnc2[nH]c(C3CC3)cc12)C1c2ccccc2CCC1N1C[C@H]2COC[C@H]2C1] |
| EP-0039746 | 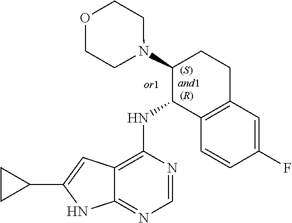 |
| EP-0039747 | 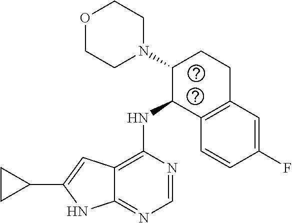 [SMILES: Fc1ccc2c(c1)CC[C@@H](N1CCOCC1)[C@@H]2Nc1ncnc2[nH]c(C3CC3)cc12] |
| EP-0040078 | 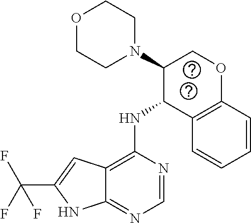 [SMILES: FC(F)(F)c1cc2c(N[C@H]3c4ccccc4OC[C@@H]3N3CCOCC3)ncnc2[nH]1] |
| EP-0040080 | 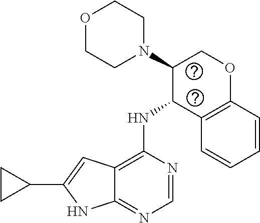 [SMILES: c1ccc2c(c1)OC[C@H](N1CCOCC1)[C@H]2Nc1ncnc2[nH]c(C3CC3)cc12] |
| EP-0040139 | 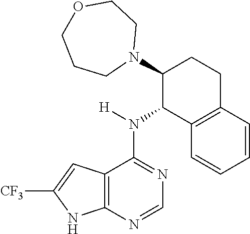 [SMILES: FC(F)(F)c1cc2c(N[C@H]3c4ccccc4CC[C@@H]3N3CCCOCC3)ncnc2[nH]1] |
| | 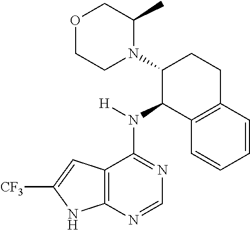 [SMILES: C[C@@H]1COCCN1[C@@H]1CCc2ccccc2[C@H]1Nc1ncnc2[nH]c(C(F)(F)F)cc12] |
| EP-0040120 | 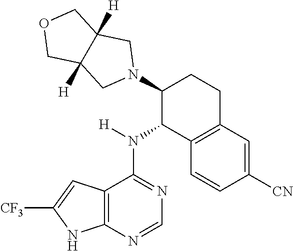 [SMILES: N#Cc1ccc2c(c1)CC[C@H](N1C[C@H]3COC[C@H]3C1)[C@H]2Nc1ncnc2[nH]c(C(F)(F)F)cc12] |
| EP-0039748 | 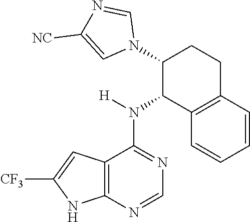 [SMILES: N#Cc1cn([C@@H]2CCc3ccccc3[C@@H]2Nc2ncnc3[nH]c(C(F)(F)F)cc23)cn1] |
| EP-0038523 | 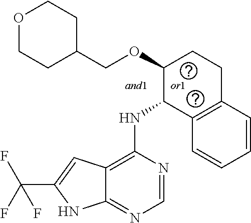 [SMILES: FC(F)(F)c1cc2c(N[C@H]3c4ccccc4CC[C@@H]3OCC3CCOCC3)ncnc2[nH]1] |
| EP-0039732 | 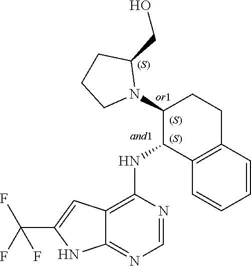 |
| EP-0039733 | 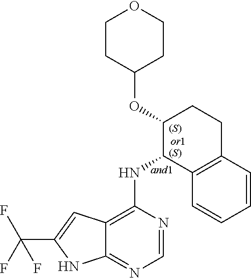 |
| EP-0039754 | 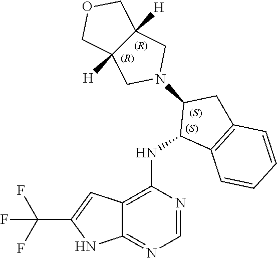 |
| EP-0040075 | 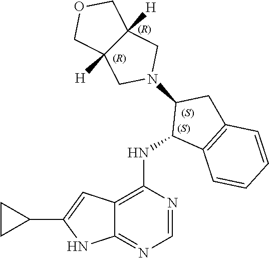 |
| EP-0040078 | 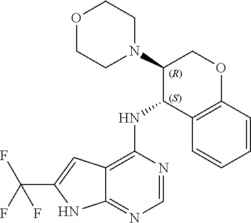 |
| EP-0040080 | 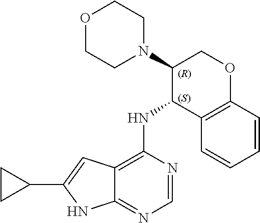 |
| EP-0040084 | 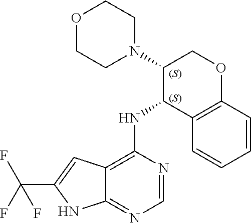 |
| EP-0040085 | 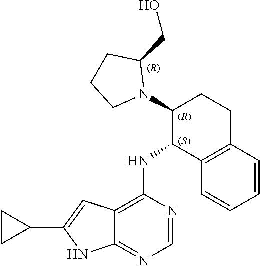 |
| EP-0040108 | 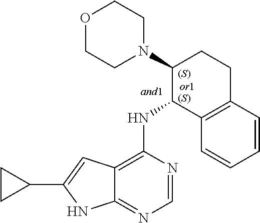 |
| EP-0040109 | 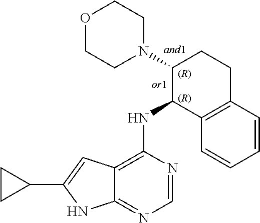 |
| EP-0040121 | 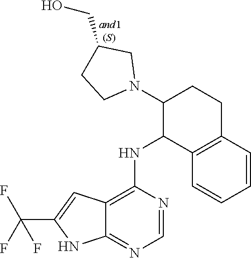 |
| EP-0040138 | 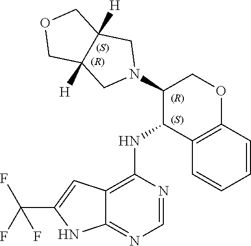 |
| EP-0040140 | 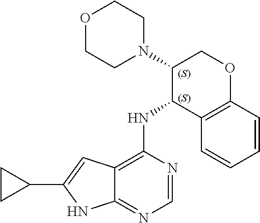 |
| EP-0040180 | 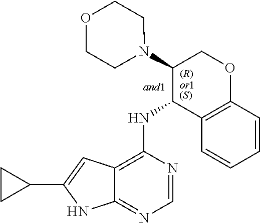 |
| EP-0040181 | 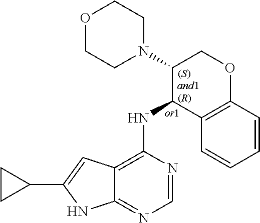 |
| EP-0040182 | 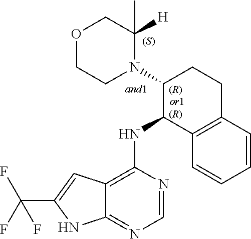 |
| EP-0040193 | 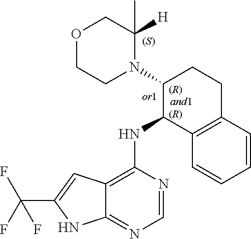 |
| EP-0040195 | 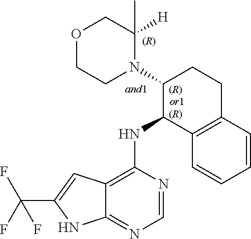 |
| EP-0040197 | 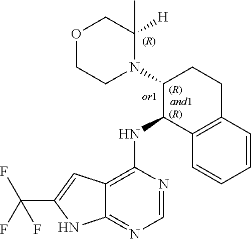 |
| EP-0040248 | 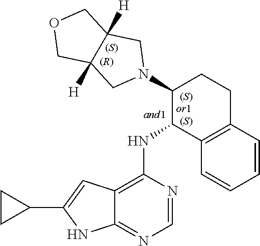 |
| EP-0040249 | 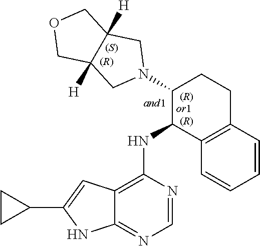 |
| EP-0040251 | 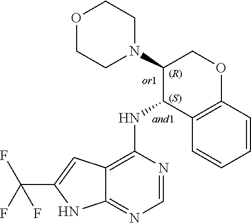 |
| EP-0040252 | 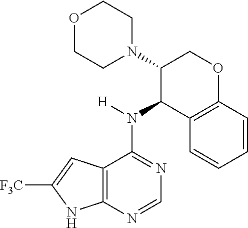 [SMILES: FC(F)(F)c1cc2c(N[C@@H]3c4ccccc4OC[C@H]3N3CCOCC3)ncnc2[nH]1] |
| EP-0040268 | 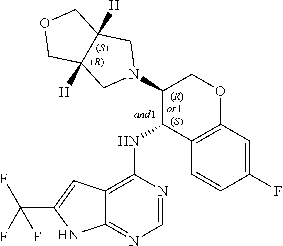 |
| EP-0040269 | 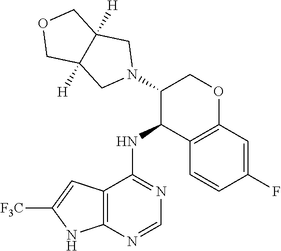 [SMILES: Fc1ccc2c(c1)OC[C@@H](N1C[C@H]3COC[C@H]3C1)[C@@H]2Nc1ncnc2[nH]c(C(F)(F)F)cc12] |
| EP-0040270 | 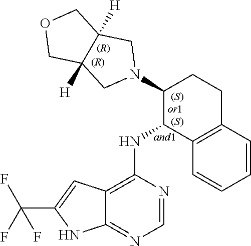 |
| EP-0040271 | 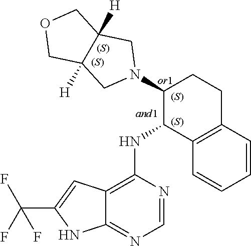 |
| EP-0040272 | 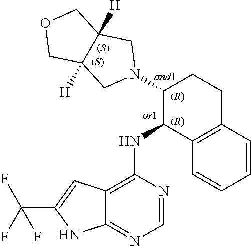 |
| EP-0040273 | 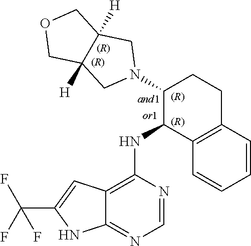 |
| EP-0040285 | 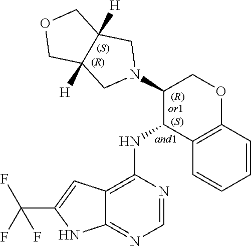 |
| EP-0040286 | 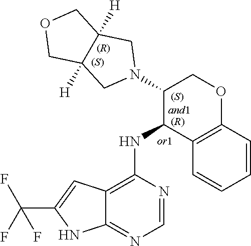 |
| EP-0040310 | 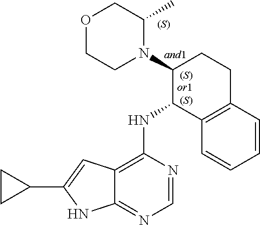 |
| EP-0040323 | 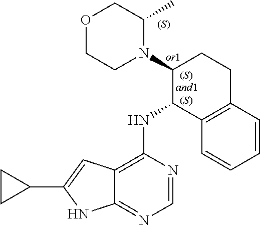 |
| EP-0040326 | 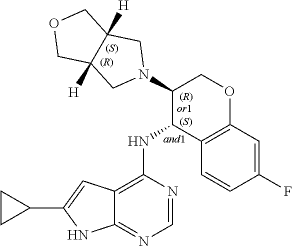 |
| EP-0040328 | 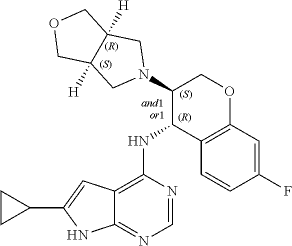 |
| EP-0040339 | 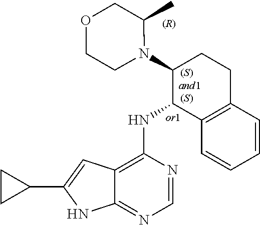 |
| EP-0040358 | 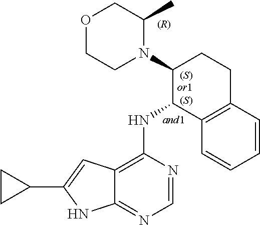 |
| EP-0040391 | 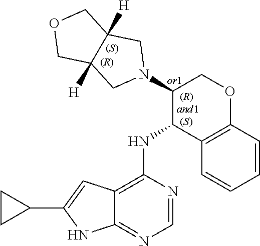 |
| EP-0040392 | 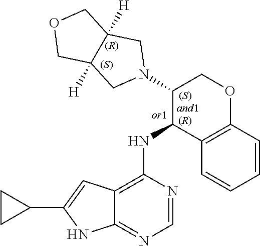 |
| EP-0040395 | 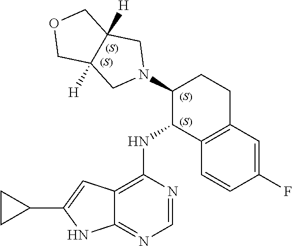 |
| EP-0040491 | 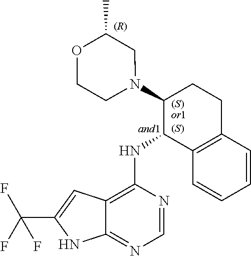 |
| EP-0040492 | 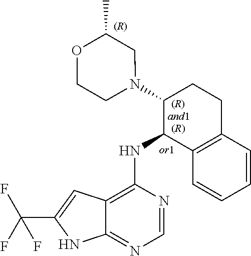 |
| EP-0040493 | 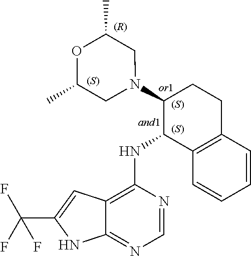 |
| EP-0040494 | 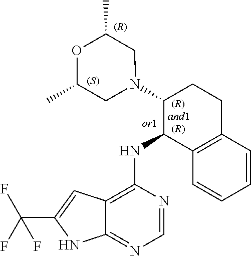 |
| EP-0040496 | 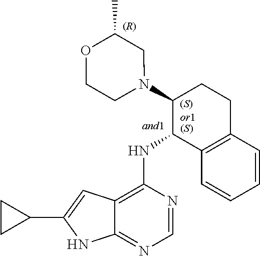 |
| EP-0040497 | 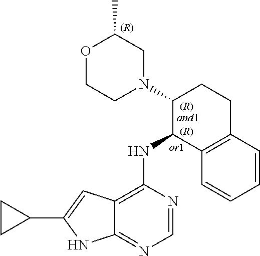 |
| EP-0040498 | 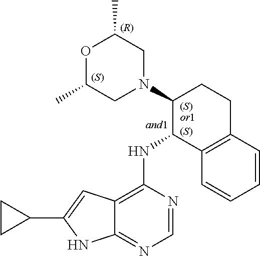 |
| EP-0040499 | 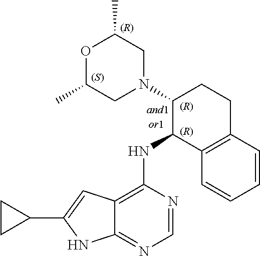 |
| EP-0040500 | 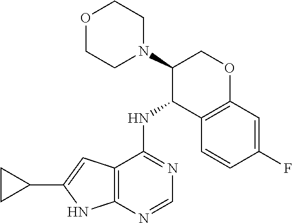 [SMILES: Fc1ccc2c(c1)OC[C@H](N1CCOCC1)[C@H]2Nc1ncnc2[nH]c(C3CC3)cc12] |
| EP-0040501 | 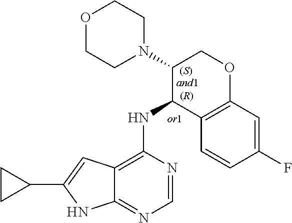 |
| EP-0040503 | 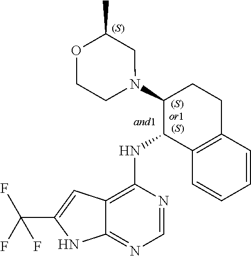 |
| EP-0040504 | 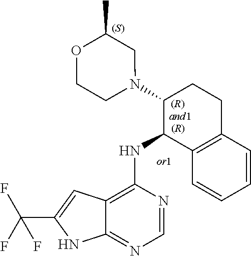 |
| EP-0040505 | 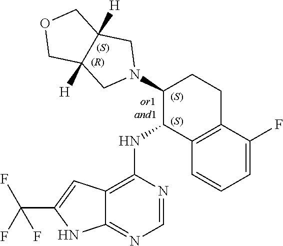 |
| EP-0040506 | 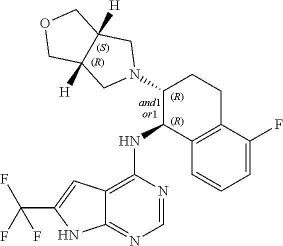 |
| EP-0040511 | 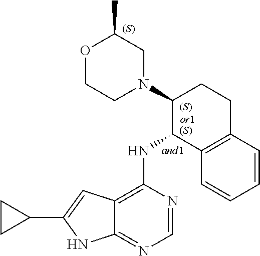 |
| EP-0040512 | 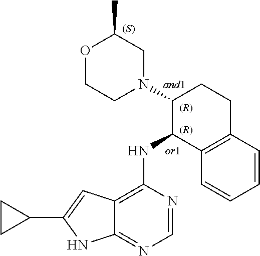 |
| EP-0040523 | 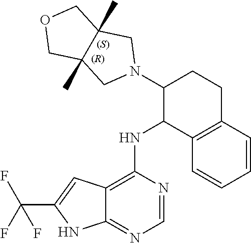 |
| EP-0040524 | 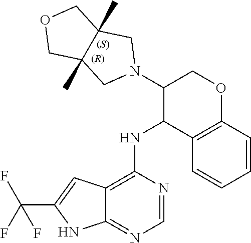 |
| EP-0040542 | 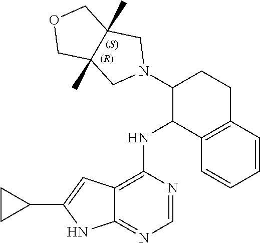 |
| EP-0040545 | 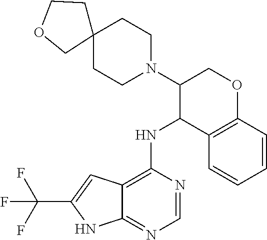 [SMILES: FC(F)(F)c1cc2c(NC3c4ccccc4OCC3N3CCC4(CCOC4)CC3)ncnc2[nH]1] |
| EP-0040557 | 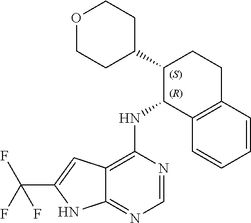 |
| EP-0040558 | 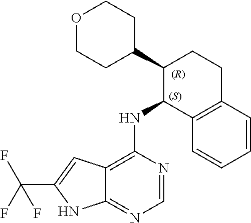 |
| EP-0040562 | 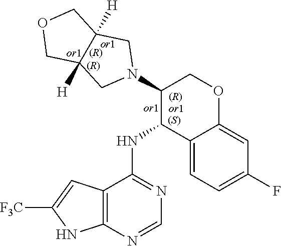 |
| EP-0040563 | 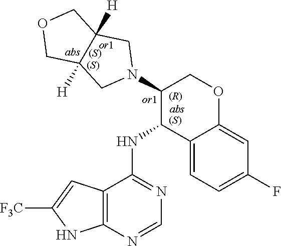 |
| EP-0040576 | 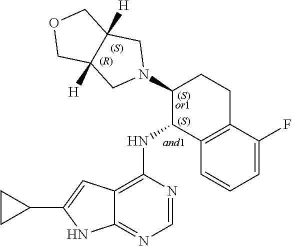 |
| EP-0040577 | 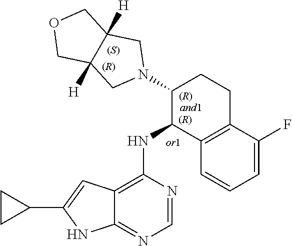 |
| EP-0040578 | 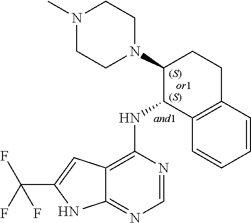 |
| EP-0040579 | 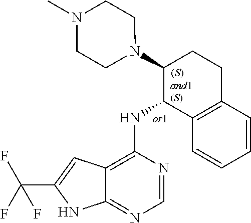 |
| EP-0040580 | 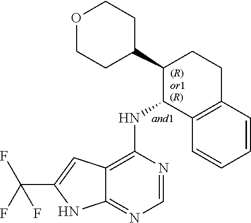 |
| EP-0040581 | 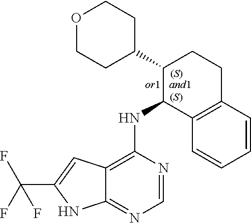 |
| EP-0040582 | 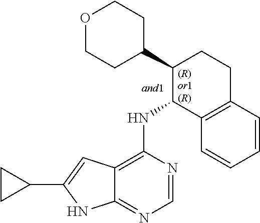 |
| EP-0040583 | 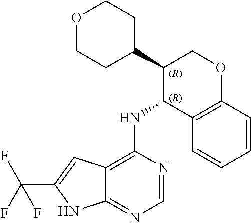 |
| EP-0040586 | 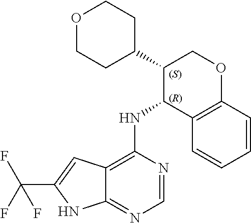 |
| EP-0040587 | 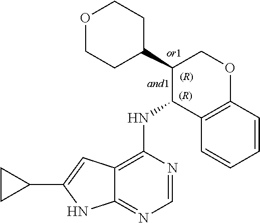 |
| EP-0040589 | 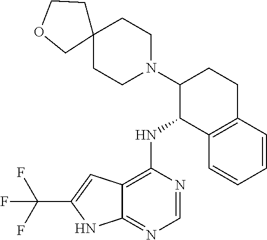 [SMILES: FC(F)(F)c1cc2c(N[C@H]3c4ccccc4CCC3N3CCC4(CCOC4)CC3)ncnc2[nH]1] |
| EP-0040601 | 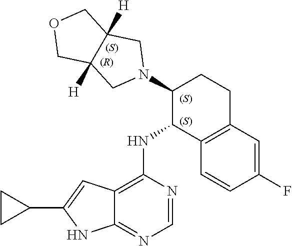 |
| EP-0040626 | 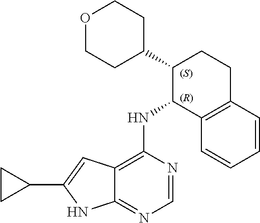 |
| EP-0040627 | 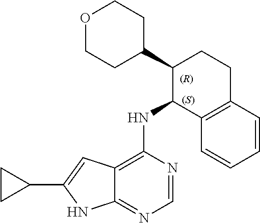 |
| EP-0040644 | 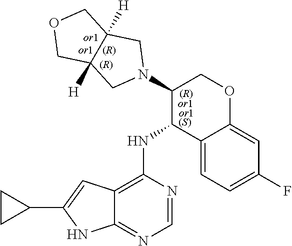 |
| EP-0040665 | 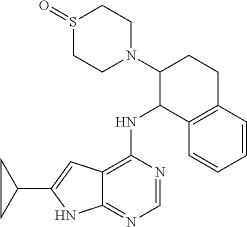 [SMILES: O=S1CCN(C2CCc3ccccc3C2Nc2ncnc3[nH]c(C4CC4)cc23)CC1] |
| EP-0040669 | 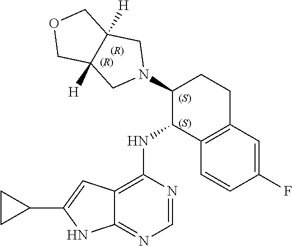 |
| EP-0040677 | 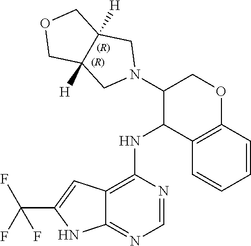 |
| EP-0040678 | 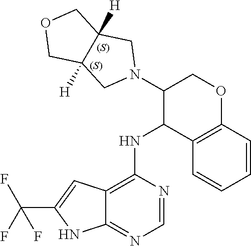 |
| EP-0040688 | 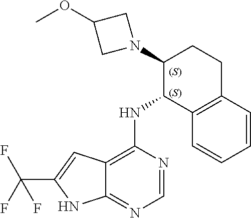 |
| EP-0040695 | 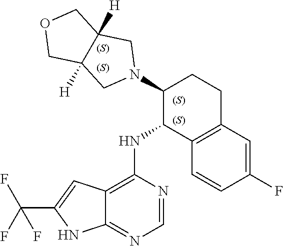 |
| EP-0040696 | 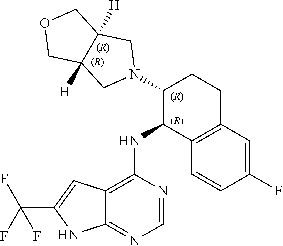 |
| EP-0040697 | 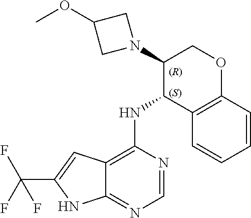 |
| EP-0040699 | 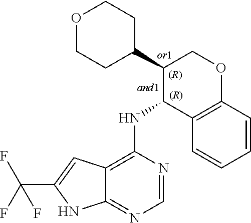 |
| EP-0040700 | 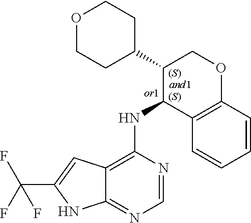 |
| EP-0040702 | 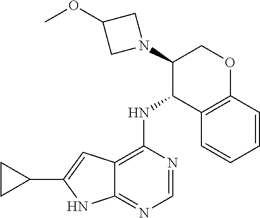 [SMILES: COC1CN([C@H]2COc3ccccc3[C@@H]2Nc2ncnc3[nH]c(C4CC4)cc23)C1] |
| EP-0040720 | 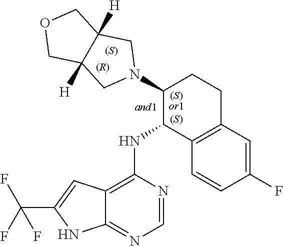 |
| EP-0040721 | 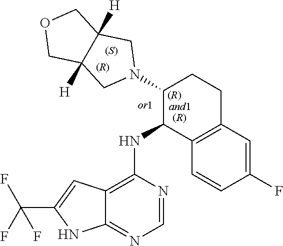 |
| EP-0040725 | 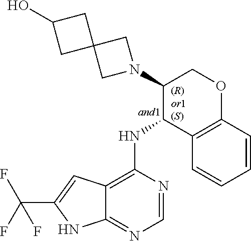 |
| EP-0040726 | 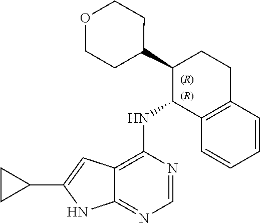 |
| EP-0040727 | 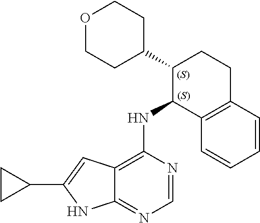 |
| EP-0040728 | 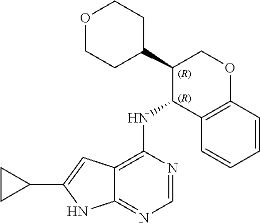 |
| EP-0040729 | 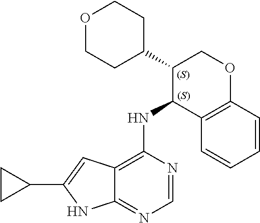 |
| EP-0040730 | 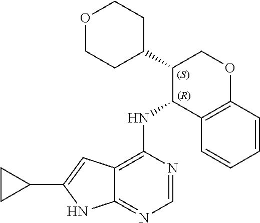 |
| EP-0040731 | 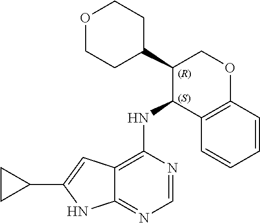 |
| EP-0040747 | 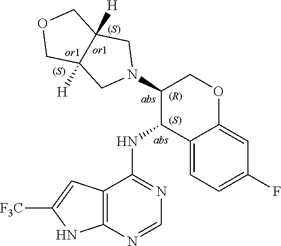 |
| EP-0040748 | 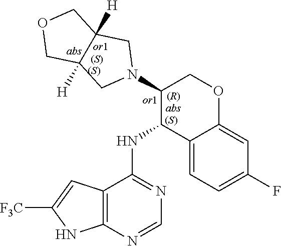 |
| EP-0040749 | 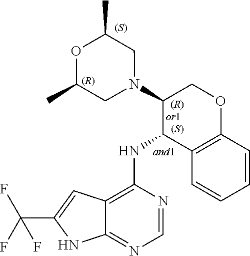 |
| EP-0040750 | 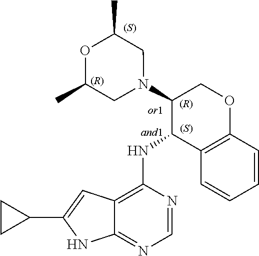 |
| EP-0040767 | 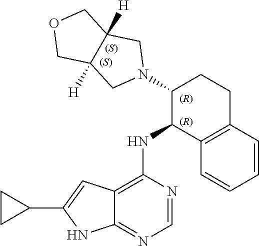 |
| EP-0040768 | 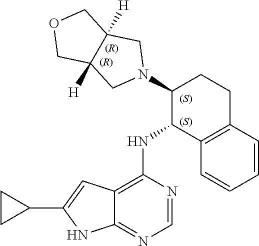 |
| EP-0040773 | 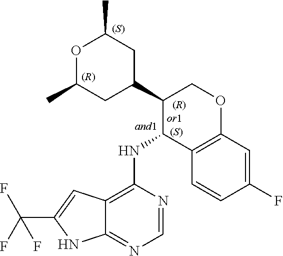 |
| EP-0040842 | 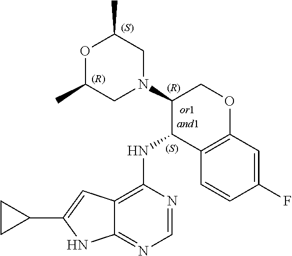 |
| EP-0040847 | 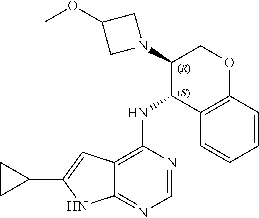 |
| EP-0040848 | 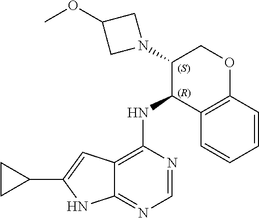 |
| EP-0040849 | 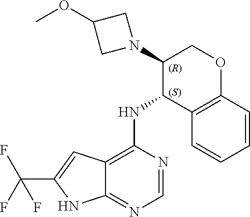 |
| EP-0040850 | 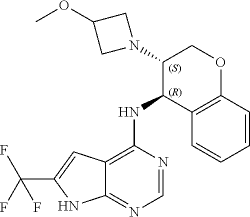 |
| EP-0040857 | 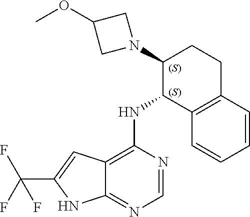 |
| EP-0040858 | 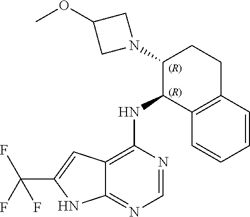 |
| EP-0040873 | 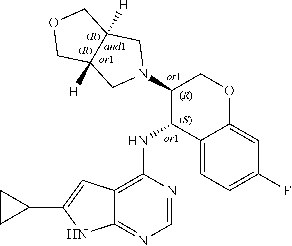 |
| EP-0040874 | 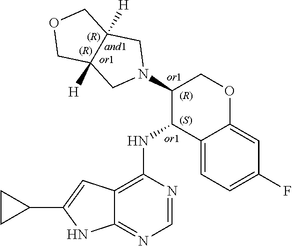 |
| EP-0040942 | 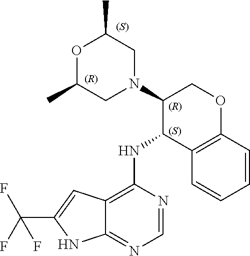 |
| EP-0040943 | 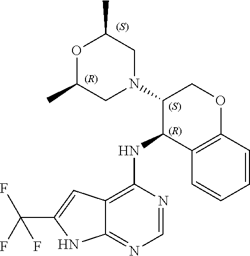 |
| EP-0040944 | 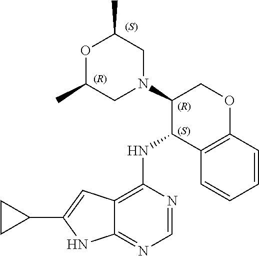 |
| EP-0040945 | 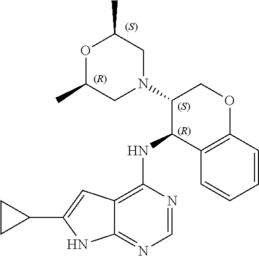 |
| EP-0040946 | 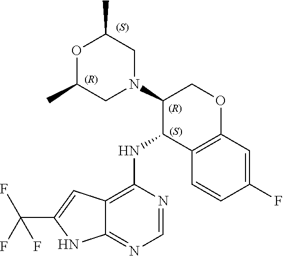 |
| EP-0040947 | 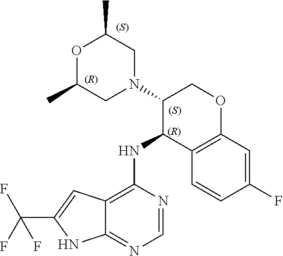 |
| EP-0040948 | 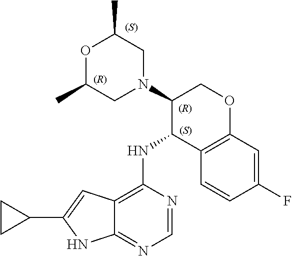 |
| EP-0040949 | 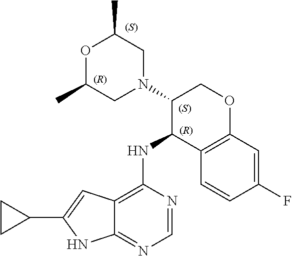 |
| EP-0040955 | 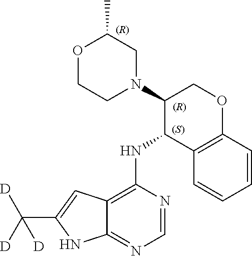 |
| EP-0040956 | 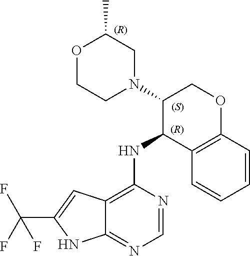 |
| EP-0040957 | 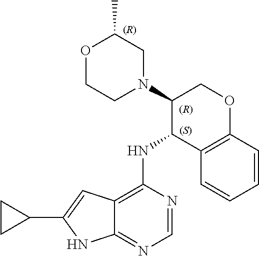 |
| EP-0040958 | 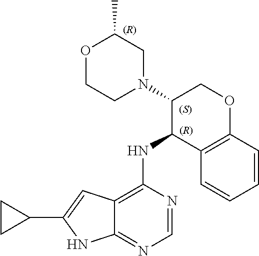 |
| EP-0040979 | 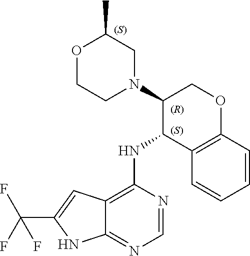 |
| EP-0040980 | 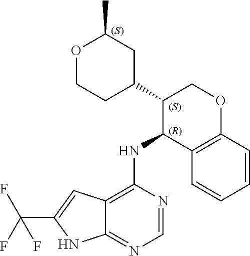 |
| EP-0040981 | 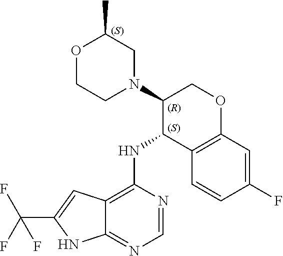 |
| EP-0040982 | 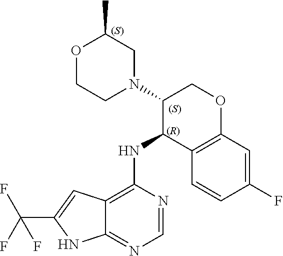 |
| EP-0040999 | 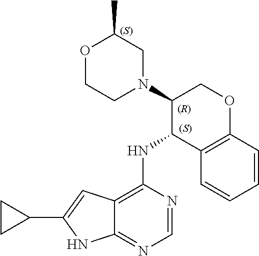 |
| EP-0041000 | 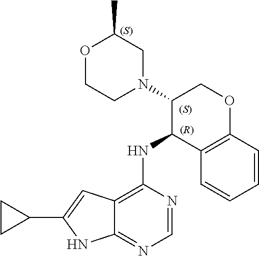 |
| EP-0041002 | 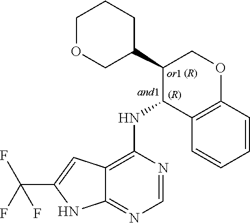 |
| EP-0041003 | 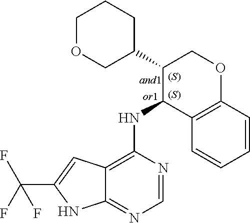 |
| EP-0041004 | 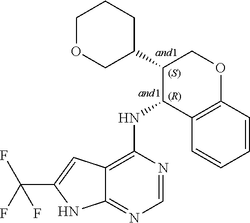 |
| EP-0041005 | 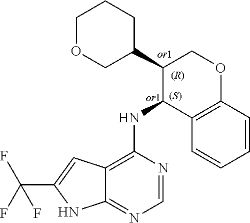 |
| EP-0041021 | 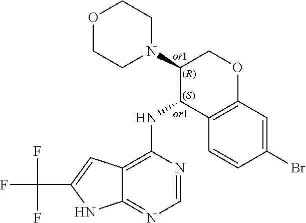 |
| EP-0041045 | 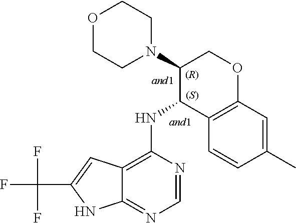 |
| EP-0041087 | 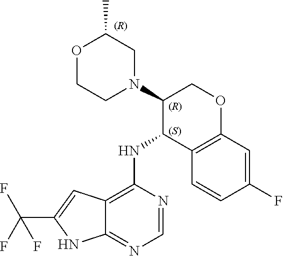 |
| EP-0041088 | 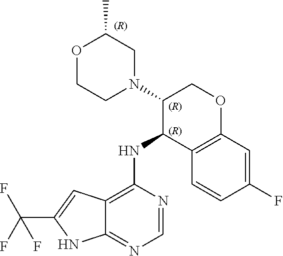 |
| EP-0041089 | 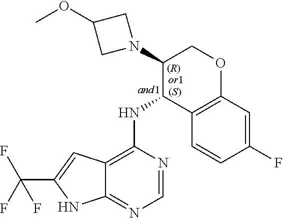 |
| EP-0041122 | 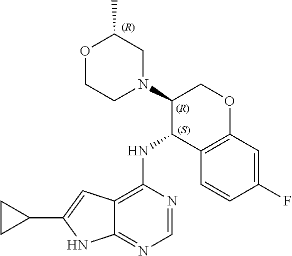 |
| EP-0041123 | 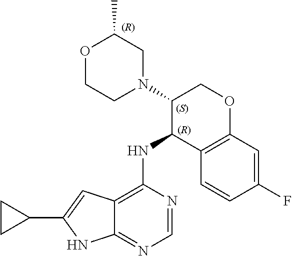 |
| EP-0041124 | 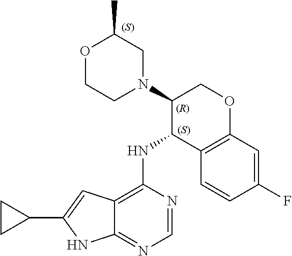 |
| EP-0041125 | 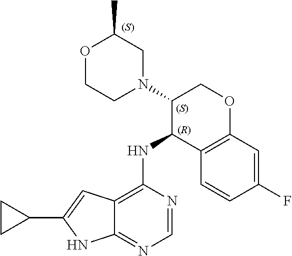 |
| EP-0041161 | 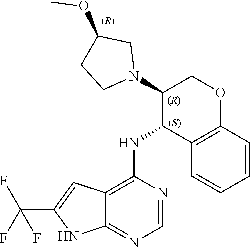 |
| EP-0041162 | 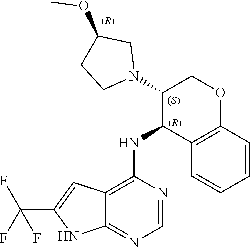 |
| EP-0041172 | 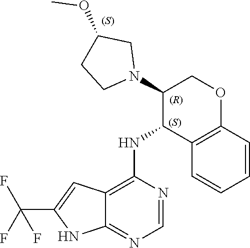 |
| EP-0041173 | 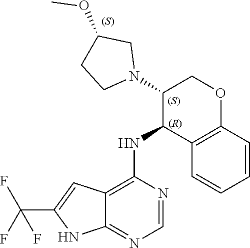 |
| EP-0041264 | 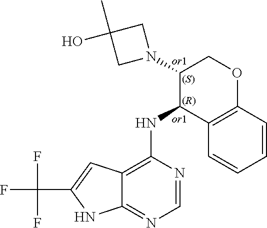 |
| EP-0041300 | 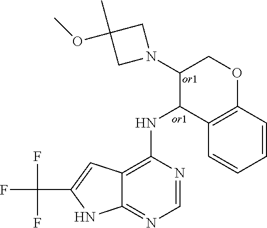 [SMILES: COC1(C)CN(C2COc3ccccc3C2Nc2ncnc3[nH]c(C(F)(F)F)cc23)C1] |
| EP-0041322 | 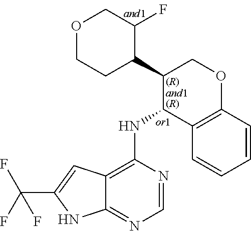 |
| EP-0041323 | 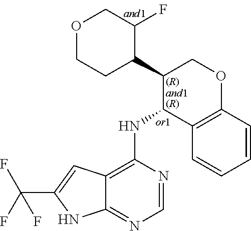 |
| EP-0041324 | 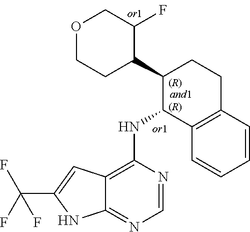 |
| EP-0041325 | 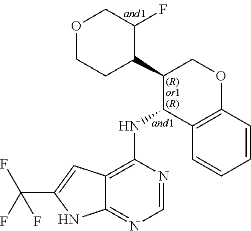 |
| EP-0041332 | 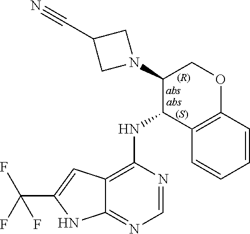 |
| EP-0041354 | 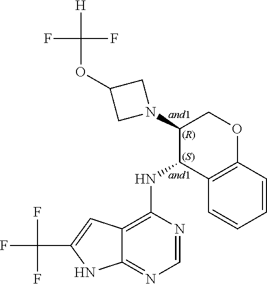 |
| EP-0041451 | 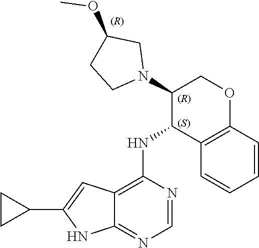 |
| EP-0041465 | 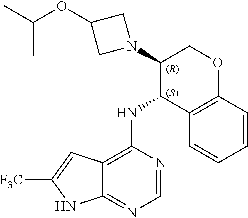 |
| EP-0041490 | 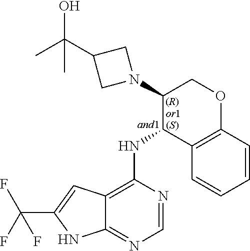 |
| EP-0041517 | 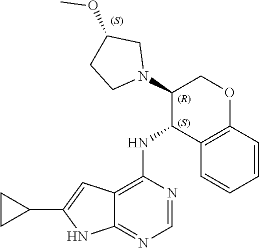 |
| EP-0041518 | 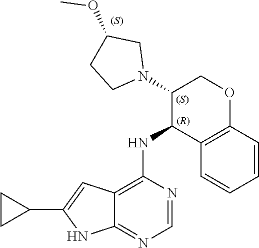 |
| EP-0041541 | 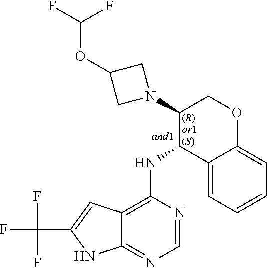 |
| EP-0041542 | 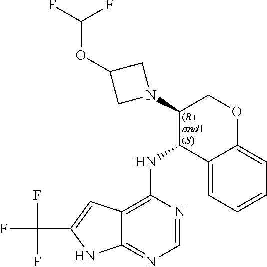 |
| EP-0041558 | 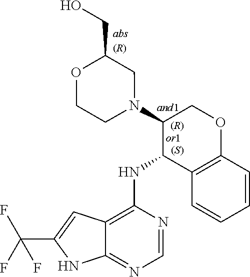 |
| EP-0041559 | 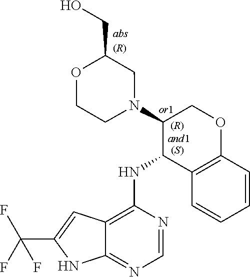 |
| EP-0041566 | 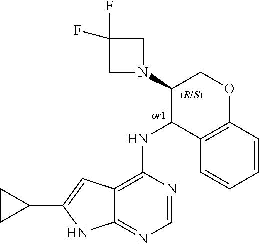 |
| EP-0041585 | 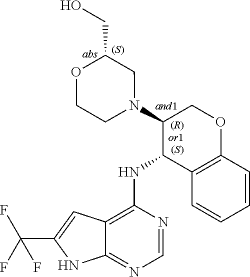 |
| EP-0041586 | 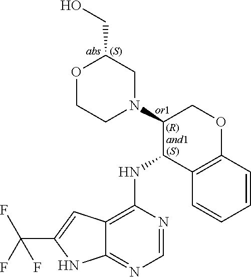 |
| EP-0041600 | 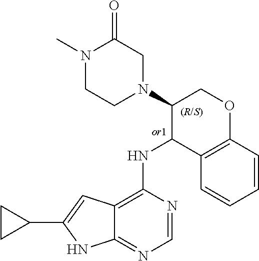 |
| EP-0041667 | 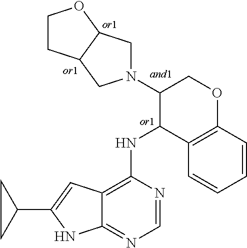 [SMILES: c1ccc2c(c1)OCC(N1CC3CCOC3C1)C2Nc1ncnc2[nH]c(C3CC3)cc12] |
| EP-0041668 | 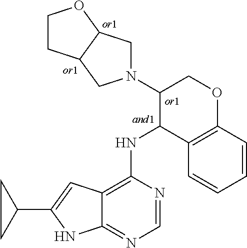 [SMILES: c1ccc2c(c1)OCC(N1CC3CCOC3C1)C2Nc1ncnc2[nH]c(C3CC3)cc12] |
| EP-0041669 | 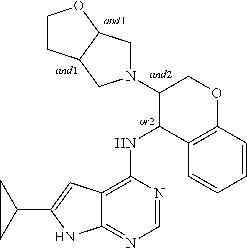 [SMILES: c1ccc2c(c1)OCC(N1CC3CCOC3C1)C2Nc1ncnc2[nH]c(C3CC3)cc12] |
| EP-0041670 | 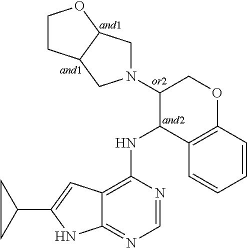 [SMILES: c1ccc2c(c1)OCC(N1CC3CCOC3C1)C2Nc1ncnc2[nH]c(C3CC3)cc12] |
| EP-0041071 | 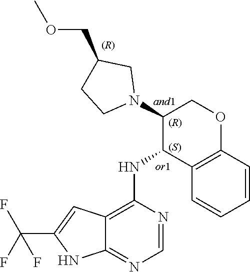 |
| EP-0041072 | 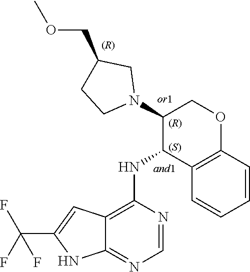 |
| EP-0041135 | 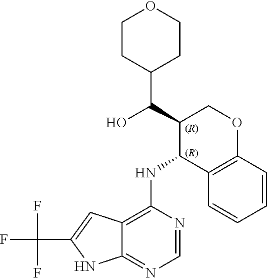 |
| EP-0041174 | 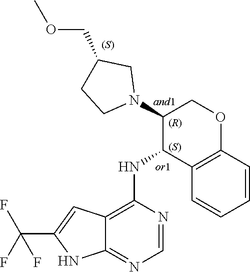 |
| EP-0041175 | 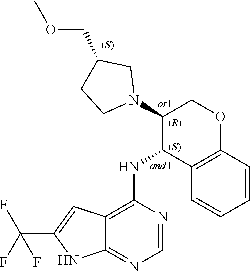 |
| EP-0041248 | 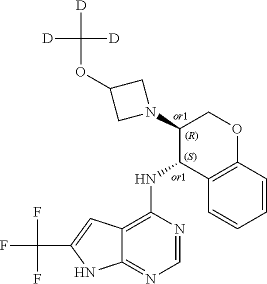 |
| EP-0041260 | 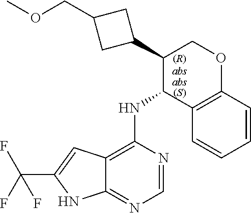 |
| EP-0041269 | 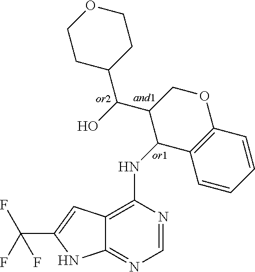 [SMILES: OC(C1CCOCC1)C1COc2ccccc2C1Nc1ncnc2[nH]c(C(F)(F)F)cc12] |
| EP-0041270 | 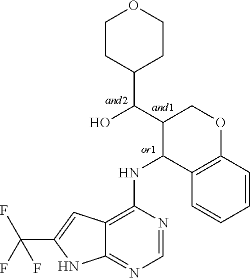 [SMILES: OC(C1CCOCC1)C1COc2ccccc2C1Nc1ncnc2[nH]c(C(F)(F)F)cc12] |
| EP-0041326 | 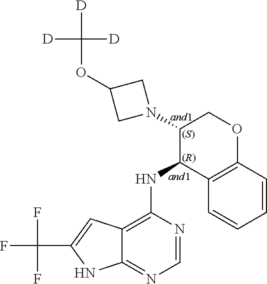 |
| EP-0041327 | 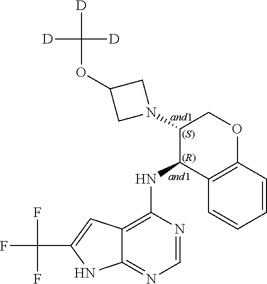 |
| EP-0041480 | 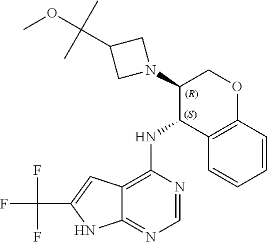 |
| EP-0041491 | 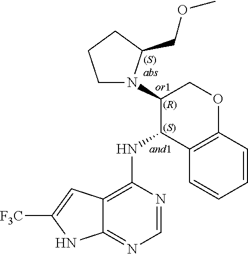 |
| EP-0041492 | 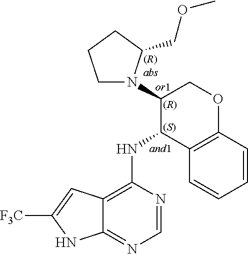 |
| EP-0041495 | 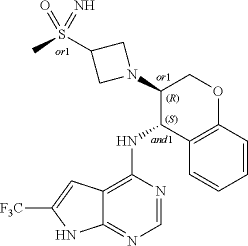 |
| EP-0041496 | 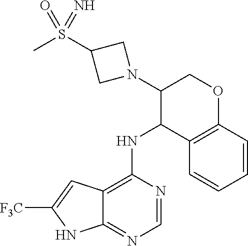 [SMILES: CS(=N)(=O)C1CN(C2COc3ccccc3C2Nc2ncnc3[nH]c(C(F)(F)F)cc23)C1] |
| EP-0041497 | 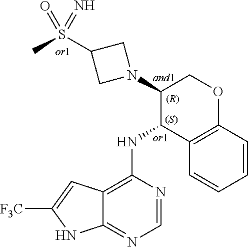 |
| EP-0041498 | 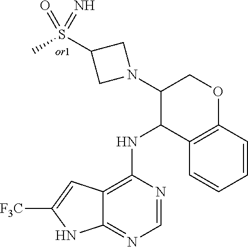 [SMILES: C[S@@](=N)(=O)C1CN(C2COc3ccccc3C2Nc2ncnc3[nH]c(C(F)(F)F)cc23)C1] |
| EP-0041514 | 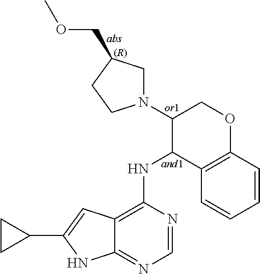 |
| EP-0041515 | 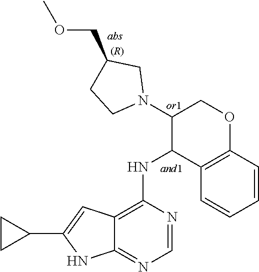 |
| EP-0041516 | 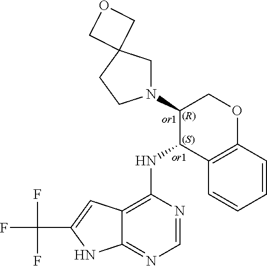 |
| EP-0041546 | 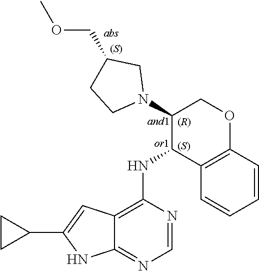 |
| EP-0041547 | 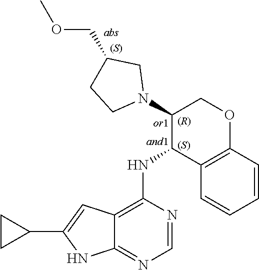 |
| EP-0041553 | 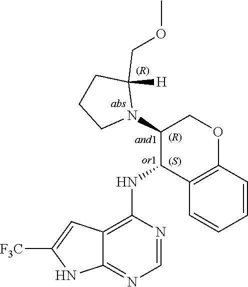 |
| EP-0041596 | 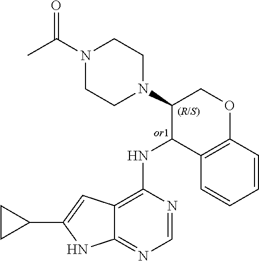 |

*The abbreviations “or1” and “and1” represent a trans relationship, wherein the stereochemistry is relative;
“abs” represents absolute stereochemistry.

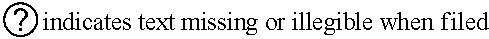

TABLE 2A

| | | E101 | E102 max | E103 max | E104 | E105 |
|---|---|---|---|---|---|---|
| | E100 | lowest | mitophagy, | death | death at | death at |
| | EC₅₀ | effective | fold over | with 1 μM | 25 μM | 8.3 μM |
| No. | (μM) | dose (μM) | DMSO | FO | (no FO) | (no FO) |
| EP-0035985 | 0.33 | 0.31 | 4.43 | 8.14 | 3.01 | 2.54 |
| EP-0038463 | 2.59 | 2.78 | 3.95 | 87.81 | 59.77 | 4.21 |
| EP-0038504 | 14.22 | 8.33 | 4.67 | 4.59 | 3.10 | 2.93 |
| EP-0038461 | 0.11 | 0.93 | 5.44 | 7.97 | | |
| EP-0038503 | 0.58 | 8.33 | 5.11 | 4.37 | | |
| EP-0038521 | 0.37 | 2.78 | 5.24 | 6.77 | | |
| EP-0038098 | 0.22 | 0.10 | 9.78 | 24.02 | 5.29 | 2.59 |
| EP-0038099 | N/A | 25.00 | 7.78 | 13.71 | 4.11 | 2.41 |
| EP-0037821 | 0.45 | 0.30 | 9.53 | 62.82 | 25.17 | 2.87 |
| EP-0038461 | 0.1 ± 0.01 | | | | | |
| (average) | | | | | | |
| EP-0038521 | 0.30 ± 0.07 | | | | | |
| (average) | | | | | | |
| EP-0038503 | 0.48 ± 0.1 | | | | | |

TABLE 2B

| | Mitophagy | Max cell | Cell | | |
|---|---|---|---|---|---|
| | EC₅₀ | death at | death at | Mitotox | |
| | (HeLa | 25 μM | 25 μM | safety margin | Kinetic |
| | mKeima | compound, | (no FO) | (Therapeutic | Solubility |
| No. | assay) (μM) | 1 uM FO (%) | (%) | window)* | (μM) |
| EP-0037820 | 17.4 | 58 | 41.4 | + | 14.5 |
| EP-0037821 | 0.40 | 71.62 | 41.03 | ++++ | 14.1 |
| (Racemic mixture) | | | | | |
| EP-0038098 | 0.22 | 24 | 5.29 | ++++ | 14.1 |
| EP-0038099 | Minimal activity | 13.7 | 4.11 | | 14.1 |
| EP-0038249 | 2.73 | 37.3 | 3.69 | | 157 |
| EP-0038282 | Minimal activity | 97.5 | 66.6 | | 134 |
| EP-0038283 | Minimal activity | 98 | 9.6 | | 1 |
| EP-0038378 | 2.77 | 15.4 | 4.55 | | 3.02 |
| EP-0038392 | 2.77 | 23.2 | 3.1 | | 187 |
| EP-0038393 | 2.43 | 17.6 | 1.98 | | 1 |
| EP-0038394 | 8.26 | 61 | 11.4 | | 7.05 |
| EP-0038461 | 0.11 | 7.97 | 4.31 | +++ | 1.56 |
| EP-0038463 | 2.59 | 87.805 | 59.8 | + | 153 |
| EP-0038503 | 0.38 | 8.15 | 2.53 | ++ | 1.56 |
| EP-0038504 | 14.20 | 4.59 | 3.1 | + | 1.56 |
| EP-0038521 | 0.37 | 6.77 | 3.42 | +++ | 1.56 |
| EP-0038582 | Minimal activity | 5.87 | 3.77 | | 1.56 |
| EP-0038583 | 0.07 | 39.5 | 6.03 | + | 1.56 |
| EP-0039677 | 0.21 | 10.2 | 3.02 | ++++ | 1.56 |
| EP-0039678 | Minimal activity | 2.27 | 1.77 | | 164 |
| EP-0039713 | 0.10 | 19.2 | 12.9 | + | |
| EP-0039742 | 3.33 | 13.8 | 2.45 | | |
| EP-0039743 | 3.88 | 33.7 | 11.6 | | |
| EP-0039746 | 0.25 | 15.3 | 3.3 | +++ | |
| EP-0039747 | 9.31 | 24.4 | 12.9 | | |
| EP-0040078 | 1.77 | 18 | 5.92 | | |
| EP-0040080 | 0.59 | 16 | 4.73 | ++ | |

*+ = <10;
++ = 10 < x < 20;
+++ = 20 < x < 30;
++++ = 30<

TABLE 2C

| | | | Highest safe | | |
|---|---|---|---|---|---|
| | | | dose | | |
| | | | (cytotoxicity; | Highest | |
| | | Max % | no FO; safe | safe | Mito-Safety |
| | Mitophagy | death | means | dose (μM) | Index |
| | induction | with 1 | DAPI+ | (mitotox or | (highest safe |
| No. | EC₅₀ (μM) | μM FO | <20%) (μM) | cytotox) | dose/EC₅₀) |
| EP- | 20.30 | 60.7 | 16.7 | n/a | n/a |
| 0037820 | | | | | |
| EP- | 0.38 | 68.5 | 13.9 | 8.3 | 22.05 |
| 0037821 | | | | | |
| EP- | >25 | 37.3 | 25.0 | n/a | n/a |
| 0038249 | | | | | |
| EP- | >25 | 97.5 | 2.8 | n/a | n/a |
| 0038282 | | | | | |
| EP- | >25 | 98.0 | 8.3 | n/a | n/a |
| 0038283 | | | | | |
| EP- | >25 | 15.4 | 25.0 | n/a | n/a |
| 0038378 | | | | | |
| EP- | >25 | 23.2 | 25.0 | n/a | n/a |
| 0038392 | | | | | |
| EP- | >25 | 17.6 | 25.0 | n/a | n/a |
| 0038393 | | | | | |
| EP- | >25 | 61.0 | 8.3 | n/a | n/a |
| 0038394 | | | | | |
| EP- | 0.10 | 9.7 | 25.0 | 2.8 | 27.53 |
| 0038461 | | | | | |
| EP- | 2.59 | 87.8 | 8.3 | 8.3 | 3.20 |
| 0038463 | | | | | |
| EP- | 0.48 | 6.3 | 25.0 | 8.3 | 17.40 |
| 0038503 | | | | | |
| EP- | 14.22 | 4.6 | 25.0 | 25.0 | 1.76 |
| 0038504 | | | | | |
| EP- | 0.34 | 7.6 | 25.0 | 8.3 | 24.49 |
| 0038521 | | | | | |
| EP- | 4.45 | 3.3 | 25.0 | n/a | n/a |
| 0038523 | | | | | |
| EP- | >25 | 4.3 | 25.0 | 25.0 | n/a |
| 0038582 | | | | | |
| EP- | 0.06 | 18.1 | 25.0 | 9.1 | 158.93 |
| 0038583 | | | | | |
| EP- | 0.21 | 10.2 | 25.0 | 2.8 | 13.51 |
| 0039677 | | | | | |
| EP- | 0.10 | 26.3 | 25.0 | 0.9 | 9.46 |
| 0039713 | | | | | |
| EP- | >25 | 6.2 | 25.0 | n/a | n/a |
| 0039732 | | | | | |
| EP- | 9.33 | 3.1 | 25.0 | n/a | n/a |
| 0039733 | | | | | |
| EP- | 3.36 | 20.4 | 25.0 | 25.0 | 7.45 |
| 0039742 | | | | | |
| EP- | 4.17 | 28.2 | 25.0 | 25.0 | 5.99 |
| 0039743 | | | | | |
| EP- | 0.29 | 13.4 | 25.0 | 2.8 | 9.48 |
| 0039746 | | | | | |
| EP- | 9.31 | 24.4 | 25.0 | n/a | n/a |
| 0039747 | | | | | |
| EP- | 8.07 | 6.5 | 25.0 | n/a | n/a |
| 0039748 | | | | | |
| EP- | >25 | 17.7 | 25.0 | n/a | n/a |
| 0039754 | | | | | |
| EP- | >25 | 6.5 | 25.0 | n/a | n/a |
| 0040075 | | | | | |
| EP- | 1.90 | 18.5 | 25.0 | n/a | n/a |
| 0040078 | | | | | |
| EP- | 0.48 | 13.9 | 25.0 | 25.0 | 51.66 |
| 0040080 | | | | | |
| EP- | 15.60 | 3.7 | 25.0 | n/a | n/a |
| 0040084 | | | | | |
| EP- | >25 | 38.1 | 8.3 | n/a | n/a |
| 0040085 | | | | | |
| EP- | 0.13 | 19.1 | 25.0 | 18.3 | 138.83 |
| 0040108 | | | | | |
| EP- | 24.83 | 7.9 | 25.0 | 25.0 | 1.01 |
| 0040109 | | | | | |
| EP- | 2.30 | 93.5 | 8.3 | 8.3 | n/a |
| 0040120 | | | | | |
| EP- | 1.29 | 33.6 | 25.0 | n/a | n/a |
| 0040121 | | | | | |
| EP- | 0.77 | 9.4 | 25.0 | 25.0 | 32.50 |
| 0040138 | | | | | |
| EP- | 3.66 | 8.5 | 25.0 | n/a | n/a |
| 0040139 | | | | | |
| EP- | 8.71 | 11.1 | 25.0 | n/a | n/a |
| 0040140 | | | | | |
| EP- | 0.26 | 10.4 | 25.0 | 25.0 | 97.54 |
| 0040180 | | | | | |
| EP | >25 | 5.9 | 25.0 | n/a | n/a |
| 0040181 | | | | | |
| EP- | 21.23 | 26.8 | 25.0 | n/a | n/a |
| 0040182 | | | | | |
| EP- | 9.46 | 29.7 | 25.0 | n/a | n/a |
| 0040193 | | | | | |
| EP- | 18.54 | 12.0 | 25.0 | n/a | n/a |
| 0040195 | | | | | |
| EP- | 22.65 | 8.4 | 25.0 | n/a | n/a |
| 0040197 | | | | | |
| EP- | >25 | 3.7 | 25.0 | n/a | n/a |
| 0040248 | | | | | |
| EP- | 0.09 | 12.3 | 25.0 | n/a | n/a |
| 0040249 | | | | | |
| EP- | >25 | 3.1 | 25.0 | n/a | n/a |
| 0040251 | | | | | |
| EP- | 1.11 | 9.1 | 25.0 | n/a | n/a |
| 0040252 | | | | | |
| EP- | >25 | 3.2 | 25.0 | n/a | n/a |
| 0040268 | | | | | |
| EP- | > 25 | 3.6 | 25.0 | n/a | n/a |
| 0040326 | | | | | |
| EP- | 0.19 | 16.0 | 25.0 | 16.0 | 84.78 |
| 0040328 | | | | | |
| EP- | >25 | 16.5 | 25.0 | n/a | n/a |
| 0040339 | | | | | |
| EP- | >25 | 97.8 | 2.8 | n/a | n/a |
| 0040358 | | | | | |
| EP- | >25 | 3.9 | 25.0 | n/a | n/a |
| 0040391 | | | | | |
| EP- | 0.11 | 11.8 | 25.0 | 16.6 | 153.83 |
| 0040392 | | | | | |
| EP- | 0.21 | 8.1 | 25.0 | 11.7 | 56.09 |
| 0040395 | | | | | |
| EP- | 0.07 | 19.4 | 25.0 | 3.7 | 49.72 |
| 0040491 | | | | | |
| EP- | >25 | 1.8 | 25.0 | n/a | n/a |
| 0040492 | | | | | |
| EP- | 0.02 | 14.8 | 25.0 | 1.7 | 103.09 |
| 0040493 | | | | | |
| EP- | >25 | 4.1 | 25.0 | n/a | n/a |
| 0040494 | | | | | |
| EP- | 0.02 | 15.6 | 25.0 | 1.3 | 82.87 |
| 0040496 | | | | | |
| EP- | 6.97 | 4.3 | 25.0 | n/a | n/a |
| 0040497 | | | | | |
| EP- | 0.02 | 16.5 | 25.0 | 0.7 | 30.45 |
| 0040498 | | | | | |
| EP- | 5.20 | 4.3 | 25.0 | n/a | n/a |
| 0040499 | | | | | |
| EP- | 0.99 | 8.6 | 25.0 | n/a | n/a |
| 0040500 | | | | | |
| EP- | 14.26 | 5.0 | 25.0 | n/a | n/a |
| 0040501 | | | | | |
| EP- | 0.13 | 7.4 | 25.0 | 9.1 | 69.05 |
| 0040503 | | | | | |
| EP- | 9.33 | 2.0 | 25.0 | n/a | n/a |
| 0040504 | | | | | |
| EP- | 13.49 | 14.3 | 25.0 | n/a | n/a |
| 0040505 | | | | | |
| EP- | 0.05 | 41.0 | 25.0 | 2.2 | 44.10 |
| 0040506 | | | | | |
| EP- | 0.07 | 13.1 | 25.0 | 3.5 | 53.66 |
| 0040511 | | | | | |
| EP- | 12.11 | 26.7 | 25.0 | n/a | n/a |
| 0040512 | | | | | |
| EP- | 2.73 | 39.0 | 25.0 | n/a | n/a |
| 0040523 | | | | | |
| EP- | 2.68 | 27.7 | 25.0 | n/a | n/a |
| 0040524 | | | | | |
| EP- | 1.45 | 15.3 | 25.0 | n/a | n/a |
| 0040542 | | | | | |
| EP- | >25 | 3.7 | 25.0 | n/a | n/a |
| 0040545 | | | | | |
| EP- | >25 | 2.7 | 25.0 | n/a | n/a |
| 0040557 | | | | | |
| EP- | >25 | 3.0 | 25.0 | n/a | n/a |
| 0040558 | | | | | |
| EP- | 1.50 | 7.9 | 25.0 | n/a | n/a |
| 0040562 | | | | | |
| EP- | 1.05 | 9.7 | 25.0 | n/a | n/a |
| 0040563 | | | | | |
| EP- | 2.79 | 21.2 | 25.0 | n/a | n/a |
| 0040576 | | | | | |
| EP- | 0.02 | 28.6 | 25.0 | 1.3 | 86.01 |
| 0040577 | | | | | |
| EP- | 3.54 | 14.4 | 25.0 | n/a | n/a |
| 0040578 | | | | | |
| EP- | 9.02 | 20.3 | 25.0 | n/a | n/a |
| 0040579 | | | | | |
| EP- | 0.08 | 17.1 | 25.0 | 5.5 | 68.72 |
| 0040580 | | | | | |
| EP- | 2.69 | 8.1 | 25.0 | n/a | n/a |
| 0040581 | | | | | |
| EP- | 0.08 | 17.7 | 25.0 | 5.1 | 68.01 |
| 0040582 | | | | | |
| EP- | 0.24 | 17.8 | 25.0 | 25.0 | 104.01 |
| 0040583 | | | | | |
| EP- | >25 | 16.9 | 8.3 | n/a | n/a |
| 0040586 | | | | | |
| EP- | 0.09 | 21.5 | 25.0 | 21.5 | 230.81 |
| 0040587 | | | | | |
| EP- | >25 | 19.4 | 25.0 | n/a | n/a |
| 0040589 | | | | | |
| EP- | 0.19 | 45.3 | 25.0 | 10.3 | 55.12 |
| 0040601 | | | | | |
| EP- | 7.11 | 29.3 | 25.0 | n/a | n/a |
| 0040626 | | | | | |
| EP- | 24.43 | 4.8 | 25.0 | n/a | n/a |
| 0040627 | | | | | |
| EP- | 0.34 | 8.1 | 25.0 | 25.0 | 72.77 |
| 0040644 | | | | | |
| EP- | 9.86 | 3.7 | 25.0 | n/a | n/a |
| 0040665 | | | | | |
| EP- | 0.35 | 29.9 | 25.0 | 25.0 | 71.36 |
| 0040669 | | | | | |
| EP- | 0.32 | 11.1 | 25.0 | 25.0 | 77.08 |
| 0040677 | | | | | |
| EP- | 0.86 | 9.2 | 25.0 | 25.0 | 28.90 |
| 0040678 | | | | | |
| EP- | 0.31 | 82.1 | 8.3 | 8.3 | 26.96 |
| 0040688 | | | | | |
| EP- | 0.10 | 20.7 | 25.0 | 9.1 | 89.34 |
| 0040695 | | | | | |
| EP- | 23.44 | 14.9 | 25.0 | n/a | n/a |
| 0040696 | | | | | |
| EP- | 0.54 | 57.6 | 8.3 | 8.3 | 15.51 |
| 0040697 | | | | | |
| EP- | >25 | 3.7 | 25.0 | n/a | n/a |
| 0040699 | | | | | |
| EP- | 0.11 | 14.1 | 25.0 | 4.7 | 41.37 |
| 0040700 | | | | | |
| EP- | 0.32 | 7.7 | 25.0 | 25.0 | 77.26 |
| 0040702 | | | | | |
| EP- | 6.25 | 12.1 | 25.0 | n/a | n/a |
| 0040720 | | | | | |
| EP- | 0.18 | 36.3 | 8.3 | 8.3 | 46.94 |
| 0040721 | | | | | |
| EP- | >25 | 3.4 | 25.0 | n/a | n/a |
| 0040725 | | | | | |
| EP- | 0.02 | 20.5 | 25.0 | 2.1 | 100.89 |
| 0040726 | | | | | |
| EP- | 2.85 | 7.7 | 25.0 | n/a | n/a |
| 0040727 | | | | | |
| EP- | 9.40 | 3.7 | 25.0 | n/a | n/a |
| 0040728 | | | | | |
| EP- | 0.06 | 17.1 | 25.0 | 3.2 | 52.06 |
| 0040729 | | | | | |
| EP- | >25 | 8.3 | 25.0 | n/a | n/a |
| 0040730 | | | | | |
| EP- | >25 | 2.7 | 25.0 | n/a | n/a |
| 0040731 | | | | | |
| EP- | >25 | 5.8 | 25.0 | n/a | n/a |
| 0040747 | | | | | |
| EP- | 0.21 | 25.3 | 25.0 | 9.9 | 46.81 |
| 0040748 | | | | | |
| EP- | 0.15 | 34.3 | 25.0 | 7.1 | 48.24 |
| 0040749 | | | | | |
| EP- | 0.05 | 27.7 | 25.0 | 2.3 | 43.07 |
| 0040750 | | | | | |
| EP- | >25 | 7.9 | 25.0 | n/a | n/a |
| 0040767 | | | | | |
| EP- | 0.05 | 33.1 | 25.0 | 3.0 | 57.75 |
| 0040768 | | | | | |
| EP- | 0.13 | 27.3 | 25.0 | 9.9 | 77.04 |
| 0040773 | | | | | |
| EP | 0.02 | 22.5 | 25.0 | 2.0 | 82.00 |
| 0040842 | | | | | |
| EP- | >25 | 5.2 | 25.0 | 25.0 | n/a |
| 0040847 | | | | | |
| EP- | 0.16 | 10.6 | 25.0 | 25.0 | 152.94 |
| 0040848 | | | | | |
| EP- | 9.59 | 5.2 | 25.0 | 25.0 | 2.61 |
| 0040849 | | | | | |
| EP- | 0.24 | 10.3 | 25.0 | 21.4 | 88.53 |
| 0040850 | | | | | |
| EP- | 0.16 | 17.0 | 25.0 | 18.7 | 116.38 |
| 0040857 | | | | | |
| EP- | 28.84 | 6.6 | 25.0 | n/a | n/a |
| 0040858 | | | | | |
| EP- | >25 | 4.3 | 25.0 | n/a | n/a |
| 0040873 | | | | | |
| EP- | 0.15 | 14.4 | 25.0 | 13.9 | 94.72 |
| 0040874 | | | | | |
| EP- | >25 | 4.7 | 25.0 | n/a | n/a |
| 0040942 | | | | | |
| EP- | 0.05 | 20.9 | 25.0 | 2.3 | 47.50 |
| 0040943 | | | | | |
| EP- | 1.22 | 6.3 | 25.0 | 25.0 | 20.51 |
| 0040944 | | | | | |
| EP- | 0.04 | 18.4 | 25.0 | 1.2 | 27.95 |
| 0040945 | | | | | |
| EP- | >25 | 4.8 | error | 25.0 | n/a |
| 0040946 | | | | | |
| EP- | 0.02 | 16.6 | 25.0 | 2.2 | 98.61 |
| 0040947 | | | | | |
| EP- | >25 | 5.6 | 25.0 | 25.0 | n/a |
| 0040948 | | | | | |
| EP- | 0.01 | 21.1 | 25.0 | 1.2 | 114.83 |
| 0040949 | | | | | |
| EP- | >25 | 3.4 | 25.0 | n/a | n/a |
| 0040955 | | | | | |
| EP- | 0.19 | 24.7 | 25.0 | 9.7 | 52.10 |
| 0040956 | | | | | |
| EP- | 22.75 | 3.8 | 25.0 | n/a | n/a |
| 0040957 | | | | | |
| EP- | 0.06 | 14.1 | 25.0 | 1.7 | 29.33 |
| 0040958 | | | | | |
| EP- | >25 | 4.0 | 25.0 | n/a | n/a |
| 0040979 | | | | | |
| EP- | 0.38 | 14.7 | 19.4 | 19.4 | 50.83 |
| 0040980 | | | | | |
| EP- | 2.44 | 7.0 | 25.0 | n/a | n/a |
| 0040981 | | | | | |
| EP- | 7.62 | 6.1 | 25.0 | n/a | n/a |
| 0040982 | | | | | |
| EP- | 0.14 | 12.1 | 25.0 | 15.1 | 105.78 |
| 0040999 | | | | | |
| EP- | 1.32 | 6.5 | 25.0 | n/a | n/a |
| 0041000 | | | | | |
| EP- | 6.18 | 4.5 | 25.0 | n/a | n/a |
| 0041002 | | | | | |
| EP- | >25 | 5.3 | 25.0 | n/a | n/a |
| 0041003 | | | | | |
| EP- | 0.98 | 71.7 | 25.0 | 25.0 | 25.46 |
| 0041004 | | | | | |
| EP- | 1.45 | 24.8 | 25.0 | 25.0 | 17.30 |
| 0041005 | | | | | |
| EP- | 3.38 | 13.2 | 25.0 | n/a | n/a |
| 0041021 | | | | | |
| EP- | 1.56 | 7.7 | 25.0 | n/a | n/a |
| 0041045 | | | | | |
| EP- | 0.07 | 12.6 | 25.0 | 3.3 | 47.29 |
| 0041071 | | | | | |
| EP- | >25 | 8.0 | 25.0 | n/a | n/a |
| 0041072 | | | | | |
| EP- | 9.38 | 5.1 | 25.0 | n/a | n/a |
| 0041087 | | | | | |
| EP- | 0.12 | 16.2 | 25.0 | 15.7 | 128.93 |
| 0041088 | | | | | |
| EP- | 1.97 | 10.2 | 25.0 | n/a | n/a |
| 0041089 | | | | | |
| EP- | 0.05 | 16.9 | 16.7 | 2.0 | 37.43 |
| 0041122 | | | | | |
| EP- | 10.96 | 7.3 | 25.0 | n/a | n/a |
| 0041123 | | | | | |
| EP- | 5.32 | 12.7 | 25.0 | n/a | n/a |
| 0041124 | | | | | |
| EP- | 0.16 | 9.6 | 25.0 | 7.4 | 45.69 |
| 0041125 | | | | | |
| EP- | 1.53 | 15.7 | 25.0 | n/a | n/a |
| 0041135 | | | | | |
| EP- | 0.05 | 8.5 | 25.0 | 3.4 | 75.49 |
| 0041161 | | | | | |
| EP- | >25 | 4.2 | 25.0 | n/a | n/a |
| 0041162 | | | | | |
| EP- | 0.01 | 8.4 | 25.0 | 1.1 | 121.36 |
| 0041172 | | | | | |
| EP- | 4.09 | 6.1 | 25.0 | n/a | n/a |
| 0041173 | | | | | |
| EP- | 15.35 | 4.6 | 25.0 | n/a | n/a |
| 0041174 | | | | | |
| EP- | 0.15 | 8.8 | 25.0 | 4.5 | 30.68 |
| 0041175 | | | | | |
| EP- | 0.49 | 10.5 | 25.0 | n/a | n/a |
| 0041248 | | | | | |
| EP- | 0.85 | 10.2 | 25.0 | n/a | n/a |
| 0041260 | | | | | |
| EP- | >25 | 2.5 | 25.0 | n/a | n/a |
| 0041264 | | | | | |
| EP- | >25 | 2.9 | 25.0 | n/a | n/a |
| 0041269 | | | | | |
| EP- | 0.86 | 11.6 | 25.0 | n/a | n/a |
| 0041270 | | | | | |
| EP- | 4.35 | 11.1 | 25.0 | n/a | n/a |
| 0041300 | | | | | |
| EP- | >25 | 11.0 | 25.0 | n/a | n/a |
| 0041322 | | | | | |
| EP- | 11.02 | 9.2 | 25.0 | n/a | n/a |
| 0041323 | | | | | |
| EP- | 12.30 | 10.4 | 25.0 | n/a | n/a |
| 0041324 | | | | | |
| EP- | 18.75 | 5.5 | 25.0 | n/a | n/a |
| 0041325 | | | | | |
| EP- | 0.35 | 7.5 | 25.0 | 19.8 | 56.49 |
| 0041326 | | | | | |
| EP- | >25 | 4.1 | 25.0 | n/a | n/a |
| 0041327 | | | | | |
| EP- | 1.54 | 11.7 | 25.0 | n/a | n/a |
| 0041332 | | | | | |
| EP- | 1.08 | 12.4 | 25.0 | n/a | n/a |
| 0041354 | | | | | |
| EP- | 0.01 | 6.5 | 25.0 | 0.7 | 87.93 |
| 0041451 | | | | | |
| EP- | 6.85 | 4.6 | 25.0 | n/a | n/a |
| 0041465 | | | | | |
| EP- | 2.69 | 4.6 | 25.0 | n/a | n/a |
| 0041480 | | | | | |
| EP- | >25 | 3.4 | 25.0 | n/a | n/a |
| 0041490 | | | | | |
| EP- | 10.16 | 3.7 | 25.0 | n/a | n/a |
| 0041491 | | | | | |
| EP- | 1.78 | 8.3 | 25.0 | n/a | n/a |
| 0041492 | | | | | |
| EP- | 2.52 | 2.9 | 25.0 | n/a | n/a |
| 0041495 | | | | | |
| EP- | >25 | 3.4 | 25.0 | n/a | n/a |
| 0041496 | | | | | |
| EP- | 21.98 | 2.6 | 25.0 | n/a | n/a |
| 0041497 | | | | | |
| EP- | >25 | 3.7 | 25.0 | n/a | n/a |
| 0041498 | | | | | |
| EP- | 0.03 | 8.1 | 25.0 | 1.4 | 38.63 |
| 0041514 | | | | | |
| EP- | 5.06 | 5.6 | 25.0 | n/a | n/a |
| 0041515 | | | | | |
| EP- | 3.24 | 11.8 | 25.0 | n/a | n/a |
| 0041516 | | | | | |
| EP- | 0.03 | 4.8 | 25.0 | 1.1 | 34.73 |
| 0041517 | | | | | |
| EP- | >25 | 7.4 | 25.0 | n/a | n/a |
| 0041518 | | | | | |
| EP- | 0.29 | 19.1 | 25.0 | 8.3 | 28.61 |
| 0041541 | | | | | |
| EP- | >25 | 3.8 | 25.0 | n/a | n/a |
| 0041542 | | | | | |
| EP- | 0.17 | 6.6 | 25.0 | 3.6 | 21.52 |
| 0041546 | | | | | |
| EP- | 7.29 | 8.2 | 25.0 | n/a | n/a |
| 0041547 | | | | | |
| EP- | 15.24 | 5.7 | 25.0 | n/a | n/a |
| 0041553 | | | | | |
| EP- | 1.42 | 10.6 | 25.0 | n/a | n/a |
| 0041558 | | | | | |
| EP- | 10.28 | 3.7 | 25.0 | n/a | n/a |
| 0041559 | | | | | |
| EP- | 1.00 | 30.0 | 25.0 | n/a | n/a |
| 0041566 | | | | | |
| EP- | 16.14 | 6.6 | 25.0 | n/a | n/a |
| 0041585 | | | | | |
| EP- | 9.59 | 10.7 | 25.0 | n/a | n/a |
| 0041586 | | | | | |
| EP- | >25 | 6.2 | 25.0 | n/a | n/a |
| 0041595 | | | | | |
| EP- | 2.72 | 15.3 | 25.0 | n/a | n/a |
| 0041596 | | | | | |
| EP- | >25 | 4.9 | 25.0 | n/a | n/a |
| 0041600 | | | | | |
| EP- | >25 | 3.1 | 25.0 | n/a | n/a |
| 0041667 | | | | | |
| EP- | 0.14 | 8.0 | 25.0 | n/a | n/a |
| 0041668 | | | | | |
| EP- | >25 | 2.5 | 25.0 | n/a | n/a |
| 0041669 | | | | | |
| EP- | 0.46 | 5.4 | 25.0 | n/a | n/a |
| 0041670 | | | | | |

### 4. Performed Fibril MODEL

Mouse and human alpha-synuclein monomers have been sourced commercially, then pre-formed fibrils have been generated as per the detailed protocol provided by the MJFF. For in vitro experiments primary hippocampal neurons isolated from P0 pups were grown for 7 days after which PFFs at 5 μg/ml were introduced Pre-formed fibrils will be introduced via stereotactic injection into the striatum of wt (C57BL/6J, Jax #000664) and transgenic A53T mice (B6;C3-Tg(Pmp-SNCA*A53T)83Vle/J, Jax #004479). Cohorts of drug-treated (oral gavage) and untreated animals will then be allowed to age for up to 6 months, at which point they will be sacrificed and perfused. Brains will be removed and fixed, then sectioned for analysis. We anticipate that untreated A53T animals will show significant spreading of aggregated alpha-synuclein pathology and pS129 staining, as well as some possible neurodegeneration; wt animals are also expected to show pS129 synuclein staining and synuclein aggregation, albeit less than that seen in the A53T background. Drug treated animals are expected to show significantly less pS129 staining and reduced synuclein spreading.

### 5. Inspection of Crystallization and Round Cells

Briefly, the Hela MKYP (Mito-Keima/YFP-Parkin) cells were seeded at 10K cells/well. EP/MTK compounds were added at seeding (cells were still in suspension). The cells were incubated with the EP/MTK compounds for 16 hours, then 1 μM FCCP/oligomycin was added for 6 hours. Prior to harvesting, cells were scored by eye under 20× magnification for the presence or absence of crystalline or aggregated compound, or round cells.

TABLE 3

| μM | Log [M] |
|---|---|
| 25.000 | −4.6 |
| 8.333 | −5.1 |
| 2.778 | −5.6 |
| 0.926 | −6.0 |
| 0.309 | −6.5 |
| 0.103 | −7.0 |
| 0.01 | −8 |
| (used for 0) | |

Data corresponding to the visual inspection of crystallization (1=yes crystals; 0=no crystals) is shown in Table 4 below.

TABLE 4

| | Compound No. | | | | |
|---|---|---|---|---|---|
| μM | Kinetin | 35985 | 38461 | 38463 | 48503 |
| 25.0 | 0 | 0 | 1 | 0 | 0 |
| 8.3 | 0 | 0 | 0 | 0 | 0 |
| 2.8 | 0 | 0 | 0 | 0 | 0 |
| 0.9 | 0 | 0 | 0 | 0 | 0 |
| 0.3 | 0 | 0 | 0 | 0 | 0 |
| 0.1 | 0 | 0 | 0 | 0 | 0 |

### 6. Human Phospho-Ubiquitin (Ps65) Ub Assay

Briefly, HeLa MKYP cells were plated at 1,300,000 cells/plate in 10 cm plates in 10 mL of medium containing compound at various concentrations. Following 16 hrs of incubation, cells were treated with 0.5 uM FCCP/oligomycin for 2 hours, then harvested. Mitochondria were then isolated according to published protocols (Ordureau et al, 2014; https://doi.org/10.1016/j.molcel.2014.09.007). Equal amounts of samples were loaded on 26 well gradient gels, and a western blot analysis was performed using commercially available antibodies for various markers, including phospho serine 65 (pS65) ubiquitin, MFN2, PINK1, Parkin and actin.

### 7. Human Mitophagy Assay

Briefly, HeLa MKYP cells were plated at 10,000 cells/well in 96 well plates along with compounds at various concentrations. Following 16 hrs of incubation, cells were treated with 1 uM FCCP/oligomycin for 6 hours, then analyzed via FACS for the presence of mitochondria in lysosomes (as determined by an emissions spectrum shift from the pH-sensitive mtKeima tag).

### 8. Cisplatin-Related Protocols

a. In-Life Procedures Cisplatin Challenge and Dosing Regimen

Mice were provided at least one week of acclimation to the animal facility and group housed. Mice were injected intraperitoneally with 1 mg/ml cisplatin solution (BluePoint Labs) or 10 ml/kg sterile-filtered saline using 29G insulin syringes. Mice were weighed and administered vehicle, 35985 or 40180 by oral gavage per the dosing regimens noted in the figures. Mice were monitored for excessive weight loss and euthanized if moribund.

### b. 35985 and 40180 Formulation

35985 and 40180 are formulated at ten-fold the dosing concentration in NMP (N-methylpyrrolidone) followed by dilution with solutol-15 and water for a final vehicle concentration of 10% NVP/10% solutol-15/80% water.

c. Sacrifice and Tissue Collection and Storage

For tissue harvest, mice were anesthetized using isofluorane. Cardiac puncture was performed to withdraw blood for serum collection. Blood was deposited into serum separator tubes and left undisturbed for 30 min to 1 hr at room temperature to allow clotting prior to serum separation by centrifugation for 2 min (10,000g, room temperature). Collected serum was transferred to Eppendorf tubes and frozen on dry ice. After cervical dislocation, left and right kidneys were extracted and frozen until analysis.

d. Kidney Homogenate Preparation and Mitochondrial Isolation

Kidneys were removed from −80 °C and minced on an ice block. Minced tissues were transferred to a dounce homogenizer and homogenized with 20× strokes of the ‘loose’ pestle and 20× strokes of the ‘tight’ pestle using 1 ml ul of cold mitochondrial isolation buffer (MIB, 50 mM Tris-HCl (pH 7.5), 70 mM sucrose, 210 mM sorbitol, 1 mM EDTA, 1 mM EGTA, 100 mM chloroacetamide, Halt™ Protease and Phosphatase Inhibitor Cocktail, EDTA-free (100×) (PI), 10 μM PR619). Kidney homogenate was transferred to a 1.5 ml Eppendorf tube and were centrifuged at 300×g for 5 min at 4 °C. Approximately 800 ul of supernatant was transferred to a new 1.5 ml microcentrifuge tube. The supernatant (cytosol+mitochondria) was transferred to a new tube and centrifuged at 10,000 g for 20 min at 4 °C to pellet the mitochondrial fraction. After removing residual supernatant, mitochondria were resuspended in lysis buffer (100 mM Bicine pH 8.0, 0.27M Sucrose, 1 mM EDTA, 1 mM EGTA, 5 mM. Na4P2O7, 100 mM Tris pH 7.5, 1% Triton X-100), containing benzonase (1:1000), HALT protease/phosphatase inhibitors (1:100), and PR-619 de-ubiquitinase inhibitor (1:1000).

e. Blood Urea Nitrogen (Bun) Determination

Serum was thawed on ice and subsequently diluted 1:50 in MilliQ water. BUN levels in the serum sample were analyzed using ThermoFisher's Urea Nitrogen (BUN) Colorimetric Detection Kit. Assay was performed following manufacturer's published protocol.

f. Kidney Injury Marker (Im-1) Determination

Urine was collected from scruffed mice (serial collection) or directly from bladder using insulin syringe during harvest (terminal collection). KIM-1 was measured in mouse urine using R&D System's Mouse TIM-L/KIM-1/HAVCR DuoSet ELISA following manufacturer's published protocol.

g. Kidney RNA Extraction and Quantitative PCR

RNA was isolated from kidney samples using Rneasy Mini kit (Qiagen) according to its product manual. RNA concentration was measured using NanoDrop™ 2000/2000c Spectrophotometers (Thermo Scientific). 50 ng of RNA for each sample was used to generate cDNA. cDNA was synthesized using High-Capacity RNA-to-cDNA™ Kit (Thermo Scientific) according to its product manual. Quantitative PCR was performed using Power SYBR™ Green PCR Master Mix (Applied Biosystems) according to its product manual. The following primers were used to analyze gene expression levels in the kidney:

Tnfrsf12a,
(SEQ ID NO: 4)
5′-GTGTTGGGATTCGGCTTGGT-3′
and
(SEQ ID NO: 5)
5′-GTCCATGCACTTGTCGAGGTC-3′,
Atf3;
(SEQ ID NO: 6)
5′-GAGGATTTTGCTAACCTGACACC-3′
and
(SEQ ID NO: 7)
5′-TTGACGGTAACTGACTCCAGC-3′,
Plk3;
(SEQ ID NO: 8)
5′-GCACATCCATCGGTCATCCAG-3′
and
(SEQ ID NO: 9)
5′-GCCACAGTCAAACCTTCTTCAA-3′,
Gdf15;
(SEQ ID NO: 10)
5′-CTGGCAATGCCTGAACAACG-3′
and
(SEQ ID NO: 11)
5′-GGTCGGGACTTGGTTCTGAG-3′,
b-act;
(SEQ ID NO: 12)
5′-GGGCATCCTGACCCTC AAG-3′
and
(SEQ ID NO: 13)
5′-TCCATGTCGTCCCAGTTGGT-3′.

All gene expression levels were normalized to expression levels of beta-actin using ΔΔCt and expressed as fold change relative to cisplatin vehicle treated mice.

h. Mtdna/Nucdna Ratio

A small piece of frozen kidney tissue (~12 mg) was homogenized and DNA extracted using the Qiagen QIAamp DNA mini kit. mtDNA/nucDNA ratio was determined using a qPCR protocol from the Aurwex lab (Quiros et al, 2017), using the following primers:

16S rRNA
(SEQ ID NO: 14)
5′-CCGCAAGGGAAAGATGAAAGAC-3′
and
(SEQ ID NO: 15)
5′-TCGTTTGGTTTCGGGGTTTC-3′;
ND1
(SEQ ID NO: 16)
5′-CTAGCAGAAACAAACCGGGC-3′
and
(SEQ ID NO: 17)
5′-CCGGCTGCGTATTCTACGTT-3;
HK2
(SEQ ID NO: 18)
5′-GCCAGCCTCTCCTGATTTTAGTGT-3′
and
(SEQ ID NO: 19)
5′-GGGAACACAAAAGACCTCTTCTGG-3′.

i. Ps65-Ubelisa

For pS65-Ub ELISA, capture monoclonal rabbit antibody anti-pS65-Ub was diluted to 1 μg/ml in PBS and pipetted into 96 well half-area polystyrene plates (50 ul/well). Sealed plates were shaken at 800 rpm for 5 minutes and incubated overnight at 4 °C on an even surface. The next day, blocking solution (5% BSA in TBST, sterile filtered) was added to each well (100 ul/well) and shaken for 1 hr at 800 rpm at RT. Plates were either used immediately or stored sealed at 4 °C for maximum one week. Samples were diluted in lysis buffer to a concentration of 10 ug/ul and 50 ul were loaded onto plates in duplicate after washing 5× with TBST using an automated plate washer (used for all subsequent wash steps). Standard protein recombinant pS65-Ub was diluted in lysis buffer+0.1% BSA and serial dilutions (4000 ng/ml-0 ng/ml) were added in duplicate to the sample plate (50 ul/well). Plates were shaken at 800 rpm at RT for 2 hr. After washing 5× with TBST, 50 ul of mouse anti-Ub detection antibody (1 ug/ml in 5% BSA in TBST) was added to the wells. Plates were shaken at 800 rpm at RT for 1 hr, followed by washing 5× with TEST, and shaking at 800 rpm at RT for 45 minutes with goat anti-mouse peroxidase-conjugated IgG antibody (1:10,000 dilution in 5% BSA in TBST) (50 ul/well). For peroxidase reaction, 50 ul of TMB reagent (Pierce #34029) was added to the wells after washing and wells were monitored closely for reaction development. To stop the ELISA reaction, 50 ul 2N sulfuric acid was added. Absorbance was measured at 450 nm using LifeTechnologies SpectraMax).

j. Western Blotting

The total protein concentration of kidney mitopreps was measured with the Thermo Scientific Pierce BCA Protein Assay Kit (Thermo Scientific), according to its product manual. These samples were normalized with their respective lysis buffers. For SDS-PAGE, the samples were prepared with 4× Laemmli Sample Buffer with the reducing agent 2 mercaptoethanol. For each lane of a 26 well gel (4-20% Criterion™ Tris-HCl Protein Gel, Bio-Rad Laboratories), 10 μg per sample was loaded and analyzed by Western Blotting. Indicated bands were quantified using ImageStudio Lite and normalized to beta actin band intensity.

### 9. In Vitro Data

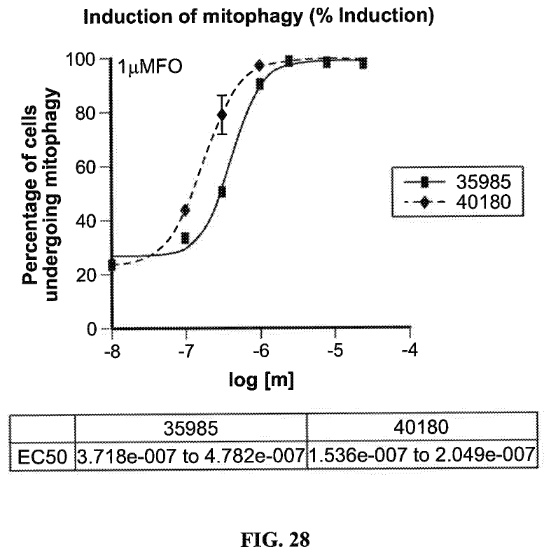

As shown in FIG. 28, the addition of 35985 or 40180 results in a dose responsive increase in the percentage of cells undergoing mitophagy. Briefly, HeLa cells expressing a mitophagy indicator (mtKeima) protein were treated with 1 mM of FCCP and Oligoimycin followed by the specified dose of compound, then analyzed by FACS to quantify the percentage of cells undergoing milophagy.

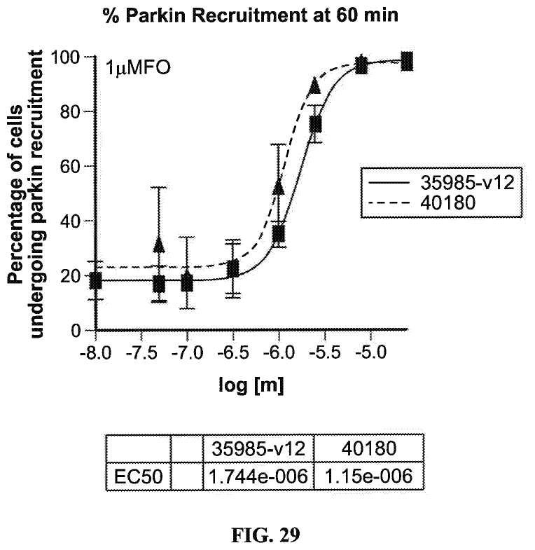

As shown in FIG. 29, addition of 35985 or 40180 results in a dose responsive increase in the rate of Parkin recruitment to mitochondria. HeLa cells expressing a YFP-tagged Parkin were treated with 1 mM of FCCP and Oligomycin followed by the specified dose of compound, then analyzed by longitudinal imaging. Percentage of cells with Parkin recruited to mitochondria at 60 minutes is shown.

### 10. Pathway Engagement Data

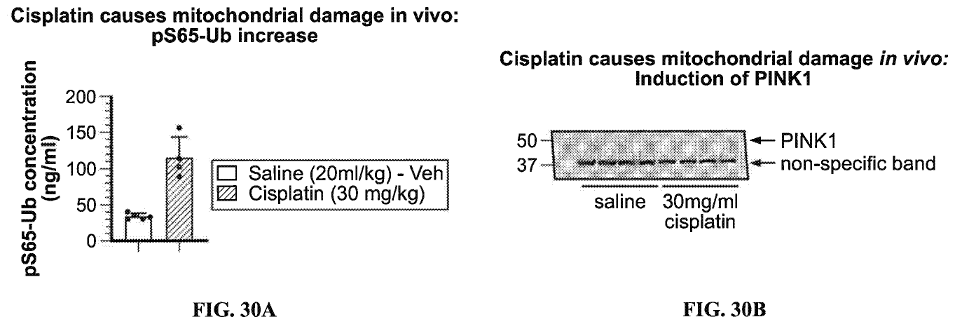

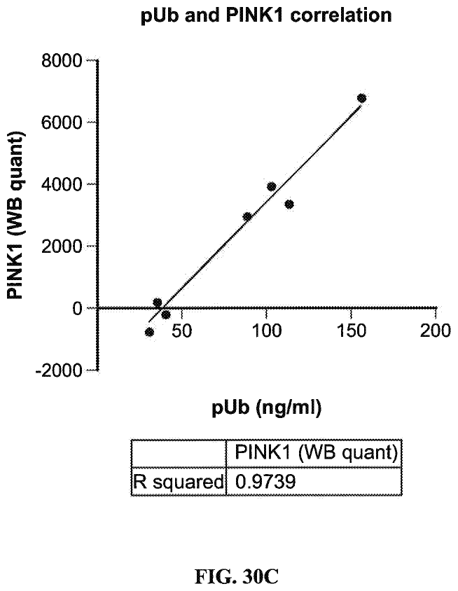

Mice (C57Bl/6) were challenged with a single intraperitoneal dose of 30 mg/kg cisplatin. Mitochondrial preparations were examined using pS65-Ub ELISA (FIG. 30A) or Western blot for PINK1 (FIG. 30B). Referring to FIG. 30C, there is a high degree of correlation between PINK1 protein concentration and its direct target, pS65-Ub, in kidney mitochondria.

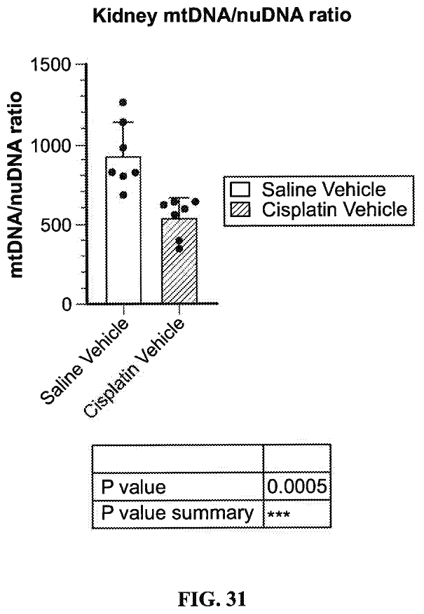

Referring to FIG. 31, mice (C57Bl/6) were challenged with a single intraperitoneal dose of 10 mg/kg cisplatin. Tissue lysates were examined for mitochondrial gene NDI and nuclear gene beta actin by quantitative PCR. A significant reduction in mitochondrial DNA was observed upon cisplatin challenge.

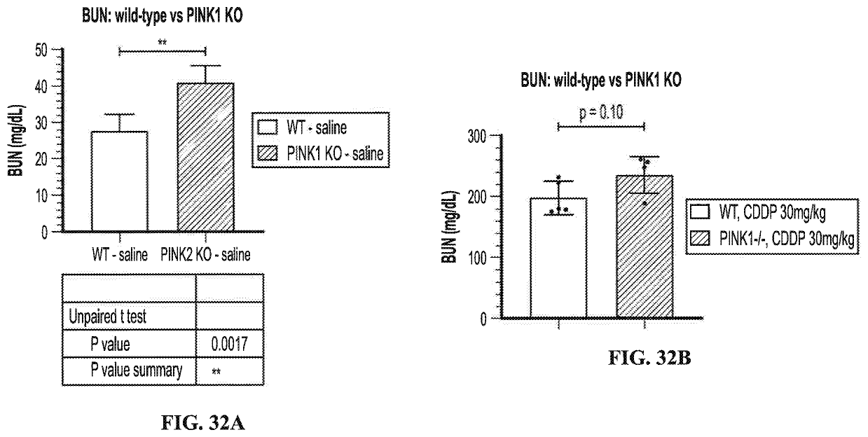

Referring to FIG. 32A and FIG. 32B, mice (C57Bl/6 or PINK1 knockout in C57BL/6 background) were challenged with a single dose of saline or 30 mg/kg cisplatin (intraperitoneal). Blood urea nitrogen (BUN), a commonly used clinical marker for kidney dysfunction, is increased in the PINK1 knockout mice relative to the wild-type mice.

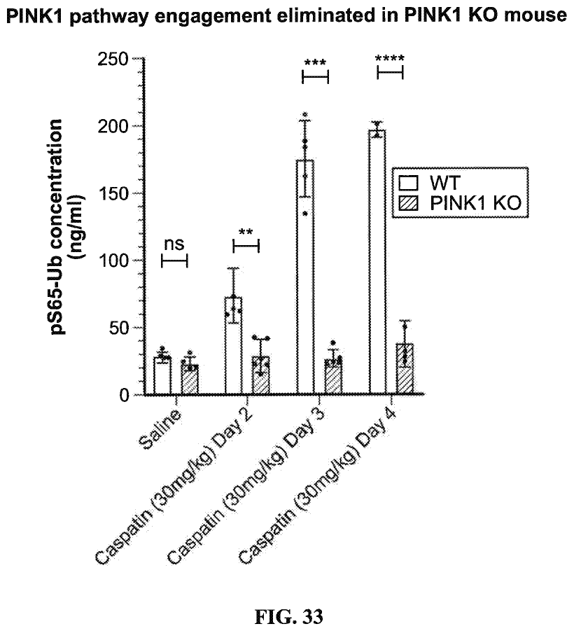

Referring to FIG. 33, mice (C57Bl/6 or PINK1 knockout in C57Bl/6 background) were challenged with a single intraperitoneal dose of 30 mg/kg cisplatin. Kidney mitochondria pS65-Ub levels were examined after different times as indicated. The fact that pS65 ubiquitin remains unchanged in PINK KO mice after cisplatin challenge indicates that PINK1 function is completely eliminated in those animals.

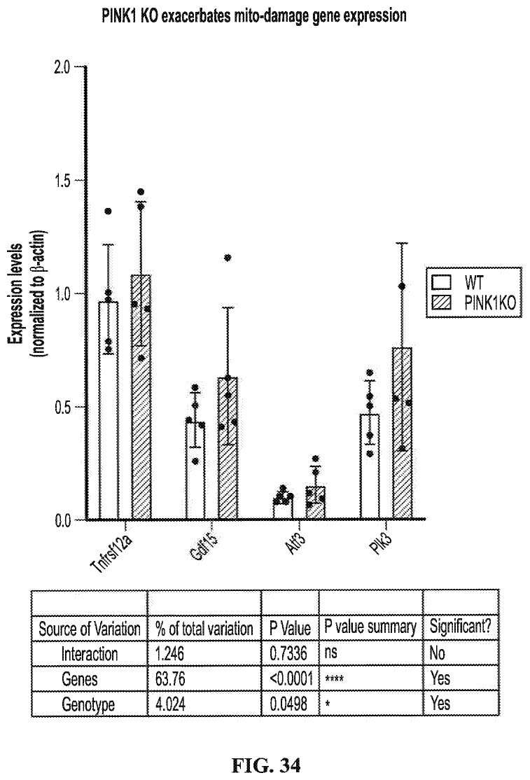

Referring to FIG. 34, mice (C57Bl/6 or PINK1 knockout in C57Bl/6 background) were challenged with a single intraperitoneal dose of 30 mg/kg cisplatin. Gene expression levels in the kidney for mitochondrial stress-responsive genes were examined using quantitative PCR. Mitochondrial stress gene expression is significantly increased in PINK1 knockout mice.

### 11. Pk Data

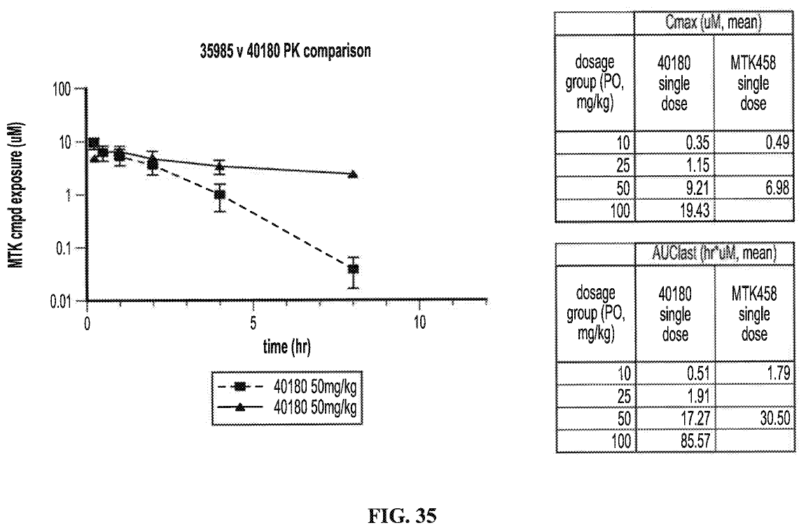

Briefly, mice (C57Bl/6, fed) were dosed by oral gavage with either 35985 or 40180 in NMP/solutol vehicle. Plasma concentrations of 35985 or 40180 were determined by mass spectrometry in at least 3 mice per study. Data for 50 mg/kg dose-level is shown in the graph of FIG. 35; computed pharmacokinetic parameters for different dose-levels are illustrated in the tables.

### 12. 40180 IN Vivo DATA

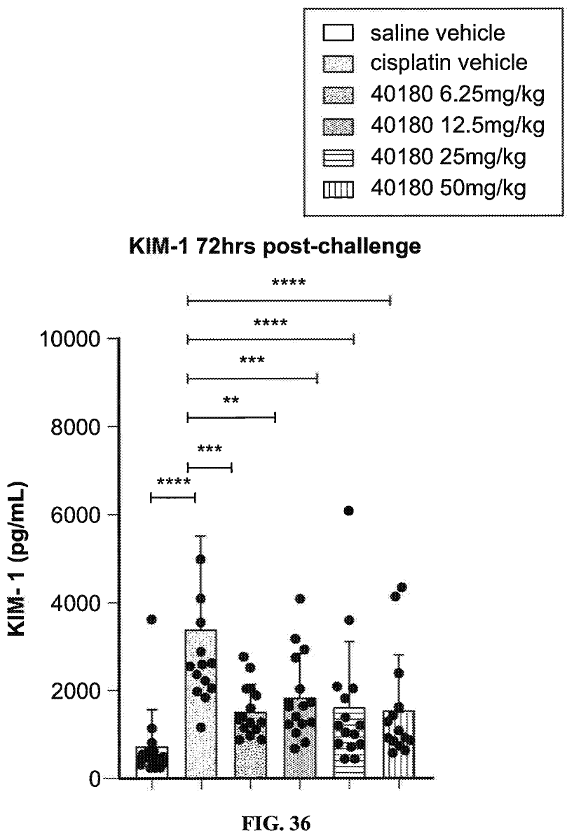

Referring to FIG. 36, mice (C57Bl/6) were challenged with a single intraperitoneal dose of 10 mg/kg cisplatin or saline and dosed by oral gavage once per day (QD) with 40180. On day 3 post-challenge, 40180 demonstrated a dose-dependent rescue of KIM-1 (kidney-injury marker 1), a urine biomarker specific for kidney injury.

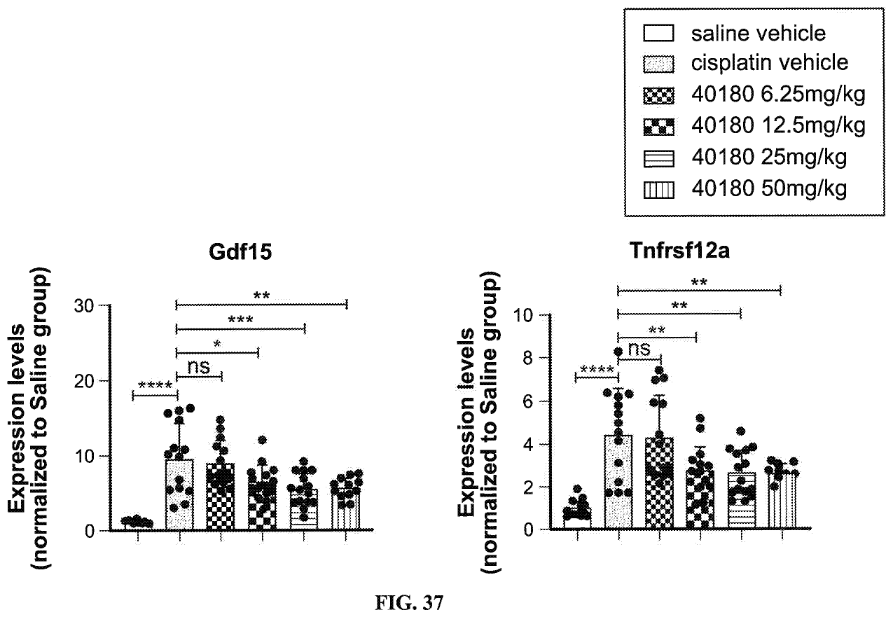

Referring to FIG. 37, mice (C57Bl/6) were challenged with a single intraperitoneal dose of 10 mg/kg cisplatin or saline and dosed by oral gavage, once per day (QD) with 40180 for three days. Cisplatin challenge significant increased expression of mitochondrial-stress related genes Gdf15 (left graph) and Tnfrsf12a (right graph); 40180 treatment reduced expression of these genes in a dose-responsive manner.

### G. PROPHETIC EXPERIMENTAL METHODS

### 1. Lipopolysaccharide (Lps) Assay

Briefly, P0 to P2 mice will be sacrificed and their cortical tissue dissected and plated according to standard methods to obtain primary mixed cortical cultures. Cultures will be maintained for 14 days. On or around Day 15, MTK compound will be added and allowed to incubate for 24 hours. After incubation with compound, the cells will be challenged with 100 ng/ml LPS. 24 hours after challenge initiation, cellular media is collected for analysis of cytokine levels via ELISA. A commercial ELISA kit for IL-6, TNF-α, and IL1-β will be used.

### 2. Ornithine Carbamoyltransferase (Dotc) Assay

The expression of a deletion mutant of dOTC yields Triton X-100 insoluble protein aggregates in the mitochondrial matrix. This misfolded protein expression is capable of recruiting PINK1/Parkin to mitochondria without depolarizing the inner mitochondrial membrane. Thus, without wishing to be bound by theory, it may represent a more physiological mechanism of PINK1 stabilization.

Here, HeLa cells stably expressing YFP parkin, containing doxycycline inducible expression of dOTC, are obtained. The cells are seeded at 20000 cells/well plus doxycycline (1 μg/mL) plus MTK on a 96-well plate. On Day 3, the cells are fixed and permeabilized and bound with OTC antibody. DAPI and cell mask are added. There is no wash off of dox. The results are imaged at 40×, non-confocal. 85-600 cells are analyzed per well. Each condition has 1-3 wells.

### 3. Effect of Mtk Compounds 35985 and 40180 on Cisplatin-Mediated Kidney Fibrosis Model

Repeated low-level tissue damage can lead to fibrosis and chronic disease in the affected tissue. Cisplatin can cause lung and kidney fibrosis in humans (Guinee et al., Cancer 1993), and repeated low-dose cisplatin challenge in mice causes fibrosis in mice (Sharp et al, AJP Nephrology, 2016; Katagiri et al, Kidney International, 2015). By reducing cisplatin-mediated mtDNA damage, though a PINK1-dependent mechanism identical for evidence provided above, MTK compounds 35985 and 40180 will be shown to be protective for kidney fibrosis.

Sharp et al. describe a protocol by which mice (FVB strain) are injected weekly with 7 mg/kg cisplatin by intraperitoneal injection. N=12-15 mice per group will be injected with saline or 7 mg/kg cisplatin weekly for four weeks, and dosed with either vehicle or MTK compounds by oral gavage at doses of about 1 mg/kg, about 2 mg/kg, about 5 mg/kg, about 10 mg/kg, about 20 mg/kg, or about 50 mg/kg, either once a day or twice a day. Then, blood urea nitrogen or creatinine (urine), and kidney-injury marker-1 (KIM-1) will be assessed to evaluate kidney function and injury, respectively. Furthermore, quantitative PCR (qPCR) will be used to measure the expression of inflammatory markers such TNFalpha, IL-11beta, and 1L-6. TGFbeta and fibronectin will be measured using western blot or commercially available ELISA kit as the principle readout for fibrosis, and count the number of infiltrating reactive immune cells in kidney sections by immunofluorescence or immunohistochemistry as a secondary measure of kidney fibrosis. Without wishing to be bound by theory, it is expected that administration of 35985 or 40180 will reduce fibrosis by 50% or more at therapeutic doses.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 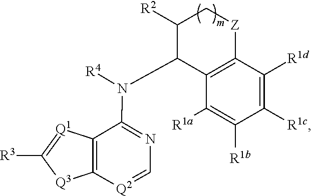
wherein m is 0 or 1;
wherein each of Q¹ and Q² is independently N or CH;
wherein Q³ is CH₂ or NH;
wherein Z is CR^{11a}R^{11b}, NR¹², or O;
wherein each of R^{11a} and R^{11b}, when present, is independently selected from hydrogen, halogen, —OH, and C1-C4 alkyloxy,
or wherein each of R^{11a} and R^{11b}, when present, together comprise ═O;
wherein R¹², when present, is hydrogen, C1-C4 alkyl, C3-C6 cycloalkyl, or —(C1-C4 alkyl)(C3-C6 cycloalkyl);
wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino;
wherein R² is selected from —(CH₂)ₙCy¹, —O(CH₂)ₙCy¹, —NR¹³(CH₂)ₙCy¹, —CH(OH)Cy¹, and Cy¹;
wherein n, when present, is 0, 1, or 2;
wherein R¹³, when present, is selected from hydrogen and C1-C4 alkyl;
wherein Cy¹ is a C4-C9 cycloalkyl, a C3-C9 heterocycle having at least one O, S, or N atom, or a C2-C9 heteroaryl having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —(C1-C4)-O—(C1-C4 alkyl), —C(O)(C1-C4 alkyl), —S(O)R¹⁴, C1-C4 alkylamino, and (C1-C4)(C1-C4) dialkylamino;
wherein R¹⁴, when present, is selected from —OH, —NH₂, —O(C1-C4 alkyl), —NH(C1-C4 alkyl), and —N(C1-C4 alkyl)(C1-C4 alkyl);
wherein R³ is a 3- to 6-membered cycloalkyl, a C1-C6 haloalkyl, C1-C6 haloalkoxy, or C1-C6 halohydroxyalkyl; and
wherein R⁴ is selected from hydrogen and C1-C4 alkyl,
or a pharmaceutically acceptable salt thereof.

2. The compound of claim 1, wherein R² is selected from —O(CH₂)ₙCy¹, —NR₁₃(CH₂)ₙCy¹, and Cy¹; Cy¹ is a C3-C9 heterocycle having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and R⁴ is hydrogen.

3. The compound of claim 1, wherein m is 1.

4. The compound of claim 1, wherein Q¹ is CH.

5. The compound of claim 1, wherein Q² is N.

6. The compound of claim 1, wherein Q³ is NH.

7. The compound of claim 1, wherein Q¹ is CH, Q² is N, and Q³ is NH.

8. The compound of claim 1, wherein Z is CH₂.

9. The compound of claim 1, wherein each of R^{1a}, R^{1b}, R^{1c}, and R^{1d} is independently hydrogen, halogen, or C1-C4 alkyl.

10. The compound of claim 1, wherein R² is selected from —O(CH₂)ₙCy¹, —NR¹³(CH₂)ₙCy¹, —CH(OH)Cy¹, and Cy¹;

11. The compound of claim 1, wherein R² is Cy¹.

12. The compound of claim 11, wherein Cy¹ is a C3-C9 heterocycle having at least one O, S, or N atom and unsubstituted.

13. The compound of claim 11, wherein Cy¹ is a structure represented by a formula selected from:

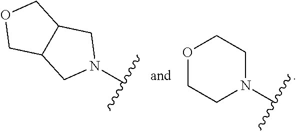 [SMILES: *N1CC2COCC2C1 ; *N1CCOCC1]

14. The compound of claim 1, wherein Cy¹ is a C3-C9 heterocycle having at least one O, S, or N atom.

15. The compound of claim 14, wherein the C3-C9 heterocycle is a monocyclic heterocycle.

16. The compound of claim 14, wherein the C3-C9 heterocycle is a bicyclic heterocycle.

17. The compound of claim 14, wherein the C3-C9 heterocycle is a spirocyclic heterocycle.

18. The compound of claim 14, wherein the C3-C9 heterocycle is a fused heterocycle.

19. The compound of claim 1, wherein Cy¹ is a C2-C9 heteroacyl having at least one O, S, or N atom.

20. The compound of claim 1, wherein Cy¹ is a C3-C9 heterocycle having at least one O, S, or N atom, and substituted with 0, 1, 2, or 3 groups independently selected from halogen, —CN, —NH₂, —OH, —NO₂, ═O, C3-C6 cycloalkyl, C2-C5 heterocycloalkyl, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

21. The compound of claim 1, wherein R³ is a 3- to 6-membered cycloalkyl or a C1-C6 haloalkyl.

22. The compound of claim 1, wherein R³ is a 3-membered cycloalkyl or —CF₃.

23. The compound of claim 1, wherein R⁴ is hydrogen.

24. The compound of claim 1, wherein the compound has a structure represented by a formula:

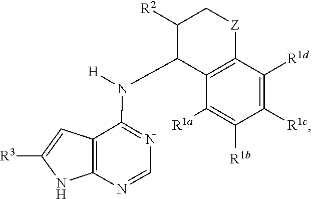

25. The compound of claim 1, wherein the compound has a structure represented by a formula:

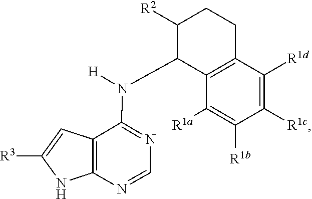

26. The compound of claim 1, wherein the compound has a structure represented by a formula selected from:

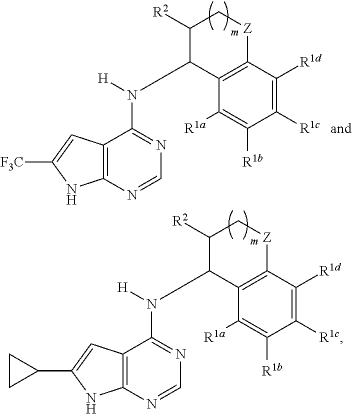

27. The compound of claim 1, wherein the compound has a structure represented by a formula selected from:

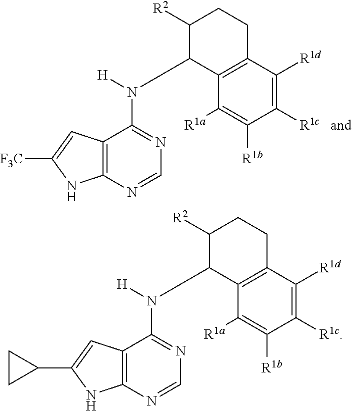

28. The compound of claim 1, wherein the compound is selected from:

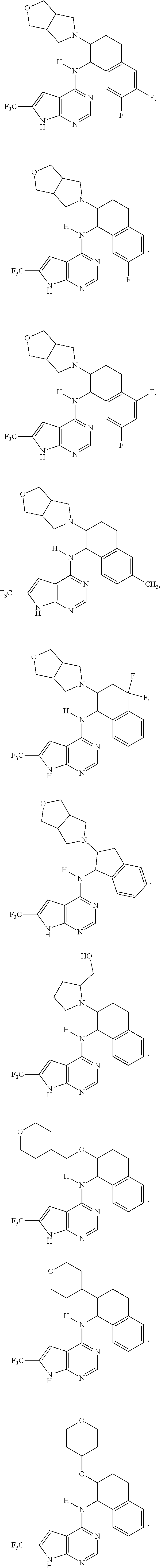

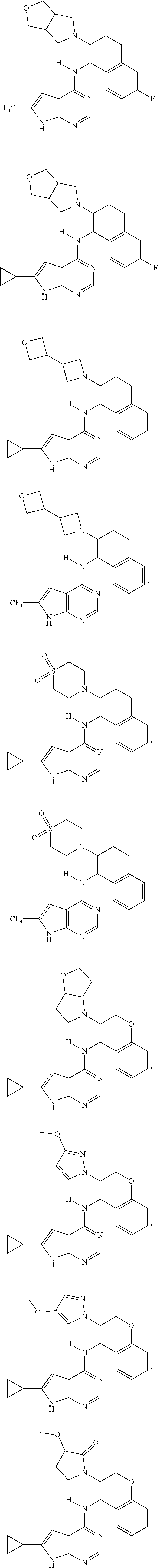

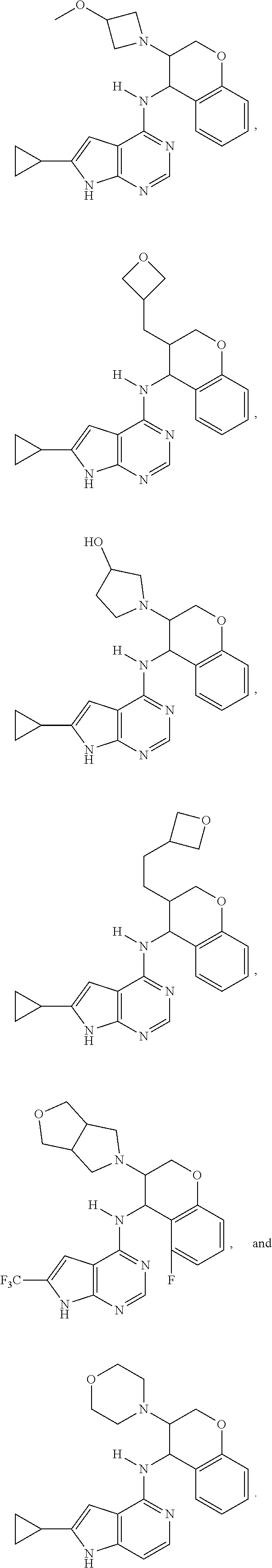 [SMILES: COC1CN(C2COc3ccccc3C2Nc2ncnc3[nH]c(C4CC4)cc23)C1 ; c1ccc2c(c1)OCC(CC1COC1)C2Nc1ncnc2[nH]c(C3CC3)cc12 ; OC1CCN(C2COc3ccccc3C2Nc2ncnc3[nH]c(C4CC4)cc23)C1 ; c1ccc2c(c1)OCC(CCC1COC1)C2Nc1ncnc2[nH]c(C3CC3)cc12 ; Fc1cccc2c1C(Nc1ncnc3[nH]c(C(F)(F)F)cc13)C(N1CC3COCC3C1)CO2 ; c1ccc2c(c1)OCC(N1CCOCC1)C2Nc1nccc2[nH]c(C3CC3)cc12]

29. The compound of claim 1, wherein the compound is selected from:

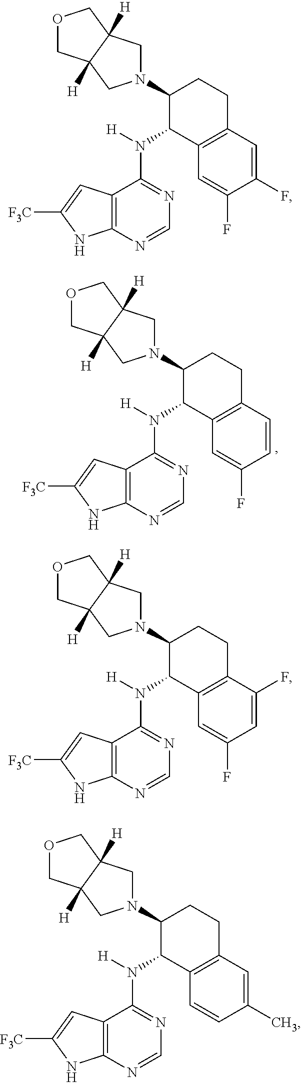

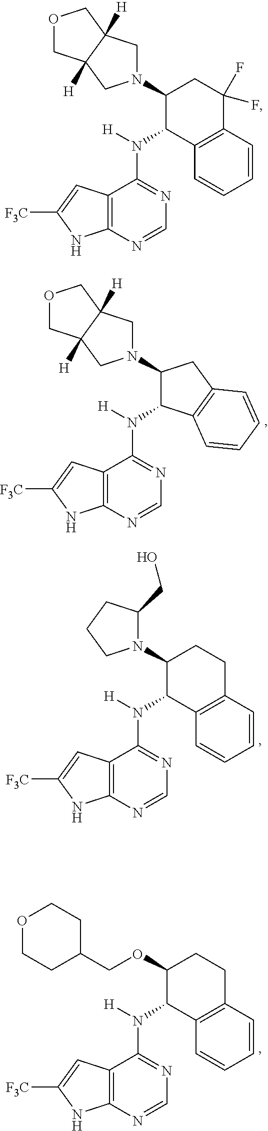

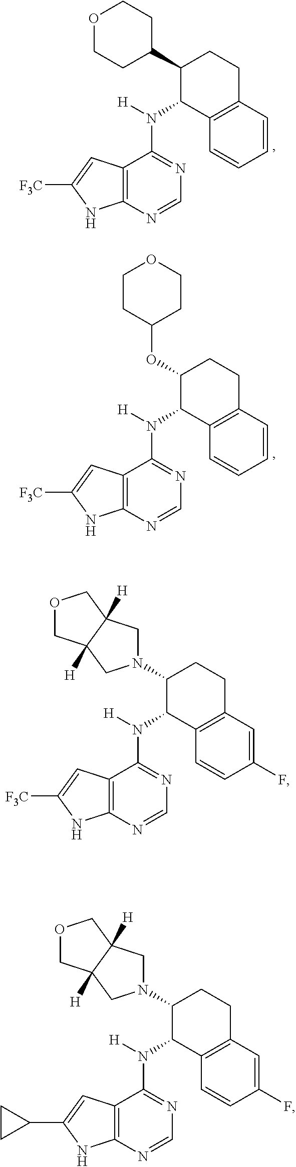

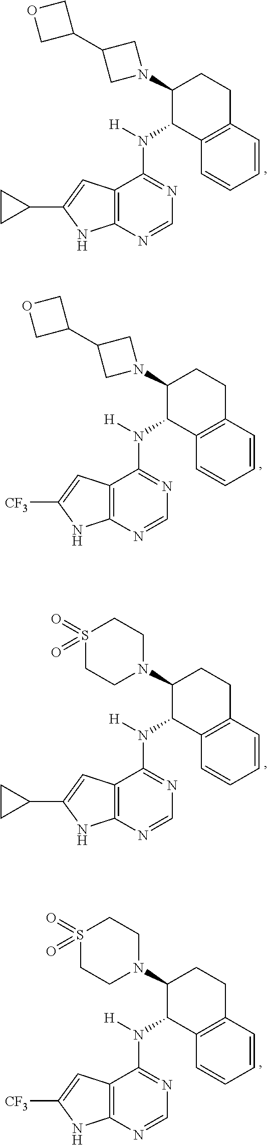

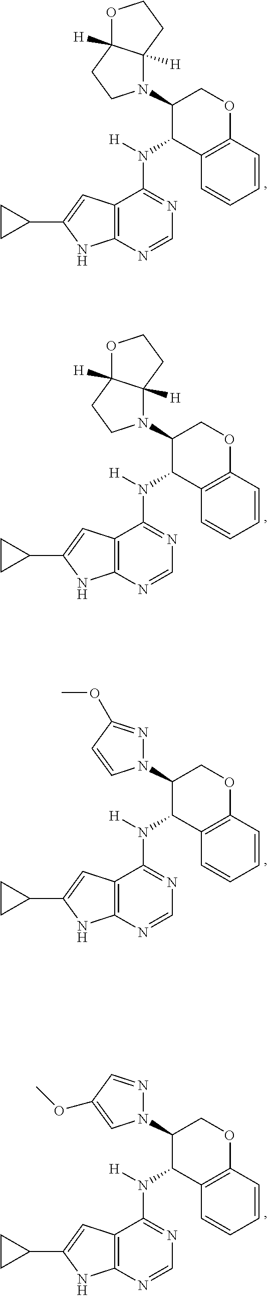

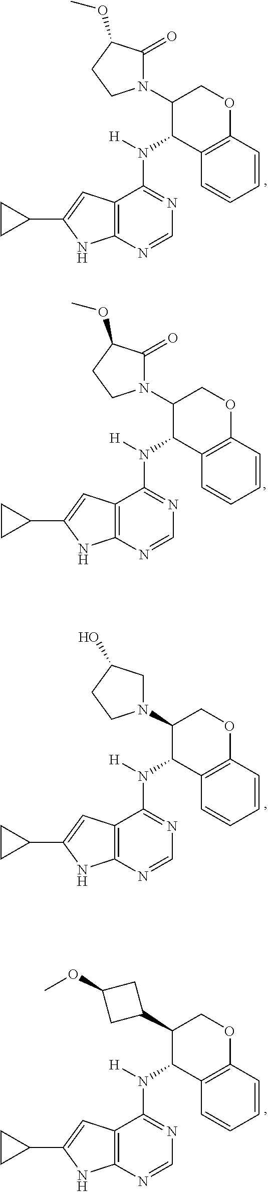

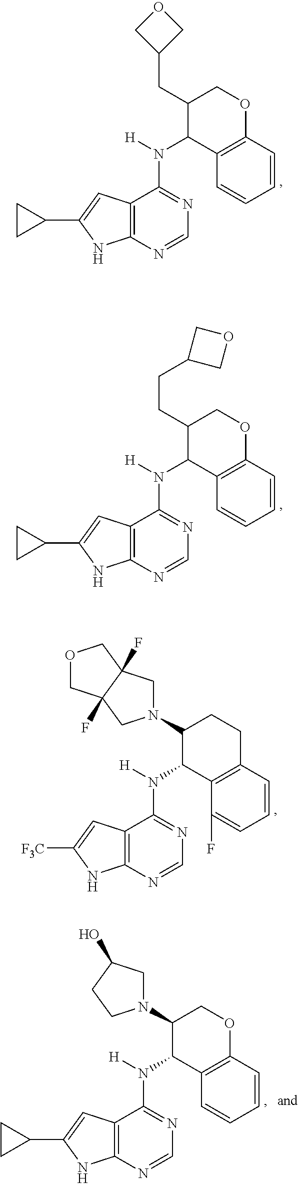

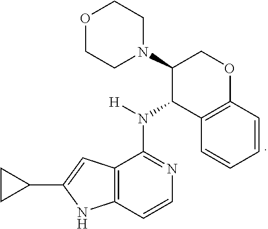

30. The compound of claim 1, wherein the compound is selected from:

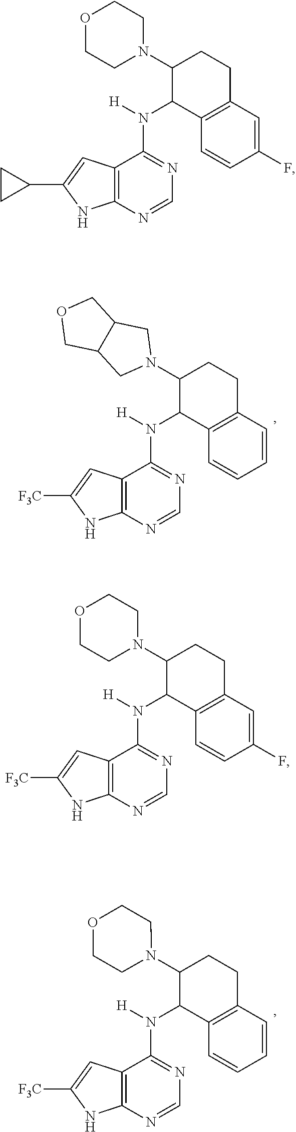 [SMILES: Fc1ccc2c(c1)CCC(N1CCOCC1)C2Nc1ncnc2[nH]c(C3CC3)cc12 ; FC(F)(F)c1cc2c(NC3c4ccccc4CCC3N3CC4COCC4C3)ncnc2[nH]1 ; Fc1ccc2c(c1)CCC(N1CCOCC1)C2Nc1ncnc2[nH]c(C(F)(F)F)cc12 ; FC(F)(F)c1cc2c(NC3c4ccccc4CCC3N3CCOCC3)ncnc2[nH]1]

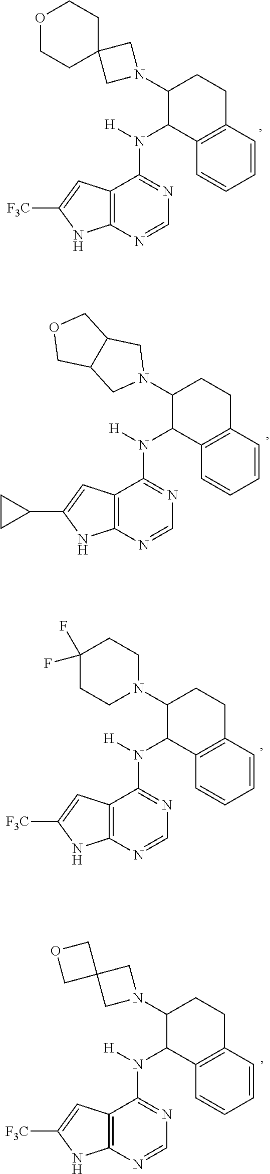 [SMILES: FC(F)(F)c1cc2c(NC3c4ccccc4CCC3N3CC4(CCOCC4)C3)ncnc2[nH]1 ; c1ccc2c(c1)CCC(N1CC3COCC3C1)C2Nc1ncnc2[nH]c(C3CC3)cc12 ; FC1(F)CCN(C2CCc3ccccc3C2Nc2ncnc3[nH]c(C(F)(F)F)cc23)CC1 ; FC(F)(F)c1cc2c(NC3c4ccccc4CCC3N3CC4(COC4)C3)ncnc2[nH]1]

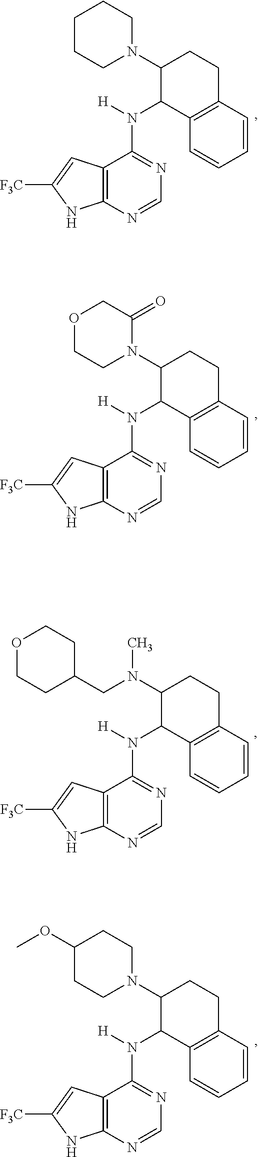 [SMILES: FC(F)(F)c1cc2c(NC3c4ccccc4CCC3N3CCCCC3)ncnc2[nH]1 ; O=C1COCCN1C1CCc2ccccc2C1Nc1ncnc2[nH]c(C(F)(F)F)cc12 ; CN(CC1CCOCC1)C1CCc2ccccc2C1Nc1ncnc2[nH]c(C(F)(F)F)cc12 ; COC1CCN(C2CCc3ccccc3C2Nc2ncnc3[nH]c(C(F)(F)F)cc23)CC1]

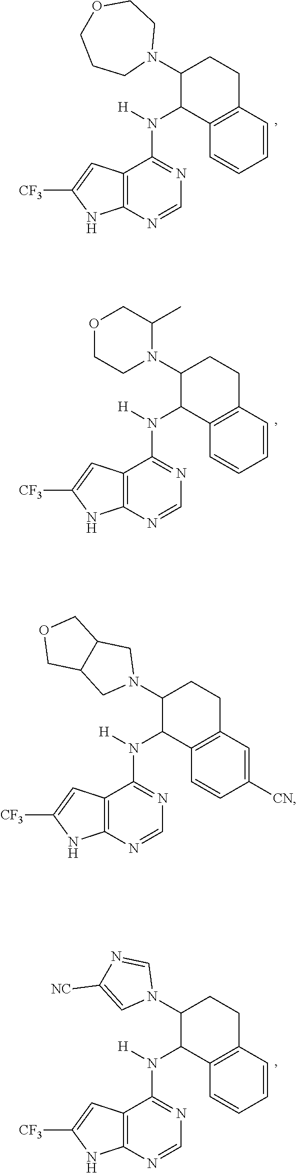 [SMILES: FC(F)(F)c1cc2c(NC3c4ccccc4CCC3N3CCCOCC3)ncnc2[nH]1 ; CC1COCCN1C1CCc2ccccc2C1Nc1ncnc2[nH]c(C(F)(F)F)cc12 ; N#Cc1ccc2c(c1)CCC(N1CC3COCC3C1)C2Nc1ncnc2[nH]c(C(F)(F)F)cc12 ; N#Cc1cn(C2CCc3ccccc3C2Nc2ncnc3[nH]c(C(F)(F)F)cc23)cn1]

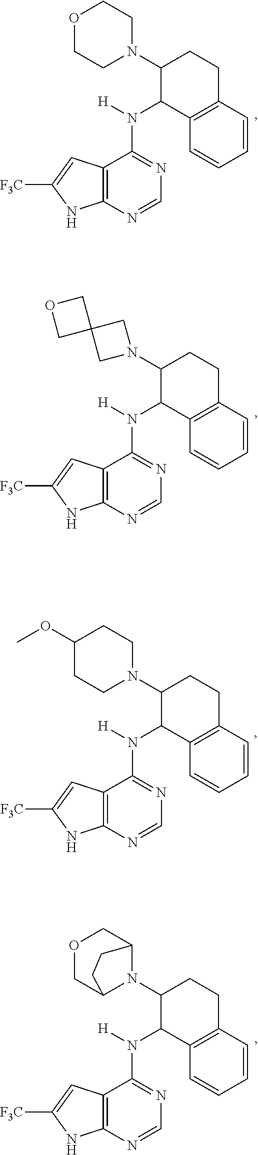 [SMILES: FC(F)(F)c1cc2c(NC3c4ccccc4CCC3N3CCOCC3)ncnc2[nH]1 ; FC(F)(F)c1cc2c(NC3c4ccccc4CCC3N3CC4(COC4)C3)ncnc2[nH]1 ; COC1CCN(C2CCc3ccccc3C2Nc2ncnc3[nH]c(C(F)(F)F)cc23)CC1 ; FC(F)(F)c1cc2c(NC3c4ccccc4CCC3N3C4CCC3COC4)ncnc2[nH]1]

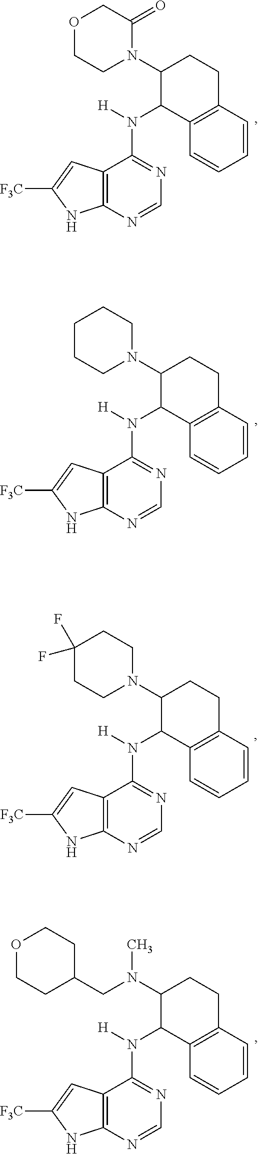 [SMILES: O=C1COCCN1C1CCc2ccccc2C1Nc1ncnc2[nH]c(C(F)(F)F)cc12 ; FC(F)(F)c1cc2c(NC3c4ccccc4CCC3N3CCCCC3)ncnc2[nH]1 ; FC1(F)CCN(C2CCc3ccccc3C2Nc2ncnc3[nH]c(C(F)(F)F)cc23)CC1 ; CN(CC1CCOCC1)C1CCc2ccccc2C1Nc1ncnc2[nH]c(C(F)(F)F)cc12]

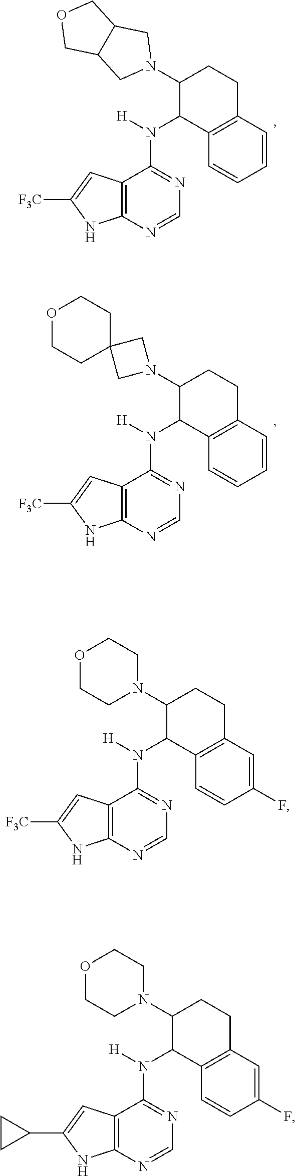 [SMILES: FC(F)(F)c1cc2c(NC3c4ccccc4CCC3N3CC4COCC4C3)ncnc2[nH]1 ; FC(F)(F)c1cc2c(NC3c4ccccc4CCC3N3CC4(CCOCC4)C3)ncnc2[nH]1 ; Fc1ccc2c(c1)CCC(N1CCOCC1)C2Nc1ncnc2[nH]c(C(F)(F)F)cc12 ; Fc1ccc2c(c1)CCC(N1CCOCC1)C2Nc1ncnc2[nH]c(C3CC3)cc12]

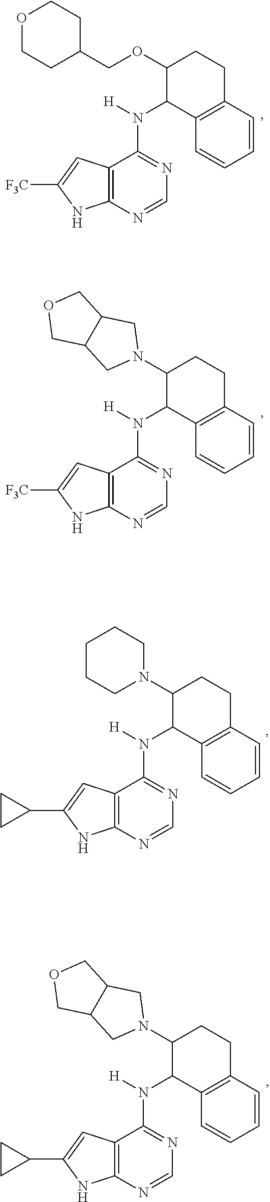 [SMILES: FC(F)(F)c1cc2c(NC3c4ccccc4CCC3OCC3CCOCC3)ncnc2[nH]1 ; FC(F)(F)c1cc2c(NC3c4ccccc4CCC3N3CC4COCC4C3)ncnc2[nH]1 ; c1ccc2c(c1)CCC(N1CCCCC1)C2Nc1ncnc2[nH]c(C3CC3)cc12 ; c1ccc2c(c1)CCC(N1CC3COCC3C1)C2Nc1ncnc2[nH]c(C3CC3)cc12]

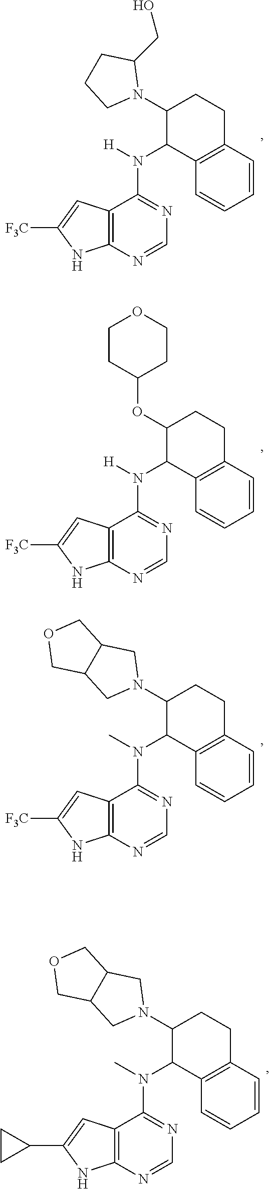 [SMILES: OCC1CCCN1C1CCc2ccccc2C1Nc1ncnc2[nH]c(C(F)(F)F)cc12 ; FC(F)(F)c1cc2c(NC3c4ccccc4CCC3OC3CCOCC3)ncnc2[nH]1 ; CN(c1ncnc2[nH]c(C(F)(F)F)cc12)C1c2ccccc2CCC1N1CC2COCC2C1 ; CN(c1ncnc2[nH]c(C3CC3)cc12)C1c2ccccc2CCC1N1CC2COCC2C1]

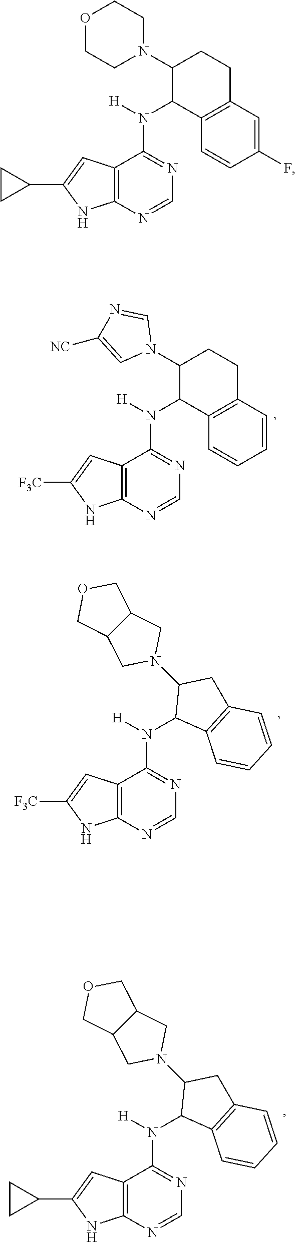 [SMILES: Fc1ccc2c(c1)CCC(N1CCOCC1)C2Nc1ncnc2[nH]c(C3CC3)cc12 ; N#Cc1cn(C2CCc3ccccc3C2Nc2ncnc3[nH]c(C(F)(F)F)cc23)cn1 ; FC(F)(F)c1cc2c(NC3c4ccccc4CC3N3CC4COCC4C3)ncnc2[nH]1 ; c1ccc2c(c1)CC(N1CC3COCC3C1)C2Nc1ncnc2[nH]c(C3CC3)cc12]

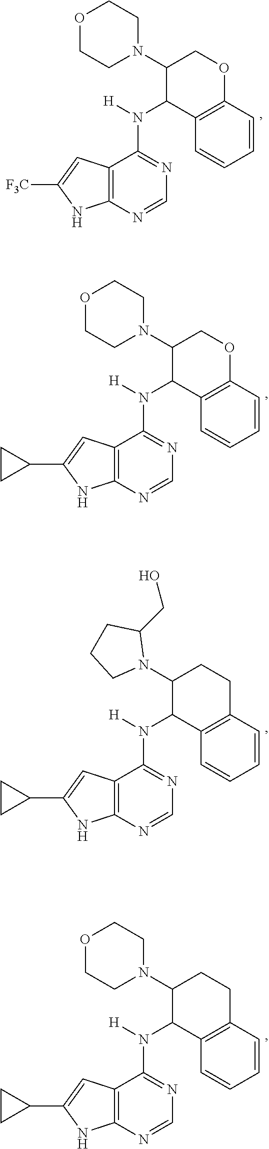 [SMILES: FC(F)(F)c1cc2c(NC3c4ccccc4OCC3N3CCOCC3)ncnc2[nH]1 ; c1ccc2c(c1)OCC(N1CCOCC1)C2Nc1ncnc2[nH]c(C3CC3)cc12 ; OCC1CCCN1C1CCc2ccccc2C1Nc1ncnc2[nH]c(C3CC3)cc12 ; c1ccc2c(c1)CCC(N1CCOCC1)C2Nc1ncnc2[nH]c(C3CC3)cc12]

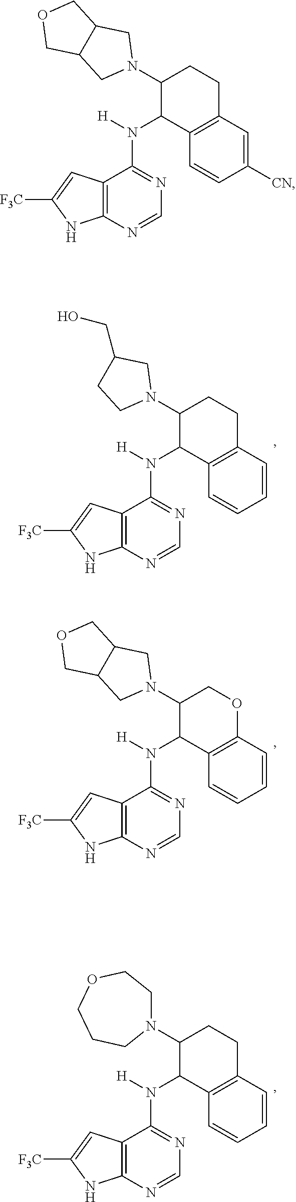 [SMILES: N#Cc1ccc2c(c1)CCC(N1CC3COCC3C1)C2Nc1ncnc2[nH]c(C(F)(F)F)cc12 ; OCC1CCN(C2CCc3ccccc3C2Nc2ncnc3[nH]c(C(F)(F)F)cc23)C1 ; FC(F)(F)c1cc2c(NC3c4ccccc4OCC3N3CC4COCC4C3)ncnc2[nH]1 ; FC(F)(F)c1cc2c(NC3c4ccccc4CCC3N3CCCOCC3)ncnc2[nH]1]

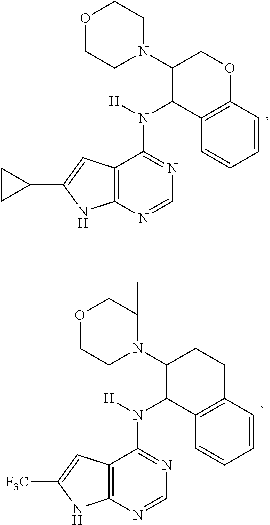 [SMILES: c1ccc2c(c1)OCC(N1CCOCC1)C2Nc1ncnc2[nH]c(C3CC3)cc12 ; CC1COCCN1C1CCc2ccccc2C1Nc1ncnc2[nH]c(C(F)(F)F)cc12]

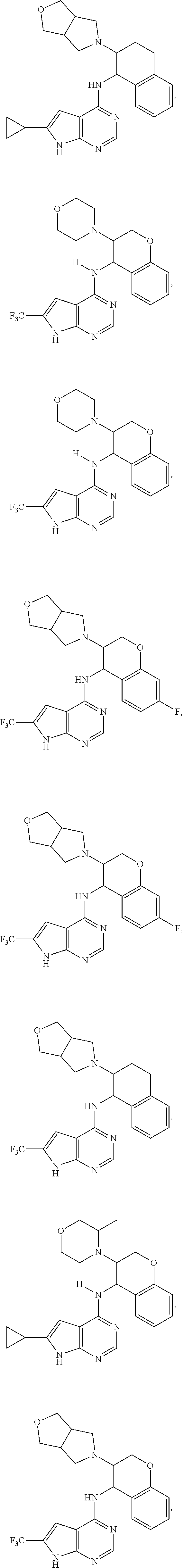

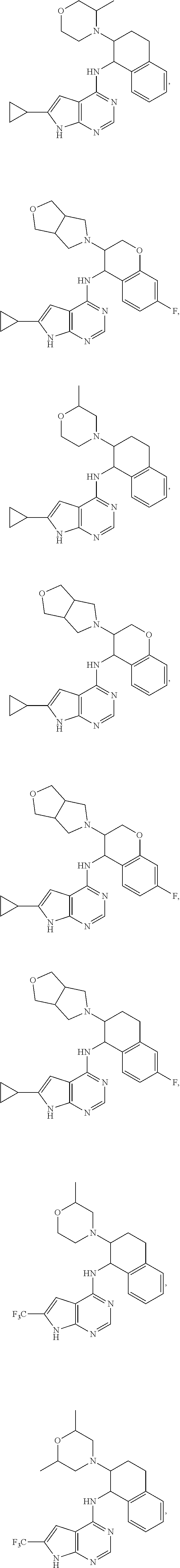

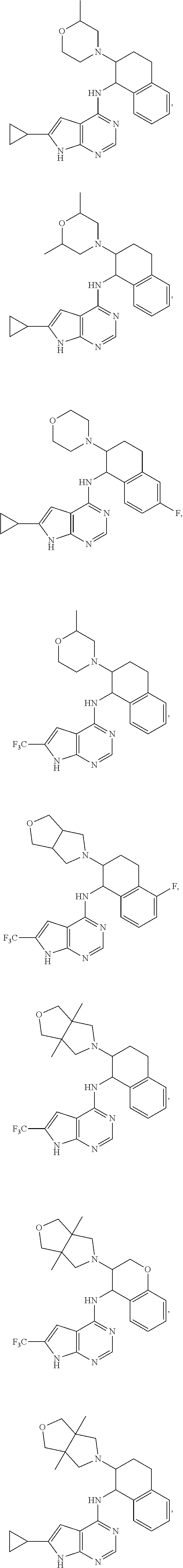

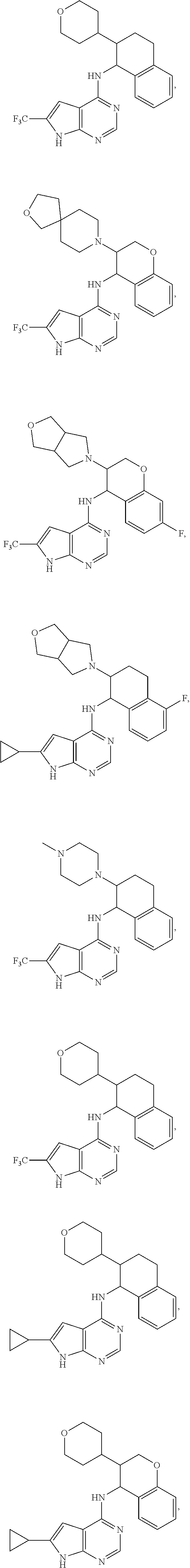

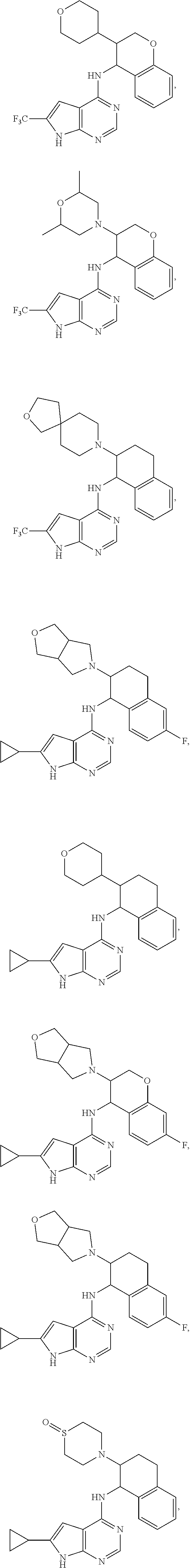

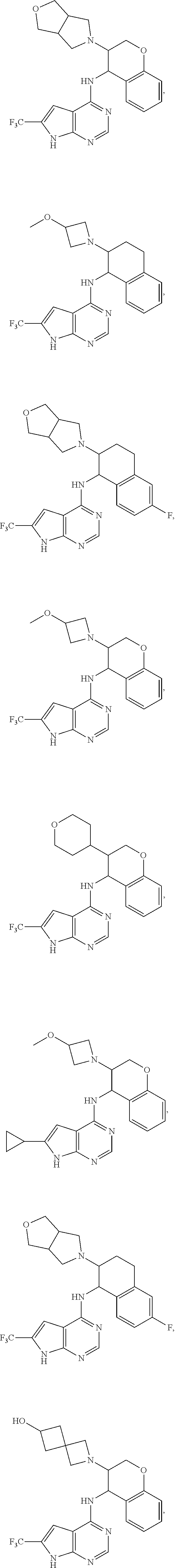

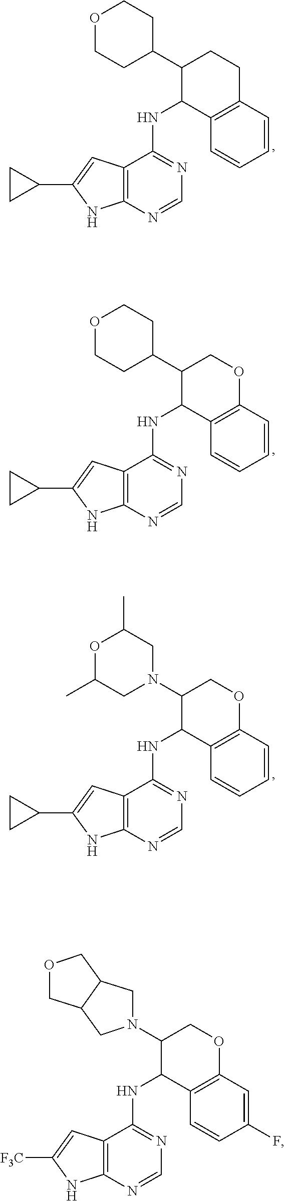 [SMILES: c1ccc2c(c1)CCC(C1CCOCC1)C2Nc1ncnc2[nH]c(C3CC3)cc12 ; c1ccc2c(c1)OCC(C1CCOCC1)C2Nc1ncnc2[nH]c(C3CC3)cc12 ; CC1CN(C2COc3ccccc3C2Nc2ncnc3[nH]c(C4CC4)cc23)CC(C)O1 ; Fc1ccc2c(c1)OCC(N1CC3COCC3C1)C2Nc1ncnc2[nH]c(C(F)(F)F)cc12]

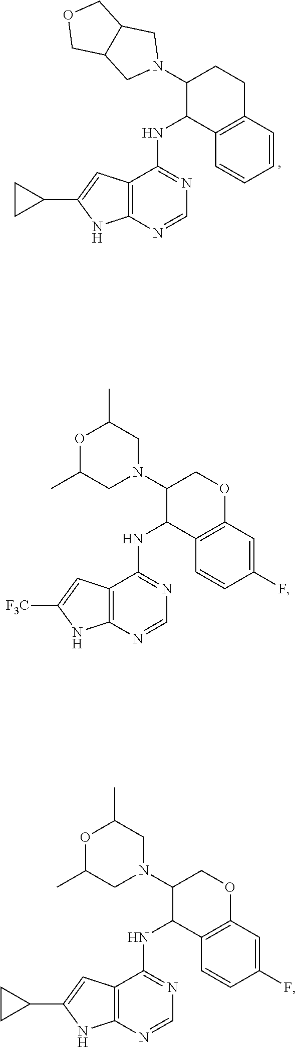 [SMILES: c1ccc2c(c1)CCC(N1CC3COCC3C1)C2Nc1ncnc2[nH]c(C3CC3)cc12 ; CC1CN(C2COc3cc(F)ccc3C2Nc2ncnc3[nH]c(C(F)(F)F)cc23)CC(C)O1 ; CC1CN(C2COc3cc(F)ccc3C2Nc2ncnc3[nH]c(C4CC4)cc23)CC(C)O1]

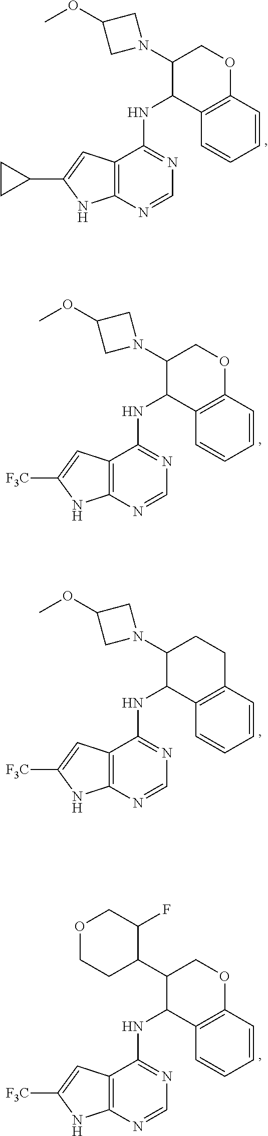 [SMILES: COC1CN(C2COc3ccccc3C2Nc2ncnc3[nH]c(C4CC4)cc23)C1 ; COC1CN(C2COc3ccccc3C2Nc2ncnc3[nH]c(C(F)(F)F)cc23)C1 ; COC1CN(C2CCc3ccccc3C2Nc2ncnc3[nH]c(C(F)(F)F)cc23)C1 ; FC1COCCC1C1COc2ccccc2C1Nc1ncnc2[nH]c(C(F)(F)F)cc12]

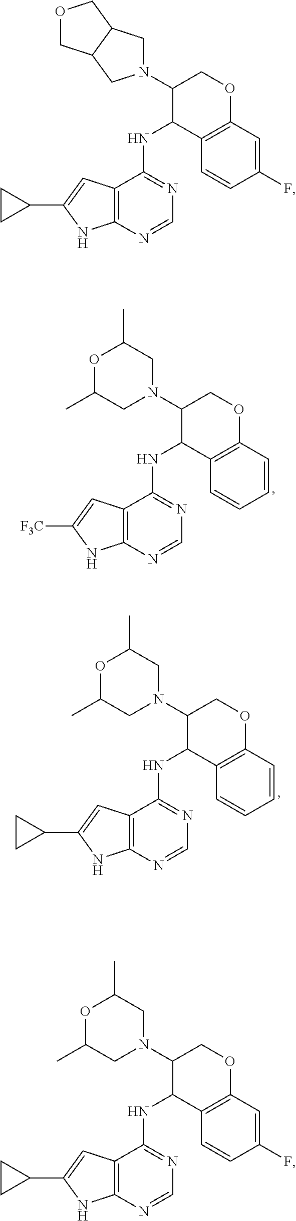 [SMILES: Fc1ccc2c(c1)OCC(N1CC3COCC3C1)C2Nc1ncnc2[nH]c(C3CC3)cc12 ; CC1CN(C2COc3ccccc3C2Nc2ncnc3[nH]c(C(F)(F)F)cc23)CC(C)O1 ; CC1CN(C2COc3ccccc3C2Nc2ncnc3[nH]c(C4CC4)cc23)CC(C)O1 ; CC1CN(C2COc3cc(F)ccc3C2Nc2ncnc3[nH]c(C4CC4)cc23)CC(C)O1]

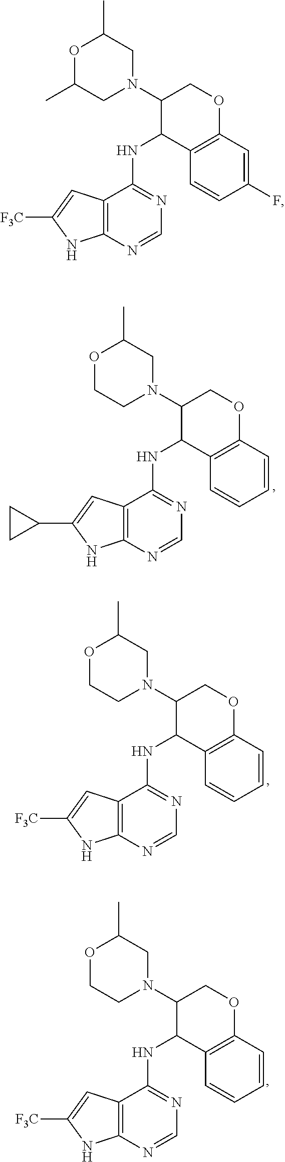 [SMILES: CC1CN(C2COc3cc(F)ccc3C2Nc2ncnc3[nH]c(C(F)(F)F)cc23)CC(C)O1 ; CC1CN(C2COc3ccccc3C2Nc2ncnc3[nH]c(C4CC4)cc23)CCO1 ; CC1CN(C2COc3ccccc3C2Nc2ncnc3[nH]c(C(F)(F)F)cc23)CCO1 ; CC1CN(C2COc3ccccc3C2Nc2ncnc3[nH]c(C(F)(F)F)cc23)CCO1]

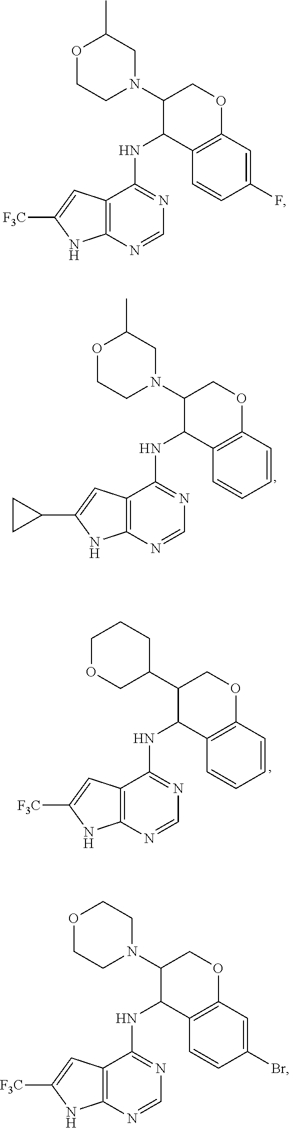 [SMILES: CC1CN(C2COc3cc(F)ccc3C2Nc2ncnc3[nH]c(C(F)(F)F)cc23)CCO1 ; CC1CN(C2COc3ccccc3C2Nc2ncnc3[nH]c(C4CC4)cc23)CCO1 ; FC(F)(F)c1cc2c(NC3c4ccccc4OCC3C3CCCOC3)ncnc2[nH]1 ; FC(F)(F)c1cc2c(NC3c4ccc(Br)cc4OCC3N3CCOCC3)ncnc2[nH]1]

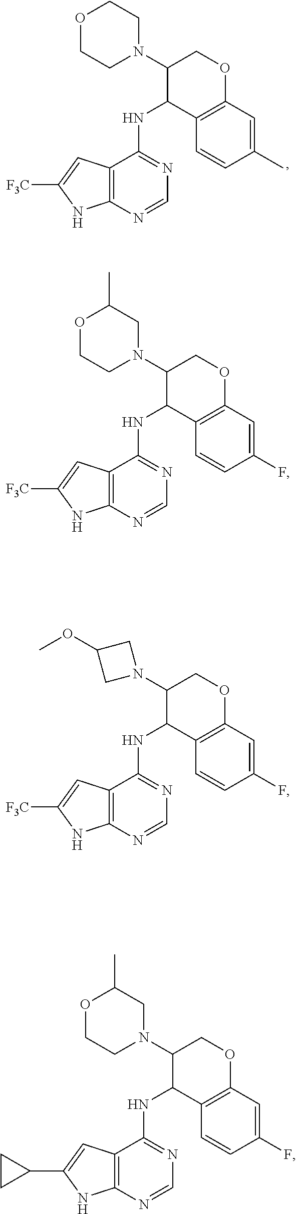 [SMILES: Cc1ccc2c(c1)OCC(N1CCOCC1)C2Nc1ncnc2[nH]c(C(F)(F)F)cc12 ; CC1CN(C2COc3cc(F)ccc3C2Nc2ncnc3[nH]c(C(F)(F)F)cc23)CCO1 ; COC1CN(C2COc3cc(F)ccc3C2Nc2ncnc3[nH]c(C(F)(F)F)cc23)C1 ; CC1CN(C2COc3cc(F)ccc3C2Nc2ncnc3[nH]c(C4CC4)cc23)CCO1]

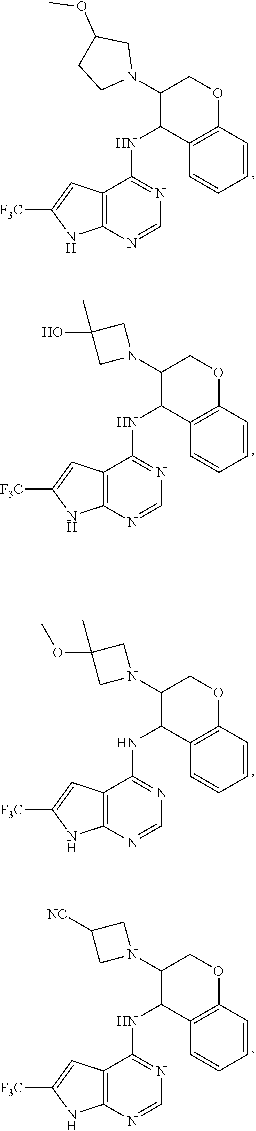 [SMILES: COC1CCN(C2COc3ccccc3C2Nc2ncnc3[nH]c(C(F)(F)F)cc23)C1 ; CC1(O)CN(C2COc3ccccc3C2Nc2ncnc3[nH]c(C(F)(F)F)cc23)C1 ; COC1(C)CN(C2COc3ccccc3C2Nc2ncnc3[nH]c(C(F)(F)F)cc23)C1 ; N#CC1CN(C2COc3ccccc3C2Nc2ncnc3[nH]c(C(F)(F)F)cc23)C1]

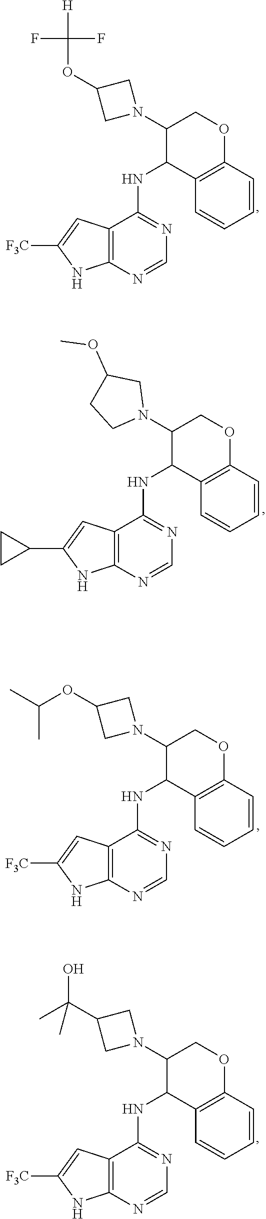 [SMILES: FC(F)OC1CN(C2COc3ccccc3C2Nc2ncnc3[nH]c(C(F)(F)F)cc23)C1 ; COC1CCN(C2COc3ccccc3C2Nc2ncnc3[nH]c(C4CC4)cc23)C1 ; CC(C)OC1CN(C2COc3ccccc3C2Nc2ncnc3[nH]c(C(F)(F)F)cc23)C1 ; CC(C)(O)C1CN(C2COc3ccccc3C2Nc2ncnc3[nH]c(C(F)(F)F)cc23)C1]

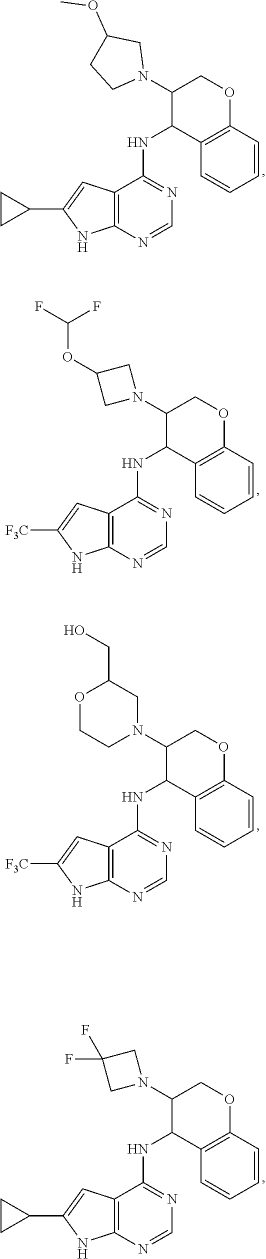 [SMILES: COC1CCN(C2COc3ccccc3C2Nc2ncnc3[nH]c(C4CC4)cc23)C1 ; FC(F)OC1CN(C2COc3ccccc3C2Nc2ncnc3[nH]c(C(F)(F)F)cc23)C1 ; OCC1CN(C2COc3ccccc3C2Nc2ncnc3[nH]c(C(F)(F)F)cc23)CCO1 ; FC1(F)CN(C2COc3ccccc3C2Nc2ncnc3[nH]c(C4CC4)cc23)C1]

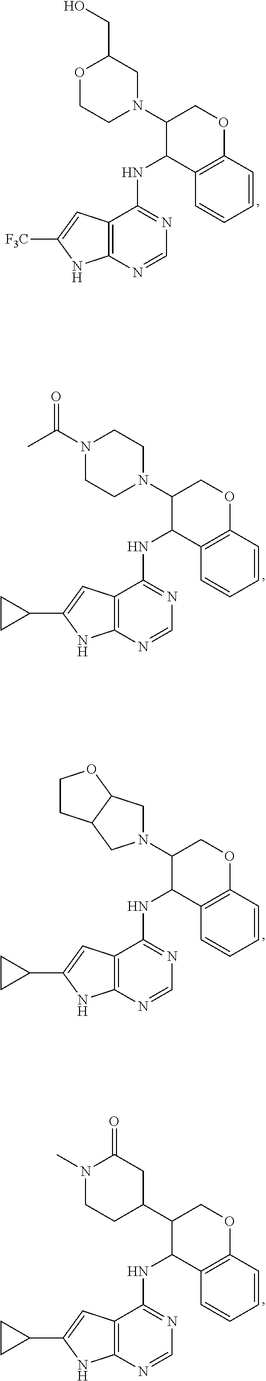 [SMILES: OCC1CN(C2COc3ccccc3C2Nc2ncnc3[nH]c(C(F)(F)F)cc23)CCO1 ; CC(=O)N1CCN(C2COc3ccccc3C2Nc2ncnc3[nH]c(C4CC4)cc23)CC1 ; c1ccc2c(c1)OCC(N1CC3CCOC3C1)C2Nc1ncnc2[nH]c(C3CC3)cc12 ; CN1CCC(C2COc3ccccc3C2Nc2ncnc3[nH]c(C4CC4)cc23)CC1=O]

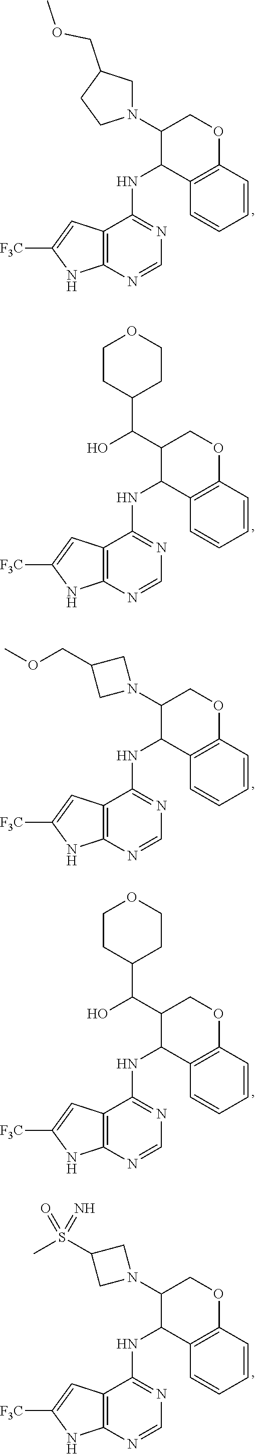

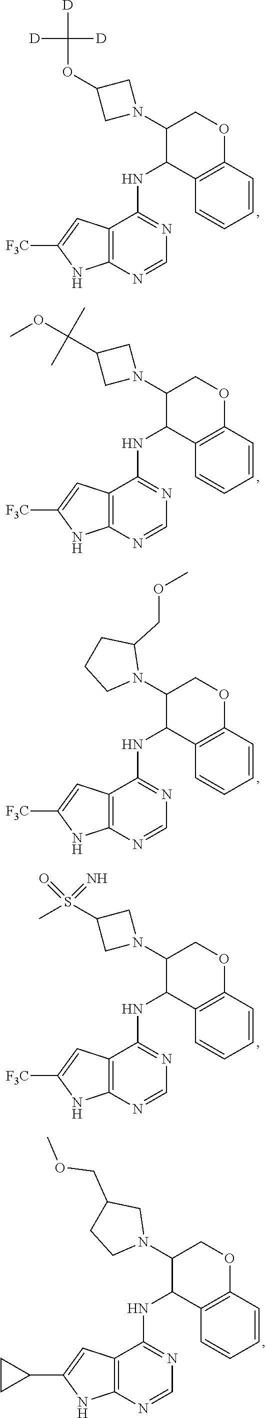

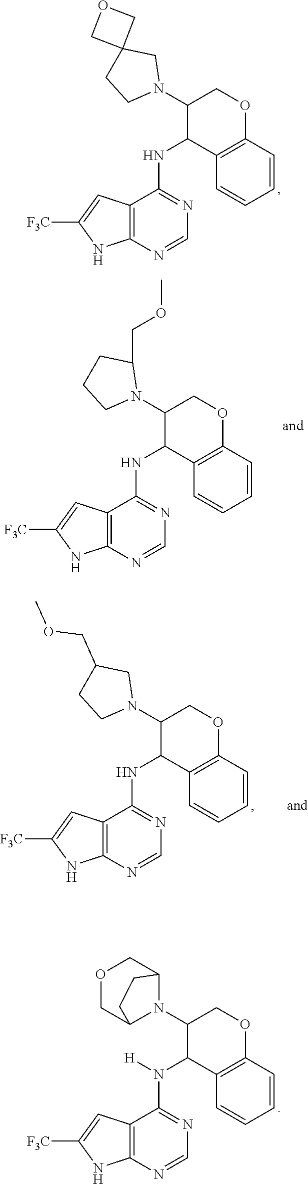

31. The compound of claim 1, wherein the compound is selected from:

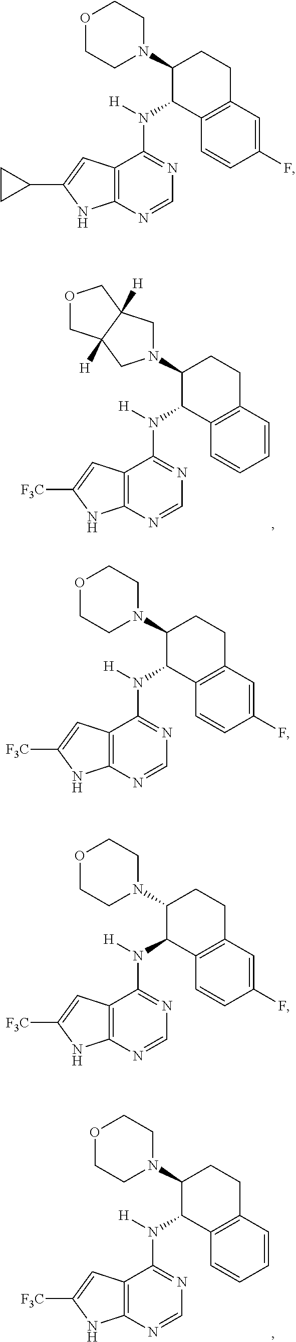 [SMILES: Fc1ccc2c(c1)CC[C@H](N1CCOCC1)[C@H]2Nc1ncnc2[nH]c(C3CC3)cc12 ; FC(F)(F)c1cc2c(N[C@H]3c4ccccc4CC[C@@H]3N3C[C@H]4COC[C@H]4C3)ncnc2[nH]1 ; Fc1ccc2c(c1)CC[C@H](N1CCOCC1)[C@H]2Nc1ncnc2[nH]c(C(F)(F)F)cc12 ; Fc1ccc2c(c1)CC[C@@H](N1CCOCC1)[C@@H]2Nc1ncnc2[nH]c(C(F)(F)F)cc12 ; FC(F)(F)c1cc2c(N[C@H]3c4ccccc4CC[C@@H]3N3CCOCC3)ncnc2[nH]1]

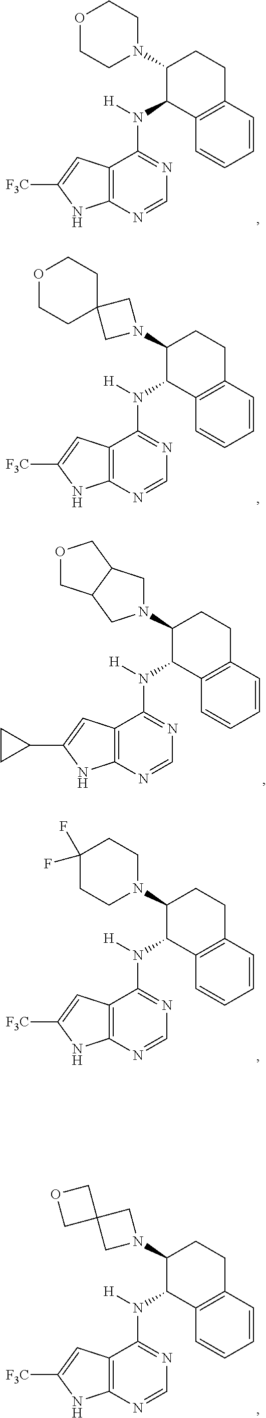 [SMILES: FC(F)(F)c1cc2c(N[C@@H]3c4ccccc4CC[C@H]3N3CCOCC3)ncnc2[nH]1 ; FC(F)(F)c1cc2c(N[C@H]3c4ccccc4CC[C@@H]3N3CC4(CCOCC4)C3)ncnc2[nH]1 ; c1ccc2c(c1)CC[C@H](N1CC3COCC3C1)[C@H]2Nc1ncnc2[nH]c(C3CC3)cc12 ; FC1(F)CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CC4(COC4)C3)ncnc2[nH]1]

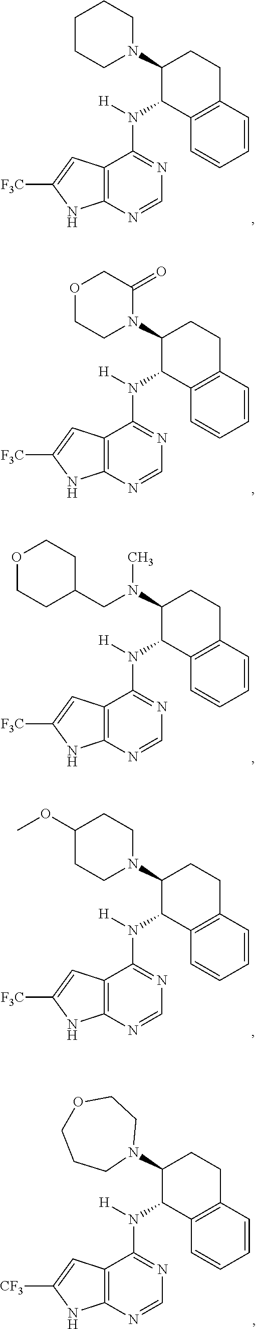 [SMILES: FC(F)(F)c1cc2c(N[C@H]3c4ccccc4CC[C@@H]3N3CCCCC3)ncnc2[nH]1 ; O=C1COCCN1[C@H]1CCc2ccccc2[C@@H]1Nc1ncnc2[nH]c(C(F)(F)F)cc12 ; CN(CC1CCOCC1)[C@H]1CCc2ccccc2[C@@H]1Nc1ncnc2[nH]c(C(F)(F)F)cc12 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3CCCOCC3)ncnc2[nH]1]

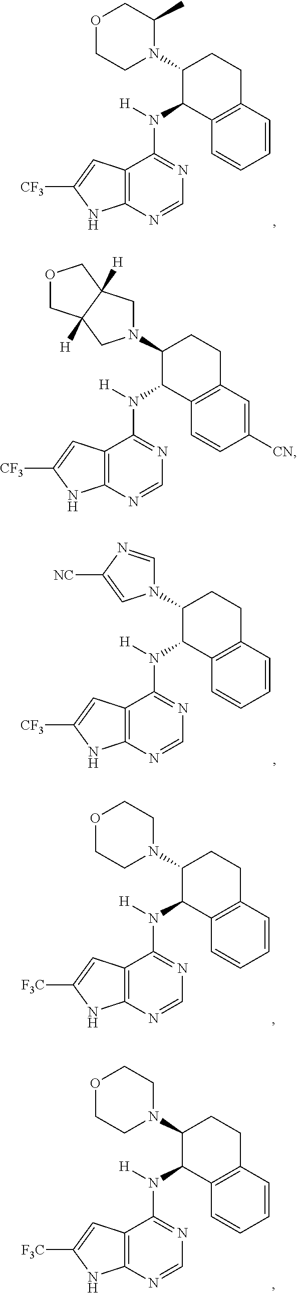 [SMILES: C[C@@H]1COCCN1[C@@H]1CCc2ccccc2[C@H]1Nc1ncnc2[nH]c(C(F)(F)F)cc12 ; N#Cc1ccc2c(c1)CC[C@H](N1C[C@H]3COC[C@H]3C1)[C@H]2Nc1ncnc2[nH]c(C(F)(F)F)cc12 ; N#Cc1cn([C@@H]2CCc3ccccc3[C@@H]2Nc2ncnc3[nH]c(C(F)(F)F)cc23)cn1 ; FC(F)(F)c1cc2c(N[C@@H]3c4ccccc4CC[C@H]3N3CCOCC3)ncnc2[nH]1 ; FC(F)(F)c1cc2c(N[C@@H]3c4ccccc4CC[C@@H]3N3CCOCC3)ncnc2[nH]1]

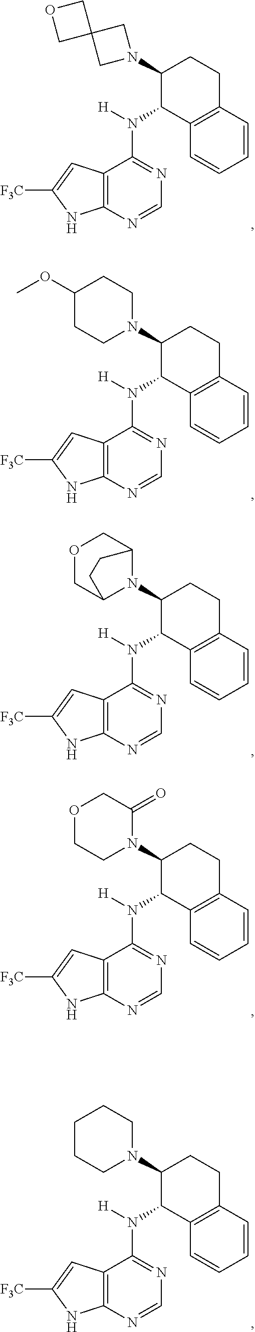 [SMILES: FC(F)(F)c1cc2c(N[C@H]3c4ccccc4CC[C@@H]3N3CC4(COC4)C3)ncnc2[nH]1 ; COC1CCN([C@H]2CCc3ccccc3[C@@H]2Nc2ncnc3[nH]c(C(F)(F)F)cc23)CC1 ; FC(F)(F)c1cc2c(N[C@H]3c4ccccc4CC[C@@H]3N3C4CCC3COC4)ncnc2[nH]1 ; O=C1COCCN1[C@H]1CCc2ccccc2[C@@H]1Nc1ncnc2[nH]c(C(F)(F)F)cc12 ; FC(F)(F)c1cc2c(N[C@H]3c4ccccc4CC[C@@H]3N3CCCCC3)ncnc2[nH]1]

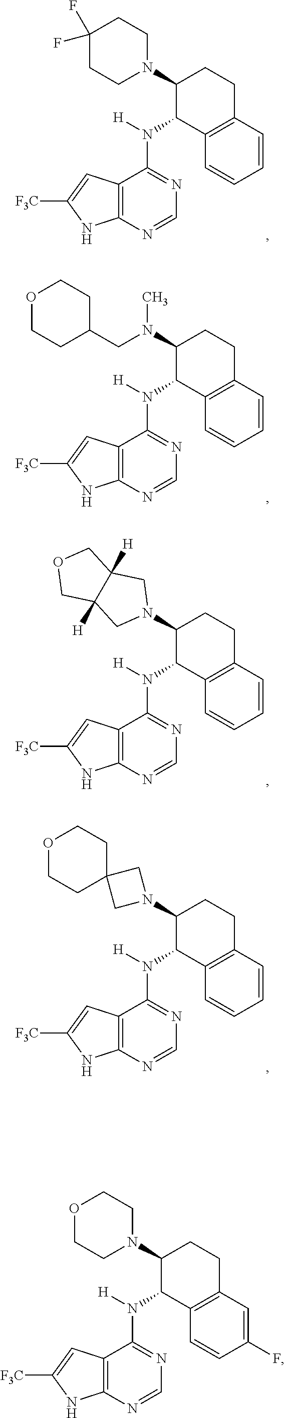 [SMILES: FC1(F)CCN([C@H]2CCc3ccccc3[C@@H]2Nc2ncnc3[nH]c(C(F)(F)F)cc23)CC1 ; CN(CC1CCOCC1)[C@H]1CCc2ccccc2[C@@H]1Nc1ncnc2[nH]c(C(F)(F)F)cc12 ; FC(F)(F)c1cc2c(N[C@H]3c4ccccc4CC[C@@H]3N3C[C@H]4COC[C@H]4C3)ncnc2[nH]1 ; FC(F)(F)c1cc2c(N[C@H]3c4ccccc4CC[C@@H]3N3CC4(CCOCC4)C3)ncnc2[nH]1 ; Fc1ccc2c(c1)CC[C@H](N1CCOCC1)[C@H]2Nc1ncnc2[nH]c(C(F)(F)F)cc12]

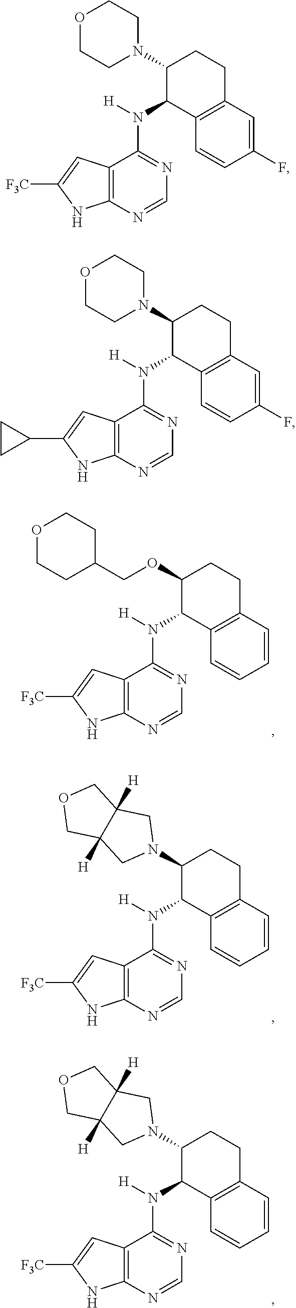

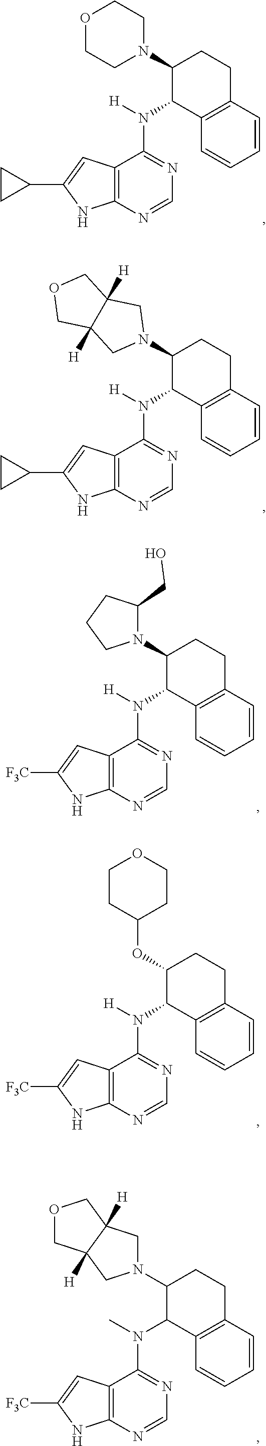 [SMILES: c1ccc2c(c1)CC[C@H](N1CCOCC1)[C@H]2Nc1ncnc2[nH]c(C3CC3)cc12 ; c1ccc2c(c1)CC[C@H](N1C[C@H]3COC[C@H]3C1)[C@H]2Nc1ncnc2[nH]c(C3CC3)cc12 ; OC[C@@H]1CCCN1[C@H]1CCc2ccccc2[C@@H]1Nc1ncnc2[nH]c(C(F)(F)F)cc12 ; FC(F)(F)c1cc2c(N[C@H]3c4ccccc4CC[C@H]3OC3CCOCC3)ncnc2[nH]1 ; CN(c1ncnc2[nH]c(C(F)(F)F)cc12)C1c2ccccc2CCC1N1C[C@H]2COC[C@H]2C1]

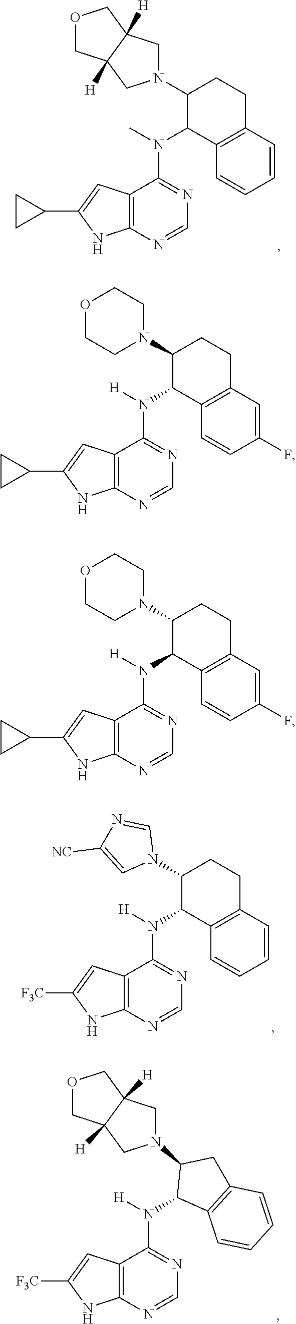 [SMILES: CN(c1ncnc2[nH]c(C3CC3)cc12)C1c2ccccc2CCC1N1C[C@H]2COC[C@H]2C1 ; Fc1ccc2c(c1)CC[C@H](N1CCOCC1)[C@H]2Nc1ncnc2[nH]c(C3CC3)cc12 ; Fc1ccc2c(c1)CC[C@@H](N1CCOCC1)[C@@H]2Nc1ncnc2[nH]c(C3CC3)cc12 ; N#Cc1cn([C@@H]2CCc3ccccc3[C@@H]2Nc2ncnc3[nH]c(C(F)(F)F)cc23)cn1 ; FC(F)(F)c1cc2c(N[C@H]3c4ccccc4C[C@@H]3N3C[C@H]4COC[C@H]4C3)ncnc2[nH]1]

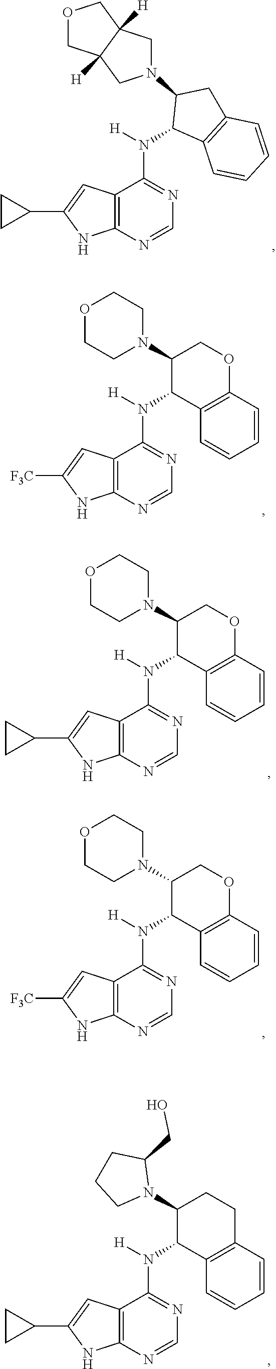 [SMILES: c1ccc2c(c1)C[C@H](N1C[C@H]3COC[C@H]3C1)[C@H]2Nc1ncnc2[nH]c(C3CC3)cc12 ; FC(F)(F)c1cc2c(N[C@H]3c4ccccc4OC[C@@H]3N3CCOCC3)ncnc2[nH]1 ; c1ccc2c(c1)OC[C@H](N1CCOCC1)[C@H]2Nc1ncnc2[nH]c(C3CC3)cc12 ; FC(F)(F)c1cc2c(N[C@H]3c4ccccc4OC[C@H]3N3CCOCC3)ncnc2[nH]1 ; OC[C@@H]1CCCN1[C@H]1CCc2ccccc2[C@@H]1Nc1ncnc2[nH]c(C3CC3)cc12]

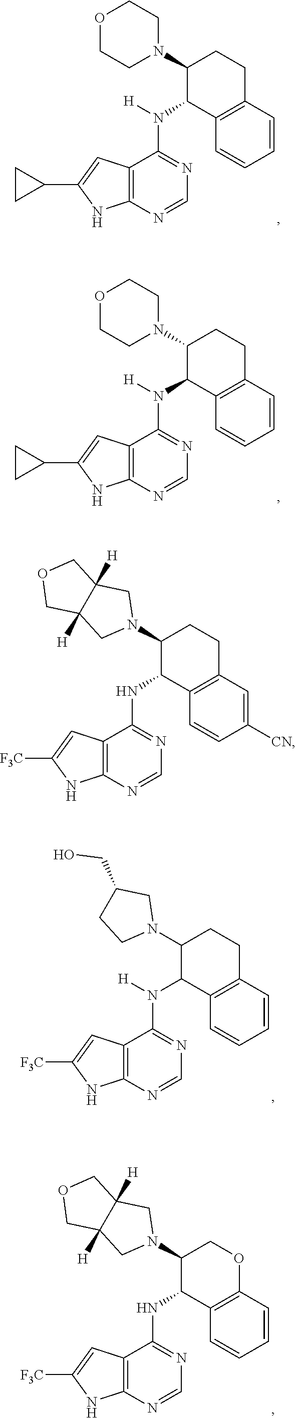 [SMILES: c1ccc2c(c1)CC[C@H](N1CCOCC1)[C@H]2Nc1ncnc2[nH]c(C3CC3)cc12 ; c1ccc2c(c1)CC[C@@H](N1CCOCC1)[C@@H]2Nc1ncnc2[nH]c(C3CC3)cc12 ; N#Cc1ccc2c(c1)CC[C@H](N1C[C@H]3COC[C@H]3C1)[C@H]2Nc1ncnc2[nH]c(C(F)(F)F)cc12 ; OC[C@H]1CCN(C2CCc3ccccc3C2Nc2ncnc3[nH]c(C(F)(F)F)cc23)C1 ; FC(F)(F)c1cc2c(N[C@H]3c4ccccc4OC[C@@H]3N3C[C@H]4COC[C@H]4C3)ncnc2[nH]1]

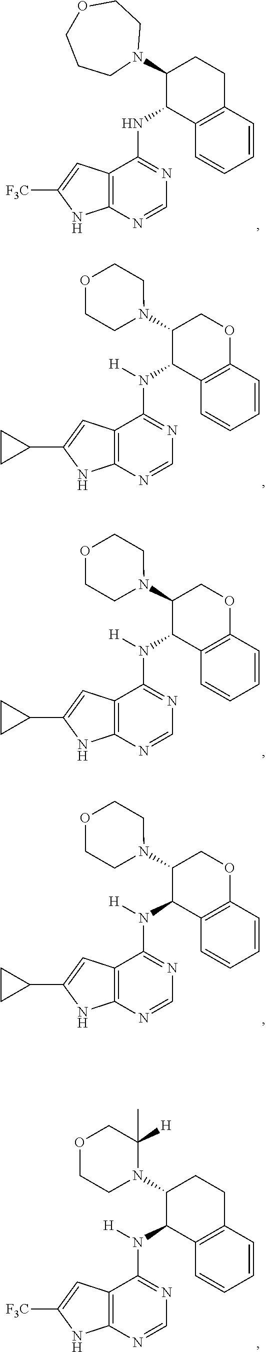

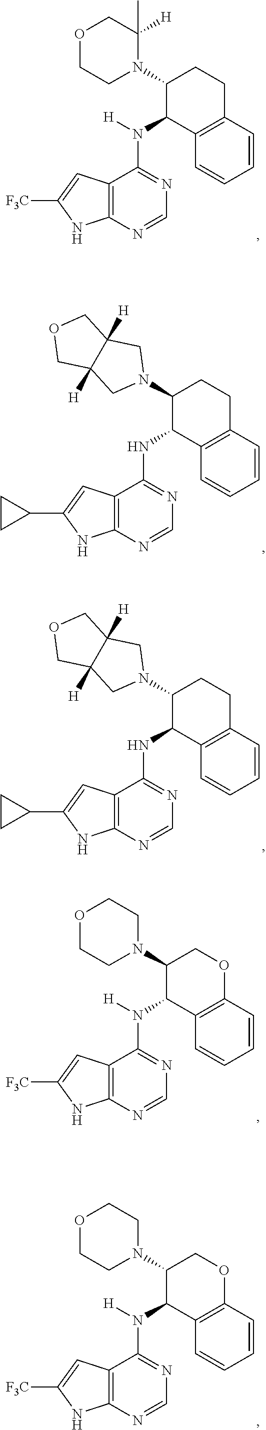 [SMILES: C[C@@H]1COCCN1[C@@H]1CCc2ccccc2[C@H]1Nc1ncnc2[nH]c(C(F)(F)F)cc12 ; c1ccc2c(c1)CC[C@H](N1C[C@H]3COC[C@H]3C1)[C@H]2Nc1ncnc2[nH]c(C3CC3)cc12 ; c1ccc2c(c1)CC[C@@H](N1C[C@H]3COC[C@H]3C1)[C@@H]2Nc1ncnc2[nH]c(C3CC3)cc12 ; FC(F)(F)c1cc2c(N[C@H]3c4ccccc4OC[C@@H]3N3CCOCC3)ncnc2[nH]1 ; FC(F)(F)c1cc2c(N[C@@H]3c4ccccc4OC[C@H]3N3CCOCC3)ncnc2[nH]1]

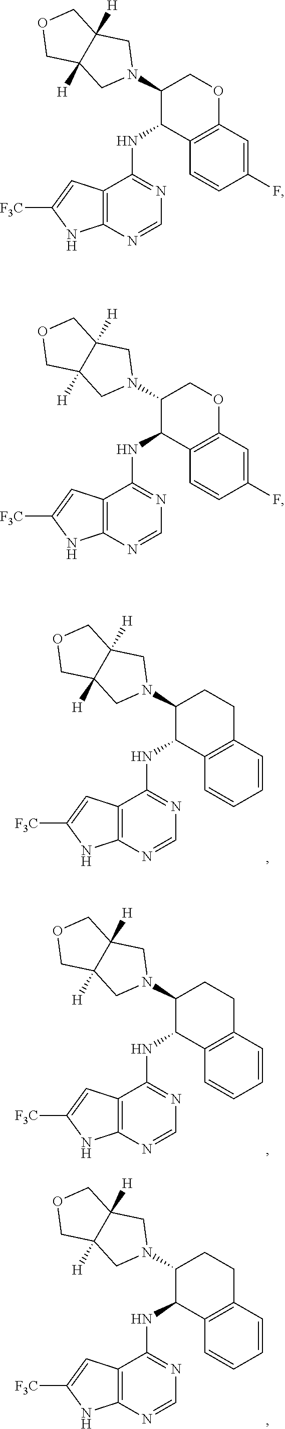

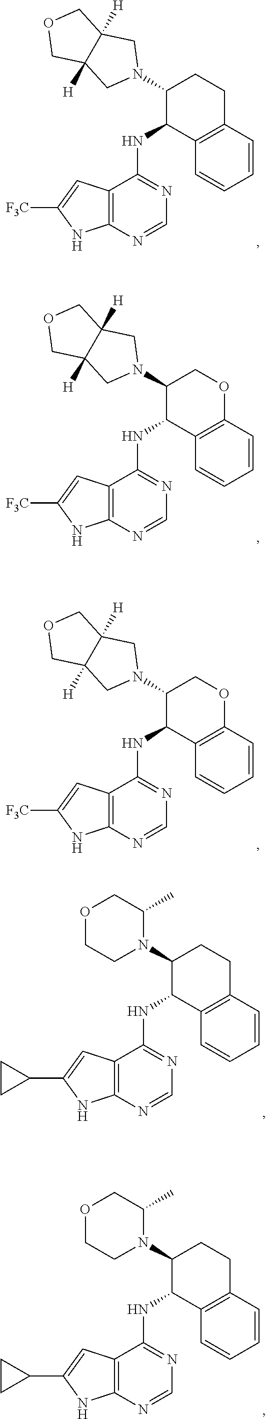 [SMILES: FC(F)(F)c1cc2c(N[C@@H]3c4ccccc4CC[C@H]3N3C[C@@H]4COC[C@H]4C3)ncnc2[nH]1 ; FC(F)(F)c1cc2c(N[C@H]3c4ccccc4OC[C@@H]3N3C[C@H]4COC[C@H]4C3)ncnc2[nH]1 ; FC(F)(F)c1cc2c(N[C@@H]3c4ccccc4OC[C@H]3N3C[C@H]4COC[C@H]4C3)ncnc2[nH]1 ; C[C@H]1COCCN1[C@H]1CCc2ccccc2[C@@H]1Nc1ncnc2[nH]c(C3CC3)cc12 ; C[C@H]1COCCN1[C@H]1CCc2ccccc2[C@@H]1Nc1ncnc2[nH]c(C3CC3)cc12]

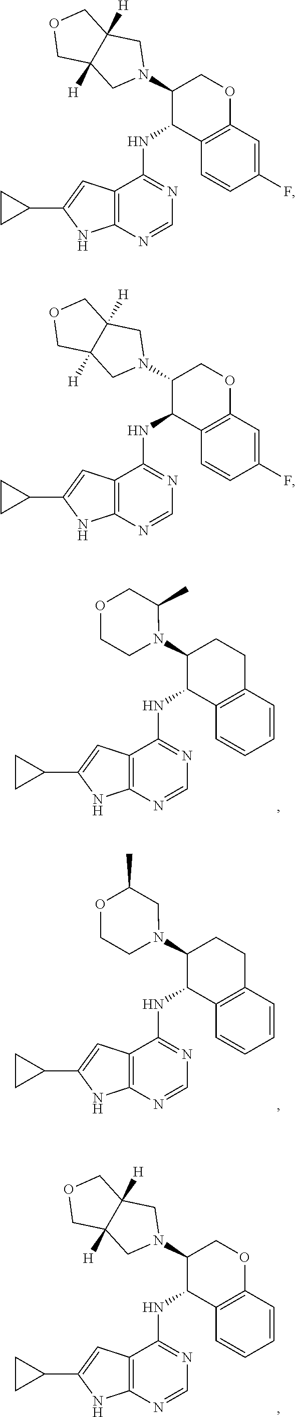 [SMILES: Fc1ccc2c(c1)OC[C@H](N1C[C@H]3COC[C@H]3C1)[C@H]2Nc1ncnc2[nH]c(C3CC3)cc12 ; Fc1ccc2c(c1)OC[C@@H](N1C[C@H]3COC[C@H]3C1)[C@@H]2Nc1ncnc2[nH]c(C3CC3)cc12 ; C[C@@H]1COCCN1[C@H]1CCc2ccccc2[C@@H]1Nc1ncnc2[nH]c(C3CC3)cc12 ; C[C@H]1CN([C@H]2CCc3ccccc3[C@@H]2Nc2ncnc3[nH]c(C4CC4)cc23)CCO1 ; c1ccc2c(c1)OC[C@H](N1C[C@H]3COC[C@H]3C1)[C@H]2Nc1ncnc2[nH]c(C3CC3)cc12]

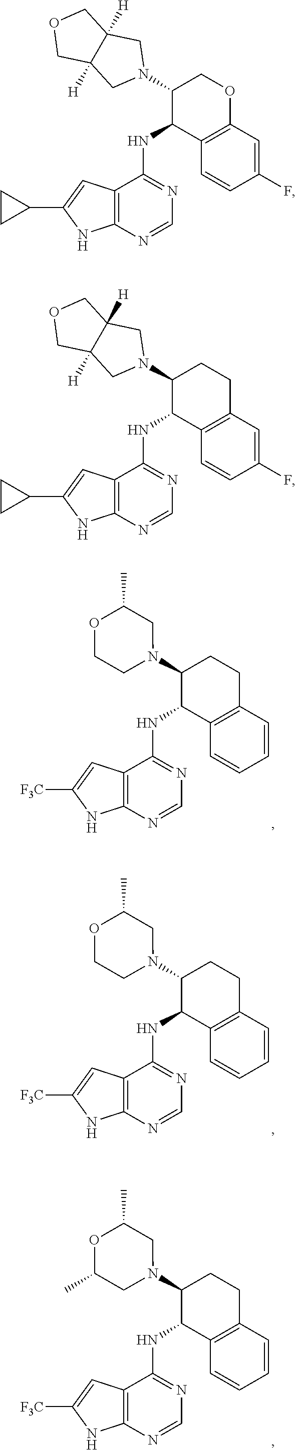 [SMILES: Fc1ccc2c(c1)OC[C@@H](N1C[C@H]3COC[C@H]3C1)[C@@H]2Nc1ncnc2[nH]c(C3CC3)cc12 ; Fc1ccc2c(c1)CC[C@H](N1C[C@H]3COC[C@@H]3C1)[C@H]2Nc1ncnc2[nH]c(C3CC3)cc12 ; C[C@@H]1CN([C@H]2CCc3ccccc3[C@@H]2Nc2ncnc3[nH]c(C(F)(F)F)cc23)CCO1 ; C[C@@H]1CN([C@@H]2CCc3ccccc3[C@H]2Nc2ncnc3[nH]c(C(F)(F)F)cc23)CCO1 ; C[C@@H]1CN([C@H]2CCc3ccccc3[C@@H]2Nc2ncnc3[nH]c(C(F)(F)F)cc23)C[C@H](C)O1]

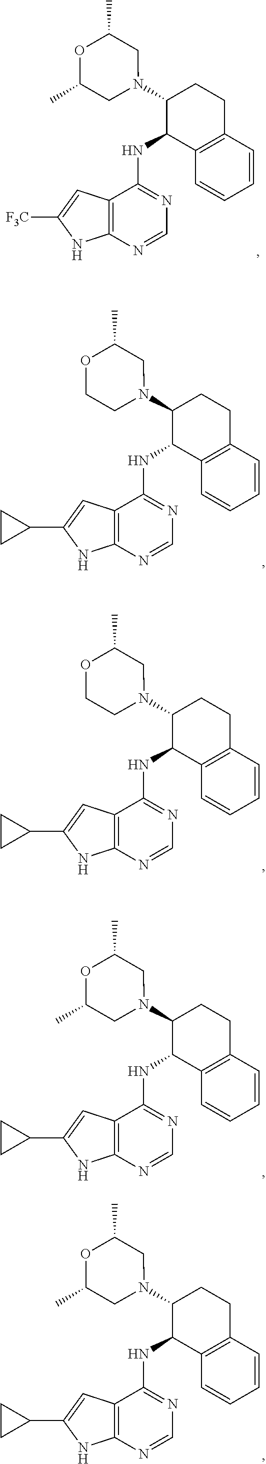

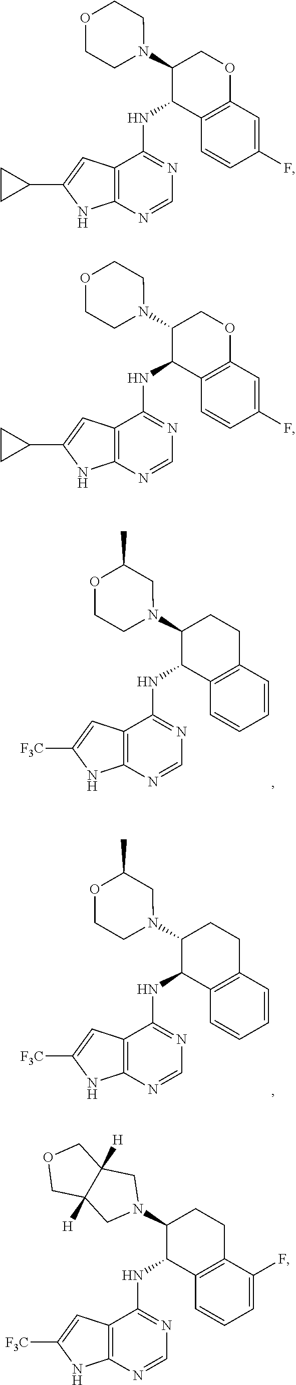 [SMILES: Fc1ccc2c(c1)OC[C@H](N1CCOCC1)[C@H]2Nc1ncnc2[nH]c(C3CC3)cc12 ; Fc1ccc2c(c1)OC[C@@H](N1CCOCC1)[C@@H]2Nc1ncnc2[nH]c(C3CC3)cc12 ; C[C@H]1CN([C@H]2CCc3ccccc3[C@@H]2Nc2ncnc3[nH]c(C(F)(F)F)cc23)CCO1 ; C[C@H]1CN([C@@H]2CCc3ccccc3[C@H]2Nc2ncnc3[nH]c(C(F)(F)F)cc23)CCO1 ; Fc1cccc2c1CC[C@H](N1C[C@H]3COC[C@H]3C1)[C@H]2Nc1ncnc2[nH]c(C(F)(F)F)cc12]

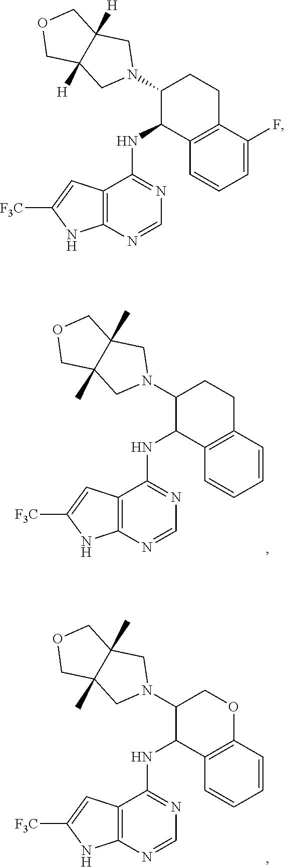 [SMILES: Fc1cccc2c1CC[C@@H](N1C[C@H]3COC[C@H]3C1)[C@@H]2Nc1ncnc2[nH]c(C(F)(F)F)cc12 ; C[C@@]12COC[C@]1(C)CN(C1CCc3ccccc3C1Nc1ncnc3[nH]c(C(F)(F)F)cc13)C2 ; C[C@@]12COC[C@]1(C)CN(C1COc3ccccc3C1Nc1ncnc3[nH]c(C(F)(F)F)cc13)C2]

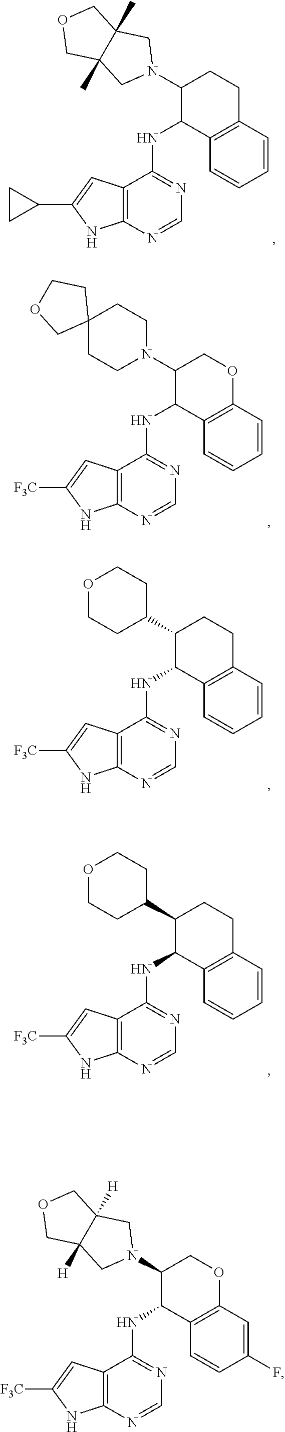 [SMILES: C[C@@]12COC[C@]1(C)CN(C1CCc3ccccc3C1Nc1ncnc3[nH]c(C4CC4)cc13)C2 ; FC(F)(F)c1cc2c(NC3c4ccccc4OCC3N3CCC4(CCOC4)CC3)ncnc2[nH]1 ; FC(F)(F)c1cc2c(N[C@H]3c4ccccc4CC[C@H]3C3CCOCC3)ncnc2[nH]1 ; FC(F)(F)c1cc2c(N[C@@H]3c4ccccc4CC[C@@H]3C3CCOCC3)ncnc2[nH]1 ; Fc1ccc2c(c1)OC[C@H](N1C[C@@H]3COC[C@H]3C1)[C@H]2Nc1ncnc2[nH]c(C(F)(F)F)cc12]

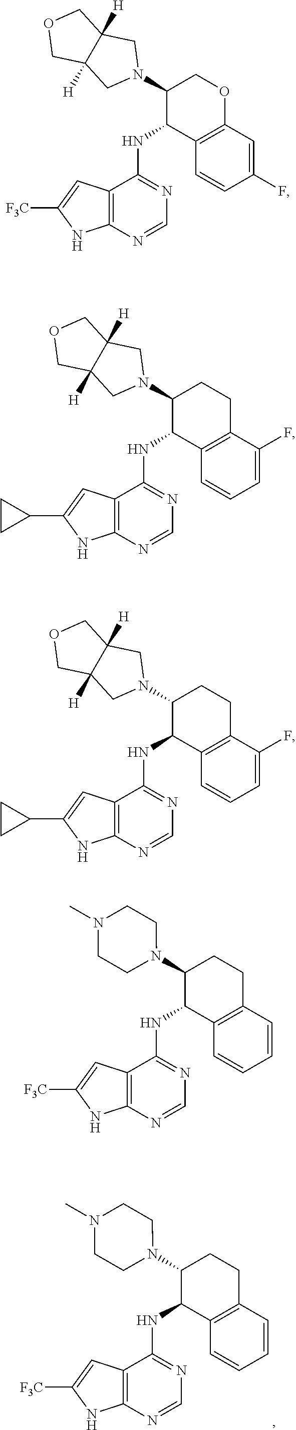 [SMILES: Fc1ccc2c(c1)OC[C@H](N1C[C@H]3COC[C@@H]3C1)[C@H]2Nc1ncnc2[nH]c(C(F)(F)F)cc12 ; Fc1cccc2c1CC[C@H](N1C[C@H]3COC[C@H]3C1)[C@H]2Nc1ncnc2[nH]c(C3CC3)cc12 ; Fc1cccc2c1CC[C@@H](N1C[C@H]3COC[C@H]3C1)[C@@H]2Nc1ncnc2[nH]c(C3CC3)cc12 ; CN1CCN([C@H]2CCc3ccccc3[C@@H]2Nc2ncnc3[nH]c(C(F)(F)F)cc23)CC1 ; CN1CCN([C@@H]2CCc3ccccc3[C@H]2Nc2ncnc3[nH]c(C(F)(F)F)cc23)CC1]

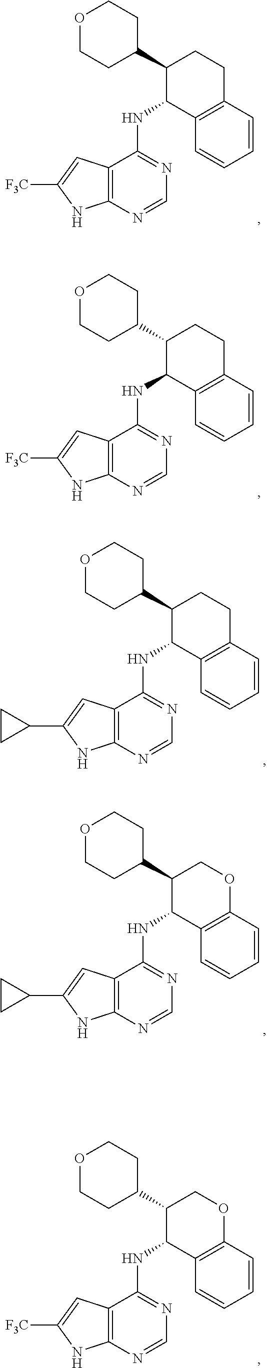 [SMILES: FC(F)(F)c1cc2c(N[C@H]3c4ccccc4CC[C@@H]3C3CCOCC3)ncnc2[nH]1 ; FC(F)(F)c1cc2c(N[C@@H]3c4ccccc4CC[C@H]3C3CCOCC3)ncnc2[nH]1 ; c1ccc2c(c1)CC[C@H](C1CCOCC1)[C@H]2Nc1ncnc2[nH]c(C3CC3)cc12 ; c1ccc2c(c1)OC[C@H](C1CCOCC1)[C@H]2Nc1ncnc2[nH]c(C3CC3)cc12 ; FC(F)(F)c1cc2c(N[C@H]3c4ccccc4OC[C@H]3C3CCOCC3)ncnc2[nH]1]

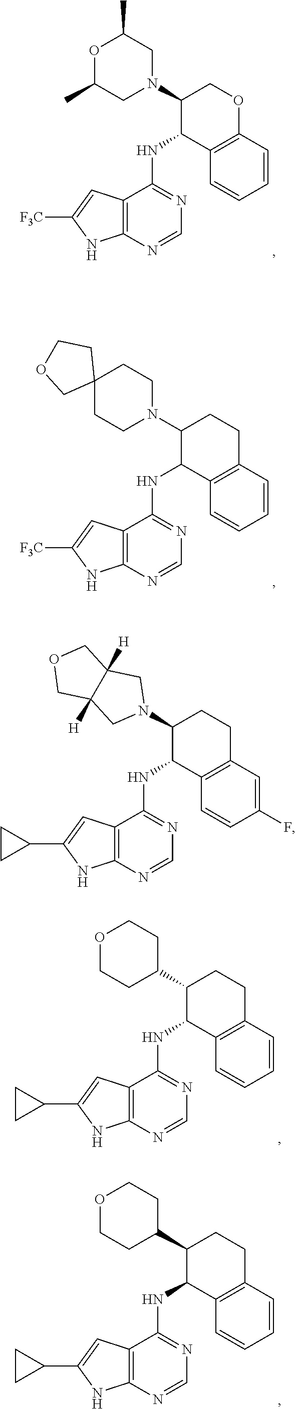

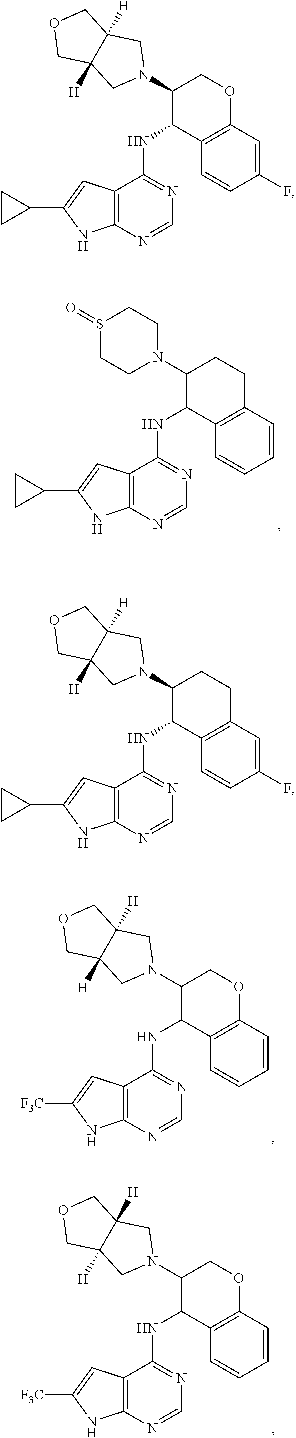 [SMILES: Fc1ccc2c(c1)OC[C@H](N1C[C@@H]3COC[C@H]3C1)[C@H]2Nc1ncnc2[nH]c(C3CC3)cc12 ; O=S1CCN(C2CCc3ccccc3C2Nc2ncnc3[nH]c(C4CC4)cc23)CC1 ; Fc1ccc2c(c1)CC[C@H](N1C[C@@H]3COC[C@H]3C1)[C@H]2Nc1ncnc2[nH]c(C3CC3)cc12 ; FC(F)(F)c1cc2c(NC3c4ccccc4OCC3N3C[C@@H]4COC[C@H]4C3)ncnc2[nH]1 ; FC(F)(F)c1cc2c(NC3c4ccccc4OCC3N3C[C@H]4COC[C@@H]4C3)ncnc2[nH]1]

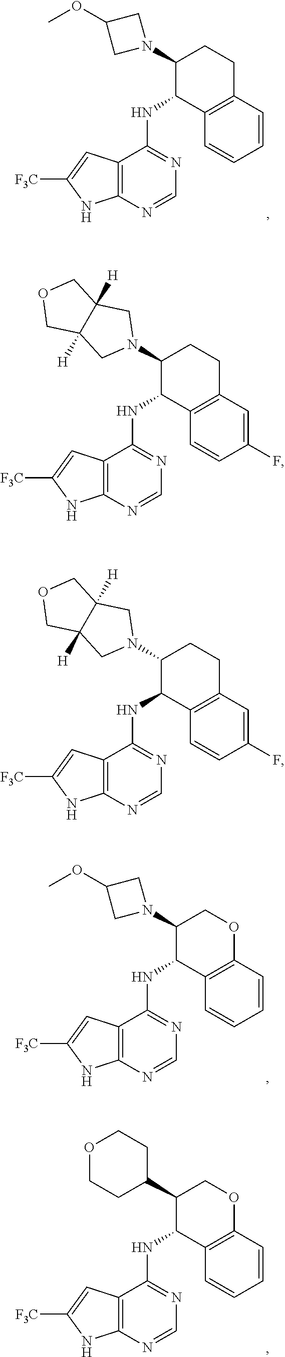 [SMILES: COC1CN([C@H]2CCc3ccccc3[C@@H]2Nc2ncnc3[nH]c(C(F)(F)F)cc23)C1 ; Fc1ccc2c(c1)CC[C@H](N1C[C@H]3COC[C@@H]3C1)[C@H]2Nc1ncnc2[nH]c(C(F)(F)F)cc12 ; Fc1ccc2c(c1)CC[C@@H](N1C[C@@H]3COC[C@H]3C1)[C@@H]2Nc1ncnc2[nH]c(C(F)(F)F)cc12 ; COC1CN([C@H]2COc3ccccc3[C@@H]2Nc2ncnc3[nH]c(C(F)(F)F)cc23)C1 ; FC(F)(F)c1cc2c(N[C@H]3c4ccccc4OC[C@@H]3C3CCOCC3)ncnc2[nH]1]

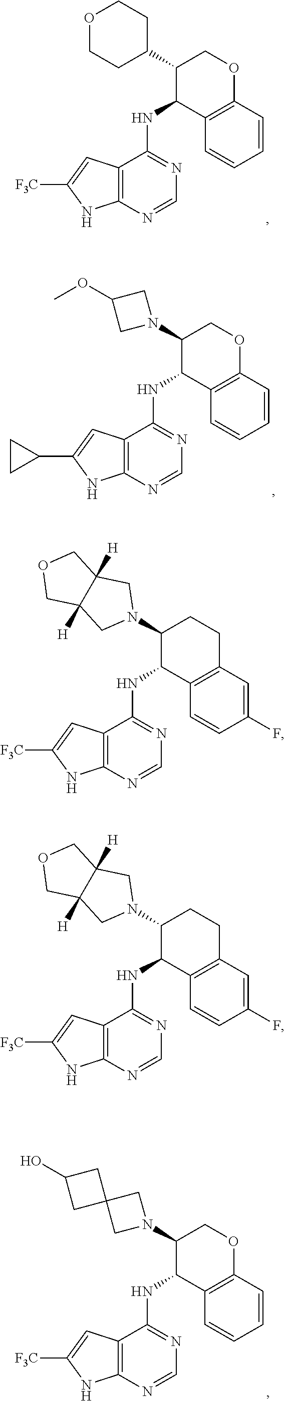 [SMILES: FC(F)(F)c1cc2c(N[C@@H]3c4ccccc4OC[C@H]3C3CCOCC3)ncnc2[nH]1 ; COC1CN([C@H]2COc3ccccc3[C@@H]2Nc2ncnc3[nH]c(C4CC4)cc23)C1 ; Fc1ccc2c(c1)CC[C@H](N1C[C@H]3COC[C@H]3C1)[C@H]2Nc1ncnc2[nH]c(C(F)(F)F)cc12 ; Fc1ccc2c(c1)CC[C@@H](N1C[C@H]3COC[C@H]3C1)[C@@H]2Nc1ncnc2[nH]c(C(F)(F)F)cc12 ; OC1CC2(C1)CN([C@H]1COc3ccccc3[C@@H]1Nc1ncnc3[nH]c(C(F)(F)F)cc13)C2]

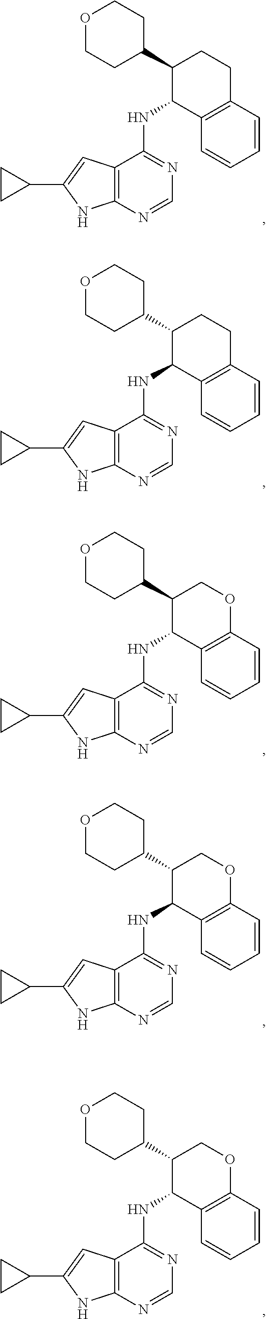 [SMILES: c1ccc2c(c1)CC[C@H](C1CCOCC1)[C@H]2Nc1ncnc2[nH]c(C3CC3)cc12 ; c1ccc2c(c1)CC[C@@H](C1CCOCC1)[C@@H]2Nc1ncnc2[nH]c(C3CC3)cc12 ; c1ccc2c(c1)OC[C@H](C1CCOCC1)[C@H]2Nc1ncnc2[nH]c(C3CC3)cc12 ; c1ccc2c(c1)OC[C@@H](C1CCOCC1)[C@@H]2Nc1ncnc2[nH]c(C3CC3)cc12 ; c1ccc2c(c1)OC[C@@H](C1CCOCC1)[C@H]2Nc1ncnc2[nH]c(C3CC3)cc12]

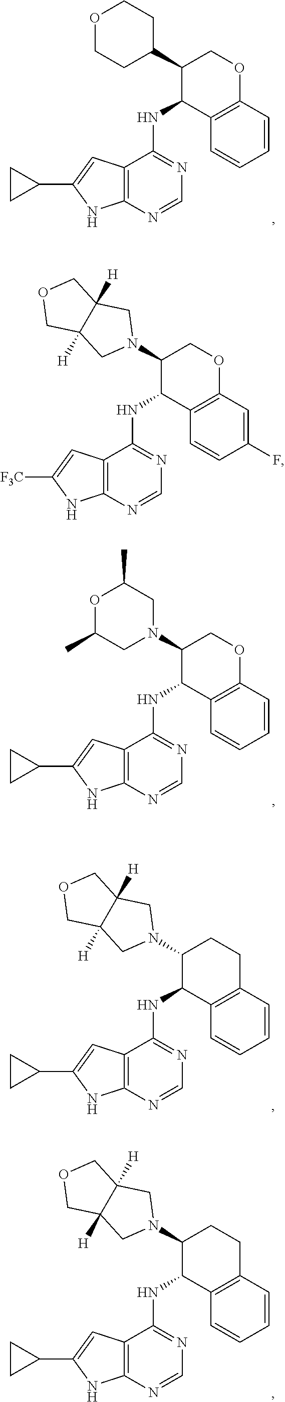

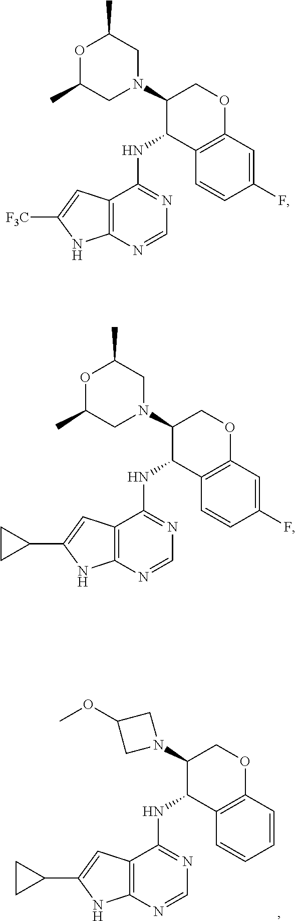 [SMILES: C[C@@H]1CN([C@H]2COc3cc(F)ccc3[C@@H]2Nc2ncnc3[nH]c(C(F)(F)F)cc23)C[C@H](C)O1 ; C[C@@H]1CN([C@H]2COc3cc(F)ccc3[C@@H]2Nc2ncnc3[nH]c(C4CC4)cc23)C[C@H](C)O1 ; COC1CN([C@H]2COc3ccccc3[C@@H]2Nc2ncnc3[nH]c(C4CC4)cc23)C1]

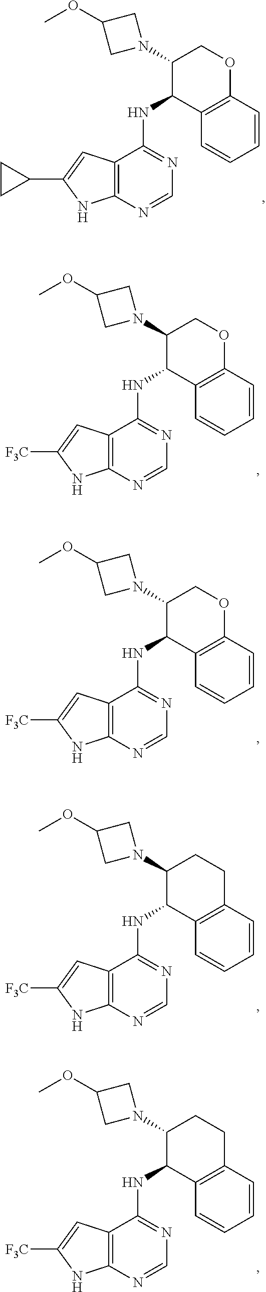 [SMILES: COC1CN([C@@H]2COc3ccccc3[C@H]2Nc2ncnc3[nH]c(C4CC4)cc23)C1 ; COC1CN([C@H]2COc3ccccc3[C@@H]2Nc2ncnc3[nH]c(C(F)(F)F)cc23)C1 ; COC1CN([C@@H]2COc3ccccc3[C@H]2Nc2ncnc3[nH]c(C(F)(F)F)cc23)C1 ; COC1CN([C@H]2CCc3ccccc3[C@@H]2Nc2ncnc3[nH]c(C(F)(F)F)cc23)C1 ; COC1CN([C@@H]2CCc3ccccc3[C@H]2Nc2ncnc3[nH]c(C(F)(F)F)cc23)C1]

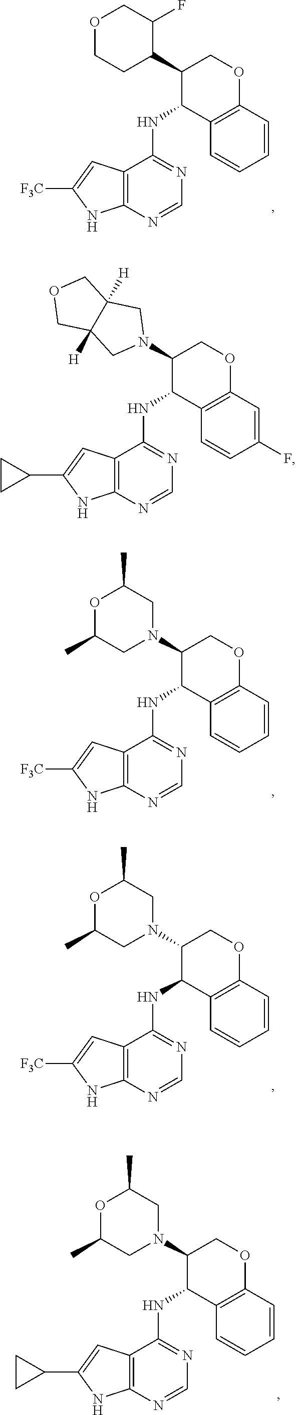 [SMILES: FC1COCCC1[C@H]1COc2ccccc2[C@@H]1Nc1ncnc2[nH]c(C(F)(F)F)cc12 ; Fc1ccc2c(c1)OC[C@H](N1C[C@@H]3COC[C@H]3C1)[C@H]2Nc1ncnc2[nH]c(C3CC3)cc12 ; C[C@@H]1CN([C@H]2COc3ccccc3[C@@H]2Nc2ncnc3[nH]c(C(F)(F)F)cc23)C[C@H](C)O1 ; C[C@@H]1CN([C@@H]2COc3ccccc3[C@H]2Nc2ncnc3[nH]c(C(F)(F)F)cc23)C[C@H](C)O1 ; C[C@@H]1CN([C@H]2COc3ccccc3[C@@H]2Nc2ncnc3[nH]c(C4CC4)cc23)C[C@H](C)O1]

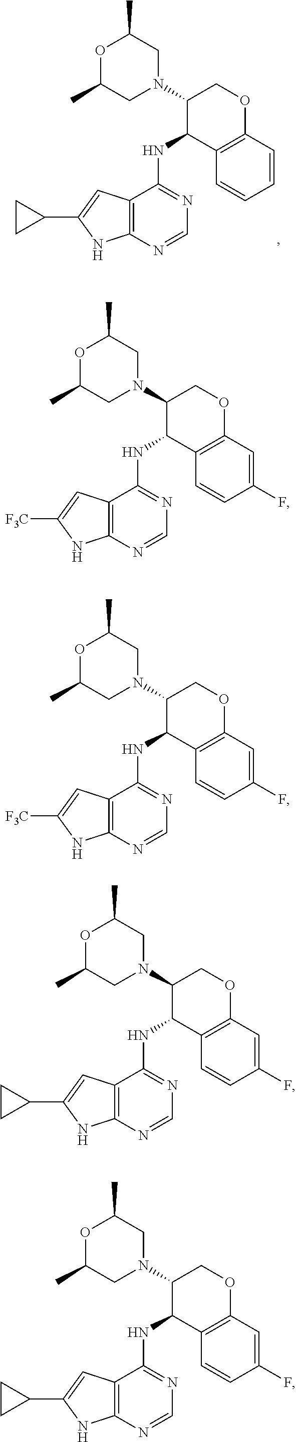

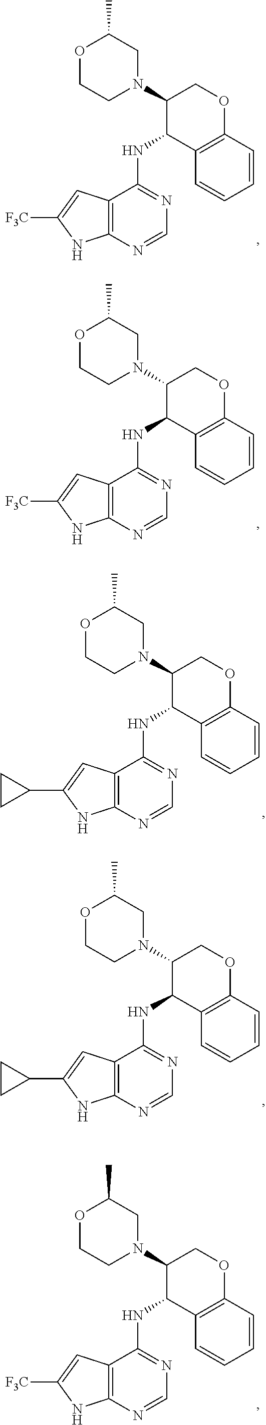

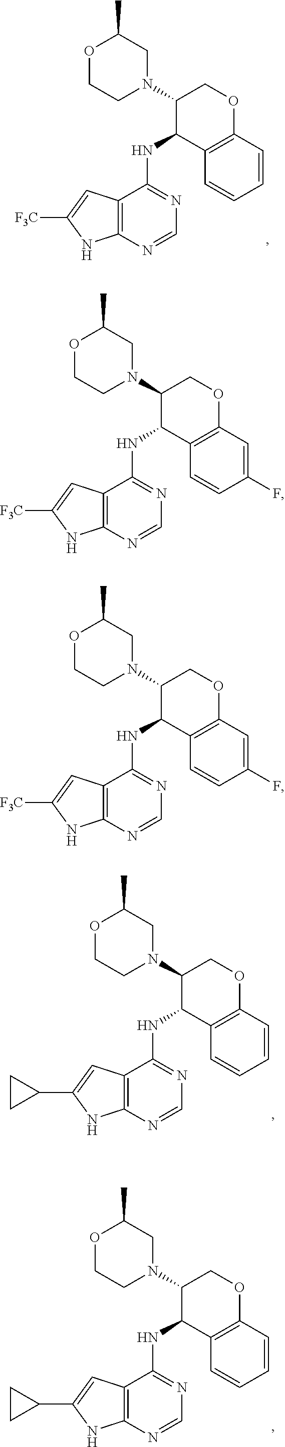

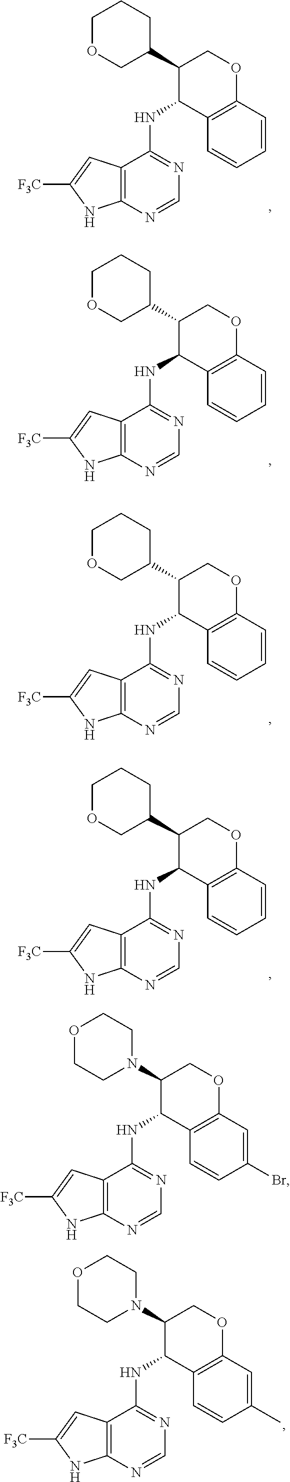

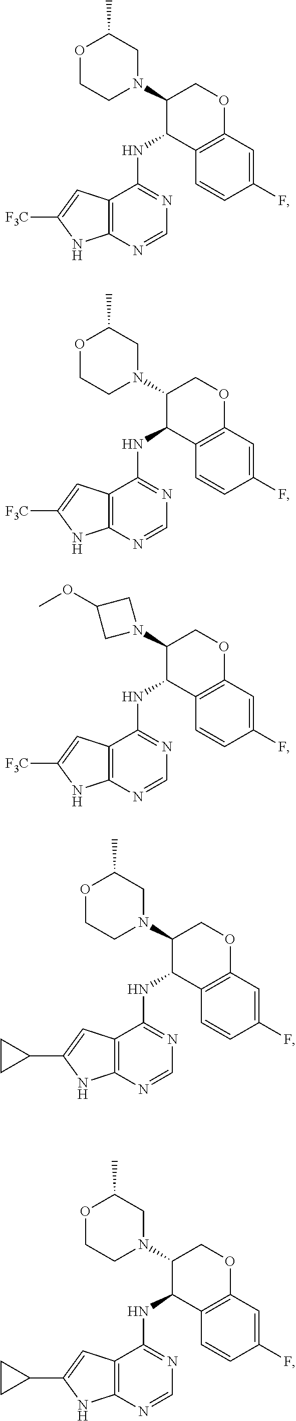 [SMILES: C[C@@H]1CN([C@H]2COc3cc(F)ccc3[C@@H]2Nc2ncnc3[nH]c(C(F)(F)F)cc23)CCO1 ; C[C@@H]1CN([C@@H]2COc3cc(F)ccc3[C@H]2Nc2ncnc3[nH]c(C(F)(F)F)cc23)CCO1 ; COC1CN([C@H]2COc3cc(F)ccc3[C@@H]2Nc2ncnc3[nH]c(C(F)(F)F)cc23)C1 ; C[C@@H]1CN([C@H]2COc3cc(F)ccc3[C@@H]2Nc2ncnc3[nH]c(C4CC4)cc23)CCO1 ; C[C@@H]1CN([C@@H]2COc3cc(F)ccc3[C@H]2Nc2ncnc3[nH]c(C4CC4)cc23)CCO1]

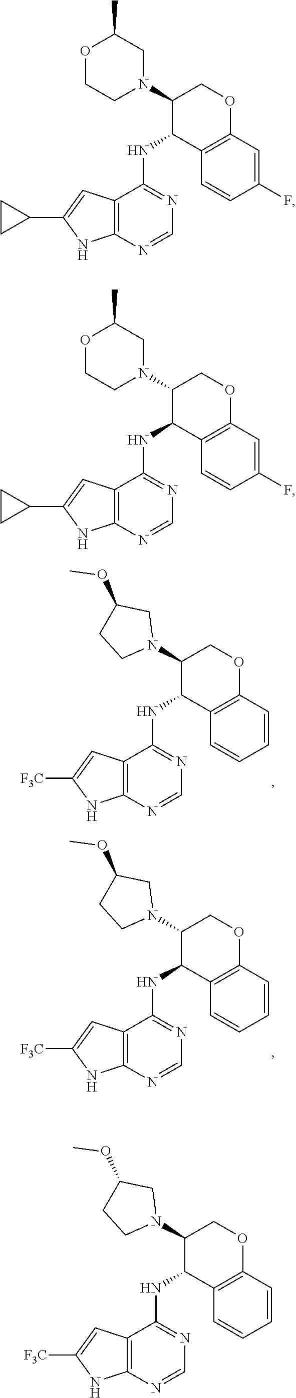

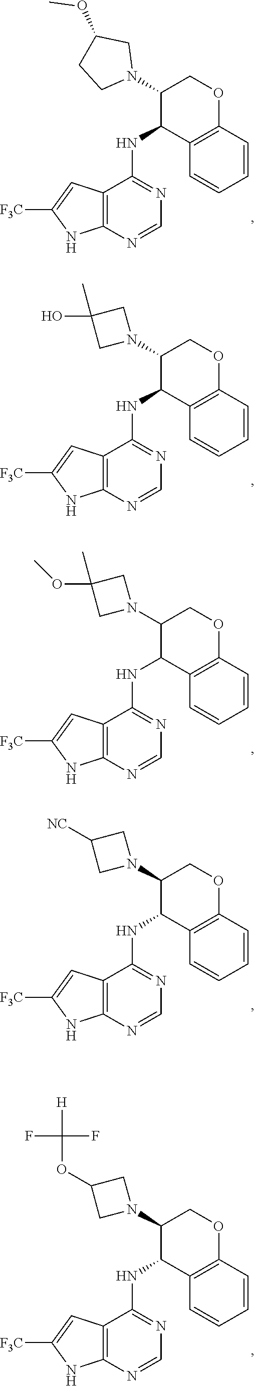 [SMILES: CO[C@H]1CCN([C@@H]2COc3ccccc3[C@H]2Nc2ncnc3[nH]c(C(F)(F)F)cc23)C1 ; CC1(O)CN([C@@H]2COc3ccccc3[C@H]2Nc2ncnc3[nH]c(C(F)(F)F)cc23)C1 ; COC1(C)CN(C2COc3ccccc3C2Nc2ncnc3[nH]c(C(F)(F)F)cc23)C1 ; N#CC1CN([C@H]2COc3ccccc3[C@@H]2Nc2ncnc3[nH]c(C(F)(F)F)cc23)C1 ; FC(F)OC1CN([C@H]2COc3ccccc3[C@@H]2Nc2ncnc3[nH]c(C(F)(F)F)cc23)C1]

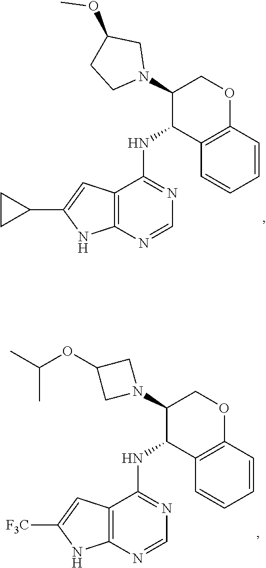 [SMILES: CO[C@@H]1CCN([C@H]2COc3ccccc3[C@@H]2Nc2ncnc3[nH]c(C4CC4)cc23)C1 ; CC(C)OC1CN([C@H]2COc3ccccc3[C@@H]2Nc2ncnc3[nH]c(C(F)(F)F)cc23)C1]

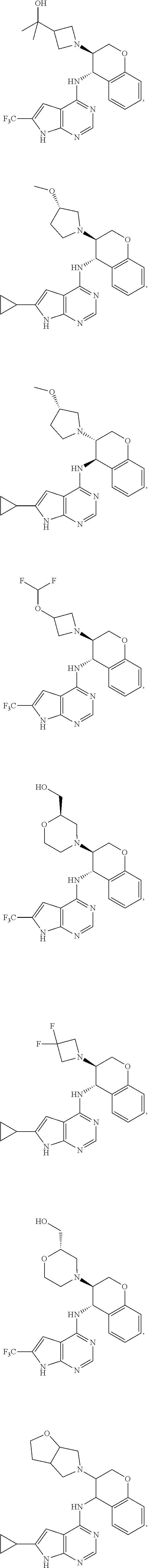

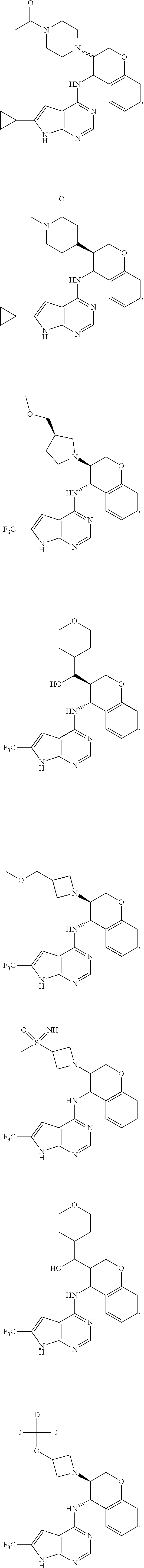

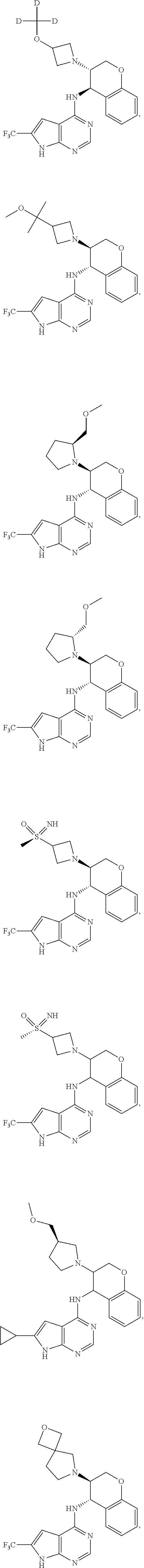

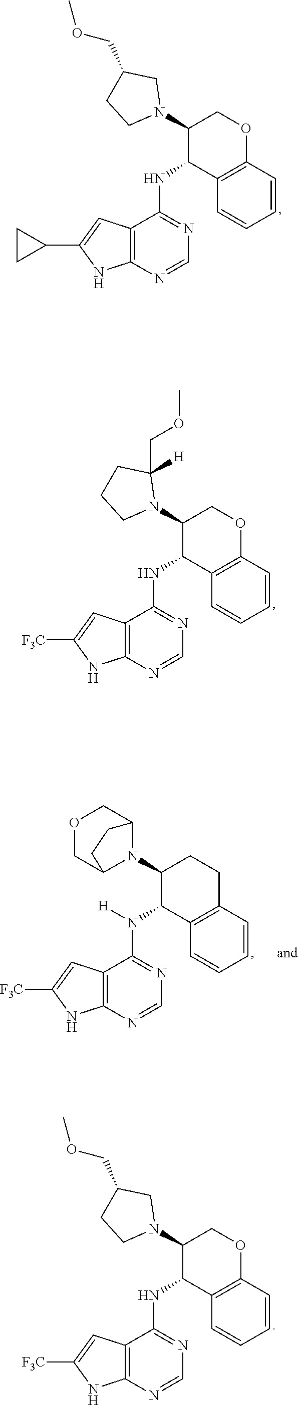 [SMILES: COC[C@H]1CCN([C@H]2COc3ccccc3[C@@H]2Nc2ncnc3[nH]c(C4CC4)cc23)C1 ; COC[C@H]1CCCN1[C@H]1COc2ccccc2[C@@H]1Nc1ncnc2[nH]c(C(F)(F)F)cc12 ; FC(F)(F)c1cc2c(N[C@H]3c4ccccc4CC[C@@H]3N3C4CCC3COC4)ncnc2[nH]1 ; COC[C@H]1CCN([C@H]2COc3ccccc3[C@@H]2Nc2ncnc3[nH]c(C(F)(F)F)cc23)C1]

32. The compound of claim 1, wherein the compound is:

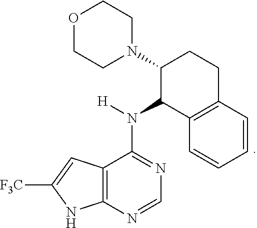

33. A pharmaceutical composition comprising a therapeutically effective amount of the compound of any one of claims 1-32, and a pharmaceutically acceptable carrier.

34. A method of modulating PINK1 kinase activity in a subject in need thereof, the method comprising administering to the subject in need thereof an effective amount of the compound of any one of claims 1-32.

35. The method of claim 34, wherein the modulating is inhibiting.

36. A method of modulating PINK1 kinase activity in at least one cell, the method comprising contacting the cell with an effective amount of the compound of any one of claims 1-32.

37. The method of claim 36, wherein the cell is mammalian.

38. The method of claim 37, wherein the cell has been isolated from a mammal prior to the contacting step.

39. The method of claim 36, wherein the cell comprises a dysfunctional PINK1 kinase activity.

40. The method of claim 36, wherein the step of contacting is performed in vitro.

41. A method of treating a disorder in a subject in need thereof, the method comprising administering to the subject in need thereof an effective amount of the compound of any one of claims 1-32, wherein the disorder is a neurodegenerative disorder, a mitochondrial disorder, a fibrosis, or cardiomyopathy.

42. The method of claim 41, wherein the subject is a mammal.

43. The method of claim 41, wherein the subject is a human.

44. The method of claim 41, wherein the subject has been diagnosed with the disorder prior to the administering step.

45. The method of claim 41, wherein the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

46. The method of claim 41, wherein the administering comprises administering from about 1 to about 2000 micrograms of expressible nucleic acid sequence.

47. The method of claim 41, wherein the neurodegenerative disorder is Parkinson's disease, Huntington's disease, or amyotrophic lateral sclerosis.

48. A kit comprising the compound of any one of claims 1-32, and one or more of:
(a) at least one agent known for the treatment of a neurodegenerative disorder, a mitochondrial disorder, a fibrosis, and cardiomyopathy;
(b) instructions for administering the compound in connection with the neurodegenerative disorder, a mitochondrial disorder, a fibrosis, or cardiomyopathy; and/or
(c) instructions for treating the disorder.


SEQUENCE LISTING (19 sequences)

SEQ ID NO: 1 - protein, 581 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 2 - protein, 580 aa, Mus musculus
MAVRQALGRGLQLGRALLLRFAPKPGPLFGWGKPGPAAAWGRGERPGQVVSPGAQPRPVGLPLPDRYRFFRQSVAGLAARIQRQFMVRARGGAGPCGRAVFLAFGLGLGLIEEKQAEGRRAASACQEIQAIFTQKTKRVSDPLDTRCWQGFRLEDYLIGQAIGKGCNAAVYEATMPTLPQHLEKAKHLGLIGKGPDVVLKGADGEQAPGTPTFPFAIKMMWNISAGSSSEAILSKMSQELVPASRVALAGEYGAVTYRRSRDGPKQLAPHPNIIRVFRAFTSSVPLLPGALADYPDMLPPHYYPEGLGHGRTLFLVMKNYPCTLRQYLEEQTPSSRLATMMTLQLLEGVDHLVQQGIAHRDLKSDNILVEWDSDGCPWLVISDFGCCLADQHVGLRLPFNSSSVERGGNGSLMAPEVSTAHSGPSAVIDYSKADTWAVGAIAYEIFGLANPFYGQGSAHLESRSYQEAQLPEMPESVPPEARRLVRSLLQREASKRPSARLAANVLHLSLWGEHLLALKNLKLDKMIAWLLQQSAATLLADRLREKSCVETKLQMLFLANLECEALCQAALLLSSWRAAP

SEQ ID NO: 3 - protein, 580 aa, Rattus norvegicus
MAVRQALGRGLQLGRALLLRFAPKPGPVSGWGKPGPGAAWGRGERPGRVSSPGAQPRPLGLPLPDRYRFFRQSVAGLAARIQRQFVVRARGGAGPCGRAVFLAFGLGLGLIEEKQAESRRAASACQEIQAIFTQKNKQVSDPLDTRRWQGFRLEDYLIGQAIGKGCNAAVYEATMPTLPQHLEKAKHLGLLGKGPDVVSKGADGEQAPGAPAFPFAIKMMWNISAGSSSEAILSKMSQELVPASRMALDGEYGAVTYRRSRDGPKQLAPHPNIIRVFRAFTSSVPLLPGALADYPDMLPPHYYPEGLGHGRTLFLVMKNYPCTLRQYLEEQTPSSRLATMMTLQLLEGVDHLVQQGIAHRDLKSDNILVEWDSDGCPWLVISDFGCCLADERVGLQLPFNSSSVERGGNGSLMAPEVSTAHSGPHAVIDYSKADTWAVGAIAYEIFGLANPFYGQGSAHLESRSYQEAQLPEMPKSVPPETRQLVRSLLQREANKRPSARIAANVLHLSLWGEHLLALKNLKLDKMIAWLLQQSAATLLADRLREKSCVETKLQMLFLANLECEALCQAALLLSSWRAAP

SEQ ID NO: 4 - protein, 20 aa, synthetic construct
  REGION 1..20: note = Synthetic construct
GTGTTGGGATTCGGCTTGGT

SEQ ID NO: 5 - protein, 21 aa, synthetic construct
  REGION 1..21: note = Synthetic construct
GTCCATGCACTTGTCGAGGTC

SEQ ID NO: 6 - protein, 23 aa, synthetic construct
  REGION 1..23: note = Synthetic construct
GAGGATTTTGCTAACCTGACACC

SEQ ID NO: 7 - protein, 21 aa, synthetic construct
  REGION 1..21: note = Synthetic construct
TTGACGGTAACTGACTCCAGC

SEQ ID NO: 8 - protein, 21 aa, synthetic construct
  REGION 1..21: note = Synthetic construct
GCACATCCATCGGTCATCCAG

SEQ ID NO: 9 - protein, 22 aa, synthetic construct
  REGION 1..22: note = Synthetic construct
GCCACAGTCAAACCTTCTTCAA

SEQ ID NO: 10 - protein, 20 aa, synthetic construct
  REGION 1..20: note = Synthetic construct
CTGGCAATGCCTGAACAACG

SEQ ID NO: 11 - protein, 20 aa, synthetic construct
  REGION 1..20: note = Synthetic construct
GGTCGGGACTTGGTTCTGAG

SEQ ID NO: 12 - protein, 19 aa, synthetic construct
  REGION 1..19: note = Synthetic construct
GGGCATCCTGACCCTCAAG

SEQ ID NO: 13 - protein, 20 aa, synthetic construct
  REGION 1..20: note = Synthetic construct
TCCATGTCGTCCCAGTTGGT

SEQ ID NO: 14 - protein, 22 aa, synthetic construct
  REGION 1..22: note = Synthetic construct
CCGCAAGGGAAAGATGAAAGAC

SEQ ID NO: 15 - protein, 20 aa, synthetic construct
  REGION 1..20: note = Synthetic construct
TCGTTTGGTTTCGGGGTTTC

SEQ ID NO: 16 - protein, 20 aa, synthetic construct
  REGION 1..20: note = Synthetic construct
CTAGCAGAAACAAACCGGGC

SEQ ID NO: 17 - protein, 20 aa, synthetic construct
  REGION 1..20: note = Synthetic construct
CCGGCTGCGTATTCTACGTT

SEQ ID NO: 18 - protein, 24 aa, synthetic construct
  REGION 1..24: note = Synthetic construct
GCCAGCCTCTCCTGATTTTAGTGT

SEQ ID NO: 19 - protein, 24 aa, synthetic construct
  REGION 1..24: note = Synthetic construct
GGGAACACAAAAGACCTCTTCTGG